SEQUENCE LISTING (88 sequences)

SEQ ID NO: 1 - protein, 1368 aa, Streptococcus pyogenes [sequence omitted]

SEQ ID NO: 2 - protein, 1334 aa, Unknown
  Description of Unknown MAD2 sequence
MSSLTKFTNKYSKQLTIKNELIPVGKTLENIKENGLIDGDEQLNENYQKAKIIVDDFLRDFINKALNNTQIGNWRELADALNKEDEDNIEKLQDKIRGIIVSKFETFDLFSSYSIKKDEKIIDDDNDVEEEELDLGKKTSSFKYIFKKNLFKLVLPSYLKTTNQDKLKIISSFDNFSTYFRGFFENRKNIFTKKPISTSIAYRIVHDNFPKFLDNIRCFNVWQTECPQLIVKADNYLKSKNVIAKDKSLANYFTVGAYDYFLSQNGIDFYNNIIGGLPAFAGHEKIQGLNEFINQECQKDSELKSKLKNRHAFKMAVLFKQILSDREKSFVIDEFESDAQVIDAVKNFYAEQCKDNNVIFNLLNLIKNIAFLSDDELDGIFIEGKYLSSVSQKLYSDWSKLRNDIEDSANSKQGNKELAKKIKTNKGDVEKAISKYEFSLSELNSIVHDNTKFSDLLSCTLHKVASEKLVKVNEGDWPKHLKNNEEKQKIKEPLDALLEIYNTLLIFNCKSFNKNGNFYVDYDRCINELSSVVYLYNKTRNYCTKKPYNTDKFKLNFNSPQLGEGFSKSKENDCLTLLFKKDDNYYVGIIRKGAKINFDDTQAIADNTDNCIFKMNYFLLKDAKKFIPKCSIQLKEVKAHFKKSEDDYILSDKEKFASPLVIKKSTFLLATAHVKGKKGNIKKFQKEYSKENPTEYRNSLNEWIAFCKEFLKTYKAATIFDITTLKKAEEYADIVEFYKDVDNLCYKLEFCPIKTSFIENLIDNGDLYLFRINNKDFSSKSTGTKNLHTLYLQAIFDERNLNNPTIMLNGGAELFYRKESIEQKNRITHKAGSILVNKVCKDGTSLDDKIRNEIYQYENKFIDTLSDEAKKVLPNVIKKEATHDITKDKRFTSDKFFFHCPLTINYKEGDTKQFNNEVLSFLRGNPDINIIGIDRGERNLIYVTVINQKGEILDSVSFNTVTNKSSKIEQTVDYEEKLAVREKERIEAKRSWDSISKIATLKEGYLSAIVHEICLLMIKHNAIVVLENLNAGFKRIRGGLSEKSVYQKFEKMLINKLNYFVSKKESDWNKPSGLLNGLQLSDQFESFEKLGIQSGFIFYVPAAYTSKIDPTTGFANVLNLSKVRNVDAIKSFFSNFNEISYSKKEALFKFSFDLDSLSKKGFSSFVKFSKSKWNVYTFGERIIKPKNKQGYREDKRINLTFEMKKLLNEYKVSFDLENNLIPNLTSANLKDTFWKELFFIFKTTLQLRNSVTNGKEDVLISPVKNAKGEFFVSGTHNKTLPQDCDANGAYHIALKGLMILERNNLVREEKDTKKIMAISNVDWFEYVQKRRGVL

SEQ ID NO: 3 - protein, 1263 aa, Artificial Sequence
  Description of Artificial Sequence Synthetic polypeptide
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 4 - protein, 16 aa, Artificial Sequence
  Description of Artificial Sequence Synthetic peptide
RQIKIWFQNRRMKWKK

SEQ ID NO: 5 - protein, 36 aa, Artificial Sequence
  Description of Artificial Sequence Synthetic polypeptide
DEVDFQGPCNDSSDPLVVAASIIGILHLILWILDRL

SEQ ID NO: 6 - protein, 4 aa, Artificial Sequence
  Description of Artificial Sequence Synthetic peptide
DEVD

SEQ ID NO: 7 - RNA, 27 nt, Artificial Sequence
  Description of Artificial Sequence Synthetic oligonucleotide
acuuuugacaguuauacgauuccagaa

SEQ ID NO: 8 - RNA, 27 nt, Artificial Sequence
  Description of Artificial Sequence Synthetic oligonucleotide
ugggcuguaccuuccucaggugucaaa

SEQ ID NO: 9 - RNA, 27 nt, Artificial Sequence
  Description of Artificial Sequence Synthetic oligonucleotide
gcuguaccuggucguucaaaucuuacc

SEQ ID NO: 10 - DNA, 4162 bp, Artificial Sequence
  Description of Artificial Sequence Synthetic polynucleotide
aaaaaagaatggttctaccaagactatatacagctacaagtcgtgctgctctgtcgaccgacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagtcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagactga

SEQ ID NO: 11 - DNA, 4145 bp, Artificial Sequence
  Description of Artificial Sequence Synthetic polynucleotide
gatccatgaaaagcttcattacaaggaacaagacagccattgacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagactga

SEQ ID NO: 12 - DNA, 23 bp, Artificial Sequence
  Description of Artificial Sequence Synthetic oligonucleotide
ttctttgaagtatcaggaggtgg

SEQ ID NO: 13 - DNA, 23 bp, Artificial Sequence
  Description of Artificial Sequence Synthetic oligonucleotide
atgattattgcaattccaacagg

SEQ ID NO: 14 - DNA, 23 bp, Artificial Sequence
  Description of Artificial Sequence Synthetic oligonucleotide
gctatttttagtggtatggcagg

SEQ ID NO: 15 - DNA, 24 bp, Artificial Sequence
  Description of Artificial Sequence Synthetic oligonucleotide
accatgtaaatattgtgaaccagg

SEQ ID NO: 16 - DNA, 97 bp, Artificial Sequence
  Description of Artificial Sequence Synthetic oligonucleotide
ttctttgaagtatcaggagggttttagagctagaaatagcaagttaaaataaggctagtccgttatcaacttgaaaaagtggcaccgagtcggtgct

SEQ ID NO: 17 - DNA, 269 bp, Artificial Sequence
  Description of Artificial Sequence Synthetic polynucleotide
tctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatc

SEQ ID NO: 18 - DNA, 20 bp, Artificial Sequence
  Description of Artificial Sequence Synthetic oligonucleotide
tttttttgttttttatgtct

SEQ ID NO: 19 - DNA, 75 bp, Artificial Sequence
  Description of Artificial Sequence Synthetic oligonucleotide
tatatattatgtattattatataaatatatatatatattatattataagtaataataagtattatattatatata

SEQ ID NO: 20 - DNA, 75 bp, Artificial Sequence
  Description of Artificial Sequence Synthetic oligonucleotide
gcttttatagcttagtggtaaagcgataaattgaagatttatttacatgtagttcgattctcattaagggcaata

SEQ ID NO: 21 - DNA, 76 bp, Artificial Sequence
  Description of Artificial Sequence Synthetic oligonucleotide
aggagattagcttaattggtatagcattcgttttacacacgaaagattataggttcgaaccctatatttcctaaat

SEQ ID NO: 22 - DNA, 118 bp, Artificial Sequence
  Description of Artificial Sequence Synthetic polynucleotide
ttattaataattaacaataattaatatattataatttatatatatatattttatattattataataatattcttacaaatataattattatatattattccttcaaaactcctaacgg

SEQ ID NO: 23 - DNA, 76 bp, Artificial Sequence
  Description of Artificial Sequence Synthetic oligonucleotide
gagcttgtatagtttaattggttaaaacatttgtctcataaataaataatgtaaggttcaattccttctacaagta

SEQ ID NO: 24 - protein, 32 aa, Artificial Sequence
  Description of Artificial Sequence Synthetic polypeptide
MFFSAALRARAAGLTAHWGRHVRNLHKTVMQN

SEQ ID NO: 25 - DNA, 4200 bp, Artificial Sequence
  Description of Artificial Sequence Synthetic polynucleotide
atgttcttctccgcggcgctccgggcccgggcggctggcctcaccgcccactggggaagacatgtaaggaatttgcataagacagttatgcaaaatgacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagactga

SEQ ID NO: 26 - protein, 25 aa, Artificial Sequence
  Description of Artificial Sequence Synthetic peptide
MALLTAAARLLGTKNASCLVLAARH

SEQ ID NO: 27 - DNA, 4226 bp, Artificial Sequence
  Description of Artificial Sequence Synthetic polynucleotide
atggctttacttactgcggccgcccggctcttgggaaccaagaatgcatcttgtcttgttcttgcagcccggcatatggctttacttactgcggccgcccggctcttgggaaccaagaatgcagacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaagcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagactga

SEQ ID NO: 28 - DNA, 22 bp, Artificial Sequence
  Description of Artificial Sequence Synthetic oligonucleotide
gtctggtgagtagtgcatggct

SEQ ID NO: 29 - DNA, 460 bp, Artificial Sequence
  Description of Artificial Sequence Synthetic polynucleotide
agccccgcggccccgggctggcggtgtcggctgcaatccggcgggcacggccgggccgggctgggctcttggggcagccaggcgcctccttcagcgcctacggccataccaccctgaacgcgcccgatctcgtctgatctcggaagctaagcagggtcgggcctggttagtacttggatgggagaccacctgggaataccgggtgctgtaggctttttctttggctttttgctgtttctttccttttcttccagacggagtctcgccctctcgcccaggctggagtgcggtggcgccatctcggctcactgcaagctccgcctcccgggtccacgccattccccggcctcagcctcccgagtagctgggcctacaggcgcccgccaccacgcccggccactttgttctatttttcctagagacgggctttcaccctgttagccgggatggtctggagctc

SEQ ID NO: 30 - protein, 30 aa, Artificial Sequence
  Description of Artificial Sequence Synthetic polypeptide
MLSLRQSIRFFKKHKHKHKHKHKHKHKHKH

SEQ ID NO: 31 - protein, 30 aa, Artificial Sequence
  Description of Artificial Sequence Synthetic polypeptide
KKLFKKILKLYLKHKHKHKHKHKHKHKHKH

SEQ ID NO: 32 - DNA, 958 bp, Saccharomyces cerevisiae
tccttgcttcatacctttataaataaggtaatcactaatatattataataataaaaattatatatattatatataatctaaatattatatattttaataaatattaatatatatgatatgaatattattagtttttgggaagcgggaatcccgtaaggagtgagggacccctccctaacgggaggaggaccgaaggagttttagtatttttttttttttaataaaatatatatttatatgattaataatattatatatattatttataaaaataatatataattttaattatttttaataaaaaaaggtggggttgataatataatataatattttttattttaatttataatatataataataaattataaataaattttaattaaaagtagtattaacatattataaatagacaaaagagtctaaaggttaagatttattaaaatgttagatttattaagattacaattaacaacattcattatgaatgatgtaccaacaccttatgcatgttattttcaggattcagcaacaccaaatcaagaaggtattttagaattacatgataatattatgttttatttattagttattttaggtttagtatcttgaatgttatatacaattgttataacatattcaaaaaatcctattgcatataaatatattaaacatggacaaactattgaagttatttgaacaatttttccagctgtaattttattaattattgcttttccttcatttattttattatatttatgtgatgaagttatttcaccagctataactattaaagctattggatatcaatgatattgaaaatatgaatattcagattttattaatgatagtggtgaaactgttgaatttgaatcatatgttattcctgatgaattattagaagaaggtcaattaagattattagatactgatacttctatagttgtacctgta

SEQ ID NO: 33 - DNA, 629 bp, Saccharomyces cerevisiae
attatcaatgatttatattaataataaatataaataataaaaaatatatataatataatataataaatatatttcctttaatattaataaattaataataataataataataataataaaatatttaaataaattatattcaatacaaattaattatttatattattaataattgaataaataatccggtcgaaagagatattaattcgattatattatttatttaattatatttaatttaaatatataaattaatatatatatattgaattatatataaatttattttataattttataaataatatattattataaatatttaatataatttatattattattaaataaaagatttattaaattaatattattatttaattttattatatagtttaagggataatattttattaatattttttttatttatttatttaattatattatatatataatatatatataacaataaatttatgacacatttagaaagaagtagacatcaacaacatccatttcatatggttatgccttcaccatgacctattgtagtatcatttgcattattatcattagcattatcactagcattaacaatgcatggttatattggtaatatgaatatg

SEQ ID NO: 34 - DNA, 2332 bp, Artificial Sequence
  Description of Artificial Sequence Synthetic polynucleotide
aattcgagctcggtacccggggatcacctaaatatgtagcttcagctacaatatctctaaatcataaaatagaacttgttaataatacaaataatgctaaatataccatattcatattaccaatataaccatgcattgttaatgctagtgataatgctaatgataataatgcaaatgatactacaattggtcatggtgaaggcataaccatatgaaatggatgttgttgatgtctacttctttctaaatgtgtcataaatttattgttatatatatattatatatataatataattaaataaataaataaaaaaaatattaataaaatattatcctcaaaactatataataaaattaaataataatattaatttaataaatcttttatttaataataatataaattatattaaatatttataataatatattatttataaaattataaaataaatttatatataattcaatatatatatattaatttatatatttaaattaaatataattaaataaataatataatcgaattaatatctctttcgaccggattatttattcaattattaataatataaataattaatttgtattgaatataatttatttaaatattttattattattattattattattattaatttattaatattaaaggaaatatatttattatattatattatatatattttttattatttatatttattattaatataaatcattgataatatcttctttttatttatttattattataattaaaaagaatcttatattagacctgcagataaggtgattgaatagaatataaatctatatctttattatatttaagaatattattataattattattattattattatttttaataattaaaaatattaataataagtaaatattaaggatccttaagcatatacagcttcgatagctttttcgaaagcatccataccttcttcgattaattcatcttcgattgttaaagcaggtacgaatcttactgttgatttaccagctgtgatgattaataaacctaattctctagcttttttgattacttctgttggaggttctacaaattcagcacctaacattaaacctttacctctgattgttttgatttgattaggatatttagcttggatttctcttaatcttttttgtaagatatctgatttttttgatacttgttttaagaatgcttcatcagcaattgtatctaatacataatttgatactgaacaagctaaaggattaccaccatatgttgtaccatgatcacctactcttaaagcattatttactttttcatttacgattgtagcagcaatagggaaaccattacctaatgctttagctgatgtgaaaatatcaggatgagcttctgaaggtaaataagcatgagctcataatttacctgatctacctaaaccacattgaatttcatcatgaattacgattacatcattatcttgacagattttttttaaaccagttaatttttctacttctacaggaaatacaccaccttcaccttgaataggttctacgattaaaccagcgatttcatctttttttgtttcgatatatgattgtaattttgtcatttcatcatttaaatttaagaatgatacatgaggtactaaatcaccgaaaggtgttctatattttgaatttcatgttactgataaagcacccattgttctaccatggaatgaattttcgaaagctacaataccttgtttactaggatttttcatgataccatgttttttagcgaattttaatgcagcttcattagcttcggtacctgaattacataagaatactcttgaagcatcatgttgaccaccgaattgttttgttttttctacgattttttctgataaatctaaacattcttttgtgaaatataaatttgatgaatgtactaatttattagcttgatgatgtaagatttcagctactttaggattagcatgacctaaagctgttacagcaataccagctgtgaaatcgatatattctttaccatttacatcatcatataatttagcatttttacctcttgtgatacataaatcttcaggtcttgaatatgttgttacttgaaaagctttttcttctaagattgatgtgaatcttcttgatgatgttgatgataaatatcttttgaacattttaataaatcttaacctttagactcttttgtctatttataatatgttaatactacttttaattaaaatttatttataatttattattatatattataaataaaataaaaatattatattaccaaaccccaccactagaagc

SEQ ID NO: 35 - DNA, 6741 bp, Artificial Sequence
  Description of Artificial Sequence Synthetic polynucleotide
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaatttaatacgactcactatagggaattcgagctcggtacccggggatcacctaaatatgtagcttcagctacaatatctctaaatcataaaatagaacttgttaataatacaaataatgctaaatataccatattcatattaccaatataaccatgcattgttaatgctagtgataatgctaatgataataatgcaaatgatactacaattggtcatggtgaaggcataaccatatgaaatggatgttgttgatgtctacttctttctaaatgtgtcataaatttattgttatatatatattatatatataatataattaaataaataaataaaaaaaatattaataaaatattatcctcaaaactatataataaaattaaataataatattaatttaataaatcttttatttaataataatataaattatattaaatatttataataatatattatttataaaattataaaataaatttatatataattcaatatatatatattaatttatatatttaaattaaatataattaaataaataatataatcgaattaatatctctttcgaccggattatttattcaattattaataatataaataattaatttgtattgaatataatttatttaaatattttattattattattattattattattaatttattaatattaaaggaaatatatttattatattatattatatatattttttattatttatatttattattaatataaatcattgataatatcttctttttatttatttattattataattaaaaagaatcttatattagacctgcagataaggtgattgaatagaatataaatctatatctttattatatttaagaatattattataattattattattattattatttttaataattaaaaatattaataataagtaaatattaaggatccttaagcatatacagcttcgatagctttttcgaaagcatccataccttcttcgattaattcatcttcgattgttaaagcaggtacgaatcttactgttgatttaccagctgtgatgattaataaacctaattctctagcttttttgattacttctgttggaggttctacaaattcagcacctaacattaaacctttacctctgattgttttgatttgattaggatatttagcttggatttctcttaatcttttttgtaagatatctgatttttttgatacttgttttaagaatgcttcatcagcaattgtatctaatacataatttgatactgaacaagctaaaggattaccaccatatgttgtaccatgatcacctactcttaaagcattatttactttttcatttacgattgtagcagcaatagggaaaccattacctaatgctttagctgatgtgaaaatatcaggatgagcttctgaaggtaaataagcatgagctcataatttacctgatctacctaaaccacattgaatttcatcatgaattacgattacatcattatcttgacagattttttttaaaccagttaatttttctacttctacaggaaatacaccaccttcaccttgaataggttctacgattaaaccagcgatttcatctttttttgtttcgatatatgattgtaattttgtcatttcatcatttaaatttaagaatgatacatgaggtactaaatcaccgaaaggtgttctatattttgaatttcatgttactgataaagcacccattgttctaccatggaatgaattttcgaaagctacaataccttgtttactaggatttttcatgataccatgttttttagcgaattttaatgcagcttcattagcttcggtacctgaattacataagaatactcttgaagcatcatgttgaccaccgaattgttttgttttttctacgattttttctgataaatctaaacattcttttgtgaaatataaatttgatgaatgtactaatttattagcttgatgatgtaagatttcagctactttaggattagcatgacctaaagctgttacagcaataccagctgtgaaatcgatatattctttaccatttacatcatcatataatttagcatttttacctcttgtgatacataaatcttcaggtcttgaatatgttgttacttgaaaagctttttcttctaagattgatgtgaatcttcttgatgatgttgatgataaatatcttttgaacattttaataaatcttaacctttagactcttttgtctatttataatatgttaatactacttttaattaaaatttatttataatttattattatatattataaataaaataaaaatattatattaccaaaccccaccactagaagcggccgctatatattatgtattattatataaatatatatatatattatattataagtaataataagtattatattatatatagcttttatagcttagtggtaaagcgataaattgaagatttatttacatgtagttcgattctcattaagggcaatatagctatttttagtggtatggcgttttagagctagaaatagcaagttaaaataaggctagtccgttatcaacttgaaaaagtggcaccgagtcggtgctaggagattagcttaattggtatagcattcgttttacacacgaaagattataggttcgaaccctatatttcctaaataccatgtaaatattgtgaaccgttttagagctagaaatagcaagttaaaataaggctagtccgttatcaacttgaaaaagtggcaccgagtcggtgctgagcttgtatagtttaattggttaaaacatttgtctcataaataaataatgtaaggttcaattccttctacaagtattattaataattaacaataattaatatattataatttatatatatatattttatattattataataatattcttacaaatataattattatatattattccttcaaaactcctaacggcaccatggactgtttttaagtatctattgatgattcataatactaatgctagacgcatcaacgaaatatacgcactgagaggcgcaccattaaataattgtgaattaccatgtttatttatataattcatccataccatgtgtgataccagcagctgttacaaattctaataataccatatgatctcttttttcattaggatcttttgataaagctgattgtgtacttaagtaatgattatcaggtaataatacaggaccatcaccgataggtgtattttgttgataatgatctgctaattgtactgaaccgtcttcgatattatgtctgattttgaaatttactttgataccatttttttgtttatcagccatgatatatacattatgtgaattataattatattctaatttatgacctaagatattaccatcttctttgaaatcgataccttttaactcgattctatttactaatgtatcaccttcgaattttacttcagctcttgttttataattaccatcatctttgaagaagattgttctttcttgtacgtaaccttcaggcattgctgatttgaagaaatcatgttgtttcatatgatcaggatatcttgcgaaacattgtactccatatccgaatgttgttactaatgtaggtcaaggtacaggtaatttacctgttgtacagatgaattttaatgttaatttaccatatgtagcatcaccttcaccttcacctgatactgagaatttatgaccatttacatcaccatctaattctactaagataggtactactccagtgaataattcttcaccttttgacatttgtctacttctttctaaatgtgtcataccagctgctgaaccagcagaacctgataacataccactaaaaatagctaacataaaatataatactgcaatatcttttgcatttgttgaatataatcatctttgtaccattccatggaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 36 - protein, 28 aa, Artificial Sequence
  Description of Artificial Sequence Synthetic peptide
KHKHKHKHKHKHKHKHKHMFAFQYLLVM

SEQ ID NO: 37 - protein, 9 aa, Artificial Sequence
  Description of Artificial Sequence Synthetic peptide
RKKRRQRRR

SEQ ID NO: 38 - protein, 9 aa, Artificial Sequence
  Description of Artificial Sequence Synthetic peptide
RRRRRRRRR

SEQ ID NO: 39 - protein, 21 aa, Artificial Sequence
  Description of Artificial Sequence Synthetic peptide
KETWWETWWTEWSQPKKKRKV

SEQ ID NO: 40 - protein, 27 aa, Artificial Sequence
  Description of Artificial Sequence Synthetic peptide
GALFLGFLGAAGSTMGAWSQPKKKRKV

SEQ ID NO: 41 - protein, 6 aa, Artificial Sequence
  Description of Artificial Sequence Synthetic peptide
VRLPPP

SEQ ID NO: 42 - protein, 27 aa, Artificial Sequence
  Description of Artificial Sequence Synthetic peptide
GWTLNSAGYLLGKINLKALAALAKKIL

SEQ ID NO: 43 - protein, 18 aa, Artificial Sequence
  Description of Artificial Sequence Synthetic peptide
KLALKLALKALKAALKLA

SEQ ID NO: 44 - protein, 12 aa, Artificial Sequence
  Description of Artificial Sequence Synthetic peptide
PLILLRLLRGQF

SEQ ID NO: 45 - protein, 12 aa, Artificial Sequence
  Description of Artificial Sequence Synthetic peptide
PLIYLRLLRGQF

SEQ ID NO: 46 - protein, 15 aa, Artificial Sequence
  Description of Artificial Sequence Synthetic peptide
KLWMRWYSPTTRRYG

SEQ ID NO: 47 - protein, 23 aa, Artificial Sequence
  Description of Artificial Sequence Synthetic peptide
MGLGLHLLVLAAALQGAKKKRKV

SEQ ID NO: 48 - protein, 18 aa, Artificial Sequence
  Description of Artificial Sequence Synthetic peptide
KLALKLALKALKAALKLA

SEQ ID NO: 49 - protein, 18 aa, Artificial Sequence
  Description of Artificial Sequence Synthetic peptide
LLIILRRRIRKQAHAHSK

SEQ ID NO: 50 - protein, 10 aa, Artificial Sequence
  Description of Artificial Sequence Synthetic peptide
RRIPNRRPRR

SEQ ID NO: 51 - protein, 11 aa, Artificial Sequence
  Description of Artificial Sequence Synthetic peptide
KKLFKKILKYL

SEQ ID NO: 52 - protein, 18 aa, Artificial Sequence
  Description of Artificial Sequence Synthetic peptide
RKKRRQRRRRKKRRQRRR

SEQ ID NO: 53 - protein, 25 aa, Artificial Sequence
  Description of Artificial Sequence Synthetic peptide
MLSLRQSIRFFKPATRTLCSSRYLL

SEQ ID NO: 54 - protein, 1395 aa, Artificial Sequence
  Description of Artificial Sequence Synthetic polypeptide
MLSLRQSIRFFKPATRTLCSSRYLLDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDEGA

SEQ ID NO: 55 - DNA, 4212 bp, Artificial Sequence
  Description of Artificial Sequence Synthetic polynucleotide
aagcttcgccaccatgctttcactacgtcaatctataagatttttcaagccagccacaagaactttgtgtagctctagatatctgcttgacaagaagtattctatcggactggacatcgggactaatagcgtcgggtgggccgtcatcactgacgagtacaaggtgccctctaagaagttcaaggtgctcgggaacaccgaccggcattccatcaagaaaaatctgatcggagctctcctctttgattcaggggagaccgctgaagcaacccgcctcaagcggactgctagacggcggtacaccaggaggaagaaccggatttgttaccttcaagagatattctccaacgaaatggcaaaggtcgacgacagcttcttccataggctggaagaatcattcctcgtggaagaggataagaagcatgaacggcatcccatcttcggtaatatcgtcgacgaggtggcctatcacgagaaatacccaaccatctaccatcttcgcaaaaagctggtggactcaaccgacaaggcagacctccggcttatctacctggccctggcccacatgattaagttcagaggccacttcctgatcgagggcgacctcaatcctgacaatagcgatgtggataaactgttcatccagctggtgcagacttacaaccagctctttgaagagaaccccatcaatgcaagcggagtcgatgccaaggccattctgtcagcccggctgtcaaagagccgcagacttgagaatcttatcgctcagctgccgggtgaaaagaaaaatggactgttcgggaacctgattgctctttcacttgggctgactcccaatttcaagtctaatttcgacctggcagaggatgccaagctgcaactgtccaaggacacctatgatgacgatctcgacaacctcctggcccagatcggtgaccaatacgccgaccttttccttgctgctaagaatctttctgacgccatcctgctgtctgacattctccgcgtgaacactgaaatcaccaaggcccctctttcagcttcaatgattaagcggtatgatgagcaccaccaggacctgaccctgcttaaggcactcgtccggcagcagcttccggagaagtacaaggaaatcttctttgaccagtcaaagaatggatacgccggctacatcgacggaggtgcctcccaagaggaattttataagtttatcaaacctatccttgagaagatggacggcaccgaagagctcctcgtgaaactgaatcgggaggatctgctgcggaagcagcgcactttcgacaatgggagcattccccaccagatccatcttggggagcttcacgccatccttcggcgccaagaggacttctacccctttcttaaggacaacagggagaagattgagaaaattctcactttccgcatcccctactacgtgggacccctcgccagaggaaatagccggtttgcttggatgaccagaaagtcagaagaaactatcactccctggaacttcgaagaggtggtggacaagggagccagcgctcagtcattcatcgaacggatgactaacttcgataagaacctccccaatgagaaggtcctgccgaaacattccctgctctacgagtactttaccgtgtacaacgagctgaccaaggtgaaatatgtcaccgaagggatgaggaagcccgcattcctgtcaggcgaacaaaagaaggcaattgtggaccttctgttcaagaccaatagaaaggtgaccgtgaagcagctgaaggaggactatttcaagaaaattgaatgcttcgactctgtggagattagcggggtcgaagatcggttcaacgcaagcctgggtacctaccatgatctgcttaagatcatcaaggacaaggattttctggacaatgaggagaacgaggacatccttgaggacattgtcctgactctcactctgttcgaggaccgggaaatgatcgaggagaggcttaagacctacgcccatctgttcgacgataaagtgatgaagcaacttaaacggagaagatataccggatggggacgccttagccgcaaactcatcaacggaatccgggacaaacagagcggaaagaccattcttgatttccttaagagcgacggattcgctaatcgcaacttcatgcaacttatccatgatgattccctgacctttaaggaggacatccagaaggcccaagtgtctggacaaggtgactcactgcacgagcatatcgcaaatctggctggttcacccgctattaagaagggtattctccagaccgtgaaagtcgtggacgagctggtcaaggtgatgggtcgccataaaccagagaacattgtcatcgagatggccagggaaaaccagactacccagaagggacagaagaacagcagggagcggatgaaaagaattgaggaagggattaaggagctcgggtcacagatccttaaagagcacccggtggaaaacacccagcttcagaatgagaagctctatctgtactaccttcaaaatggacgcgatatgtatgtggaccaagagcttgatatcaacaggctctcagactacgacgtggaccacatcgtccctcagagcttcctcaaagacgactcaattgacaataaggtgctgactcgctcagacaagaaccggggaaagtcagataacgtgccctcagaggaagtcgtgaaaaagatgaagaactattggcgccagcttctgaacgcaaagctaatcactcagcggaagttcgacaatctcactaaggctgagaggggcggactgagcgaactggacaaagcaggattcattaaacggcaacttgtggagactcggcagattactaaacatgtcgcccaaatccttgactcacgcatgaataccaagtacgacgaaaacgacaaacttatccgcgaggtgaaggtgattaccctgaagtccaagctggtcagcgatttcagaaaggactttcaattctacaaagtgcgggagatcaataactatcatcatgctcatgacgcatatctgaatgccgtggtgggaaccgccctaatcaagaagtacccaaagctggaaagcgagttcgtgtacggagactacaaggtctacgacgtgcgcaagatgattgccaaatctgagcaggagatcggaaaggccaccgcaaagtacttcttctacagcaacatcatgaatttcttcaagaccgaaatcacccttgcaaacggtgagatccggaagaggccgctcatcgagactaatggggagactggcgaaatcgtgtgggacaagggcagagatttcgctaccgtgcgcaaagtgctttctatgcctcaagtgaacatcgtgaagaaaaccgaggtgcaaaccggaggcttttctaaggaatcaatcctccccaagcgcaactccgacaagctcattgcaaggaagaaggattgggaccctaagaagtacggcggattcgattcaccaactgtggcttattctgtcctggtcgtggctaaggtggaaaaaggaaagtctaagaagctcaagagcgtgaaggaactgctgggtatcaccattatggagcgcagctccttcgagaagaacccaattgactttctcgaagccaaaggttacaaggaagtcaagaaggaccttatcatcaagctcccaaagtatagcctgttcgaactggagaatgggcggaagcggatgctcgcctccgctggcgaacttcagaagggtaatgagctggctctcccctccaagtacgtgaatttcctctaccttgcaagccattacgagaagctgaaggggagccccgaggacaacgagcaaaagcaactgtttgtggagcagcataagcattatctggacgagatcattgagcagatttccgagttttctaaacgcgtcattctcgctgatgccaacctcgataaagtccttagcgcatacaataagcacagagacaaaccaattcgggagcaggctgagaatatcatccacctgttcaccctcaccaatcttggtgcccctgccgcattcaagtacttcgacaccaccatcgaccggaaacgctatacctccaccaaagaagtgctggacgccaccctcatccaccagagcatcaccggactttacgaaactcggattgacctctcacagctcggaggggatgagggagcttagttcgcggccgc

SEQ ID NO: 56 - DNA, 20 bp, Artificial Sequence
  Description of Artificial Sequence Synthetic oligonucleotide
ttctttgaagtatcaggagg

SEQ ID NO: 57 - DNA, 20 bp, Artificial Sequence
  Description of Artificial Sequence Synthetic oligonucleotide
atgattattgcaattccaac

SEQ ID NO: 58 - DNA, 22 bp, Artificial Sequence
  Description of Artificial Sequence Synthetic oligonucleotide
tagctatttttagtggtatggc

SEQ ID NO: 59 - DNA, 21 bp, Artificial Sequence
  Description of Artificial Sequence Synthetic oligonucleotide
accatgtaaatattgtgaacc

SEQ ID NO: 60 - DNA, 889 bp, Artificial Sequence
  Description of Artificial Sequence Synthetic polynucleotide
gcatgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcatgattattgcaattccaacgttttagagctagaaatagcaagttaaaataaggctagtccgttatcaacttgaaaaagtggcaccgagtcggtggcgcaagtggtttagtggtaaaatccaacgttgccatcgttgggcccccggttcgattccgggcttgcgcattctttgaagtatcaggagggttttagagctagaaatagcaagttaaaataaggctagtccgttatcaacttgaaaaagtggcaccgagtcggtgtttttttgttttttatgtcttcgagtcatgtaattagttataagcatgtgagctaagacactgtaattgccaatctaaacgataccacggccgctctagagaaatggggagcgatttgcaggcatttgctcggtgcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtctcgaggcggccgc

SEQ ID NO: 61 - DNA, 892 bp, Artificial Sequence
  Description of Artificial Sequence Synthetic polynucleotide
gcatgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatctagctatttttagtggtatggcgttttagagctagaaatagcaagttaaaataaggctagtccgttatcaacttgaaaaagtggcaccgagtcggtggcgcaagtggtttagtggtaaaatccaacgttgccatcgttgggcccccggttcgattccgggcttgcgcaaccatgtaaatattgtgaaccgttttagagctagaaatagcaagttaaaataaggctagtccgttatcaacttgaaaaagtggcaccgagtcggtgtttttttgttttttatgtcttcgagtcatgtaattagttataagcatgtgagctaagacactgtaattgccaatctaaacgataccacggccgctctagagaaatggggagcgatttgcaggcatttgctcggtgcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtctcgaggcggccgc

SEQ ID NO: 62 - DNA, 454 bp, Artificial Sequence
  Description of Artificial Sequence Synthetic polynucleotide
gcatgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgctatttttagtggtatggcgttttagagctagaaatagcaagttaaaataaggctagtccgttatcaacttgaaaaagtggcaccgagtcggtggcgcaagtggtttagtggtaaaatccaacgttgccatcgttgggcccccggttcgattccgggcttgcgcatgagacc

SEQ ID NO: 63 - DNA, 453 bp, Artificial Sequence
  Description of Artificial Sequence Synthetic polynucleotide
ggtctcacgcaccatgtaaatattgtgaaccgttttagagctagaaatagcaagttaaaataaggctagtccgttatcaacttgaaaaagtggcaccgagtcggtgtttttttgttttttatgtcttcgagtcatgtaattagttataagcatgtgagctaagacactgtaattgccaatctaaacgataccacggccgctctagagaaatggggagcgatttgcaggcatttgctcggtgcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtctcgaggcggccgc

SEQ ID NO: 64 - DNA, 20 bp, Artificial Sequence
  Description of Artificial Sequence Synthetic oligonucleotide
gctatttttagtggtatggc

SEQ ID NO: 65 - DNA, 20 bp, Artificial Sequence
  Description of Artificial Sequence Synthetic oligonucleotide
ccatgtaaatattgtgaacc

SEQ ID NO: 66 - DNA, 889 bp, Artificial Sequence
  Description of Artificial Sequence Synthetic polynucleotide
gcatgcggacaatctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgctatttttagtggtatggcgttttagagctagaaatagcaagttaaaataaggctagtccgttatcaacttgaaaaagtggcaccgagtcggtggcgcaagtggtttagtggtaaaatccaacgttgccatcgttgggcccccggttcgattccgggcttgcgcaccatgtaaatattgtgaaccgttttagagctagaaatagcaagttaaaataaggctagtccgttatcaacttgaaaaagtggcaccgagtcggtgtttttttgttttttatgtcttcgagtcatgtaattagttataagcatgtgagctaagacactgtaattgccaatctaaacgataccacggccgctctagagaaatggggagcgatttgcaggcatttgctcggtgcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtctcgaggcggccgc

SEQ ID NO: 67 - DNA, 987 bp, Artificial Sequence
  Description of Artificial Sequence Synthetic polynucleotide
aagctttccatggaatggtacaaagatgattatattcaacaaatgcaaaagatattgcagtattatattttatgttagctatttttagtggtatgttatcaggttctgctggttcagcagctggtatgacacatttagaaagaagtagacaaatgtcaaaaggtgaagaattattcactggagtagtacctatcttagtagaattagatggtgatgtaaatggtcataaattctcagtatcaggtgaaggtgaaggtgatgctacatatggtaaattaacattaaaattcatctgtacaacaggtaaattacctgtaccttgacctacattagtaacaacattcggatatggagtacaatgtttcgcaagatatcctgatcatatgaaacaacatgatttcttcaaatcagcaatgcctgaaggttacgtacaagaaagaacaatcttcttcaaagatgatggtaattataaaacaagagctgaagtaaaattcgaaggtgatacattagtaaatagaatcgagttaaaaggtatcgatttcaaagaagatggtaatatcttaggtcataaattagaatataattataattcacataatgtatatatcatggctgataaacaaaaaaatggtatcaaagtaaatttcaaaatcagacataatatcgaagacggttcagtacaattagcagatcattatcaacaaaatacacctatcggtgatggtcctgtattattacctgataatcattacttaagtacacaatcagctttatcaaaagatcctaatgaaaaaagagatcatatggtattattagaatttgtaacagctgctggtatcacacatggtatggatgaattatataaataaacatggtaattcacaattatttaatggtgcgcctctcagtgcgtatatttcgttgatgcgtctagcattagtattatgaatcatcaatagatacttaaaaacagtccatgttctgcag

SEQ ID NO: 68 - DNA, 25 bp, Artificial Sequence
  Description of Artificial Sequence Synthetic primer
agaatcaggtgctggtacagggtga

SEQ ID NO: 69 - DNA, 22 bp, Artificial Sequence
  Description of Artificial Sequence Synthetic primer
ctattcaggcacattcaggacc

SEQ ID NO: 70 - DNA, 23 bp, Artificial Sequence
  Description of Artificial Sequence Synthetic primer
agaggtataccaacacaagattc

SEQ ID NO: 71 - DNA, 27 bp, Artificial Sequence
  Description of Artificial Sequence Synthetic primer
agatagataatcaattcaaccatctgt

SEQ ID NO: 72 - DNA, 41 bp, Artificial Sequence
  Description of Artificial Sequence Synthetic primer
attagttcggtttagttggtattttgtaatgagtaaaaagt

SEQ ID NO: 73 - DNA, 34 bp, Artificial Sequence
  Description of Artificial Sequence Synthetic primer
cctacactaatcataggtgttttatgacatgcta

SEQ ID NO: 74 - DNA, 27 bp, Artificial Sequence
  Description of Artificial Sequence Synthetic primer
agattatgaaagagagtattaatatca

SEQ ID NO: 75 - DNA, 23 bp, Artificial Sequence
  Description of Artificial Sequence Synthetic primer
taaagttagcccctactgagtta

SEQ ID NO: 76 - DNA, 22 bp, Artificial Sequence
  Description of Artificial Sequence Synthetic primer
caggtgaaggtgaaggtgatgc

SEQ ID NO: 77 - DNA, 23 bp, Artificial Sequence
  Description of Artificial Sequence Synthetic primer
gatctgctaattgtactgaaccg

SEQ ID NO: 78 - DNA, 24 bp, Artificial Sequence
  Description of Artificial Sequence Synthetic primer
cagcaatgcctgaaggttacgtac

SEQ ID NO: 79 - DNA, 23 bp, Artificial Sequence
  Description of Artificial Sequence Synthetic primer
actaatgtaggtcaaggtacagg

SEQ ID NO: 80 - DNA, 1504 bp, Artificial Sequence
  Description of Artificial Sequence Synthetic polynucleotide
aagcttccatcgaatggtacaaagatgattatattcaacaaatgcaaaagatattgcagtattatattttatgttagctatttttagtggtatgttatcaggttctgctggttcagcagctggtttcaaaagatatttatcatcaacatcatcaagaagattcacatcaatcttagaagaaaaagcatttcaagtaacaacatattcaagacctgaagatttatgtatcacaagaggtaaaaatgctaaattatatgatgatgtaaatggtaaagaatatatcgatttcacagctggtattgctgtaacagctttaggtcatgctaatcctaaagtagctgaaatcttacatcatcaagctaataaattagtacattcatcaaatttatatttcacaaaagaatgtttagatttatcagaaaaaatcgtagaaaaaacaaaacaattcggtggtcaacatgatgcttcaagagtattcttatgtaattcaggtaccgaagctaatgaagctgcattaaaattcgctaaaaaacatggtatcatgaaaaatcctagtaaacaaggtattgtagctttcgaaaattcatttcatggtagaacaatgggtgctttatcagtaacatgaaattcaaaatatagaacacctttcggtgatttagtacctcatgtatcattcttaaatttaaatgatgaaatgacaaaattacaatcatatatcgaaacaaaaaaagatgaaatcgctggtttaatcgtagaacctattcaaggtgaaggtggtgtatttcctgtagaagtagaaaaattaactggtttaaaaaaaatctgtcaagataatgatgtaatcgtaattcatgatgaaattcaatgtggtttaggtagatcaggtaaattatgagctcatgcttatttaccttcagaagctcatcctgatattttcacatcagctaaagcattaggtaatggtttccctattgctgctacaatcgtaaatgaaaaagtaaataatgctttaagagtaggtgatcatggtacaacatatggtggtaatcctttagcttgttcagtatcaaattatgtattagatacaattgctgatgaagcattcttaaaacaagtatcaaaaaaatcagatatcttacaaaaaagattaagagaaatccaagctaaatatcctaatcaaatcaaaacaatcagaggtaaaggtttaatgttaggtgctgaatttgtagaacctccaacagaagtaatcaaaaaagctagagaattaggtttattaatcatcacagctggtaaatcaacagtaagattcgtacctgctttaacaatcgaagatgaattaatcgaagaaggtatggatgctttcgaaaaagctatcgaagctgtatatgcttaaacatggtaattcacaattatttaatggtgcgcctctcagtgcgtatatttcgttgatgcgtctagcattagtattatgaatcatcaatagatacttaaaaacagtccatgg

SEQ ID NO: 81 - protein, 422 aa, Artificial Sequence
  Description of Artificial Sequence Synthetic polypeptide
FKRYLSSTSSRRFTSILEEKAFQVTTYSRPEDLCITRGKNAKLYDDVNGKEYIDFTAGIAVTALGHANPKVAEILHHQANKLVHSSNLYFTKECLDLSEKIVEKTKQFGGQHDASRVFLCNSGTEANEAALKFAKKHGIMKNPSKQGIVAFENSFHGRTMGALSVTWNSKYRTPFGDLVPHVSFLNLNDEMTKLQSYIETKKDEIAGLIVEPIQGEGGVFPVEVEKLTGLKKICQDNDVIVIHDEIQCGLGRSGKLWAHAYLPSEAHPDIFTSAKALGNGFPIAATIVNEKVNNALRVGDHGTTYGGNPLACSVSNYVLDTIADEAFLKQVSKKSDILQKRLREIQAKYPNQIKTIRGKGLMLGAEFVEPPTEVIKKARELGLLIITAGKSTVRFVPALTIEDELIEEGMDAFEKAIEAVYA

SEQ ID NO: 82 - DNA, 630 bp, Artificial Sequence
  Description of Artificial Sequence Synthetic polynucleotide
gcggccgctatatattatgtattattatataaatatatatatatattatattataagtaataataagtattatattatatatagcttttatagcttagtggtaaagcgataaattgaagatttatttacatgtagttcgattctcattaagggcaatagctatttttagtggtatggcgttttagagctagaaatagcaagttaaaataaggctagtccgttatcaacttgaaaaagtggcaccgagtcggtgctaggagattagcttaattggtatagcattcgttttacacacgaaagattataggttcgaaccctatatttcctaaatccatgtaaatattgtgaaccgttttagagctagaaatagcaagttaaaataaggctagtccgttatcaacttgaaaaagtggcaccgagtcggtgctgagcttgtatagtttaattggttaaaacatttgtctcataaataaataatgtaaggttcaattccttctacaagtattattaataattaacaataattaatatattataatttatatatatatattttatattattataataatattcttacaaatataattattatatattattccttcaaaactcctaacggcaccatgg

SEQ ID NO: 83 - DNA, 300 bp, Artificial Sequence
  Description of Artificial Sequence Synthetic polynucleotide
tctagatcaaatatataagtaatagggggagggggtgggtgataataaccagaatattaaataaatacagagcacacatttgttaatatttaataatataatcaataaatatattataataatataatataattaataatagatataaagtataaacaatataataaattatataaaataaatataaattaaaaataataaccaaataattaatataataaatgataaacaagaagatatccgggtcccaataataattattattgaaaataataattgggacccccacaatgcggccgc

SEQ ID NO: 84 - DNA, 31 bp, Artificial Sequence
  Description of Artificial Sequence Synthetic primer
gaaaagtgccacctgggtccttttcatcacg

SEQ ID NO: 85 - DNA, 27 bp, Artificial Sequence
  Description of Artificial Sequence Synthetic primer
gacgaaagggcctcgtgatacgcctat

SEQ ID NO: 86 - DNA, 471 bp, Artificial Sequence
  Description of Artificial Sequence Synthetic polynucleotide
cacattcaggacctagtgtagatttagcaatttttgcattacatttaacatcaatttcatcattattaggtgctattaatttcattgtaacaacattaaatatgagaacaaatggtatgacaatgcataaattaccattatttgtatgatcaattttcattacagcgttcttattattattatcattacctgtattatctgctggtattacaatgttattattagatagaaacttcaatacttcatttttcggagtttctggtggaggtggtggaatgacacatttagaaagaagtagacaaatgtcaaaaggtgaagaattattcactggagtagtacctatcttagtagaattagatggtgatgtaaatggtcataaattctcagtatcaggtaaaggtgaaggtgatgctacatatggtaaattaacattaaaattcatctgtacaacaggtaaattacctgtacctt

SEQ ID NO: 87 - DNA, 713 bp, Artificial Sequence
  Description of Artificial Sequence Synthetic polynucleotide
gaggttacgtacaagaaagaacaatcttcttcaaagatgatggtaattataaaacaagagctgaagtaaaattcgaaggtgatacattagtaaatagaatcgagttaaaaggtatcgatttcaaagaagatggtaatatcttaggtcataaattagaatataattataattcacataatgtatatatcatggctgataaacaaaaaaatggtatcaaagtaaatttcaaaatcagacataatatcgaagacggttcagtacaattagcagatcattatcaacaaaatacacctatcggtgatggtcctgtattattacctgataatcattacttaagtacacaatcagctttatcaaaagatcctaatgaaaaaagagatcatatggtattattagaatttgtaacagctgctggtatcacacatggtatggatgaattatataaataacaacaggaattaaaattttctcatgattaataaatccctttagcaaggataaaaataaaaataaaaataaaaagttgatcagaaattatcaaaaaataaataataataatataataaaaacatatttaaataataataatataattataataaatatatataaaggtaatttatatgatatttatccaagatcaaatagaaattatattcaaccaaataatattaataaagaattagtagtatatggttataatttagaatctt

SEQ ID NO: 88 - protein, 8 aa, Artificial Sequence
  Description of Artificial Sequence Synthetic peptide
RRRRRRRR

(19) United States
(12) Patent Application Publication
(10) Pub. No.: US 2022/0372523 A1
(43) Pub. Date: Nov. 24, 2022

(54) ORGANELLE GENOME MODIFICATION

(71) Applicant: Napigen, Inc., Wilmington, DE (US)

(72) Inventors: Emil Meyer OROZCO, JR., Cochranville, PA (US); Narendra Singh YADAV, Wilmington, DE (US); Hajime SAKAI, Newark, DE (US)

(21) Appl. No.: 17/561,291

(22) Filed: Dec. 23, 2021

Related U.S. Application Data
(63) Continuation of application No. PCT/US2020/040730, filed on Jul. 2, 2020.
(60) Provisional application No. 62/870,441, filed on Jul. 3, 2019.
(60) Provisional application No. 62/899,495, filed on Sep. 12, 2019.

(51) Int. Cl. C12N 15/90; C12N 9/22; C12N 15/11; C12N 15/82

(52) U.S. Cl. CPC C12N 15/907; C12N 15/905; C12N 9/22; C12N 15/11; C12N 15/8213; C12N 15/8214; C12N 2310/20; C12N 2800/80

(57) ABSTRACT

Provided herein are methods and compositions for modifying cells. Provided herein are methods and compositions for modifying an organism of a cell. Provided herein are methods and compositions for introducing polynucleotides and/or polypeptides into a nucleus of a cell.

### CROSS-REFERENCE

This application claims the benefit of PCT application PCT/US2020/040730 filed Jul. 2, 2020, U.S. Provisional Application No. 62/870,441, filed Jul. 3, 2019, and U.S. Provisional Application No. 62/899,495, filed Sep. 12, 2019, which are incorporated herein by reference.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Disclosed herein are methods for altering an organellar genome. In some embodiments, a method can comprise introducing into a nucleus of a cell a first polynucleotide. In some embodiments, a first polynucleotide can encode at least in part a modified polynucleotide guided polypeptide. In some embodiments, a modified polynucleotide guided polypeptide can comprise a polynucleotide guided polypeptide operably linked to an organellar targeting peptide. In some embodiments, a polynucleotide guided polypeptide when associated with a guide RNA, can cleave at least one target sequence present in an organellar genome. In some embodiments, a method can comprise introducing into a nucleus of a cell a second polynucleotide. In some embodiments, a second polynucleotide can comprise at least in part at least one guide RNA. In some embodiments, at least one guide RNA can direct a polynucleotide guided polypeptide to cleave at least one target sequence present in an organellar genome. In some embodiments, a method can comprise introducing into an organelle of a cell, a third polynucleotide. In some embodiments, a third polynucleotide can comprise at least in part at least one homologous organellar DNA sequence. In some embodiments, a method can comprise introducing into a nucleus of a cell a second polynucleotide at least one homologous organellar DNA can be capable of homologous recombination. In some embodiments, a method can comprise introducing into a nucleus of a cell a second polynucleotide integration of at least one homologous organellar DNA sequence into an organellar genome. In some embodiments, introducing into a nucleus of a cell a second polynucleotide can result in a recombined organellar genome lacking at least one target sequence. In some embodiments, a method can comprise growing a cell comprising a nucleus of (a) and an organelle of (b) under conditions in which a first polynucleotide and a second polynucleotide are expressed. In some embodiments, a method can comprise selecting a cell comprising an altered organellar genome.

In some embodiments, a polynucleotide guided polypeptide can comprise at least one member selected from a group consisting of: a Cas9 protein, a MAD2 protein, a MAD7 protein, a CRISPR nuclease, a nuclease domain of a Cas protein, a Cpf1 protein, an Argonaute, modified versions thereof, and any combination thereof. In some embodiments, an at least one guide RNA can be processed from a polycistronic RNA after transcription by use of at least one member selected from a group consisting of: an RNA cleavage site, a ribozyme cleavage site, a polynucleotide guided polypeptide cleavage site, a presence of a tRNA sequence, and any combination thereof. In some embodiments, an at least one guide RNA can be processed from a polycistronic RNA after transcription by use of a presence of a tRNA sequence, wherein an at least one guide RNA can be processed from a polycistronic RNA by having a first tRNA sequence 5′ to an at least one guide RNA and a second tRNA sequence 3′ to an at least one guide RNA. In some embodiments, (a) and (b) can occur in separate cells. In some embodiments, a nucleus of (a) and an organelle of (b) can be brought together into a cell by sexual crossing, cell fusion, microinjection, or any combination thereof. In some embodiments, a method can further comprise: (e) selecting a cell that is homoplasmic for an altered organellar genome. In some embodiments, a third polynucleotide can comprise an at least one homologous organellar DNA sequence operably linked to an origin of replication that is functional in an organelle. In some embodiments, a third polynucleotide can comprise at least one homologous organellar DNA sequence comprising a fourth polynucleotide encoding at least one selectable marker or at least one screenable marker, or both. In some embodiments, a fourth polynucleotide, after integration into an organellar genome, can be operably linked to a promoter that is functional in an organelle. In some embodiments, a third polynucleotide can comprise at least one homologous organellar DNA sequence comprising a fifth polynucleotide and a sixth polynucleotide. In some embodiments, a fifth polynucleotide and a sixth polynucleotide can each comprise a region of homology in an organellar genome. In some embodiments, a region of homology in a fifth polynucleotide and a region of homology in a sixth polynucleotide can correspond to two adjacent regions of homology in an organellar genome. In some embodiments, a fifth polynucleotide and a sixth polynucleotide can be separated by a seventh polynucleotide, wherein a seventh polynucleotide can comprise a sequence that is heterologous to an organellar genome. In some embodiments, a seventh polynucleotide can encode at least one selected from a group consisting of: a cytoplasmic male sterility factor, a dsRNA, a siRNA, a miRNA, and any combination thereof. In some embodiments, a dsRNA, a siRNA or an miRNA can suppress at least one target gene necessary for male fertility in a plant. In some embodiments, a fourth polynucleotide can comprise a first sequence encoding a positive selectable marker. In some embodiments, a fourth polynucleotide can comprise a second sequence encoding a negative selectable marker. In some embodiments, a first sequence and a second sequence can each be operably linked to a promoter that is functional in an organelle. In some embodiments, a third polynucleotide can be single stranded. In some embodiments, a third polynucleotide can be double stranded. In some embodiments, a third polynucleotide can comprise a length of at least 100, 150, 200, 250, 300, 400, 500, 100, 1500 or 2000 nucleotides. In some embodiments, a cell can be selected from a group consisting of: a yeast cell, an algal cell, a plant cell, an insect cell, a non-human animal cell, an isolated and purified human cell, a mammalian tissue culture cell, and any combination thereof. In some embodiments, an organelle can be a mitochondrion. In some embodiments, an organelle can be a plastid. In some embodiments, an organelle can be a chloroplast. In some embodiments, at least one member selected from a group consisting of: a first polynucleotide, a second polynucleotide, a third polynucleotide, and any combination thereof, can be introduced into a cell via at least one method from a group consisting of: microinjection, meristem transformation, electroporation, Agrobacterium-mediated transformation, viral based gene transfer, transfection, vacuum infiltration, biolistic particle bombardment, and any combination thereof. In some embodiments, at least one member selected from a group consisting of: a first polynucleotide, a second polynucleotide, a third polynucleotide, and any combination thereof, can be introduced into a cell as a peptide-polynucleotide complex. In some embodiments, at least one peptide of a peptide-polynucleotide complex can comprise at least one member selected from a group consisting of: a cell penetrating peptide (CPP), an organellar targeting peptide, a histidine rich peptide, a lysine-rich peptide, and any combination thereof. In some embodiments, a method can comprise growing a cell produced by a method as disclosed herein. In some embodiments, a method can further comprise growing a cell in a presence of a positive selection agent and selecting a cell that is homoplasmic for an altered organellar genome. In some embodiments, a method further comprising growing a cell in an absence of a positive selection agent, followed by selecting a cell that lacks a non-integrated recombinant DNA construct. In some embodiments, a method can further comprise growing a cell in an absence of a positive selection agent, followed by growing a cell in a presence of a negative selection agent, followed by selecting a cell that lacks a non-integrated recombinant DNA construct. Disclosed herein in some embodiments, is a composition comprising a cell produced by a method as disclosed herein, wherein a cell can comprise a yeast cell, an algal cell, a plant cell, an insect cell, a non-human animal cell, an isolated and purified human cell, a mammalian tissue culture cell, or any combination thereof. In some embodiments, a composition can comprise a plant, a seed, a root, a stem, a leaf, a flower, a fruit, or any combination thereof produced from a cell as disclosed herein, wherein a cell is a plant cell, wherein a plant, a seed, a root, a stem, a leaf, a flower, a fruit, or a combination thereof can comprise an altered organellar genome.

Disclosed herein is a method for altering an organellar genome. In some embodiments, a method can comprise introducing into a nucleus of a cell a first polynucleotide. In some embodiments, a first polynucleotide can encode at least in part a modified polynucleotide guided polypeptide. In some embodiments, a modified polynucleotide guided polypeptide can comprise a polynucleotide guided polypeptide operably linked to an organellar targeting peptide. In some embodiments, a polynucleotide guided polypeptide when associated with a guide RNA, can cleave at least one target sequence present in an organellar genome. In some embodiments, a method can comprise introducing into an organelle of a cell a second polynucleotide. In some embodiments, a second polynucleotide can comprise at least in part at least one guide RNA. In some embodiments, at least one guide RNA can direct a polynucleotide guided polypeptide to cleave at least one target sequence present in an organellar genome. In some embodiments, a growing cell can comprise a nucleus of (a) and an organelle of (b) under conditions in which a first polynucleotide and a second polynucleotide are expressed; and selecting a cell comprising an altered organellar genome. In some embodiments, (b) can further comprise introducing into an organelle of a cell a third polynucleotide. In some embodiments, a third polynucleotide can comprise at least in part at least one homologous organellar DNA sequence. In some embodiments, at least one homologous organellar DNA sequence can capable of homologous recombination. In some embodiments, an integration of at least one homologous organellar DNA sequence into an organellar genome can result in a recombined organellar genome lacking at least one target sequence. In some embodiments, a polynucleotide guided polypeptide can comprise at least one member selected from a group consisting of: a Cas9 protein, a MAD2 protein, a MAD7 protein, a CRISPR nuclease, a nuclease domain of a Cas protein, a Cpf1 protein, an Argonaute, modified versions thereof, and any combination thereof. In some embodiments, a polynucleotide guided polypeptide can comprise at least one member selected from a group consisting of: a Cas9 protein, a MAD2 protein, a MAD7 protein, a CRISPR nuclease, a nuclease domain of a Cas protein, a Cpf1 protein, an Argonaute, modified versions thereof, and any combination thereof. In some embodiments, an at least one guide RNA can be processed from a polycistronic RNA after transcription by use of at least one member selected from a group consisting of: an RNA cleavage site, a ribozyme cleavage site, a polynucleotide guided polypeptide cleavage site, a presence of a tRNA sequence, and any combination thereof. In some embodiments, an at least one guide RNA can be processed from a polycistronic RNA after transcription by use of a presence of a tRNA sequence, wherein an at least one guide RNA can be processed from a polycistronic RNA by having a first tRNA sequence 5′ to an at least one guide RNA and a second tRNA sequence 3′ to an at least one guide RNA. In some embodiments, (a) and (b) can occur in separate cells. In some embodiments, a nucleus of (a) and an organelle of (b) can be brought together into a cell by sexual crossing, cell fusion, microinjection, or any combination thereof. In some embodiments, a method can further comprise: (e) selecting a cell that is homoplasmic for an altered organellar genome. In some embodiments, a third polynucleotide can comprise an at least one homologous organellar DNA sequence operably linked to an origin of replication that is functional in an organelle. In some embodiments, a third polynucleotide can comprise at least one homologous organellar DNA sequence comprising a fourth polynucleotide encoding at least one selectable marker or at least one screenable marker, or both. In some embodiments, a fourth polynucleotide, after integration into an organellar genome, can be operably linked to a promoter that is functional in an organelle. In some embodiments, a third polynucleotide can comprise at least one homologous organellar DNA sequence comprising a fifth polynucleotide and a sixth polynucleotide. In some embodiments, a fifth polynucleotide and a sixth polynucleotide can each comprise a region of homology in an organellar genome. In some embodiments, a region of homology in a fifth polynucleotide and a region of homology in a sixth polynucleotide can correspond to two adjacent regions of homology in an organellar genome. In some embodiments, a fifth polynucleotide and a sixth polynucleotide can be separated by a seventh polynucleotide, wherein a seventh polynucleotide can comprise a sequence that is heterologous to an organellar genome. In some embodiments, a seventh polynucleotide can encode at least one selected from a group consisting of: a cytoplasmic male sterility factor, a dsRNA, a siRNA, a miRNA, and any combination thereof. In some embodiments, a dsRNA, a siRNA or an miRNA can suppress at least one target gene necessary for male fertility in a plant. In some embodiments, a fourth polynucleotide can comprise a first sequence encoding a positive selectable marker. In some embodiments, a fourth polynucleotide can comprise a second sequence encoding a negative selectable marker. In some embodiments, a first sequence and a second sequence can each be operably linked to a promoter that is functional in an organelle. In some embodiments, a third polynucleotide can be single stranded. In some embodiments, a third polynucleotide can be double stranded. In some embodiments, a third polynucleotide can comprise a length of at least 100, 150, 200, 250, 300, 400, 500, 100, 1500 or 2000 nucleotides. In some embodiments, a cell can be selected from a group consisting of: a yeast cell, an algal cell, a plant cell, an insect cell, a non-human animal cell, an isolated and purified human cell, a mammalian tissue culture cell, and any combination thereof. In some embodiments, an organelle can be a mitochondrion. In some embodiments, an organelle can be a plastid. In some embodiments, an organelle can be a chloroplast. In some embodiments, at least one member selected from a group consisting of: a first polynucleotide, a second polynucleotide, a third polynucleotide, and any combination thereof, can be introduced into a cell via at least one method from a group consisting of: microinjection, meristem transformation, electroporation, Agrobacterium-mediated transformation, viral based gene transfer, transfection, vacuum infiltration, biolistic particle bombardment, and any combination thereof. In some embodiments, at least one member selected from a group consisting of: a first polynucleotide, a second polynucleotide, a third polynucleotide, and any combination thereof, can be introduced into a cell as a peptide-polynucleotide complex. In some embodiments, at least one peptide of a peptide-polynucleotide complex can comprise at least one member selected from a group consisting of: a cell penetrating peptide (CPP), an organellar targeting peptide, a histidine rich peptide, a lysine-rich peptide, and any combination thereof. In some embodiments, a method can comprise growing a cell produced by a method as disclosed herein. In some embodiments, a method can further comprise growing a cell in a presence of a positive selection agent and selecting a cell that is homoplasmic for an altered organellar genome. In some embodiments, a method further comprising growing a cell in an absence of a positive selection agent, followed by selecting a cell that lacks a non-integrated recombinant DNA construct. In some embodiments, a method can further comprise growing a cell in an absence of a positive selection agent, followed by growing a cell in a presence of a negative selection agent, followed by selecting a cell that lacks a non-integrated recombinant DNA construct. Disclosed herein in some embodiments, is a composition comprising a cell produced by a method as disclosed herein, wherein a cell can comprise a yeast cell, an algal cell, a plant cell, an insect cell, a non-human animal cell, an isolated and purified human cell, a mammalian tissue culture cell, or any combination thereof. In some embodiments, a composition can comprise a plant, a seed, a root, a stem, a leaf, a flower, a fruit, or any combination thereof produced from a cell as disclosed herein, wherein a cell is a plant cell, wherein a plant, a seed, a root, a stem, a leaf, a flower, a fruit, or a combination thereof can comprise an altered organellar genome.

Disclosed herein in some embodiments, is a method for altering an organellar genome. In some embodiments, a method can comprise introducing into a nucleus of a cell a first polynucleotide. In some embodiments, a first polynucleotide can encode a modified site-directed nuclease. In some embodiments, a modified site-directed nuclease can comprise a site-directed nuclease operably linked to an organellar targeting peptide. In some embodiments, a site-directed nuclease can cleave at least one target sequence present in an organellar genome. In some embodiments, a method can comprise introducing into an organelle of a cell, a third polynucleotide. In some embodiments, a third polynucleotide can comprise at least one homologous organellar DNA sequence. In some embodiments, at least one homologous organellar DNA can be capable of homologous recombination. In some embodiments, integration of at least one homologous organellar DNA sequence into an organellar genome can result in a recombined organellar genome lacking at least one target sequence. In some embodiments, a method can comprise growing a cell comprising a nucleus of (a) and an organelle of (b) under conditions in which a first polynucleotide is expressed. In some embodiments, a method can comprise selecting a cell comprising an altered organellar genome. In some embodiments, a polynucleotide guided polypeptide can comprise at least one member selected from a group consisting of: a Cas9 protein, a MAD2 protein, a MAD7 protein, a CRISPR nuclease, a nuclease domain of a Cas protein, a Cpf1 protein, an Argonaute, modified versions thereof, and any combination thereof. In some embodiments, an at least one guide RNA can be processed from a polycistronic RNA after transcription by use of at least one member selected from a group consisting of: an RNA cleavage site, a ribozyme cleavage site, a polynucleotide guided polypeptide cleavage site, a presence of a tRNA sequence, and any combination thereof. In some embodiments, an at least one guide RNA can be processed from a polycistronic RNA after transcription by use of a presence of a tRNA sequence, wherein an at least one guide RNA can be processed from a polycistronic RNA by having a first tRNA sequence 5′ to an at least one guide RNA and a second tRNA sequence 3′ to an at least one guide RNA. In some embodiments, (a) and (b) can occur in separate cells. In some embodiments, a nucleus of (a) and an organelle of (b) can be brought together into a cell by sexual crossing, cell fusion, microinjection, or any combination thereof. In some embodiments, a method can further comprise: (e) selecting a cell that is homoplasmic for an altered organellar genome. In some embodiments, a third polynucleotide can comprise an at least one homologous organellar DNA sequence operably linked to an origin of replication that is functional in an organelle. In some embodiments, a third polynucleotide can comprise at least one homologous organellar DNA sequence comprising a fourth polynucleotide encoding at least one selectable marker or at least one screenable marker, or both. In some embodiments, a fourth polynucleotide, after integration into an organellar genome, can be operably linked to a promoter that is functional in an organelle. In some embodiments, a third polynucleotide can comprise at least one homologous organellar DNA sequence comprising a fifth polynucleotide and a sixth polynucleotide. In some embodiments, a fifth polynucleotide and a sixth polynucleotide can each comprise a region of homology in an organellar genome. In some embodiments, a region of homology in a fifth polynucleotide and a region of homology in a sixth polynucleotide can correspond to two adjacent regions of homology in an organellar genome. In some embodiments, a fifth polynucleotide and a sixth polynucleotide can be separated by a seventh polynucleotide, wherein a seventh polynucleotide can comprise a sequence that is heterologous to an organellar genome. In some embodiments, a seventh polynucleotide can encode at least one selected from a group consisting of: a cytoplasmic male sterility factor, a dsRNA, a siRNA, a miRNA, and any combination thereof. In some embodiments, a dsRNA, a siRNA or an miRNA can suppress at least one target gene necessary for male fertility in a plant. In some embodiments, a fourth polynucleotide can comprise a first sequence encoding a positive selectable marker. In some embodiments, a fourth polynucleotide can comprise a second sequence encoding a negative selectable marker. In some embodiments, a first sequence and a second sequence can each be operably linked to a promoter that is functional in an organelle. In some embodiments, a third polynucleotide can be single stranded. In some embodiments, a third polynucleotide can be double stranded. In some embodiments, a third polynucleotide can comprise a length of at least 100, 150, 200, 250, 300, 400, 500, 100, 1500 or 2000 nucleotides. In some embodiments, a cell can be selected from a group consisting of: a yeast cell, an algal cell, a plant cell, an insect cell, a non-human animal cell, an isolated and purified human cell, a mammalian tissue culture cell, and any combination thereof. In some embodiments, an organelle can be a mitochondrion. In some embodiments, an organelle can be a plastid. In some embodiments, an organelle can be a chloroplast. In some embodiments, at least one member selected from a group consisting of: a first polynucleotide, a second polynucleotide, a third polynucleotide, and any combination thereof, can be introduced into a cell via at least one method from a group consisting of: microinjection, meristem transformation, electroporation, Agrobacterium-mediated transformation, viral based gene transfer, transfection, vacuum infiltration, biolistic particle bombardment, and any combination thereof. In some embodiments, at least one member selected from a group consisting of: a first polynucleotide, a second polynucleotide, a third polynucleotide, and any combination thereof, can be introduced into a cell as a peptide-polynucleotide complex. In some embodiments, at least one peptide of a peptide-polynucleotide complex can comprise at least one member selected from a group consisting of: a cell penetrating peptide (CPP), an organellar targeting peptide, a histidine rich peptide, a lysine-rich peptide, and any combination thereof. In some embodiments, a method can comprise growing a cell produced by a method as disclosed herein. In some embodiments, a method can further comprise growing a cell in a presence of a positive selection agent and selecting a cell that is homoplasmic for an altered organellar genome. In some embodiments, a method further comprising growing a cell in an absence of a positive selection agent, followed by selecting a cell that lacks a non-integrated recombinant DNA construct. In some embodiments, a method can further comprise growing a cell in an absence of a positive selection agent, followed by growing a cell in a presence of a negative selection agent, followed by selecting a cell that lacks a non-integrated recombinant DNA construct. Disclosed herein in some embodiments, is a composition comprising a cell produced by a method as disclosed herein, wherein a cell can comprise a yeast cell, an algal cell, a plant cell, an insect cell, a non-human animal cell, an isolated and purified human cell, a mammalian tissue culture cell, or any combination thereof. In some embodiments, a composition can comprise a plant, a seed, a root, a stem, a leaf, a flower, a fruit, or any combination thereof produced from a cell as disclosed herein, wherein a cell is a plant cell, wherein a plant, a seed, a root, a stem, a leaf, a flower, a fruit, or a combination thereof can comprise an altered organellar genome.

Disclosed herein in some embodiments, is a method for altering an organellar genome. In some embodiments, a method can comprise introducing into a nucleus of a cell a first polynucleotide encoding a modified polynucleotide guided polypeptide, wherein a modified polynucleotide guided polypeptide comprises a polynucleotide guided polypeptide operably linked to an organellar targeting peptide, wherein a polynucleotide guided polypeptide when associated with a guide RNA, cleaves at least one target sequence present in an organellar genome; and a second polynucleotide encoding at least one guide RNA, wherein at least one guide RNA directs a polynucleotide guided polypeptide to cleave at least one target sequence present in an organellar genome; introducing into an organelle of a cell a replacement DNA; growing a cell comprising a nucleus of (a) and an organelle of (b) under conditions in which a first polynucleotide and a second polynucleotide are expressed; and selecting a cell comprising an altered organellar genome.

Disclosed herein in some embodiments, is a method for altering an organellar genome. In some embodiments, a method can comprise introducing into a nucleus of a cell a first polynucleotide encoding a modified polynucleotide guided polypeptide, wherein a modified polynucleotide guided polypeptide can comprise a polynucleotide guided polypeptide operably linked to an organellar targeting peptide, wherein a polynucleotide guided polypeptide when associated with a guide RNA, can cleave at least one target sequence present in an organellar genome. Disclosed herein in some embodiments, a method can comprise introducing into an organelle of a cell a second polynucleotide encoding at least one guide RNA, wherein at least one guide RNA can direct a polynucleotide guided polypeptide to cleave at least one target sequence present in an organellar genome; and a replacement DNA; growing a cell comprising a nucleus of (a) and an organelle of (b) under conditions in which a first polynucleotide and a second polynucleotide are each expressed; and selecting a cell comprising an altered organellar genome. In some embodiments, a replacement DNA can comprise fragments of organellar DNA or a complete organellar DNA from a cultivar, line, sub-species or other species. In some embodiments, a replacement DNA can be distinct from an organellar genome of a cell. In some embodiments, at least one target sequence is not present in a replacement DNA. In some embodiments, after (a) and prior to (b), a cell can be selected in which a native organellar genome has been eliminated. In some embodiments, an organelle can be a mitochondrion. In some embodiments, an organelle can be a plastid. In some embodiments, an organelle can be a chloroplast. In some embodiments a cell produced by a method disclosed herein can be a yeast cell, an algal cell, a plant cell, an insect cell, a non-human animal cell, an isolated and purified human cell, or a mammalian tissue culture cell. Disclosed herein in some embodiments, is a plant, seed, root, stem, leaf, flower, or fruit produced from a cell as disclosed herein, wherein the plant, seed, root, stem, leaf, flower, or fruit comprises an altered organellar genome.

Disclosed herein are methods for altering an organellar genome. In some embodiments, a method can comprise (a) introducing into a nucleus of a cell: a first polynucleotide encoding at least in part a modified polynucleotide guided polypeptide. In some embodiments, a modified polynucleotide guided polypeptide can comprise a polynucleotide guided polypeptide operably linked to an organellar targeting peptide. In some embodiments, a polynucleotide guided polypeptide when associated with a guide RNA can cleave at least one target sequence present in an organellar genome. In some embodiments, a method can comprise, introducing into a nucleus of a cell a second polynucleotide comprising at least in part a guide RNA. In some embodiments, a guide RNA can direct a polynucleotide guided polypeptide to cleave at least one target sequence present in an organellar genome. In some embodiments, a method can comprise (b) introducing into an organelle of a cell, a third polynucleotide encoding at least in part at least one homologous organellar DNA sequence. In some embodiments, an at least one homologous organellar DNA can be capable of homologous recombination. In some embodiments, integration of an at least one homologous organellar DNA sequence into an organellar genome can result in a recombined organellar genome lacking at least one target sequence. In some embodiments, a method can comprise (c) growing a cell comprising a nucleus of (a) and an organelle of (b) under conditions in which a first polynucleotide and a second polynucleotide are expressed. In some embodiments, a method can comprise (d) selecting a cell comprising an altered organellar genome. In some embodiments, (a) and (b) can occur in separate cells. In some embodiments, a nucleus of (a) and an organelle of (b) can be brought together into a cell by sexual crossing, cell fusion, microinjection, or any combination thereof. In some embodiments, a method can further comprise (e) selecting a cell that is homoplasmic for an altered organellar genome. In some embodiments, a third polynucleotide encoding at least one homologous organellar DNA sequence can be operably linked to an origin of replication that can be functional in an organelle. In some embodiments, a third polynucleotide may comprise RNA, DNA or a combination of RNA and DNA. In some embodiments, a third polynucleotide may be single-stranded or may be double-stranded. In some embodiments, a third polynucleotide may have a length of at least about 100, at least about 150, at least about 200, at least about 250, at least about 300, at least about 400, at least about 500, at least about 1000, at least about 1500 or at least about 2000 nucleotides. In some embodiments, a third polynucleotide may be linear or may be circular. In some embodiments, a third polynucleotide encoding at least one homologous organellar DNA sequence can comprise a fourth polynucleotide encoding at least one selectable marker or at least one screenable marker, or both. In some embodiments, a fourth polynucleotide, after integration into an organellar genome can be operably linked to a promoter that can be functional in an organelle. In some embodiments, a third polynucleotide encoding at least one homologous organellar DNA sequence can comprise a fifth polynucleotide or a sixth polynucleotide. In some embodiments, a third polynucleotide encoding at least one homologous organellar DNA sequence can comprise a fifth polynucleotide and a sixth polynucleotide. In some embodiments, a third polynucleotide encoding at least one homologous organellar DNA sequence can comprise a fifth polynucleotide. In some embodiments, a fifth polynucleotide can correspond to a region of homology in an organellar genome. In some embodiments, a fifth polynucleotide can correspond to an adjacent region of homology in an organellar genome. In some embodiments, a sixth polynucleotide can correspond to a region of homology in an organellar genome. In some embodiments, a third polynucleotide encoding at least one homologous organellar DNA sequence can comprise a sixth polynucleotide wherein a sixth polynucleotide can correspond to an adjacent region of homology in an organellar genome. In some embodiments, a fifth polynucleotide and a sixth polynucleotide can correspond to two adjacent regions of homology in an organellar genome. In some embodiments, a fifth polynucleotide and a sixth polynucleotide can be separated by a seventh polynucleotide. In some embodiments, a seventh polynucleotide can comprise a sequence that can be heterologous to an organellar genome. In some embodiments, a seventh polynucleotide can encode an RNA that can be heterologous to an organelle. In some embodiments, a seventh polynucleotide can encode a cytoplasmic male sterility factor, a dsRNA, a siRNA, a miRNA, or any combination thereof. In some embodiments, a dsRNA, siRNA or a miRNA can suppress at least one target gene necessary for male fertility in a plant. In some embodiments, a seventh polynucleotide can encode a herbicide tolerance protein, a pesticidal protein, an accessory protein that binds to a pesticidal protein, a dsRNA, a siRNA, or a miRNA. In some embodiments, a seventh polynucleotide can encode a herbicide tolerance protein. In some embodiments, a herbicide tolerance protein can comprise a 4-hydroxphenylpyruvate dioxygenase (HPPD), a sulfonylurea-tolerant acetolactate synthase (ALS), an imidazolinone-tolerant acetolactate synthase (ALS), a glyphosate-tolerant 5-enolpyruvylshikimate-3-phosphate synthase (EPSPS), a glyphosate-tolerant glyphosate oxidoreductase (GOX), a glyphosate N-acetyltransferase (GAT), a phosphinothricin acetyl transferase (PAT), a protoporphyrinogen oxidase (PROTOX), an auxin enzyme or receptor, a P450 polypeptide, an acetyl coenzyme A carboxylase (ACCase), or any combination thereof. In some embodiments, a seventh polynucleotide can encode a pesticidal protein. In some embodiments, a pesticidal protein can comprise Cry1Ac, Cyt1Aa, Cry1Ab, Cry2Aa, Cry1I, Cry1C, Cry1D, Cry1E, Cry1Be, Cry1Fa, Vip3A, or any combination thereof. In some embodiments, a seventh polynucleotide can encode an accessory protein. In some embodiments, an accessory protein can bind to a pesticidal protein. In some embodiments, a pesticidal protein can comprise a 20 kDa accessory protein, a 19 kDa accessory protein or any combination thereof. In some embodiments, a dsRNA, a siRNA, a miRNA or any combination thereof can suppress at least one target gene present in a plant pest. In some embodiments, a seventh polynucleotide can encode β-ketothiolase, polyhydroxybutyrate synthase, acetoacetyl-CoA reductase, anthranilate synthase, chorismate pyruvate lyase, large subunit of a RUBISCO, or any combination thereof. In some embodiments, a seventh polynucleotide can be operably linked to at least one regulatory element that can be active in an organelle. In some embodiments, a regulatory element can comprise a maize clpP promoter combined with a maize clpP 5′-UTR, a maize clpP promoter combined with a 5′-UTR from gene 10 of bacteriophage T7, a tomato psbA promoter combined with a 5′-UTR from gene 10 of bacteriophage T7, a tomato rm16 promoter combined with a modified accD 5′-UTR, or any combination thereof. In some embodiments, a fourth polynucleotide can comprise a first sequence encoding a positive selectable marker. In some embodiments, a fourth polynucleotide can comprise a second sequence encoding a negative selectable marker. In some embodiments, a first sequence and a second sequence can each be operably linked to a promoter that can be functional in an organelle. In some embodiments, a polynucleotide guided polypeptide can comprise a Cas9 protein, a MAD2 protein, a MAD7 protein, a CRISPR nuclease, a nuclease domain of a Cas protein, a Cpf1 protein, an Argonaute, modified versions thereof, or any combination thereof. In some embodiments, a third polynucleotide can further comprise an eighth polynucleotide and/or a ninth polynucleotide can have 100 percent sequence identity to each other and have sufficient length for homologous recombination. In some embodiments, an eighth polynucleotide or a ninth polynucleotide can have at least about 50, 60, 70 80, 90, 100 percent sequence identity to each other, or to an endogenous sequence in an organellar genome. In some embodiments, an eighth polynucleotide or a ninth polynucleotide can have at least about 50, 60, 70 80, 90, 100 percent sequence identity to each other, or to an endogenous sequence in an organellar genome, and have sufficient length for homologous recombination. In some embodiments, a third polynucleotide can further comprise an eighth polynucleotide and a ninth polynucleotide. In some embodiments, an eight polynucleotide and a ninth polynucleotide can have at least about 50, 60, 70 80, 90, 100 percent sequence identity to each other or to an endogenous sequence in an organellar genome and have sufficient length for homologous recombination. In some embodiments, a length for homologous recombination can be at least about 10, at least about 15, at least about 20, at least about 25, at least about 30, at least about 35, at least about 40, at least about 50, at least about 60, at least about 70, at least about 80, at least about 90, at least about 100, at least about 150, at least about 200, or at least about 250 nucleotides. In some embodiments, a length for homologous recombination can be at most about 10, at most about 15, at most about 20, at most about 25, at most about 30, at most about 35, at most about 40, at most about 50, at most about 60, at most about 70, at most about 80, at most about 90, at most about 100, at most about 150, at most about 200, or at most about 250 nucleotides. In some embodiments, an eighth polynucleotide and a ninth polynucleotide can be arranged as direct repeats in a recombinant DNA construct. In some embodiments, a repeat can comprise a site-specific recombinase site. In some embodiments, a site-specific recombinase can comprise loxP, attP, attB or a combination thereof. In some embodiments, growing a cell can be under conditions wherein a site-specific recombinase can be expressed in an organelle. In some embodiments, a site-specific recombinase can comprise Cre, phiC31, Bxb1 or a combination thereof. In some embodiments, a third polynucleotide can be circular. In some embodiments, a third polynucleotide can comprise RNA, DNA or a combination thereof. In some embodiments, a third polynucleotide can be single stranded. In some embodiments, a third polynucleotide can be double stranded. In some embodiments, a polynucleotide can comprise a length of at least about 100, 150, 200, 250, 300, 400, 500, 100, 1500 or 2000 nucleotides. In some embodiments, an eighth polynucleotide and a ninth polynucleotide can be arranged as direct repeats in a recombinant DNA construct. In some embodiments, a third polynucleotide can be linear. In some embodiments, an eighth polynucleotide and a ninth polynucleotide can be present at a 5′ and 3′ ends of a third polynucleotide. In some embodiments, a cell can be selected from: a yeast cell, an algal cell, a plant cell, an insect cell, a non-human animal cell, an isolated and purified human cell, or mammalian tissue culture cell. In some embodiments, an alteration of an organellar genome can comprise an insertion of an expression cassette. In some embodiments, an expression cassette can be a polycistronic expression cassette. In some embodiments, a polycistronic expression cassette can encode a selectable marker or a screenable marker, or both. In some embodiments, an organelle can be a mitochondrion. In some embodiments, an organelle can be a plastid. In some embodiments, an organelle can be a chloroplast. In some embodiments, a cell can be produced by a method disclosed herein. In some embodiments, a second polynucleotide can comprise at least 17 nucleotides. In some embodiments, a guide RNA can comprise a single guide RNA or a duplex guide RNA. In some embodiments, a guide RNA can comprise one guide RNA or a plurality of guide RNAs. In some embodiments, a plurality of guide RNAs can be encoded on separate transcription units or on a polycistronic transcription unit. In some embodiments, a plurality of guide RNAs can be encoded on a polycistronic transcription unit. In some embodiments, a guide RNA can be processed from a polycistronic RNA after transcription by use of an RNA cleavage site, a ribozyme cleavage site, a polynucleotide guided polypeptide cleavage site or a presence of a tRNA sequence. In some embodiments, a guide RNA can be processed from a polycistronic RNA by having a first tRNA sequence 5′ to a guide RNA and a second tRNA sequence 3′ to a guide RNA. In some embodiments, a polynucleotide guided polypeptide can be codon-optimized for a human, a yeast, an alga, or a plant species. In some embodiments, a first polynucleotide, a second polynucleotide or a third polynucleotide can be introduced into a cell via microinjection, meristem transformation, electroporation, Agrobacterium-mediated transformation, viral based gene transfer, transfection, vacuum infiltration, biolistic particle bombardment or any combination thereof. In some embodiments, a first polynucleotide, a second polynucleotide or a third polynucleotide can be introduced into a cell as a peptide-polynucleotide complex. In some embodiments, a peptide of a peptide-polynucleotide complex can comprise a cell penetrating peptide (CPP), an organellar targeting peptide, a histidine rich peptide, a lysine-rich peptide or any combination thereof. In some embodiments, a peptide of a peptide-polynucleotide complex can comprise a CPP. In some embodiments, a CPP can comprise penetratin, TAT, R9, Pep-1, MPG, gamma-ZEIN, Transporant, MAP, Pept 1, Pept 2, IVV-14, Ig(v), Amphiphilic model peptide, pVEC, HRSV, Bp100, TAT₂ or any combination thereof. In some embodiments, a CPP can comprise at least about 80%, 90%, 95%, 100% sequence identity to SEQ ID NO: 4, 36, 37, 38, 39, 40, 41, 42, 43, 44, 45, 46, 47, 48, 49, 50, 51, 52 or any combination thereof. In some embodiments, a method can further comprise introducing into an organelle a polynucleotide encoding a marker. In some embodiments, a marker can be a positive selectable marker, a negative selectable marker, a screenable marker, or any combination thereof. In some embodiments, a marker can be a positive selectable marker. In some embodiments, a positive selectable marker can comprise an herbicide tolerance protein. In some embodiments, described herein are methods of growing a cell produced by a method described above. In some embodiments, a method can comprise growing a cell in a presence of a positive selection agent and selecting a cell that is homoplasmic for altered organellar genome. In some embodiments, a method can comprise growing a cell in an absence of a positive selection agent, followed by selecting a cell that lacks a non-integrated recombinant DNA construct. In some embodiments, a method can further comprise growing a cell in an absence of a positive selection agent, followed by growing a cell in a presence of a negative selection agent, followed by selecting a cell that lacks a non-integrated recombinant DNA construct. In some embodiments, a cell can comprise a yeast cell, an algal cell, a plant cell, an insect cell, a non-human animal cell, an isolated and purified human cell, or a mammalian tissue culture cell. In some embodiments, a plant, seed, root, stem, leaf, flower, or fruit can be produced from a cell described herein. In some embodiments, a plant, seed, root, stem, leaf, flower, or fruit can comprise an altered organellar genome.

Disclosed herein are methods for altering an organellar genome. In some embodiments, a method can comprise (a) introducing into a nucleus of a cell: a first polynucleotide encoding a modified polynucleotide guided polypeptide. In some embodiments, a modified polynucleotide guided polypeptide can comprise a polynucleotide guided polypeptide operably linked to an organellar targeting peptide. In some embodiments, a polynucleotide guided polypeptide when associated with a guide RNA can cleave at least one target sequence present in an organellar genome. In some embodiments, a method can comprise (b) introducing into an organelle of a cell: a second polynucleotide encoding a guide RNA. In some embodiments, a guide RNA can direct a polynucleotide guided polypeptide to cleave at least one target sequence present in an organellar genome. In some embodiments, a method can comprising growing a cell comprising nucleus of (a) and organelle of (b) under conditions in which a first polynucleotide and a second polynucleotide are expressed. In some embodiments, a method can comprise selecting a cell comprising an altered organellar genome. In some embodiments, (b) can further comprise introducing into an organelle of a cell a third polynucleotide encoding at least one homologous organellar DNA sequence. In some embodiments, an at least one homologous organellar DNA can be capable of homologous recombination. In some embodiments, integration of an at least one homologous organellar DNA sequence into an organellar genome can result in a recombined organellar genome lacking at least one target sequence. In some embodiments, (a) and (b) can occur in separate cells. In some embodiments, a nucleus of (a) and an organelle of (b) can be brought together into a cell by sexual crossing, cell fusion, microinjection, or any combination thereof. In some embodiments, a method can further comprise (e) selecting a cell that is homoplasmic for an altered organellar genome. In some embodiments, a third polynucleotide encoding at least one homologous organellar DNA sequence can be operably linked to an origin of replication that can be functional in an organelle. In some embodiments, a third polynucleotide may comprise RNA, DNA or a combination of RNA and DNA. In some embodiments, a third polynucleotide may be single-stranded or may be double-stranded. In some embodiments, a third polynucleotide may have a length of at least about 100, at least about 150, at least about 200, at least about 250, at least about 300, at least about 400, at least about 500, at least about 1000, at least about 1500 or at least about 2000 nucleotides. In some embodiments, a third polynucleotide may be linear or may be circular. In some embodiments, a third polynucleotide encoding at least one homologous organellar DNA sequence can comprise a fourth polynucleotide encoding at least one selectable marker or at least one screenable marker, or both. In some embodiments, a fourth polynucleotide, after integration into an organellar genome can be operably linked to a promoter that can be functional in an organelle. In some embodiments, a third polynucleotide encoding at least one homologous organellar DNA sequence can comprise a fifth polynucleotide or a sixth polynucleotide. In some embodiments, a third polynucleotide encoding at least one homologous organellar DNA sequence can comprise a fifth polynucleotide and a sixth polynucleotide. In some embodiments, a third polynucleotide encoding at least one homologous organellar DNA sequence can comprise a fifth polynucleotide. In some embodiments, a fifth polynucleotide can correspond to a region of homology in an organellar genome. In some embodiments, a fifth polynucleotide can correspond to an adjacent region of homology in an organellar genome. In some embodiments, a sixth polynucleotide can correspond to a region of homology in an organellar genome. In some embodiments, a third polynucleotide encoding at least one homologous organellar DNA sequence can comprise a sixth polynucleotide wherein a sixth polynucleotide can correspond to an adjacent region of homology in an organellar genome. In some embodiments, a fifth polynucleotide and a sixth polynucleotide can correspond to two adjacent regions of homology in an organellar genome. In some embodiments, a fifth polynucleotide and a sixth polynucleotide can be separated by a seventh polynucleotide. In some embodiments, a seventh polynucleotide can comprise a sequence that can be heterologous to an organellar genome. In some embodiments, a seventh polynucleotide can encode an RNA that can be heterologous to an organelle. In some embodiments, a seventh polynucleotide can encode a cytoplasmic male sterility factor, a dsRNA, a siRNA, a miRNA, or any combination thereof. In some embodiments, a dsRNA, siRNA or a miRNA can suppress at least one target gene necessary for male fertility in a plant. In some embodiments, a seventh polynucleotide can encode a herbicide tolerance protein, a pesticidal protein, an accessory protein that binds to a pesticidal protein, a dsRNA, a siRNA, or a miRNA. In some embodiments, a seventh polynucleotide can encode a herbicide tolerance protein. In some embodiments, a herbicide tolerance protein can comprise a 4-hydroxphenylpyruvate dioxygenase (HPPD), a sulfonylurea-tolerant acetolactate synthase (ALS), an imidazolinone-tolerant acetolactate synthase (ALS), a glyphosate-tolerant 5-enolpyruvylshikimate-3-phosphate synthase (EPSPS), a glyphosate-tolerant glyphosate oxidoreductase (GOX), a glyphosate N-acetyltransferase (GAT), a phosphinothricin acetyl transferase (PAT), a protoporphyrinogen oxidase (PROTOX), an auxin enzyme or receptor, a P450 polypeptide, an acetyl coenzyme A carboxylase (ACCase), or any combination thereof. In some embodiments, a seventh polynucleotide can encode a pesticidal protein. In some embodiments, a pesticidal protein can comprise Cry1Ac, Cyt1Aa, Cry1Ab, Cry2Aa, Cry1I, Cry1C, Cry1D, Cry1E, Cry1Be, Cry1Fa, Vip3A, or any combination thereof. In some embodiments, a seventh polynucleotide can encode an accessory protein. In some embodiments, an accessory protein can bind to a pesticidal protein. In some embodiments, a pesticidal protein can comprise a 20 kDa accessory protein, a 19 kDa accessory protein or any combination thereof. In some embodiments, a dsRNA, a siRNA or a miRNA can suppress at least one target gene present in a plant pest. In some embodiments, a seventh polynucleotide can encode β-ketothiolase, polyhydroxybutyrate synthase, acetoacetyl-CoA reductase, anthranilate synthase, chorismate pyruvate lyase, large subunit of a RUBISCO, or any combination thereof. In some embodiments, a seventh polynucleotide can be operably linked to at least one regulatory element that can be active in an organelle. In some embodiments, a regulatory element can comprise a maize clpP promoter combined with a maize clpP 5′-UTR, a maize clpP promoter combined with a 5′-UTR from gene 10 of bacteriophage T7, a tomato psbA promoter combined with a 5′-UTR from gene 10 of bacteriophage T7, a tomato rrn16 promoter combined with a modified accD 5′-UTR, or any combination thereof. In some embodiments, a fourth polynucleotide can comprise a first sequence encoding a positive selectable marker. In some embodiments, a fourth polynucleotide can comprise a second sequence encoding a negative selectable marker. In some embodiments, a first sequence and ae second sequence can each be operably linked to a promoter that can be functional in an organelle. In some embodiments, a polynucleotide guided polypeptide can comprise a Cas9 protein, a MAD2 protein, a MAD7 protein, a CRISPR nuclease, a nuclease domain of a Cas protein, a Cpf1 protein, an Argonaute, modified versions thereof, or any combination thereof. In some embodiments, a third polynucleotide can further comprise an eighth polynucleotide and/or a ninth polynucleotide that can have 100 percent sequence identity to each other and have sufficient length for homologous recombination. In some embodiments, an eighth polynucleotide and a ninth polynucleotide can be arranged as direct repeats in a recombinant DNA construct. In some embodiments, a third polynucleotide can be linear. In some embodiments, a third polynucleotide can further comprise an eighth polynucleotide and a ninth polynucleotide. In some embodiments, an eight polynucleotide and a ninth polynucleotide can have about at least 50, 60, 70, 80, 90, or 100 percent sequence identity to each other or to an endogenous sequence in an organellar genome and have sufficient length for homologous recombination. In some embodiments, a length for homologous recombination can be about at least 10, at least 15, at least 20, at least 25, at least 30, at least 35, at least 40, at least 50, at least 60, at least 70, at least 80, at least 90, at least 100, at least 150, at least 200, or at least 250 nucleotides. In some embodiments, a length for homologous recombination can be about at most 10, at most 15, at most 20, at most 25, at most 30, at most 35, at most 40, at most 50, at most 60, at most 70, at most 80, at most 90, at most 100, at most 150, at most 200, or at most 250 nucleotides. In some embodiments, an eighth polynucleotide and a ninth polynucleotide can be arranged as direct repeats in a recombinant DNA construct. In some embodiments, a repeat can comprise a site-specific recombinase site. In some embodiments, a site-specific recombinase can comprise loxP, attP, attB or a combination thereof. In some embodiments, growing a cell can be under conditions wherein a site-specific recombinase can be expressed in an organelle. In some embodiments, a site-specific recombinase can comprise Cre, phiC31, Bxb1 or a combination thereof. In some embodiments, a third polynucleotide can be circular. In some embodiments, a third polynucleotide can comprise RNA, DNA or a combination thereof. In some embodiments, a third polynucleotide can be single stranded. In some embodiments, a third polynucleotide can be double stranded. In some embodiments, a polynucleotide can comprise a length of at least about 100, 150, 200, 250, 300, 400, 500, 100, 1500 or 2000 nucleotides. In some embodiments, an eighth polynucleotide and a ninth polynucleotide can be present at a 5′ and 3′ ends of a third polynucleotide. In some embodiments, a cell can be selected from: a yeast cell, an algal cell, a plant cell, an insect cell, a non-human animal cell, an isolated and purified human cell, or mammalian tissue culture cell. In some embodiments, an alteration of an organellar genome can comprise an insertion of an expression cassette. In some embodiments, an expression cassette can be a polycistronic expression cassette. In some embodiments, a polycistronic expression cassette can encode a selectable marker or a screenable marker, or both. In some embodiments, an organelle can be a mitochondrion. In some embodiments, an organelle can be a plastid. In some embodiments, an organelle can be a chloroplast. In some embodiments, a second polynucleotide can comprise at least 17 nucleotides. In some embodiments, a guide RNA can comprise a single guide RNA or a duplex guide RNA. In some embodiments, a guide RNA can comprise one guide RNA or a plurality of guide RNAs. In some embodiments, a plurality of guide RNAs can be encoded on separate transcription units or on a polycistronic transcription unit. In some embodiments, a plurality of guide RNAs can be encoded on a polycistronic transcription unit. In some embodiments, a guide RNA can be processed from a polycistronic RNA after transcription by use of an RNA cleavage site, a ribozyme cleavage site, a polynucleotide guided polypeptide cleavage site or a presence of a tRNA sequence. In some embodiments, a guide RNA can be processed from a polycistronic RNA by having a first tRNA sequence 5′ to a guide RNA and a second tRNA sequence 3′ to a guide RNA. In some embodiments, a polynucleotide guided polypeptide can be codon-optimized for a human, a yeast, an alga, or a plant species. In some embodiments, a first polynucleotide, a second polynucleotide or a third polynucleotide can be introduced into a cell via microinjection, meristem transformation, electroporation, Agrobacterium-mediated transformation, viral based gene transfer, transfection, vacuum infiltration, biolistic particle bombardment or any combination thereof. In some embodiments, a first polynucleotide, a second polynucleotide or a third polynucleotide can be introduced into a cell as a peptide-polynucleotide complex. In some embodiments, a peptide of a peptide-polynucleotide complex can comprise a cell penetrating peptide (CPP), an organellar targeting peptide, a histidine rich peptide, a lysine-rich peptide or any combination thereof. In some embodiments, a peptide of a peptide-polynucleotide complex can comprise a CPP. In some embodiments, a CPP can comprise penetratin, TAT, R9, Pep-1, MPG, gamma-ZEIN, Transporant, MAP, Pept 1, Pept 2, IVV-14, Ig(v), Amphiphilic model peptide, pVEC, HRSV, Bp100, TAT₂ or any combination thereof. In some embodiments, a CPP can comprise at least about 80%, 90%, 95%, 100% sequence identity to SEQ ID NO: 4, 36, 37, 38, 39, 40, 41, 42, 43, 44, 45, 46, 47, 48, 49, 50, 51, 52 or any combination thereof. In some embodiments, a method can further comprise introducing into an organelle a polynucleotide encoding a marker. In some embodiments, a marker can be a positive selectable marker, a negative selectable marker, a screenable marker, or any combination thereof. In some embodiments, a marker can be a positive selectable marker. In some embodiments, a positive selectable marker can comprise an herbicide tolerance protein. In some embodiments, described herein are methods of growing a cell produced by a method described above. In some embodiments, a method can comprise growing a cell in a presence of a positive selection agent and selecting a cell that is homoplasmic for altered organellar genome. In some embodiments, a method can comprise growing a cell in an absence of a positive selection agent, followed by selecting a cell that lacks a non-integrated recombinant DNA construct. In some embodiments, a method can further comprise growing a cell in an absence of a positive selection agent, followed by growing a cell in a presence of a negative selection agent, followed by selecting a cell that lacks a non-integrated recombinant DNA construct. In some embodiments, a cell can be produced by a method disclosed herein. In some embodiments, a cell can comprise a yeast cell, an algal cell, a plant cell, an insect cell, a non-human animal cell, an isolated and purified human cell, or a mammalian tissue culture cell. In some embodiments, a plant, seed, root, stem, leaf, flower, or fruit can be produced from a cell described herein. In some embodiments, a plant, seed, root, stem, leaf, flower, or fruit can comprise an altered organellar genome.

Disclosed herein are methods for altering an organellar genome. In some embodiments, a method can comprise (a) introducing into a nucleus of a cell: a first polynucleotide encoding a modified polynucleotide guided polypeptide. In some embodiments, a modified polynucleotide guided polypeptide can comprise a polynucleotide guided polypeptide operably linked to an organellar targeting peptide. In some embodiments, a polynucleotide guided polypeptide when associated with a guide RNA can cleave at least one target sequence present in an organellar genome. In some embodiments, a method can further comprise introducing into a nucleus of a cell a second polynucleotide encoding a guide RNA. In some embodiments, a guide RNA can direct a polynucleotide guided polypeptide to cleave at least one target sequence present in an organellar genome. In some embodiments, a method can comprise (b) introducing into an organelle of a cell a replacement DNA. In some embodiments, a method can comprise (c) growing a cell comprising a nucleus of (a) and an organelle of (b) under conditions in which a first polynucleotide and a second polynucleotide are expressed. In some embodiments, a method can comprise (d) selecting a cell comprising an altered organellar genome. In some embodiments, a replacement DNA can comprise fragments of organellar DNA or a complete organellar DNA from a cultivar, line, sub-species or other species. In some embodiments, a replacement DNA can be distinct from an organellar genome of a cell. In some embodiments, at least one target sequence may not be present in a replacement DNA. In some embodiments, after (a) and prior to (b), a cell can be selected in which a native organellar genome may have been eliminated. In some embodiments, an organelle can be a mitochondrion. In some embodiments, a cell can be produced by a method disclosed herein. In some embodiments, organelle can be a plastid. In some embodiments, an organelle can be a chloroplast. In some embodiments, cell can comprise a yeast cell, an algal cell, a plant cell, an insect cell, a non-human animal cell, an isolated and purified human cell, or a mammalian tissue culture cell. In some embodiments, a plant, seed, root, stem, leaf, flower, or fruit produced from a cell disclosed herein. In some embodiments, a plant, seed, root, stem, leaf, flower, or fruit can comprise an altered organellar genome.

Disclosed herein are methods for altering an organellar genome. In some embodiments, a method can comprise (a) introducing into a nucleus of a cell a first polynucleotide encoding a modified polynucleotide guided polypeptide. In some embodiments, a modified polynucleotide guided polypeptide can comprise a polynucleotide guided polypeptide operably linked to an organellar targeting peptide. In some embodiments, a polynucleotide guided polypeptide when associated with a guide RNA can cleave at least one target sequence present in an organellar genome. In some embodiments, a method can comprise (b) introducing into an organelle of a cell: a second polynucleotide encoding a guide RNA. In some embodiments, a guide RNA can direct a polynucleotide guided polypeptide to cleave at least one target sequence present in an organellar genome. In some embodiments, a method can further comprise introducing into an organelle of a cell, a replacement DNA. In some embodiments, a method can comprise (c) growing a cell comprising a nucleus of (a) and an organelle of (b) under conditions in which a first polynucleotide and a second polynucleotide are expressed. In some embodiments, a method can further comprise (d) selecting a cell comprising an altered organellar genome. In some embodiments, a replacement DNA can comprise fragments of organellar DNA or a complete organellar DNA from a cultivar, line, sub-species or other species. In some embodiments, a replacement DNA can be distinct from an organellar genome of a cell. In some embodiments, at least one target sequence may not be present in a replacement DNA. In some embodiments, after (a) and prior to (b), a cell can be selected in which a native organellar genome may have been eliminated. In some embodiments, an organelle can be a mitochondrion. In some embodiments, organelle can be a plastid. In some embodiments, an organelle can be a chloroplast. In some embodiments, a cell can be produced by a method disclosed herein. In some embodiments, cell can comprise a yeast cell, an algal cell, a plant cell, an insect cell, a non-human animal cell, an isolated and purified human cell, or a mammalian tissue culture cell. In some embodiments, a plant, seed, root, stem, leaf, flower, or fruit produced from a cell disclosed herein. In some embodiments, a plant, seed, root, stem, leaf, flower, or fruit can comprise an altered organellar genome.

### BRIEF DESCRIPTION OF THE SEQUENCE LISTING

The instant application contains a Sequence Listing which has been submitted electronically in ASCII format and is hereby incorporated by reference in its entirety. Said ASCII copy, created on Apr. 13, 2022 is named 51090-703_301_SL.txt and is 124,022 bytes in size.

The disclosure is more fully understood from the following detailed description and Sequence Listing (Table 1), which form a part of this application.

SEQ ID NO: 1 corresponds to an amino acid sequence of SpCas9, a Cas9 from Streptococcus pyogenes.

SEQ ID NO: 2 corresponds to an amino acid sequence for MAD2.

SEQ ID NO: 3 corresponds to an amino acid sequence for MAD7.

SEQ ID NO: 4 corresponds to an amino acid sequence for a permeant peptide derived from a third alpha helix of Drosophila melanogaster transcription factor Antennapaedia, referred to as penetratin.

SEQ ID NO: 5 corresponds to an amino acid sequence of a hydrophobic quenching peptide that tetramerizes GFP and prevents maturation of the chromophore.

SEQ ID NO: 6 corresponds to an amino acid sequence of a caspase recognition sequence.

SEQ ID NO: 7 corresponds to a nucleotide sequence for a candidate RNA editing sequences present in a wheat mitochondrial cox2 gene at position 449 of the gene.

SEQ ID NO: 8 corresponds to a nucleotide sequence for a candidate RNA editing sequences present in a wheat mitochondrial cox2 gene at position 587 of the gene.

SEQ ID NO: 9 corresponds to a nucleotide sequence for a candidate RNA editing sequences for this purpose are present in a wheat mitochondrial cox2 gene at position 620 of the gene.

SEQ ID NO: 10 corresponds to a nucleotide sequence encoding mCAS9-A, a modified Cas9 comprising a Cas9 protein linked to an organelle targeting peptide of the ATPase beta subunit.

SEQ ID NO: 11 corresponds to a nucleotide sequence encoding mCAS9-B, a modified Cas9 comprising a Cas9 protein linked to an organelle targeting peptide of the 70KD protein.

SEQ ID NO: 12 corresponds to a nucleotide sequence of a first of four guide RNA target sites in a COX/gene.

SEQ ID NO: 13 corresponds to a nucleotide sequence of a second of four guide RNA target sites in a COX/gene.

SEQ ID NO: 14 corresponds to a nucleotide sequence of a third of four guide RNA target sites in a COX/gene.

SEQ ID NO: 15 corresponds to a nucleotide sequence of a fourth of four guide RNA target sites in a COX/gene.

SEQ ID NO: 16 corresponds to a nucleotide sequence of a tracrRNA.

SEQ ID NO: 17 corresponds to a nucleotide sequence of a SNR52 promoter.

SEQ ID NO: 18 corresponds to a nucleotide sequence of a SUP4 termination element.

SEQ ID NO: 19 corresponds to a nucleotide sequence of a minimal COX3 promoter.

SEQ ID NO: 20 corresponds to a nucleotide sequence of a tRNA gene, tF (GAA).

SEQ ID NO: 21 corresponds to a nucleotide sequence of a tRNA gene, tW (UCA).

SEQ ID NO: 22 corresponds to a nucleotide sequence of a minimal COX3 terminator element (SEQ ID NO: 22).

SEQ ID NO: 23 corresponds to a nucleotide sequence of a tRNA gene, tM (CAU).

SEQ ID NO: 24 corresponds to an amino acid sequence of a mitochondrial targeting peptide, NDUFV2 MTS.

SEQ ID NO: 25 corresponds to a nucleotide sequence encoding a modified Cas9, in which an NDUFV2 signal sequence is fused with an amino terminus of Cas9.

SEQ ID NO: 26 corresponds to an amino acid sequence of a mitochondrial targeting peptide from citrate synthase.

SEQ ID NO: 27 corresponds to a nucleotide sequence encoding a modified CAS9, in which a citrate synthase mitochondrial targeting peptide is fused with an amino terminus of Cas9.

SEQ ID NO: 28 corresponds to a nucleotide sequence of a variable targeting domain for a gRNA sequence targeting a human COX3 gene in mitochondria.

SEQ ID NO: 29 corresponds to a nucleic acid sequence of an expression cassette for a guide RNA utilizing a promoter and terminator of a human 5S rRNA gene.

SEQ ID NO: 30 corresponds to an amino acid sequence of a first 12 amino acids of yeast cytochrome C oxidase followed by 18 amino acids of alternating H and K residues.

SEQ ID NO: 31 corresponds to an amino acid sequence of a first 12 amino acids of cell penetrating peptide followed by 18 amino acids of alternating H and K residues.

SEQ ID NO: 32 corresponds to a nucleotide sequence of a 958 bp COX2 fragment, including 150 bp 5′ and 3′ homologies flanking a cox2-62 deletion; nucleotides 73313 to 74,270 of GenBank KP263414.

SEQ ID NO: 33 corresponds to a nucleotide sequence of a 629 bp Cox3 fragment, including 150 bp 5′ and 3′ flanking homologies covering cox3-10 deletion; 78734 to 79362 nucleotides of GenBank KP263414.

SEQ ID NO: 34 corresponds to a nucleotide sequence of a cox2P:ARG8m:cox2Term: Cox3 fragment to complement Cox3-10 mutation in strain MCC125.

SEQ ID NO: 35 corresponds to a nucleotide sequence of a pNY5 edit plasmid.

SEQ ID NO: 36 corresponds to an amino acid sequence for KH-AtOEP34.

SEQ ID NO: 37 corresponds to an amino acid sequence for TAT.

SEQ ID NO: 38 corresponds to an amino acid sequence for R9.

SEQ ID NO: 39 corresponds to an amino acid sequence for Pep-1.

SEQ ID NO: 40 corresponds to an amino acid sequence for MPG.

SEQ ID NO: 41 corresponds to an amino acid sequence for gamma-ZEIN.

SEQ ID NO: 42 corresponds to an amino acid sequence for Transporant.

SEQ ID NO: 43 corresponds to an amino acid sequence for MAP.

SEQ ID NO: 44 corresponds to an amino acid sequence for Pept 1.

SEQ ID NO: 45 corresponds to an amino acid sequence for Pept 2.

SEQ ID NO: 46 corresponds to an amino acid sequence for IVV-14.

SEQ ID NO: 47 corresponds to an amino acid sequence for Ig(v).

SEQ ID NO: 48 corresponds to an amino acid sequence for Amphiphilic model peptide.

SEQ ID NO: 49 corresponds to an amino acid sequence for pVEC.

SEQ ID NO: 50 corresponds to an amino acid sequence for HRSV.

SEQ ID NO: 51 corresponds to an amino acid sequence for Bp100.

SEQ ID NO: 52 corresponds to an amino acid sequence for TAT₂, a dimer of an HIV-1 Tat basic domain.

SEQ ID NO: 53 corresponds to a first 25 amino-terminal residues of a precursor of subunit IV of cytochrome c oxidase of yeast and is a mitochondrial transit sequence (MTS) for that protein.

SEQ ID NO: 54 corresponds to an amino acid sequence of an MTS-Cas9 fusion protein.

SEQ ID NO: 55 corresponds to nucleotide sequence encoding an MTS-Cas9 fusion protein presented as SEQ ID NO: 54.

SEQ ID NO: 56 corresponds to a nucleotide sequence encoding a variable region of gRNA 1.

SEQ ID NO: 57 corresponds to a nucleotide sequence encoding a variable region of gRNA 2.

SEQ ID NO: 58 corresponds to a nucleotide sequence encoding a variable region of gRNA 3.

SEQ ID NO: 59 corresponds to a nucleotide sequence encoding a variable region of gRNA 4.

SEQ ID NO: 60 corresponds to a nucleotide sequence of a expression cassette for gRNAs 1 and 2.

SEQ ID NO: 61 corresponds to a nucleotide sequence of an expression cassette for gRNAs 3 and 4.

SEQ ID NO: 62 corresponds to a nucleotide sequence of a SphI-BsaI fragment used to construct pNY95.

SEQ ID NO: 63 corresponds to a nucleotide sequence of a BsaI-NotI fragment used to construct pNY95.

SEQ ID NO: 64 corresponds to a nucleotide sequence of a variable region of gRNA 3ny.

SEQ ID NO: 65 corresponds to a nucleotide sequence of a variable region of gRNA 4ny.

SEQ ID NO: 66 corresponds to a nucleotide sequence of a guide RNA expression cassette for gRNAs 3ny and 4ny present in pNY95.

SEQ ID NO: 67 corresponds to a nucleotide sequence of a donor DNA for gRNAs 3 and 4.

SEQ ID NO: 68 corresponds to a nucleotide sequence of PCR Primer I, recognizing a mitochondrial genome region upstream of a site recognized by gRNA 1.

SEQ ID NO: 69 corresponds to a nucleotide sequence of PCR Primer C, recognizing a mitochondrial genome region upstream of a site recognized by gRNA 1.

SEQ ID NO: 70 corresponds to a nucleotide sequence of PCR Primer F, recognizing a mitochondrial genome region downstream of a site recognized by gRNA 2.

SEQ ID NO: 71 corresponds to a nucleotide sequence of PCR Primer F1, recognizing a mitochondrial genome region downstream of a site recognized by gRNA 2.

SEQ ID NO: 72 corresponds to a nucleotide sequence of PCR Primer 35, recognizing a mitochondrial genome region upstream of a site recognized by gRNA 3.

SEQ ID NO: 73 corresponds to a nucleotide sequence of PCR Primer 36, recognizing a mitochondrial genome region downstream of a site recognized by gRNA 4.

SEQ ID NO: 74 corresponds to a nucleotide sequence of PCR Primer 37, recognizing a mitochondrial genome region upstream of a site recognized by gRNA 3.

SEQ ID NO: 75 corresponds to a nucleotide sequence of PCR Primer 38, recognizing a mitochondrial genome region downstream of a site recognized by gRNA 4.

SEQ ID NO: 76 corresponds to a nucleotide sequence of PCR Primer 11, recognizing an antisense strand of a donor DNA.

SEQ ID NO: 77 corresponds to a nucleotide sequence of PCR Primer 12, recognizing a sense strand of a donor DNA.

SEQ ID NO: 78 corresponds to a nucleotide sequence of PCR Primer 13, recognizing an antisense strand of a donor DNA.

SEQ ID NO: 79 corresponds to a nucleotide sequence of PCR Primer 15, recognizing a sense strand of a donor DNA.

SEQ ID NO: 80 corresponds to a nucleotide sequence of a donor DNA carrying an ARG8m ORF with homologous regions.

SEQ ID NO: 81 corresponds to an amino acid sequence of an ARG8m protein encoded in SEQ ID NO: 80.

SEQ ID NO: 82 corresponds to a nucleotide sequence of a NcoI-NotI fragment containing an expression cassette for gRNAs 3 and 4.

SEQ ID NO: 83 corresponds to a nucleotide sequence of an ori5 element with XbaI and NotI sites at the ends.

SEQ ID NO: 84 corresponds to a nucleotide sequence of PCR Primer PRS316-FP, for amplification of an ARS-CEN element.

SEQ ID NO: 85 corresponds to a nucleotide sequence of PCR Primer PRS316-RP, for amplification of an ARS-CEN element.

SEQ ID NO: 86 corresponds to a nucleotide sequence of an amplified DNA fragment for a right junction of a donor DNA integration event.

SEQ ID NO: 87 corresponds to a nucleotide sequence of an amplified DNA fragment for a left junction of a donor DNA integration event.

TABLE 1
Sequence Listing.

| SEQ ID NO | SEQUENCE |
|---|---|
| 1 | MDKKYSIGLDIGTNSVGWAVITDEYKVPSKKFKVLGNTDRHSIKKNLIGALLFDSGETAE |
| | ATRLKRTARRRYTRRKNRICYLQEIFSNEMAKVDDSFFHRLEESFLVEEDKKHERHPIFG |
| | NIVDEVAYHEKYPTIYHLRKKLVDSTDKADLRLIYLALAHMIKFRGHFLIEGDLNPDNSD |
| | VDKLFIQLVQTYNQLFEENPINASGVDAKAILSARLSKSRRLENLIAQLPGEKKNGLFGNL |
| | IALSLGLTPNFKSNFDLAEDAKLQLSKDTYDDDLDNLLAQIGDQYADLFLAAKNLSDAIL |
| | LSDILRVNTEITKAPLSASMIKRYDEHHQDLTLLKALVRQQLPEKYKEIFFDQSKNGYAG |
| | YIDGGASQEEFYKFIKPILEKMDGTEELLVKLNREDLLRKQRTFDNGSIPHQIHLGELHAIL |
| | RRQEDFYPFLKDNREKIEKILTFRIPYYVGPLARGNSRFAWMTRKSEETITPWNFEEVVD |
| | KGASAQSFIERMTNFDKNLPNEKVLPKHSLLYEYFTVYNELTKVKYVTEGMRKPAFLSG |
| | EQKKAIVDLLFKTNRKVTVKQLKEDYFKKIECFDSVEISGVEDRFNASLGTYHDLLKIIKD |
| | KDFLDNEENEDILEDIVLTLTLFEDREMIEERLKTYAHLFDDKVMKQLKRRRYTGWGRL |
| | SRKLINGIRDKQSGKTILDFLKSDGFANRNFMQLIHDDSLTFKEDIQKAQVSGQGDSLHE |
| | HIANLAGSPAIKKGILQTVKVVDELVKVMGRHKPENIVIEMARENQTTQKGQKNSRERM |
| | KRIEEGIKELGSQILKEHPVENTQLQNEKLYLYYLQNGRDMYVDQELDINRLSDYDVDHI |
| | VPQSFLKDDSIDNKVLTRSDKNRGKSDNVPSEEVVKKMKNYWRQLLNAKLITQRKFDN |
| | LTKAERGGLSELDKAGFIKRQLVETRQITKHVAQILDSRMNTKYDENDKLIREVKVITLK |
| | SKLVSDFRKDFQFYKVREINNYHHAHDAYLNAVVGTALIKKYPKLESEFVYGDYKVYD |
| | VRKMIAKSEQEIGKATAKYFFYSNIMNFFKTEITLANGEIRKRPLIETNGETGEIVWDKGR |
| | DFATVRKVLSMPQVNIVKKTEVQTGGFSKESILPKRNSDKLIARKKDWDPKKYGGFDSP |
| | TVAYSVLVVAKVEKGKSKKLKSVKELLGITIMERSSFEKNPIDFLEAKGYKEVKKDLIIKL |
| | PKYSLFELENGRKRMLASAGELQKGNELALPSKYVNFLYLASHYEKLKGSPEDNEQKQL |
| | FVEQHKHYLDEIIEQISEFSKRVILADANLDKVLSAYNKHRDKPIREQAENIIHLFTLTNLG |
| | APAAFKYFDTTIDRKRYTSTKEVLDATLIHQSITGLYETRIDLSQLGGD |
| 2 | MSSLTKFTNKYSKQLTIKNELIPVGKTLENIKENGLIDGDEQLNENYQKAKIIVDDFLRDFI |
| | NKALNNTQIGNWRELADALNKEDEDNIEKLQDKIRGIIVSKFETFDLFSSYSIKKDEKIIDD |
| | DNDVEEEELDLGKKTSSFKYIFKKNLFKLVLPSYLKTTNQDKLKIISSFDNFSTYFRGFFEN |
| | RKNIFTKKPISTSIAYRIVHDNFPKFLDNIRCFNVWQTECPQLIVKADNYLKSKNVIAKDK |
| | SLANYFTVGAYDYFLSQNGIDFYNNIIGGLPAFAGHEKIQGLNEFINQECQKDSELKSKLK |
| | NRHAFKMAVLFKQILSDREKSFVIDEFESDAQVIDAVKNFYAEQCKDNNVIFNLLNLIKNI |
| | AFLSDDELDGIFIEGKYLSSVSQKLYSDWSKLRNDIEDSANSKQGNKELAKKIKTNKGDV |
| | EKAISKYEFSLSELNSIVHDNTKFSDLLSCTLHKVASEKLVKVNEGDWPKHLKNNEEKQK |
| | IKEPLDALLEIYNTLLIFNCKSFNKNGNFYVDYDRCINELSSVVYLYNKTRNYCTKKPYN |
| | TDKFKLNFNSPQLGEGFSKSKENDCLTLLFKKDDNYYVGIIRKGAKINFDDTQAIADNTD |
| | NCIFKMNYFLLKDAKKFIPKCSIQLKEVKAHFKKSEDDYILSDKEKFASPLVIKKSTFLLA |
| | TAHVKGKKGNIKKFQKEYSKENPTEYRNSLNEWIAFCKEFLKTYKAATIFDITTLKKAEE |
| | YADIVEFYKDVDNLCYKLEFCPIKTSFIENLIDNGDLYLFRINNKDFSSKSTGTKNLHTLY |
| | LQAIFDERNLNNPTIMLNGGAELFYRKESIEQKNRITHKAGSILVNKVCKDGTSLDDKIRN |
| | EIYQYENKFIDTLSDEAKKVLPNVIKKEATHDITKDKRFTSDKFFFHCPLTINYKEGDTKQ |
| | FNNEVLSFLRGNPDINIIGIDRGERNLIYVTVINQKGEILDSVSFNTVTNKSSKIEQTVDYEE |
| | KLAVREKERIEAKRSWDSISKIATLKEGYLSAIVHEICLLMIKHNAIVVLENLNAGFKRIR |
| | GGLSEKSVYQKFEKMLINKLNYFVSKKESDWNKPSGLLNGLQLSDQFESFEKLGIQSGFI |
| | FYVPAAYTSKIDPTTGFANVLNLSKVRNVDAIKSFFSNFNEISYSKKEALFKFSFDLDSLSK |
| | KGFSSFVKFSKSKWNVYTFGERIIKPKNKQGYREDKRINLTFEMKKLLNEYKVSFDLENN |
| | LIPNLTSANLKDTFWKELFFIFKTTLQLRNSVTNGKEDVLISPVKNAKGEFFVSGTHNKTL |
| | PQDCDANGAYHIALKGLMILERNNLVREEKDTKKIMAISNVDWFEYVQKRRGVL |
| 3 | MNNGTNNFQNFIGISSLQKTLRNALIPTETTQQFIVKNGIIKEDELRGENRQILKDIMDDYY |
| | RGFISETLSSIDDIDWTSLFEKMEIQLKNGDNKDTLIKEQTEYRKAIHKKFANDDRFKNMF |
| | SAKLISDILPEFVIHNNNYSASEKEEKTQVIKLFSRFATSFKDYFKNRANCFSADDISSSSC |
| | HRIVNDNAEIFFSNALVYRRIVKSLSNDDINKISGDMKDSLKEMSLEEIYSYEKYGEFITQE |
| | GISFYNDICGKVNSFMNLYCQKNKENKNLYKLQKLHKQILCIADTSYEVPYKFESDEEVY |
| | QSVNGFLDNISSKHIVERLRKIGDNYNGYNLDKIYIVSKFYESVSQKTYRDWETINTALEI |
| | HYNNILPGNGKSKADKVKKAVKNDLQKSITEINELVSNYKLCSDDNIKAETYIHEISHILN |
| | NFEAQELKYNPEIHLVESELKASELKNVLDVIMNAFHWCSVFMTEELVDKDNNFYAELE |
| | EIYDEIYPVISLYNLVRNYVTQKPYSTKKIKLNFGIPTLADGWSKSKEYSNNAIILMRDNL |
| | YYLGIFNAKNKPDKKIIEGNTSENKGDYKKMIYNLLPGPNKMIPKVFLSSKTGVETYKPS |
| | AYILEGYKQNKHIKSSKDFDITFCHDLIDYFKNCIAIHPEWKNFGFDFSDTSTYEDISGFYR |
| | EVELQGYKIDWTYISEKDIDLLQEKGQLYLFQIYNKDFSKKSTGNDNLHTMYLKNLFSEE |
| | NLKDIVLKLNGEAEIFFRKSSIKNPIIHKKGSILVNRTYEAEEKDQFGNIQIVRKNIPENIYQ |
| | ELYKYFNDKSDKELSDEAAKLKNVVGHHEAATNIVKDYRYTYDKYFLHMPITINFKAN |
| | KTGFINDRILQYIAKEKDLHVIGIDRGERNLIYVSVIDTCGNIVEQKSFNIVNGYDYQIKLK |
| | QQEGARQIARKEWKEIGKIKEIKEGYLSLVIHEISKMVIKYNAIIAMEDLSYGFKKGRFKV |
| | ERQVYQKFETMLINKLNYLVFKDISITENGGLLKGYQLTYIPDKLKNVGHQCGCIFYVPA |
| | AYTSKIDPTTGFVNIFKFKDLTVDAKREFIKKFDSIRYDSEKNLFCHThDYNNFITQNTVMS |
| | KSSWSVYTYGVRIKRRFVNGRFSNESDTIDITKDMEKTLEMTDINWRDGHDLRQDIIDYE |
| | IVQHIFEIFRLTVQMRNSLSELEDRDYDRLISPVLNENNIFYDSAKAGDALPKDADANGA |
| | YCIALKGLYEIKQITENWKEDGKFSRDKLKISNKDWFDFIQNKRYL |
| 4 | RQIKIWFQNRRMKWKK |
| 5 | DEVDFQGPCNDSSDPLVVAASIIGILHLILWILDRL |
| 6 | DEVD |
| 7 | ACUUUUGACAGUUAUACGAUUCCAGAA |
| 8 | UGGGCUGUACCUUCCUCAGGUGUCAAA |
| 9 | GCUGUACCUGGUCGUUCAAAUCUUACC |
| 10 | AAAAAAGAATGGTTCTACCAAGACTATATACAGCTACAAGTCGTGCTGCTCTGTCGA |
| | CCGACAAGAAGTACTCCATTGGGCTCGATATCGGCACAAACAGCGTCGGCTGGGCCG |
| | TCATTACGGACGAGTACAAGGTGCCGAGCAAAAAATTCAAAGTTCTGGGCAATACCG |
| | ATCGCCACAGCATAAAGAAGAACCTCATTGGCGCCCTCCTGTTCGACTCCGGGGAGA |
| | CGGCCGAAGCCACGCGGCTCAAAAGAACAGCACGGCGCAGATATACCCGCAGAAAG |
| | AATCGGATCTGCTACCTGCAGGAGATCTTTAGTAATGAGATGGCTAAGGTGGATGAC |
| | TCTTTCTTCCATAGGCTGGAGGAGTCCTTTTTGGTGGAGGAGGATAAAAAGCACGAG |
| | CGCCACCCAATCTTTGGCAATATCGTGGACGAGGTGGCGTACCATGAAAAGTACCCA |
| | ACCATATATCATCTGAGGAAGAAGCTTGTAGACAGTACTGATAAGGCTGACTTGCGG |
| | TTGATCTATCTCGCGCTGGCGCATATGATCAAATTTCGGGGACACTTCCTCATCGAGG |
| | GGGACCTGAACCCAGACAACAGCGATGTCGACAAACTCTTTATCCAACTGGTTCAGA |
| | CTTACAATCAGCTTTTCGAAGAGAACCCGATCAACGCATCCGGAGTTGACGCCAAAG |
| | CAATCCTGAGCGCTAGGCTGTCCAAATCCCGGCGGCTCGAAAACCTCATCGCACAGC |
| | TCCCTGGGGAGAAGAAGAACGGCCTGTTTGGTAATCTTATCGCCCTGTCACTCGGGC |
| | TGACCCCCAACTTTAAATCTAACTTCGACCTGGCCGAAGATGCCAAGCTTCAACTGA |
| | GCAAAGACACCTACGATGATGATCTCGACAATCTGCTGGCCCAGTCGGCGACCAGTA |
| | CGCAGACCTTTTTTTGGCGGCAAAGAACCTGTCAGACGCCATTCTGCTGAGTGATATT |
| | CTGCGAGTGAACACGGAGATCACCAAAGCTCCGCTGAGCGCTAGTATGATCAAGCGC |
| | TATGATGAGCACCACCAAGACTTGACTTTGCTGAAGGCCCTTGTCAGACAGCAACTG |
| | CCTGAGAAGTACAAGGAAATTTTCTTCGATCAGTCTAAAAATGGCTACGCCGGATAC |
| | ATTGACGGCGGAGCAAGCCAGGAGGAATTTTACAAATTTATTAAGCCCATCTTGGAA |
| | AAAATGGACGGCACCGAGGAGCTGCTGGTAAAGCTTAACAGAGAAGATCTGTTGCG |
| | CAAACAGCGCACTTTCGACAATGGAAGCATCCCCCACCAGATTCACCTGGGCGAACT |
| | GCACGCTATCCTCAGGCGGCAAGAGGATTTCTACCCCTTTTTGAAAGATAACAGGGA |
| | AAAGATTGAGAAAATCCTCACATTTCGGATACCCTACTATGTAGGCCCCCTCGCCCG |
| | GGGAAATTCCAGATTCGCGTGGATGACTCGCAAATCAGAAGAGACCATCACTCCCTG |
| | GAACTTCGAGGAAGTCGTGGATAAGGGGGCCTCTGCCCAGTCCTTCATCGAAAGGAT |
| | GACTAACTTTGATAAAAATCTGCCTAACGAAAAGGTGCTTCCTAAACACTCTCTGCT |
| | GTACGAGTACTTCACAGTTTATAACGAGCTCACCAAGGTCAAATACGTCACAGAAGG |
| | GATGAGAAAGCCAGCATTCCTGTCTGGAGAGCAGAAGAAAGCTATCGTGGACCTCCT |
| | CTTCAAGACGAACCGGAAAGTTACCGTGAAACAGCTCAAAGAAGACTATTTCAAAA |
| | AGATTGAATGTTTCGACTCTGTTGAAATCAGCGGAGTGGAGGATCGCTTCAACGCAT |
| | CCCTGGGAACGTATCACGATCTCCTGAAAATCATTAAAGACAAGGACTTCCTGGACA |
| | ATGAGGAGAACGAGGACATTCTTGAGGACATTGTCCTCACCCTTACGTTGTTTGAAG |
| | ATAGGGAGATGATTGAAGAACGCTTGAAAACTTACGCTCATCTCTTCGACGACAAAG |
| | TCATGAAACAGCTCAAGAGGCGCCGATATACAGGATGGGGGCGGCTGTCAAGAAAA |
| | CTGATCAATGGGATCCGAGACAAGCAGAGTGGAAAGACAATCCTGGATTTTCTTAAG |
| | TCCGATGGATTTGCCAACCGGAACTTCATGCAGTTGATCCATGATGACTCTCTCACCT |
| | TTAAGGAGGACATCCAGAAAGCACAAGTTTCTGGCCAGGGGGACAGTCTTCACGAG |
| | CACATCGCTAATCTTGCAGGTAGCCCAGCTATCAAAAAGGGAATACTGCAGACCGTT |
| | AAGGTCGTGGATGAACTCGTCAAAGTAATGGGAAGGCATAAGCCCGAGAATATCGT |
| | TATCGAGATGGCCCGAGAGAACCAAACTACCCAGAAGGGACAGAAGAACAGTAGGG |
| | AAAGGATGAAGAGGATTGAAGAGGGTATAAAAGAACTGGGGTCCCAAATCCTTAAG |
| | GAACACCCAGTTGAAAACACCCAGCTTCAGAATGAGAAGCTCTACCTGTACTACCTG |
| | CAGAACGGCAGGGACATGTACGTGGATCAGGAACTGGACATCAATCGGCTCTCCGA |
| | CTACGACGTGGATCATATCGTGCCCCAGTCTTTTCTCAAAGATGATTCTATTGATAAT |
| | AAAGTGTTGACAAGATCCGATAAAAATAGAGGGAAGAGTGATAACGTCCCCTCAGA |
| | AGAAGTTGTCAAGAAAATGAAAAATTATTGGCGGCAGCTGCTGAACGCCAAACTGA |
| | TCACACAACGGAAGTTCGATAATCTGACTAAGGCTGAACGAGGTGGCCTGTCTGAGT |
| | TGGATAAAGCCGGCTTCATCAAAAGGCAGCTTGTTGAGACACGCCAGATCACCAAGC |
| | ACGTGGCCCAAATTCTCGATTCACGCATGAACACCAAGTACGATGAAAATGACAAAC |
| | TGATTCGAGAGGTGAAAGTTATTACTCTGAAGTCTAAGCTGGTCTCAGATTTCAGAA |
| | AGGACTTTCAGTTTTATAAGGTGAGAGAGATCAACAATTACCACCATGCGCATGATG |
| | CCTACCTGAATGCAGTGGTAGGCACTGCACTTATCAAAAAATATCCCAAGCTTGAAT |
| | CTGAATTTGTTTACGGAGACTATAAAGTGTACGATGTTAGGAAAATGATCGCAAAGT |
| | CTGAGCAGGAAATAGGCAAGGCCACCGCTAAGTACTTCTTTTACAGCAATATTATGA |
| | ATTTTTTCAAGACCGAGATTACACTGGCCAATGGAGAGATTCGGAAGCGACCACTTA |
| | TCGAAACAAACGGAGAAACAGGAGAAATCGTGTGGGACAAGGGTAGGGATTTCGCG |
| | ACAGTCCGGAAGGTCCTGTCCATGCCGCAGGTGAACATCGTTAAAAAGACCGAAGTA |
| | CAGACCGGAGGCTTCTCCAAGGAAAGTATCCTCCCGAAAAGGAACAGCGACAAGCT |
| | GATCGCACGCAAAAAAGATTGGGACCCCAAGAAATACGGCGGATTCGATTCTCCTAC |
| | AGTCGCTTACAGTGTACTGGTTGTGGCCAAAGTGGAGAAAGGGAAGTCTAAAAAACT |
| | CAAAAGCGTCAAGGAACTGCTGGGCATCACAATCATGGAGCGATCAAGCTTCGAAA |
| | AAAACCCCATCGACTTTCTCGAGGCGAAAGGATATAAAGAGGTCAAAAAAGACCTC |
| | ATCATTAAGCTTCCCAAGTACTCTCTCTTTGAGCTTGAAAACGGCCGGAAACGAATG |
| | CTCGCTAGTGCGGGCGAGCTGCAGAAAGGTAACGAGCTGGCACTGCCCTCTAAATAC |
| | GTTAATTTCTTGTATCTGGCCAGCCACTATGAAAAGCTCAAAGGGTCTCCCGAAGAT |
| | AATGAGCAGAAGCAGCTGTTCGTGGAACAACACAAACACTACCTTGATGAGATCATC |
| | GAGCAAATAAGCGAATTCTCCAAAAGAGTGATCCTCGCCGACGCTAACCTCGATAAG |
| | GTGCTTTCTGCTTACAATAAG |
| | CACAGGGATAAGCCCATCAGGGAGCAGGCAGAAAACATTATCCACTTGTTTACTCT |
| | GACCAACTTGGGCGCGCCTGCAGCCTTCAAGTACTTCGACACCACCATAGACAGAA |
| | AGCGGTACACCTCTACAAAGGAGGTCCTGGACGCCACACTGATTCATCAGTCAATTA |
| | CGGGGCTCTATGAAACAAGAATCGACCTCTCTCAGCTCGGTGGAGACTGA |
| 11 | GATCCATGAAAAGCTTCATTACAAGGAACAAGACAGCCATTGACAAGAAGTACTCC |
| | ATTGGGCTCGATATCGGCACAAACAGCGTCGGCTGGGCCGTCATTACGGACGAGTA |
| | CAAGGTGCCGAGCAAAAAATTCAAAGTTCTGGGCAATACCGATCGCCACAGCATAA |
| | AGAAGAACCTCATTGGCGCCCTCCTGTTCGACTCCGGGGAGACGGCCGAAGCCACG |
| | CGGCTCAAAAGAACAGCACGGCGCAGATATACCCGCAGAAAGAATCGGATCTGCTA |
| | CCTGCAGGAGATCTTTAGTAATGAGATGGCTAAGGTGGATGACTCTTTCTTCCATAG |
| | GCTGGAGGAGTCCTTTTTGGTGGAGGAGGATAAAAAGCACGAGCGCCACCCAATCT |
| | TTGGCAATATCGTGGACGAGGTGGCGTACCATGAAAAGTACCCAACCATATATCATC |
| | TGAGGAAGAAGCTTGTAGACAGTACTGATAAGGCTGACTTGCGGTTGATCTATCTCG |
| | CGCTGGCGCATATGATCAAATTTCGGGGACACTTCCTCATCGAGGGGGACCTGAACC |
| | CAGACAACAGCGATGTCGACAAACTCTTTATCCAACTGGTTCAGACTTACAATCAGC |
| | TTTTCGAAGAGAACCCGATCAACGCATCCGGAGTTGACGCCAAAGCAATCCTGAGCG |
| | CTAGGCTGTCCAAATCCCGGCGGCTCGAAAACCTCATCGCACAGCTCCCTGGGGAGA |
| | AGAAGAACGGCCTGTTTGGTAATCTTATCGCCCTGTCACTCGGGCTGACCCCCAACTT |
| | TAAATCTAACTTCGACCTGGCCGAAGATGCCAAGCTTCAACTGAGCAAAGACACCTA |
| | CGATGATGATCTCGACAATCTGCTGGCCCAGATCGGCGACCAGTACGCAGACCTTTT |
| | TTTGGCGGCAAAGAACCTGTCAGACGCCATTCTGCTGAGTGATATTCTGCGAGTGAA |
| | CACGGAGATCACCAAAGCTCCGCTGAGCGCTAGTATGATCAAGCGCTATGATGAGCA |
| | CCACCAAGACTTGACTTTGCTGAAGGCCCTTGTCAGACAGCAACTGCCTGAGAAGTA |
| | CAAGGAAATTTTCTTCGATCAGTCTAAAAATGGCTACGCCGGATACATTGACGGCGG |
| | AGCAAGCCAGGAGGAATTTTACAAATTTATTAAGCCCATCTTGGAAAAAATGGACGG |
| | CACCGAGGAGCTGCTGGTAAAGCTTAACAGAGAAGATCTGTTGCGCAAACAGCGCA |
| | CTTTCGACAATGGAAGCATCCCCCACCAGATTCACCTGGGCGAACTGCACGCTATCC |
| | TCAGGCGGCAAGAGGATTTCTACCCCTTTTTGAAAGATAACAGGGAAAAGATTGAGA |
| | AAATCCTCACATTTCGGATACCCTACTATGTAGGCCCCCTCGCCCGGGGAAATTCCA |
| | GATTCGCGTGGATGACTCGCAAATCAGAAGAGACCATCACTCCCTGGAACTTCGAGG |
| | AAGTCGTGGATAAGGGGGCCTCTGCCCAGTCCTTCATCGAAAGGATGACTAACTTTG |
| | ATAAAAATCTGCCTAACGAAAAGGTGCTTCCTAAACACTCTCTGCTGTACGAGTACT |
| | TCACAGTTTATAACGAGCTCACCAAGGTCAAATACGTCACAGAAGGGATGAGAAAG |
| | CCAGCATTCCTGTCTGGAGAGCAGAAGAAAGCTATCGTGGACCTCCTCTTCAAGACG |
| | AACCGGAAAGTTACCGTGAAACAGCTCAAAGAAGACTATTTCAAAAAGATTGAATG |
| | TTTCGACTCTGTTGAAATCAGCGGAGTGGAGGATCGCTTCAACGCATCCCTGGGAAC |
| | GTATCACGATCTCCTGAAAATCATTAAAGACAAGGACTTCCTGGACAATGAGGAGAA |
| | CGAGGACATTCTTGAGGACATTGTCCTCACCCTTACGTTGTTTGAAGATAGGGAGAT |
| | GATTGAAGAACGCTTGAAAACTTACGCTCATCTCTTCGACGACAAAGTCATGAAACA |
| | GCTCAAGAGGCGCCGATATACAGGATGGGGGCGGCTGTCAAGAAAACTGATCAATG |
| | GGATCCGAGACAAGCAGAGTGGAAAGACAATCCTGGATTTTCTTAAGTCCGATGGAT |
| | TTGCCAACCGGAACTTCATGCAGTTGATCCATGATGACTCTCTCACCTTTAAGGAGGA |
| | CATCCAGAAAGCACAAGTTTCTGGCCAGGGGGACAGTCTTCACGAGCACATCGCTAA |
| | TCTTGCAGGTAGCCCAGCTATCAAAAAGGGAATACTGCAGACCGTTAAGGTCGTGGA |
| | TGAACTCGTCAAAGTAATGGGAAGGCATAAGCCCGAGAATATCGTTATCGAGATGGC |
| | CCGAGAGAACCAAACTACCCAGAAGGGACAGAAGAACAGTAGGGAAAGGATGAAG |
| | AGGATTGAAGAGGGTATAAAAGAACTGGGGTCCCAAATCCTTAAGGAACACCCAGT |
| | TGAAAACACCCAGCTTCAGAATGAGAAGCTCTACCTGTACTACCTGCAGAACGGCAG |
| | GGACATGTACGTGGATCAGGAACTGGACATCAATCGGCTCTCCGACTACGACGTGGA |
| | TCATATCGTGCCCCAGTCTTTTCTCAAAGATGATTCTATTGATAATAAAGTGTTGACA |
| | AGATCCGATAAAAATAGAGGGAAGAGTGATAACGTCCCCTCAGAAGAAGTTGTCAA |
| | GAAAATGAAAAATTATTGGCGGCAGCTGCTGAACGCCAAACTGATCACACAACGGA |
| | AGTTCGATAATCTGACTAAGGCTGAACGAGGTGGCCTGTCTGAGTTGGATAAAGCCG |
| | GCTTCATCAAAAGGCAGCTTGTTGAGACACGCCAGATCACCAAGCACGTGGCCCAAA |
| | TTCTCGATTCACGCATGAACACCAAGTACGATGAAAATGACAAACTGATTCGAGAGG |
| | TGAAAGTTATTACTCTGAAGTCTAAGCTGGTCTCAGATTTCAGAAAGGACTTTCAGTT |
| | TTATAAGGTGAGAGAGATCAACAATTACCACCATGCGCATGATGCCTACCTGAATGC |
| | AGTGGTAGGCACTGCACTTATCAAAAAATATCCCAAGCTTGAATCTGAATTTGTTTAC |
| | GGAGACTATAAAGTGTACGATGTTAGGAAAATGATCGCAAAGTCTGAGCAGGAAAT |
| | AGGCAAGGCCACCGCTAAGTACTTCTTTTACAGCAATATTATGAATTTTTTCAAGACC |
| | GAGATTACACTGGCCAATGGAGAGATTCGGAAGCGACCACTTATCGAAACAAACGG |
| | AGAAACAGGAGAAATCGTGTGGGACAAGGGTAGGGATTTCGCGACAGTCCGGAAGG |
| | TCCTGTCCATGCCGCAGGTGAACATCGTTAAAAAGACCGAAGTACAGACCGGAGGCT |
| | TCTCCAAGGAAAGTATCCTCCCGAAAAGGAACAGCGACAAGCTGATCGCACGCAAA |
| | AAAGATTGGGACCCCAAGAAATACGGCGGATTCGATTCTCCTACAGTCGCTTACAGT |
| | GTACTGGTTGTGGCCAAAGTGGAGAAAGGGAAGTCTAAAAAACTCAAAAGCGTCAA |
| | GGAACTGCTGGGCATCACAATCATGGAGCGATCAAGCTTCGAAAAAAACCCCATCG |
| | ACTTTCTCGAGGCGAAAGGATATAAAGAGGTCAAAAAAGACCTCATCATTAAGCTTC |
| | CCAAGTACTCTCTCTTTGAGCTTGAAAACGGCCGGAAACGAATGCTCGCTAGTGCGG |
| | GCGAGCTGCAGAAAGGTAACGAGCTGGCACTGCCCTCTAAATACGTTAATTTCTTGT |
| | ATCTGGCCAGCCACTATGAAAAGCTCAAAGGGTCTCCCGAAGATAATGAGCAGAAG |
| | CAGCTGTTCGTGGAACAACACAAACACTACCTTGATGAGATCATCGAGCAAATAAGC |
| | GAATTCTCCAAAAGAGTGATCCTCGCCGACGCTAACCTCGATAAGGTGCTTTCTGCTT |
| | ACAATAAGCACAGGGATAAGCCCATCAGGGAGCAGGCAGAAAACATTATCCACTTG |
| | TTTACTCTGACCAACTTGGGCGCGCCTGCAGCCTTCAAGTACTTCGACACCACCATAG |
| | ACAGAAAGCGGTACACCTCTACAAAGGAGGTCCTGGACGCCACACTGATTCATCAGT |
| | CAATTACGGGGCTCTATGAAACAAGAATCGACCTCTCTCAGCTCGGTGGAGACTGA |
| 12 | TTCTTTGAAGTATCAGGAGGTGG |
| 13 | ATGATTATTGCAATTCCAACAGG |
| 14 | GCTATTTTTAGTGGTATGGCAGG |
| 15 | ACCATGTAAATATTGTGAACCAGG |
| 16 | TTCTTTGAAGTATCAGGAGGGTTTTAGAGCTAGAAATAGCAAGTTAAAATAAGGCTA |
| | GTCCGTTATCAACTTGAAAAAGTGGCACCGAGTCGGTGCT |
| 17 | TCTTTGAAAAGATAATGTATGATTATGCTTTCACTCATATTTATACAGAAACTTGATG |
| | TTTTCTTTCGAGTATATACAAGGTGATTACATGTACGTTTGAAGTACAACTCTAGATT |
| | TTGTAGTGCCCTCTTGGGCTAGCGGTAAAGGTGCGCATTTTTTCACACCCTACAATGT |
| | TCTGTTCAAAAGATTTTGGTCAAACGCTGTAGAAGTGAAAGTTGGTGCGCATGTTTC |
| | GGCGTTCGAAACTTCTCCGCAGTGAAAGATAAATGATC |
| 18 | TTTTTTTGTTTTTTATGTCT |
| 19 | TATATATTATGTATTATTATATAAATATATATATATATTATATTATAAGTAATAATAA |
| | GTATTATATTATATATA |
| 20 | GCTTTTATAGCTTAGTGGTAAAGCGATAAATTGAAGATTTATTTACATGTAGTTCGAT |
| | TCTCATTAAGGGCAATA |
| 21 | AGGAGATTAGCTTAATTGGTATAGCATTCGTTTTACACACGAAAGATTATAGGTTCG |
| | AACCCTATATTTCCTAAAT |
| 22 | TTATTAATAATTAACAATAATTAATATATTATAATTTATATATATATATTTTATATTAT |
| | TATAATAATATTCTTACAAATATAATTATTATATATTATTCCTTCAAAACTCCTAACG |
| | G |
| 23 | GAGCTTGTATAGTTTAATTGGTTAAAACATTTGTCTCATAAATAAATAATGTAAGGTT |
| | CAATTCCTTCTACAAGTA |
| 24 | MFFSAALRARAAGLTAHWGRHVRNLHKTVMQN |
| 25 | ATGTTCTTCTCCGCGGCGCTCCGGGCCCGGGCGGCTGGCCTCACCGCCCACTGGGGA |
| | AGACATGTAAGGAATTTGCATAAGACAGTTATGCAAAATGACAAGAAGTACTCCAT |
| | TGGGCTCGATATCGGCACAAACAGCGTCGGCTGGGCCGTCATTACGGACGAGTACA |
| | AGGTGCCGAGCAAAAAATTCAAAGTTCTGGGCAATACCGATCGCCACAGCATAAAG |
| | AAGAACCTCATTGGCGCCCTCCTGTTCGACTCCGGGGAGACGGCCGAAGCCACGCG |
| | GCTCAAAAGAACAGCACGGCGCAGATATACCCGCAGAAAGAATCGGATCTGCTACC |
| | TGCAGGAGATCTTTAGTAATGAGATGGCTAAGGTGGATGACTCTTTCTTCCATAGGC |
| | TGGAGGAGTCCTTTTTGGTGGAGGAGGATAAAAAGCACGAGCGCCACCCAATCTTT |
| | GGCAATATCGTGGACGAGGTGGCGTACCATGAAAAGTACCCAACCATATATCATCT |
| | GAGGAAGAAGCTTGTAGACAGTACTGATAAGGCTGACTTGCGGTTGATCTATCTCGC |
| | GCTGGCGCATATGATCAAATTTCGGGGACACTTCCTCATCGAGGGGGACCTGAACCC |
| | AGACAACAGCGATGTCGACAAACTCTTTATCCAACTGGTTCAGACTTACAATCAGCT |
| | TTTCGAAGAGAACCCGATCAACGCATCCGGAGTTGACGCCAAAGCAATCCTGAGCGC |
| | TAGGCTGTCCAAATCCCGGCGGCTCGAAAACCTCATCGCACAGCTCCCTGGGGAGAA |
| | GAAGAACGGCCTGTTTGGTAATCTTATCGCCCTGTCACTCGGGCTGACCCCCAACTTT |
| | AAATCTAACTTCGACCTGGCCGAAGATGCCAAGCTTCAACTGAGCAAAGACACCTAC |
| | GATGATGATCTCGACAATCTGCTGGCCCAGATCGGCGACCAGTACGCAGACCTTTTT |
| | TTGGCGGCAAAGAACCTGTCAGACGCCATTCTGCTGAGTGATATTCTGCGAGTGAAC |
| | ACGGAGATCACCAAAGCTCCGCTGAGCGCTAGTATGATCAAGCGCTATGATGAGCAC |
| | CACCAAGACTTGACTTTGCTGAAGGCCCTTGTCAGACAGCAACTGCCTGAGAAGTAC |
| | AAGGAAATTTTCTTCGATCAGTCTAAAAATGGCTACGCCGGATACATTGACGGCGGA |
| | GCAAGCCAGGAGGAATTTTACAAATTTATTAAGCCCATCTTGGAAAAAATGGACGGC |
| | ACCGAGGAGCTGCTGGTAAAGCTTAACAGAGAAGATCTGTTGCGCAAACAGCGCAC |
| | TTTCGACAATGGAAGCATCCCCCACCAGATTCACCTGGGCGAACTGCACGCTATCCT |
| | CAGGCGGCAAGAGGATTTCTACCCCTTTTTGAAAGATAACAGGGAAAAGATTGAGA |
| | AAATCCTCACATTTCGGATACCCTACTATGTAGGCCCCCTCGCCCGGGGAAATTCCA |
| | GATTCGCGTGGATGACTCGCAAATCAGAAGAGACCATCACTCCCTGGAACTTCGAGG |
| | AAGTCGTGGATAAGGGGGCCTCTGCCCAGTCCTTCATCGAAAGGATGACTAACTTTG |
| | ATAAAAATCTGCCTAACGAAAAGGTGCTTCCTAAACACTCTCTGCTGTACGAGTACT |
| | TCACAGTTTATAACGAGCTCACCAAGGTCAAATACGTCACAGAAGGGATGAGAAAG |
| | CCAGCATTCCTGTCTGGAGAGCAGAAGAAAGCTATCGTGGACCTCCTCTTCAAGACG |
| | AACCGGAAAGTTACCGTGAAACAGCTCAAAGAAGACTATTTCAAAAAGATTGAATG |
| | TTTCGACTCTGTTGAAATCAGCGGAGTGGAGGATCGCTTCAACGCATCCCTGGGAAC |
| | GTATCACGATCTCCTGAAAATCATTAAAGACAAGGACTTCCTGGACAATGAGGAGAA |
| | CGAGGACATTCTTGAGGACATTGTCCTCACCCTTACGTTGTTTGAAGATAGGGAGAT |
| | GATTGAAGAACGCTTGAAAACTTACGCTCATCTCTTCGACGACAAAGTCATGAAACA |
| | GCTCAAGAGGCGCCGATATACAGGATGGGGGCGGCTGTCAAGAAAACTGATCAATG |
| | GGATCCGAGACAAGCAGAGTGGAAAGACAATCCTGGATTTTCTTAAGTCCGATGGAT |
| | TTGCCAACCGGAACTTCATGCAGTTGATCCATGATGACTCTCTCACCTTTAAGGAGGA |
| | CATCCAGAAAGCACAAGTTTCTGGCCAGGGGGACAGTCTTCACGAGCACATCGCTAA |
| | TCTTGCAGGTAGCCCAGCTATCAAAAAGGGAATACTGCAGACCGTTAAGGTCGTGGA |
| | TGAACTCGTCAAAGTAATGGGAAGGCATAAGCCCGAGAATATCGTTATCGAGATGGC |
| | CCGAGAGAACCAAACTACCCAGAAGGGACAGAAGAACAGTAGGGAAAGGATGAAG |
| | AGGATTGAAGAGGGTATAAAAGAACTGGGGTCCCAAATCCTTAAGGAACACCCAGT |
| | TGAAAACACCCAGCTTCAGAATGAGAAGCTCTACCTGTACTACCTGCAGAACGGCAG |
| | GGACATGTACGTGGATCAGGAACTGGACATCAATCGGCTCTCCGACTACGACGTGGA |
| | TCATATCGTGCCCCAGTCTTTTCTCAAAGATGATTCTATTGATAATAAAGTGTTGACA |
| | AGATCCGATAAAAATAGAGGGAAGAGTGATAACGTCCCCTCAGAAGAAGTTGTCAA |
| | GAAAATGAAAAATTATTGGCGGCAGCTGCTGAACGCCAAACTGATCACACAACGGA |
| | AGTTCGATAATCTGACTAAGGCTGAACGAGGTGGCCTGTCTGAGTTGGATAAAGCCG |
| | GCTTCATCAAAAGGCAGCTTGTTGAGACACGCCAGATCACCAAGCACGTGGCCCAAA |
| | TTCTCGATTCACGCATGAACACCAAGTACGATGAAAATGACAAACTGATTCGAGAGG |
| | TGAAAGTTATTACTCTGAAGTCTAAGCTGGTCTCAGATTTCAGAAAGGACTTTCAGTT |
| | TTATAAGGTGAGAGAGATCAACAATTACCACCATGCGCATGATGCCTACCTGAATGC |
| | AGTGGTAGGCACTGCACTTATCAAAAAATATCCCAAGCTTGAATCTGAATTTGTTTAC |
| | GGAGACTATAAAGTGTACGATGTTAGGAAAATGATCGCAAAGTCTGAGCAGGAAAT |
| | AGGCAAGGCCACCGCTAAGTACTTCTTTTACAGCAATATTATGAATTTTTTCAAGACC |
| | GAGATTACACTGGCCAATGGAGAGATTCGGAAGCGACCACTTATCGAAACAAACGG |
| | AGAAACAGGAGAAATCGTGTGGGACAAGGGTAGGGATTTCGCGACAGTCCGGAAGG |
| | TCCTGTCCATGCCGCAGGTGAACATCGTTAAAAAGACCGAAGTACAGACCGGAGGCT |
| | TCTCCAAGGAAAGTATCCTCCCGAAAAGGAACAGCGACAAGCTGATCGCACGCAAA |
| | AAAGATTGGGACCCCAAGAAATACGGCGGATTCGATTCTCCTACAGTCGCTTACAGT |
| | GTACTGGTTGTGGCCAAAGTGGAGAAAGGGAAGTCTAAAAAACTCAAAAGCGTCAA |
| | GGAACTGCTGGGCATCACAATCATGGAGCGATCAAGCTTCGAAAAAAACCCCATCG |
| | ACTTTCTCGAGGCGAAAGGATATAAAGAGGTCAAAAAAGACCTCATCATTAAGCTTC |
| | CCAAGTACTCTCTCTTTGAGCTTGAAAACGGCCGGAAACGAATGCTCGCTAGTGCGG |
| | GCGAGCTGCAGAAAGGTAACGAGCTGGCACTGCCCTCTAAATACGTTAATTTCTTGT |
| | ATCTGGCCAGCCACTATGAAAAGCTCAAAGGGTCTCCCGAAGATAATGAGCAGAAG |
| | CAGCTGTTCGTGGAACAACACAAACACTACCTTGATGAGATCATCGAGCAAATAAGC |
| | GAATTCTCCAAAAGAGTGATCCTCGCCGACGCTAACCTCGATAAGGTGCTTTCTGCTT |
| | ACAATAAGCACAGGGATAAGCCCATCAGGGAGCAGGCAGAAAACATTATCCACTTG |
| | TTTACTCTGACCAACTTGGGCGCGCCTGCAGCCTTCAAGTACTTCGACACCACCATAG |
| | ACAGAAAGCGGTACACCTCTACAAAGGAGGTCCTGGACGCCACACTGATTCATCAGT |
| | CAATTACGGGGCTCTATGAAACAAGAATCGACCTCTCTCAGCTCGGTGGAGACTGA |
| 26 | MALLTAAARLLGTKNASCLVLAARH |
| 27 | ATGGCTTTACTTACTGCGGCCGCCCGGCTCTTGGGAACCAAGAATGCATCTTGTCTT |
| | GTTCTTGCAGCCCGGCATATGGCTTTACTTACTGCGGCCGCCCGGCTCTTGGGAACC |
| | AAGAATGCAGACAAGAAGTACTCCATTGGGCTCGATATCGGCACAAACAGCGTCGG |
| | CTGGGCCGTCATTACGGACGAGTACAAGGTGCCGAGCAAAAAATTCAAAGTTCTGG |
| | GCAATACCGATCGCCACAGCATAAAGAAGAACCTCATTGGCGCCCTCCTGTTCGACT |
| | CCGGGGAGACGGCCGAAGCCACGCGGCTCAAAAGAACAGCACGGCGCAGATATACC |
| | CGCAGAAAGAATCGGATCTGCTACCTGCAGGAGATCTTTAGTAATGAGATGGCTAAG |
| | GTGGATGACTCTTTCTTCCATAGGCTGGAGGAGTCCTTTTTGGTGGAGGAGGATAAA |
| | AAGCACGAGCGCCACCCAATCTTTGGCAATATCGTGGACGAGGTGGCGTACCATGAA |
| | AAGTACCCAACCATATATCATCTGAGGAAGAAGCTTGTAGACAGTACTGATAAGGCT |
| | GACTTGCGGTTGATCTATCTCGCGCTGGCGCATATGATCAAATTTCGGGGACACTTCC |
| | TCATCGAGGGGGACCTGAACCCAGACAACAGCGATGTCGACAAACTCTTTATCCAAC |
| | TGGTTCAGACTTACAATCAGCTTTTCGAAGAGAACCCGATCAACGCATCCGGAGTTG |
| | ACGCCAAAGCAATCCTGAGCGCTAGGCTGTCCAAATCCCGGCGGCTCGAAAACCTCA |
| | TCGCACAGCTCCCTGGGGAGAAGAAGAACGGCCTGTTTGGTAATCTTATCGCCCTGT |
| | CACTCGGGCTGACCCCCAACTTTAAATCTAACTTCGACCTGGCCGAAGATGCCAAGC |
| | TTCAACTGAGCAAAGACACCTACGATGATGATCTCGACAATCTGCTGGCCCAGATCG |
| | GCGACCAGTACGCAGACCTTTTTTTGGCGGCAAAGAACCTGTCAGACGCCATTCTGC |
| | TGAGTGATATTCTGCGAGTGAACACGGAGATCACCAAAGCTCCGCTGAGCGCTAGTA |
| | TGATCAAGCGCTATGATGAGCACCACCAAGACTTGACTTTGCTGAAGGCCCTTGTCA |
| | GACAGCAACTGCCTGAGAAGTACAAGGAAATTTTCTTCGATCAGTCTAAAAATGGCT |
| | ACGCCGGATACATTGACGGCGGAGCAAGCCAGGAGGAATTTTACAAATTTATTAAGC |
| | CCATCTTGGAAAAAATGGACGGCACCGAGGAGCTGCTGGTAAAGCTTAACAGAGAA |
| | GATCTGTTGCGCAAACAGCGCACTTTCGACAATGGAAGCATCCCCCACCAGATTCAC |
| | CTGGGCGAACTGCACGCTATCCTCAGGCGGCAAGAGGATTTCTACCCCTTTTTGAAA |
| | GATAACAGGGAAAAGATTGAGAAAATCCTCACATTTCGGATACCCTACTATGTAGGC |
| | CCCCTCGCCCGGGGAAATTCCAGATTCGCGTGGATGACTCGCAAATCAGAAGAGACC |
| | ATCACTCCCTGGAACTTCGAGGAAGTCGTGGATAAGGGGGCCTCTGCCCAGTCCTTC |
| | ATCGAAAGGATGACTAACTTTGATAAAAATCTGCCTAACGAAAAGGTGCTTCCTAAA |
| | CACTCTCTGCTGTACGAGTACTTCACAGTTTATAACGAGCTCACCAAGGTCAAATAC |
| | GTCACAGAAGGGATGAGAAAGCCAGCATTCCTGTCTGGAGAGCAGAAGAAAGCTAT |
| | CGTGGACCTCCTCTTCAAGACGAACCGGAAAGTTACCGTGAAACAGCTCAAAGAAG |
| | ACTATTTCAAAAAGATTGAATGTTTCGACTCTGTTGAAATCAGCGGAGTGGAGGATC |
| | GCTTCAACGCATCCCTGGGAACGTATCACGATCTCCTGAAAATCATTAAAGACAAGG |
| | ACTTCCTGGACAATGAGGAGAACGAGGACATTCTTGAGGACATTGTCCTCACCCTTA |
| | CGTTGTTTGAAGATAGGGAGATGATTGAAGAACGCTTGAAAACTTACGCTCATCTCT |
| | TCGACGACAAAGTCATGAAACAGCTCAAGAGGCGCCGATATACAGGATGGGGGCGG |
| | CTGTCAAGAAAACTGATCAATGGGATCCGAGACAAGCAGAGTGGAAAGACAATCCT |
| | GGATTTTCTTAAGTCCGATGGATTTGCCAACCGGAACTTCATGCAGTTGATCCATGAT |
| | GACTCTCTCACCTTTAAGGAGGACATCCAGAAAGCACAAGTTTCTGGCCAGGGGGAC |
| | AGTCTTCACGAGCACATCGCTAATCTTGCAGGTAGCCCAGCTATCAAAAAGGGAATA |
| | CTGCAGACCGTTAAGGTCGTGGATGAACTCGTCAAAGTAATGGGAAGGCATAAGCCC |
| | GAGAATATCGTTATCGAGATGGCCCGAGAGAACCAAACTACCCAGAAGGGACAGAA |
| | GAACAGTAGGGAAAGGATGAAGAGGATTGAAGAGGGTATAAAAGAACTGGGGTCCC |
| | AAATCCTTAAGGAACACCCAGTTGAAAACACCCAGCTTCAGAATGAGAAGCTCTACC |
| | TGTACTACCTGCAGAACGGCAGGGACATGTACGTGGATCAGGAACTGGACATCAATC |
| | GGCTCTCCGACTACGACGTGGATCATATCGTGCCCCAGTCTTTTCTCAAAGATGATTC |
| | TATTGATAATAAAGTGTTGACAAGATCCGATAAAAATAGAGGGAAGAGTGATAACG |
| | TCCCCTCAGAAGAAGTTGTCAAGAAAATGAAAAATTATTGGCGGCAGCTGCTGAACG |
| | CCAAACTGATCACACAACGGAAGTTCGATAATCTGACTAAGGCTGAACGAGGTGGCC |
| | TGTCTGAGTTGGATAAAGCCGGCTTCATCAAAAGGCAGCTTGTTGAGACACGCCAGA |
| | TCACCAAGCACGTGGCCCAAATTCTCGATTCACGCATGAACACCAAGTACGATGAAA |
| | ATGACAAACTGATTCGAGAGGTGAAAGTTATTACTCTGAAGTCTAAGCTGGTCTCAG |
| | ATTTCAGAAAGGACTTTCAGTTTTATAAGGTGAGAGAGATCAACAATTACCACCATG |
| | CGCATGATGCCTACCTGAAGCAGTGGTAGGCACTGCACTTATCAAAAAATATCCCAA |
| | GCTTGAATCTGAATTTGTTTACGGAGACTATAAAGTGTACGATGTTAGGAAAATGAT |
| | CGCAAAGTCTGAGCAGGAAATAGGCAAGGCCACCGCTAAGTACTTCTTTTACAGCAA |
| | TATTATGAATTTTTTCAAGACCGAGATTACACTGGCCAATGGAGAGATTCGGAAGCG |
| | ACCACTTATCGAAACAAACGGAGAAACAGGAGAAATCGTGTGGGACAAGGGTAGGG |
| | ATTTCGCGACAGTCCGGAAGGTCCTGTCCATGCCGCAGGTGAACATCGTTAAAAAGA |
| | CCGAAGTACAGACCGGAGGCTTCTCCAAGGAAAGTATCCTCCCGAAAAGGAACAGC |
| | GACAAGCTGATCGCACGCAAAAAAGATTGGGACCCCAAGAAATACGGCGGATTCGA |
| | TTCTCCTACAGTCGCTTACAGTGTACTGGTTGTGGCCAAAGTGGAGAAAGGGAAGTC |
| | TAAAAAACTCAAAAGCGTCAAGGAACTGCTGGGCATCACAATCATGGAGCGATCAA |
| | GCTTCGAAAAAAACCCCATCGACTTTCTCGAGGCGAAAGGATATAAAGAGGTCAAA |
| | AAAGACCTCATCATTAAGCTTCCCAAGTACTCTCTCTTTGAGCTTGAAAACGGCCGG |
| | AAACGAATGCTCGCTAGTGCGGGCGAGCTGCAGAAAGGTAACGAGCTGGCACTGCC |
| | CTCTAAATACGTTAATTTCTTGTATCTGGCCAGCCACTATGAAAAGCTCAAAGGGTCT |
| | CCCGAAGATAATGAGCAGAAGCAGCTGTTCGTGGAACAACACAAACACTACCTTGAT |
| | GAGATCATCGAGCAAATAAGCGAATTCTCCAAAAGAGTGATCCTCGCCGACGCTAAC |
| | CTCGATAAGGTGCTTTCTGCTTACAATAAGCACAGGGATAAGCCCATCAGGGAGCAG |
| | GCAGAAAACATTATCCACTTGTTTACTCTGACCAACTTGGGCGCGCCTGCAGCCTTCA |
| | AGTACTTCGACACCACCATAGACAGAAAGCGGTACACCTCTACAAAGGAGGTCCTGG |
| | ACGCCACACTGATTCATCAGTCAATTACGGGGCTCTATGAAACAAGAATCGACCTCT |
| | CTCAGCTCGGTGGAGACTGA |
| 28 | GTCTGGTGAGTAGTGCATGGCT |
| 29 | AGCCCCGCGGCCCCGGGCTGGCGGTGTCGGCTGCAATCCGGCGGGCACGGCCGGGC |
| | CGGGCTGGGCTCTTGGGGCAGCCAGGCGCCTCCTTCAGCGCCTACGGCCATACCACC |
| | CTGAACGCGCCCGATCTCGTCTGATCTCGGAAGCTAAGCAGGGTCGGGCCTGGTTAG |
| | TACTTGGATGGGAGACCACCTGGGAATACCGGGTGCTGTAGGCTTTTTCTTTGGCTTT |
| | TTGCTGTTTCTTTCCTTTTCTTCCAGACGGAGTCTCGCCCTCTCGCCCAGGCTGGAGTG |
| | CGGTGGCGCCATCTCGGCTCACTGCAAGCTCCGCCTCCCGGGTCCACGCCATTCCCCG |
| | GCCTCAGCCTCCCGAGTAGCTGGGCCTACAGGCGCCCGCCACCACGCCCGGCCACTT |
| | TGTTCTATTTTTCCTAGAGACGGGCTTTCACCCTGTTAGCCGGGATGGTCTGGAGCTC |
| 30 | MLSLRQSIRFFKKHKHKHKHKHKHKHKHKH |
| 31 | KKLFKKILKLYLKHKHKHKHKHKHKHKHKH |
| 32 | TCCTTGCTTCATACCTTTATAAATAAGGTAATCACTAATATATTATAATAATAAAAAT |
| | TATATATATTATATATAATCTAAATATTATATATTTTAATAAATATTAATATATATGA |
| | TATGAATATTATTAGTTTTTGGGAAGCGGGAATCCCGTAAGGAGTGAGGGACCCCTC |
| | CCTAACGGGAGGAGGACCGAAGGAGTTTTAGTATTTTTTTTTTTTTAATAAAATATAT |
| | ATTTATATGATTAATAATATTATATATATTATTTATAAAAATAATATATAATTTTAATT |
| | ATTTTTAATAAAAAAAGGTGGGGTTGATAATATAATATAATATTTTTTATTTTAATTT |
| | ATAATATATAATAATAAATTATAAATAAATTTTAATTAAAAGTAGTATTAACATATTA |
| | TAAATAGACAAAAGAGTCTAAAGGTTAAGATTTATTAAAATGTTAGATTTATTAAGA |
| | TTACAATTAACAACATTCATTATGAATGATGTACCAACACCTTATGCATGTTATTTTC |
| | AGGATTCAGCAACACCAAATCAAGAAGGTATTTTAGAATTACATGATAATATTATGT |
| | TTTATTTATTAGTTATTTTAGGTTTAGTATCTTGAATGTTATATACAATTGTTATAACA |
| | TATTCAAAAAATCCTATTGCATATAAATATATTAAACATGGACAAACTATTGAAGTT |
| | ATTTGAACAATTTTTCCAGCTGTAATTTTATTAATTATTGCTTTTCCTTCATTTATTTTA |
| | TTATATTTATGTGATGAAGTTATTTCACCAGCTATAACTATTAAAGCTATTGGATATC |
| | AATGATATTGAAAATATGAATATTCAGATTTTATTAATGATAGTGGTGAAACTGTTG |
| | AATTTGAATCATATGTTATTCCTGATGAATTATTAGAAGAAGGTCAATTAAGATTATT |
| | AGATACTGATACTTCTATAGTTGTACCTGTA |
| 33 | ATTATCAATGATTTATATTAATAATAAATATAAATAATAAAAAATATATATAATATA |
| | ATATAATAAATATATTTCCTTTAATATTAATAAATTAATAATAATAATAATAATAATA |
| | ATAAAATATTTAAATAAATTATATTCAATACAAATTAATTATTTATATTATTAATAAT |
| | TGAATAAATAATCCGGTCGAAAGAGATATTAATTCGATTATATTATTTATTTAATTAT |
| | ATTTAATTTAAATATATAAATTAATATATATATATTGAATTATATATAAATTTATTTTA |
| | TAATTTTATAAATAATATATTATTATAAATATTTAATATAATTTATATTATTATTAAAT |
| | AAAAGATTTATTAAATTAATATTATTATTTAATTTTATTATATAGTTTAAGGGATAAT |
| | ATTTTATTAATATTTTTTTTATTTATTTATTTAATTATATTATATATATAATATATATAT |
| | AACAATAAATTTATGACACATTTAGAAAGAAGTAGACATCAACAACATCCATTTCAT |
| | ATGGTTATGCCTTCACCATGACCTATTGTAGTATCATTTGCATTATTATCATTAGCATT |
| | ATCACTAGCATTAACAATGCATGGTTATATTGGTAATATGAATATG |
| 34 | AATTCGAGCTCGGTACCCGGGGATCACCTAAATATGTAGCTTCAGCTACAATATCTCT |
| | AAATCATAAAATAGAACTTGTTAATAATACAAATAATGCTAAATATACCATATTCAT |
| | ATTACCAATATAACCATGCATTGTTAATGCTAGTGATAATGCTAATGATAATAATGC |
| | AAATGATACTACAATTGGTCATGGTGAAGGCATAACCATATGAAATGGATGTTGTTG |
| | ATGTCTACTTCTTTCTAAATGTGTCATAAATTTATTGTTATATATATATTATATATATA |
| | ATATAATTAAATAAATAAATAAAAAAAATATTAATAAAATATTATCCTCAAAACTAT |
| | ATAATAAAATTAAATAATAATATTAATTTAATAAATCTTTTATTTAATAATAATATAA |
| | ATTATATTAAATATTTATAATAATATATTATTTATAAAATTATAAAATAAATTTATAT |
| | ATAATTCAATATATATATATTAATTTATATATTTAAATTAAATATAATTAAATAAATA |
| | ATATAATCGAATTAATATCTCTTTCGACCGGATTATTTATTCAATTATTAATAATATA |
| | AATAATTAATTTGTATTGAATATAATTTATTTAAATATTTTATTATTATTATTATTATT |
| | ATTATTAATTTATTAATATTAAAGGAAATATATTTATTATATTATATTATATATATTTT |
| | TTATTATTTATATTTATTATTAATATAAATCATTGATAATATCTTCTTTTTATTTATTTA |
| | TTATTATAATTAAAAAGAATCTTATATTAGACCTGCAGATAAGGTGATTGAATAGAA |
| | TATAAATCTATATCTTTATTATATTTAAGAATATTATTATAATTATTATTATTATTATT |
| | ATTTTTAATAATTAAAAATATTAATAATAAGTAAATATTAAGGATCCTTAAGCATATA |
| | CAGCTTCGATAGCTTTTTCGAAAGCATCCATACCTTCTTCGATTAATTCATCTTCGATT |
| | GTTAAAGCAGGTACGAATCTTACTGTTGATTTACCAGCTGTGATGATTAATAAACCTA |
| | ATTCTCTAGCTTTTTTGATTACTTCTGTTGGAGGTTCTACAAATTCAGCACCTAACATT |
| | AAACCTTTACCTCTGATTGTTTTGATTTGATTAGGATATTTAGCTTGGATTTCTCTTAA |
| | TCTTTTTTGTAAGATATCTGATTTTTTTGATACTTGTTTTAAGAATGCTTCATCAGCAA |
| | TTGTATCTAATACATAATTTGATACTGAACAAGCTAAAGGATTACCACCATATGTTGT |
| | ACCATGATCACCTACTCTTAAAGCATTATTTACTTTTTCATTTACGATTGTAGCAGCA |
| | ATAGGGAAACCATTACCTAATGCTTTAGCTGATGTGAAAATATCAGGATGAGCTTCT |
| | GAAGGTAAATAAGCATGAGCTCATAATTTACCTGATCTACCTAAACCACATTGAATT |
| | TCATCATGAATTACGATTACATCATTATCTTGACAGATTTTTTTTAAACCAGTTAATTT |
| | TTCTACTTCTACAGGAAATACACCACCTTCACCTTGAATAGGTTCTACGATTAAACCA |
| | GCGATTTCATCTTTTTTTGTTTCGATATATGATTGTAATTTTGTCATTTCATCATTTAA |
| | ATTTAAGAATGATACATGAGGTACTAAATCACCGAAAGGTGTTCTATATTTTGAATTT |
| | CATGTTACTGATAAAGCACCCATTGTTCTACCATGGAATGAATTTTCGAAAGCTACA |
| | ATACCTTGTTTACTAGGATTTTTCATGATACCATGTTTTTTAGCGAATTTTAATGCAGC |
| | TTCATTAGCTTCGGTACCTGAATTACATAAGAATACTCTTGAAGCATCATGTTGACCA |
| | CCGAATTGTTTTGTTTTTTCTACGATTTTTTCTGATAAATCTAAACATTCTTTTGTGAA |
| | ATATAAATTTGATGAATGTACTAATTTATTAGCTTGATGATGTAAGATTTCAGCTACT |
| | TTAGGATTAGCATGACCTAAAGCTGTTACAGCAATACCAGCTGTGAAATCGATATAT |
| | TCTTTACCATTTACATCATCATATAATTTAGCATTTTTACCTCTTGTGATACATAAATC |
| | TTCAGGTCTTGAATATGTTGTTACTTGAAAAGCTTTTTCTTCTAAGATTGATGTGAAT |
| | CTTCTTGATGATGTTGATGATAAATATCTTTTGAACATTTTAATAAATCTTAACCTTTA |
| | GACTCTTTTGTCTATTTATAATATGTTAATACTACTTTTAATTAAAATTTATTTATAAT |
| | TTATTATTATATATTATAAATAAAATAAAAATATTATATTACCAAACCCCACCACTAG |
| | AAGC |
| 35 | AGCGCCCAATACGCAAACCGCCTCTCCCCGCGCGTTGGCCGATTCATTAATGCAGCT |
| | GGCACGACAGGTTTCCCGACTGGAAAGCGGGCAGTGAGCGCAACGCAATTAATGTG |
| | AGTTAGCTCACTCATTAGGCACCCCAGGCTTTACACTTTATGCTTCCGGCTCGTATGT |
| | TGTGTGGAATTGTGAGCGGATAACAATTTCACACAGGAAACAGCTATGACCATGATT |
| | ACGAATTTAATACGACTCACTATAGGGAATTCGAGCTCGGTACCCGGGGATCACCTA |
| | AATATGTAGCTTCAGCTACAATATCTCTAAATCATAAAATAGAACTTGTTAATAATAC |
| | AAATAATGCTAAATATACCATATTCATATTACCAATATAACCATGCATTGTTAATGCT |
| | AGTGATAATGCTAATGATAATAATGCAAATGATACTACAATTGGTCATGGTGAAGGC |
| | ATAACCATATGAAATGGATGTTGTTGATGTCTACTTCTTTCTAAATGTGTCATAAATT |
| | TATTGTTATATATATATTATATATATAATATAATTAAATAAATAAATAAAAAAAATAT |
| | TAATAAAATATTATCCTCAAAACTATATAATAAAATTAAATAATAATATTAATTTAAT |
| | AAATCTTTTATTTAATAATAATATAAATTATATTAAATATTTATAATAATATATTATTT |
| | ATAAAATTATAAAATAAATTTATATATAATTCAATATATATATATTAATTTATATATT |
| | TAAATTAAATATAATTAAATAAATAATATAATCGAATTAATATCTCTTTCGACCGGAT |
| | TATTTATTCAATTATTAATAATATAAATAATTAATTTGTATTGAATATAATTTATTTAA |
| | ATATTTTATTATTATTATTATTATTATTATTAATTTATTAATATTAAAGGAAATATATT |
| | TATTATATTATATTATATATATTTTTTATTATTTATATTTATTATTAATATAAATCATTG |
| | ATAATATCTTCTTTTTATTTATTTATTATTATAATTAAAAAGAATCTTATATTAGACCT |
| | GCAGATAAGGTGATTGAATAGAATATAAATCTATATCTTTATTATATTTAAGAATATT |
| | ATTATAATTATTATTATTATTATTATTTTTAATAATTAAAAATATTAATAATAAGTAA |
| | ATATTAAGGATCCTTAAGCATATACAGCTTCGATAGCTTTTTCGAAAGCATCCATACC |
| | TTCTTCGATTAATTCATCTTCGATTGTTAAAGCAGGTACGAATCTTACTGTTGATTTAC |
| | CAGCTGTGATGATTAATAAACCTAATTCTCTAGCTTTTTTGATTACTTCTGTTGGAGG |
| | TTCTACAAATTCAGCACCTAACATTAAACCTTTACCTCTGATTGTTTTGATTTGATTAG |
| | GATATTTAGCTTGGATTTCTCTTAATCTTTTTTGTAAGATATCTGATTTTTTTGATACT |
| | TGTTTTAAGAATGCTTCATCAGCAATTGTATCTAATACATAATTTGATACTGAACAAG |
| | CTAAAGGATTACCACCATATGTTGTACCATGATCACCTACTCTTAAAGCATTATTTAC |
| | TTTTTCATTTACGATTGTAGCAGCAATAGGGAAACCATTACCTAATGCTTTAGCTGAT |
| | GTGAAAATATCAGGATGAGCTTCTGAAGGTAAATAAGCATGAGCTCATAATTTACCT |
| | GATCTACCTAAACCACATTGAATTTCATCATGAATTACGATTACATCATTATCTTGAC |
| | AGATTTTTTTTAAACCAGTTAATTTTTCTACTTCTACAGGAAATACACCACCTTCACCT |
| | TGAATAGGTTCTACGATTAAACCAGCGATTTCATCTTTTTTTGTTTCGATATATGATTG |
| | TAATTTTGTCATTTCATCATTTAAATTTAAGAATGATACATGAGGTACTAAATCACCG |
| | AAAGGTGTTCTATATTTTGAATTTCATGTTACTGATAAAGCACCCATTGTTCTACCAT |
| | GGAATGAATTTTCGAAAGCTACAATACCTTGTTTACTAGGATTTTTCATGATACCATG |
| | TTTTTTAGCGAATTTTAATGCAGCTTCATTAGCTTCGGTACCTGAATTACATAAGAAT |
| | ACTCTTGAAGCATCATGTTGACCACCGAATTGTTTTGTTTTTTCTACGATTTTTTCTGA |
| | TAAATCTAAACATTCTTTTGTGAAATATAAATTTGATGAATGTACTAATTTATTAGCT |
| | TGATGATGTAAGATTTCAGCTACTTTAGGATTAGCATGACCTAAAGCTGTTACAGCA |
| | ATACCAGCTGTGAAATCGATATATTCTTTACCATTTACATCATCATATAATTTAGCAT |
| | TTTTACCTCTTGTGATACATAAATCTTCAGGTCTTGAATATGTTGTTACTTGAAAAGC |
| | TTTTTCTTCTAAGATTGATGTGAATCTTCTTGATGATGTTGATGATAAATATCTTTTGA |
| | ACATTTTAATAAATCTTAACCTTTAGACTCTTTTGTCTATTTATAATATGTTAATACTA |
| | CTTTTAATTAAAATTTATTTATAATTTATTATTATATATTATAAATAAAATAAAAATA |
| | TTATATTACCAAACCCCACCACTAGAAGCGGCCGCTATATATTATGTATTATTATATA |
| | AATATATATATATATTATATTATAAGTAATAATAAGTATTATATTATATATAGCTTTT |
| | ATAGCTTAGTGGTAAAGCGATAAATTGAAGATTTATTTACATGTAGTTCGATTCTCAT |
| | TAAGGGCAATATAGCTATTTTTAGTGGTATGGCGTTTTAGAGCTAGAAATAGCAAGT |
| | TAAAATAAGGCTAGTCCGTTATCAACTTGAAAAAGTGGCACCGAGTCGGTGCTAGGA |
| | GATTAGCTTAATTGGTATAGCATTCGTTTTACACACGAAAGATTATAGGTTCGAACCC |
| | TATATTTCCTAAATACCATGTAAATATTGTGAACCGTTTTAGAGCTAGAAATAGCAA |
| | GTTAAAATAAGGCTAGTCCGTTATCAACTTGAAAAAGTGGCACCGAGTCGGTGCTGA |
| | GCTTGTATAGTTTAATTGGTTAAAACATTTGTCTCATAAATAAATAATGTAAGGTTCA |
| | ATTCCTTCTACAAGTATTATTAATAATTAACAATAATTAATATATTATAATTTATATA |
| | TATATATTTTATATTATTATAATAATATTCTTACAAATATAATTATTATATATTATTCC |
| | TTCAAAACTCCTAACGGCACCATGGACTGTTTTTAAGTATCTATTGATGATTCATAAT |
| | ACTAATGCTAGACGCATCAACGAAATATACGCACTGAGAGGCGCACCATTAAATAAT |
| | TGTGAATTACCATGTTTATTTATATAATTCATCCATACCATGTGTGATACCAGCAGCT |
| | GTTACAAATTCTAATAATACCATATGATCTCTTTTTTCATTAGGATCTTTTGATAAAG |
| | CTGATTGTGTACTTAAGTAATGATTATCAGGTAATAATACAGGACCATCACCGATAG |
| | GTGTATTTTGTTGATAATGATCTGCTAATTGTACTGAACCGTCTTCGATATTATGTCTG |
| | ATTTTGAAATTTACTTTGATACCATTTTTTTGTTTATCAGCCATGATATATACATTATG |
| | TGAATTATAATTATATTCTAATTTATGACCTAAGATATTACCATCTTCTTTGAAATCG |
| | ATACCTTTTAACTCGATTCTATTTACTAATGTATCACCTTCGAATTTTACTTCAGCTCT |
| | TGTTTTATAATTACCATCATCTTTGAAGAAGATTGTTCTTTCTTGTACGTAACCTTCAG |
| | GCATTGCTGATTTGAAGAAATCATGTTGTTTCATATGATCAGGATATCTTGCGAAACA |
| | TTGTACTCCATATCCGAATGTTGTTACTAATGTAGGTCAAGGTACAGGTAATTTACCT |
| | GTTGTACAGATGAATTTTAATGTTAATTTACCATATGTAGCATCACCTTCACCTTCAC |
| | CTGATACTGAGAATTTATGACCATTTACATCACCATCTAATTCTACTAAGATAGGTAC |
| | TACTCCAGTGAATAATTCTTCACCTTTTGACATTTGTCTACTTCTTTCTAAATGTGTCA |
| | TACCAGCTGCTGAACCAGCAGAACCTGATAACATACCACTAAAAATAGCTAACATAA |
| | AATATAATACTGCAATATCTTTTGCATTTGTTGAATATAATCATCTTTGTACCATTCCA |
| | TGGAAAGCTTGGCACTGGCCGTCGTTTTACAACGTCGTGACTGGGAAAACCCTGGCG |
| | TTACCCAACTTAATCGCCTTGCAGCACATCCCCCTTTCGCCAGCTGGCGTAATAGCGA |
| | AGAGGCCCGCACCGATCGCCCTTCCCAACAGTTGCGCAGCCTGAATGGCGAATGGGA |
| | CGCGCCCTGTAGCGGCGCATTAAGCGCGGCGGGTGTGGTGGTTACGCGCAGCGTGAC |
| | CGCTACACTTGCCAGCGCCCTAGCGCCCGCTCCTTTCGCTTTCTTCCCTTCCTTTCTCG |
| | CCACGTTCGCCGGCTTTCCCCGTCAAGCTCTAAATCGGGGGCTCCCTTTAGGGTTCCG |
| | ATTTAGTGCTTTACGGCACCTCGACCCCAAAAAACTTGATTAGGGTGATGGTTCACGT |
| | AGTGGGCCATCGCCCTGATAGACGGTTTTTCGCCCTTTGACGTTGGAGTCCACGTTCT |
| | TTAATAGTGGACTCTTGTTCCAAACTGGAACAACACTCAACCCTATCTCGGTCTATTC |
| | TTTTGATTTATAAGGGATTTTGCCGATTTCGGCCTATTGGTTAAAAAATGAGCTGATT |
| | TAACAAAAATTTAACGCGAATTTTAACAAAATATTAACGTTTACAATTTCAGGTGGC |
| | ACTTTTCGGGGAAATGTGCGCGGAACCCCTATTTGTTTATTTTTCTAAATACATTCAA |
| | ATATGTATCCGCTCATGAGACAATAACCCTGATAAATGCTTCAATAATATTGAAAAA |
| | GGAAGAGTATGAGTATTCAACATTTCCGTGTCGCCCTTATTCCCTTTTTTGCGGCATT |
| | TTGCCTTCCTGTTTTTGCTCACCCAGAAACGCTGGTGAAAGTAAAAGATGCTGAAGA |
| | TCAGTTGGGTGCACGAGTGGGTTACATCGAACTGGATCTCAACAGCGGTAAGATCCT |
| | TGAGAGTTTTCGCCCCGAAGAACGTTTTCCAATGATGAGCACTTTTAAAGTTCTGCTA |
| | TGTGGCGCGGTATTATCCCGTATTGACGCCGGGCAAGAGCAACTCGGTCGCCGCATA |
| | CACTATTCTCAGAATGACTTGGTTGAGTACTCACCAGTCACAGAAAAGCATCTTACG |
| | GATGGCATGACAGTAAGAGAATTATGCAGTGCTGCCATAACCATGAGTGATAACACT |
| | GCGGCCAACTTACTTCTGACAACGATCGGAGGACCGAAGGAGCTAACCGCTTTTTTG |
| | CACAACATGGGGGATCATGTAACTCGCCTTGATCGTTGGGAACCGGAGCTGAATGAA |
| | GCCATACCAAACGACGAGCGTGACACCACGATGCCTGTAGCAATGGCAACAACGTT |
| | GCGCAAACTATTAACTGGCGAACTACTTACTCTAGCTTCCCGGCAACAATTAATAGA |
| | CTGGATGGAGGCGGATAAAGTTGCAGGACCACTTCTGCGCTCGGCCCTTCCGGCTGG |
| | CTGGTTTATTGCTGATAAATCTGGAGCCGGTGAGCGTGGGTCTCGCGGTATCATTGCA |
| | GCACTGGGGCCAGATGGTAAGCCCTCCCGTATCGTAGTTATCTACACGACGGGGAGT |
| | CAGGCAACTATGGATGAACGAAATAGACAGATCGCTGAGATAGGTGCCTCACTGATT |
| | AAGCATTGGTAACTGTCAGACCAAGTTTACTCATATATACTTTAGATTGATTTAAAAC |
| | TTCATTTTTAATTTAAAAGGATCTAGGTGAAGATCCTTTTTGATAATCTCATGACCAA |
| | AATCCCTTAACGTGAGTTTTCGTTCCACTGAGCGTCAGACCCCGTAGAAAAGATCAA |
| | AGGATCTTCTTGAGATCCTTTTTTTCTGCGCGTAATCTGCTGCTTGCAAACAAAAAAA |
| | CCACCGCTACCAGCGGTGGTTTGTTTGCCGGATCAAGAGCTACCAACTCTTTTTCCGA |
| | AGGTAACTGGCTTCAGCAGAGCGCAGATACCAAATACTGTCCTTCTAGTGTAGCCGT |
| | AGTTAGGCCACCACTTCAAGAACTCTGTAGCACCGCCTACATACCTCGCTCTGCTAAT |
| | CCTGTTACCAGTGGCTGCTGCCAGTGGCGATAAGTCGTGTCTTACCGGGTTGGACTCA |
| | AGACGATAGTTACCGGATAAGGCGCAGCGGTCGGGCTGAACGGGGGGTTCGTGCAC |
| | ACAGCCCAGCTTGGAGCGAACGACCTACACCGAACTGAGATACCTACAGCGTGAGC |
| | ATTGAGAAAGCGCCACGCTTCCCGAAGGGAGAAAGGCGGACAGGTATCCGGTAAGC |
| | GGCAGGGTCGGAACAGGAGAGCGCACGAGGGAGCTTCCAGGGGGAAACGCCTGGTA |
| | TCTTTATAGTCCTGTCGGGTTTCGCCACCTCTGACTTGAGCGTCGATTTTTGTGATGCT |
| | CGTCAGGGGGGCGGAGCCTATGGAAAAACGCCAGCAACGCGGCCTTTTTACGGTTCC |
| | TGGCCTTTTGCTGGCCTTTTGCTCACATGTTCTTTCCTGCGTTATCCCCTGATTCTGTG |
| | GATAACCGTATTACCGCCTTTGAGTGAGCTGATACCGCTCGCCGCAGCCGAACGACC |
| | GAGCGCAGCGAGTCAGTGAGCGAGGAAGCGGAAG |
| 36 | KHKHKHKHKHKHKHKHKHMFAFQYLLVM |
| 37 | RKKRRQRRR |
| 38 | RRRRRRRRR |
| 39 | KETWWETWWTEWSQPKKKRKV |
| 40 | GALFLGFLGAAGSTMGAWSQPKKKRKV |
| 41 | VRLPPP |
| 42 | GWTLNSAGYLLGKINLKALAALAKKIL |
| 43 | KLALKLALKALKAALKLA |
| 44 | PLILLRLLRGQF |
| 45 | PLIYLRLLRGQF |
| 46 | KLWMRWYSPTTRRYG |
| 47 | MGLGLHLLVLAAALQGAKKKRKV |
| 48 | KLALKLALKALKAALKLA |
| 49 | LLIILRRRIRKQAHAHSK |
| 50 | RRIPNRRPRR |
| 51 | KKLFKKILKYL |
| 52 | RKKRRQRRRRKKRRQRRR |
| 53 | MLSLRQSIRFFKPATRTLCSSRYLL |
| 54 | MLSLRQSIRFFKPATRTLCSSRYLLDKKYSIGLDIGTNSVGWAVITDEYKVPSKKFKVLGN |
| | TDRHSIKKNLIGALLFDSGETAEATRLKRTARRRYTRRKNRICYLQEIFSNEMAKVDDSFF |
| | HRLEESFLVEEDKKHERHPIFGNIVDEVAYHEKYPTIYHLRKKLVDSTDKADLRLIYLAL |
| | AHMIKFRGHFLIEGDLNPDNSDVDKLFIQLVQTYNQLFEENPINASGVDAKAILSARLSKS |
| | RRLENLIAQLPGEKKNGLFGNLIALSLGLTPNFKSNFDLAEDAKLQLSKDTYDDDLDNLL |
| | AQIGDQYADLFLAAKNLSDAILLSDILRVNTEITKAPLSASMIKRYDEHHQDLTLLKALV |
| | RQQLPEKYKEIFFDQSKNGYAGYIDGGASQEEFYKFIKPILEKMDGTEELLVKLNREDLL |
| | RKQRTFDNGSIPHQIHLGELHAILRRQEDFYPFLKDNREKIEKILTFRIPYYVGPLARGNSR |
| | FAWMTRKSEETITPWNFEEVVDKGASAQSFIERMTNFDKNLPNEKVLPKHSLLYEYFTV |
| | YNELTKVKYVTEGMRKPAFLSGEQKKAIVDLLFKTNRKVTVKQLKEDYFKKIECFDSVE |
| | ISGVEDRFNASLGTYHDLLKIIKDKDFLDNEENEDILEDIVLTLTLFEDREMIEERLKTYAH |
| | LFDDKVMKQLKRRRYTGWGRLSRKLINGIRDKQSGKTILDFLKSDGFANRNFMQLIHDD |
| | SLTFKEDIQKAQVSGQGDSLHEHIANLAGSPAIKKGILQTVKVVDELVKVMGRHKPENIV |
| | IEMARENQTTQKGQKNSRERMKRIEEGIKELGSQILKEHPVENTQLQNEKLYLYYLQNG |
| | RDMYVDQELDINRLSDYDVDHIVPQSFLKDDSIDNKVLTRSDKNRGKSDNVPSEEVVKK |
| | MKNYWRQLLNAKLITQRKFDNLTKAERGGLSELDKAGFIKRQLVETRQITKHVAQILDS |
| | RMNTKYDENDKLIREVKVITLKSKLVSDFRKDFQFYKVREINNYHHAHDAYLNAVVGT |
| | ALIKKYPKLESEFVYGDYKVYDVRKMIAKSEQEIGKATAKYFFYSNIMNFFKTEITLANG |
| | EIRKRPLIETNGETGEIVWDKGRDFATVRKVLSMPQVNIVKKTEVQTGGFSKESILPKRNS |
| | DKLIARKKDWDPKKYGGFDSPTVAYSVLVVAKVEKGKSKKLKSVKELLGITIMERSSFE |
| | KNPIDFLEAKGYKEVKKDLIIKLPKYSLFELENGRKRMLASAGELQKGNELALPSKYVNF |
| | LYLASHYEKLKGSPEDNEQKQLFVEQHKHYLDEIIEQISEFSKRVILADANLDKVLSAYN |
| | KHRDKPIREQAENIIHLFTLTNLGAPAAFKYFDTTIDRKRYTSTKEVLDATLIHQSITGLYE |
| | TRIDLSQLGGDEGA |
| 55 | AAGCTTCGCCACCATGCTTTCACTACGTCAATCTATAAGATTTTTCAAGCCAGCCACA |
| | AGAACTTTGTGTAGCTCTAGATATCTGCTTGACAAGAAGTATTCTATCGGACTGGAC |
| | ATCGGGACTAATAGCGTCGGGTGGGCCGTCATCACTGACGAGTACAAGGTGCCCTCT |
| | AAGAAGTTCAAGGTGCTCGGGAACACCGACCGGCATTCCATCAAGAAAAATCTGATC |
| | GGAGCTCTCCTCTTTGATTCAGGGGAGACCGCTGAAGCAACCCGCCTCAAGCGGACT |
| | GCTAGACGGCGGTACACCAGGAGGAAGAACCGGATTTGTTACCTTCAAGAGATATTC |
| | TCCAACGAAATGGCAAAGGTCGACGACAGCTTCTTCCATAGGCTGGAAGAATCATTC |
| | CTCGTGGAAGAGGATAAGAAGCATGAACGGCATCCCATCTTCGGTAATATCGTCGAC |
| | GAGGTGGCCTATCACGAGAAATACCCAACCATCTACCATCTTCGCAAAAAGCTGGTG |
| | GACTCAACCGACAAGGCAGACCTCCGGCTTATCTACCTGGCCCTGGCCCACATGATT |
| | AAGTTCAGAGGCCACTTCCTGATCGAGGGCGACCTCAATCCTGACAATAGCGATGTG |
| | GATAAACTGTTCATCCAGCTGGTGCAGACTTACAACCAGCTCTTTGAAGAGAACCCC |
| | ATCAATGCAAGCGGAGTCGATGCCAAGGCCATTCTGTCAGCCCGGCTGTCAAAGAGC |
| | CGCAGACTTGAGAATCTTATCGCTCAGCTGCCGGGTGAAAAGAAAAATGGACTGTTC |
| | GGGAACCTGATTGCTCTTTCACTTGGGCTGACTCCCAATTTCAAGTCTAATTTCGACC |
| | TGGCAGAGGATGCCAAGCTGCAACTGTCCAAGGACACCTATGATGACGATCTCGACA |
| | ACCTCCTGGCCCAGATCGGTGACCAATACGCCGACCTTTTCCTTGCTGCTAAGAATCT |
| | TTCTGACGCCATCCTGCTGTCTGACATTCTCCGCGTGAACACTGAAATCACCAAGGCC |
| | CCTCTTTCAGCTTCAATGATTAAGCGGTATGATGAGCACCACCAGGACCTGACCCTG |
| | CTTAAGGCACTCGTCCGGCAGCAGCTTCCGGAGAAGTACAAGGAAATCTTCTTTGAC |
| | CAGTCAAAGAATGGATACGCCGGCTACATCGACGGAGGTGCCTCCCAAGAGGAATTT |
| | TATAAGTTTATCAAACCTATCCTTGAGAAGATGGACGGCACCGAAGAGCTCCTCGTG |
| | AAACTGAATCGGGAGGATCTGCTGCGGAAGCAGCGCACTTTCGACAATGGGAGCATT |
| | CCCCACCAGATCCATCTTGGGGAGCTTCACGCCATCCTTCGGCGCCAAGAGGACTTC |
| | TACCCCTTTCTTAAGGACAACAGGGAGAAGATTGAGAAAATTCTCACTTTCCGCATC |
| | CCCTACTACGTGGGACCCCTCGCCAGAGGAAATAGCCGGTTTGCTTGGATGACCAGA |
| | AAGTCAGAAGAAACTATCACTCCCTGGAACTTCGAAGAGGTGGTGGACAAGGGAGC |
| | CAGCGCTCAGTCATTCATCGAACGGATGACTAACTTCGATAAGAACCTCCCCAATGA |
| | GAAGGTCCTGCCGAAACATTCCCTGCTCTACGAGTACTTTACCGTGTACAACGAGCT |
| | GACCAAGGTGAAATATGTCACCGAAGGGATGAGGAAGCCCGCATTCCTGTCAGGCG |
| | AACAAAAGAAGGCAATTGTGGACCTTCTGTTCAAGACCAATAGAAAGGTGACCGTG |
| | AAGCAGCTGAAGGAGGACTATTTCAAGAAAATTGAATGCTTCGACTCTGTGGAGATT |
| | AGCGGGGTCGAAGATCGGTTCAACGCAAGCCTGGGTACCTACCATGATCTGCTTAAG |
| | ATCATCAAGGACAAGGATTTTCTGGACAATGAGGAGAACGAGGACATCCTTGAGGA |
| | CATTGTCCTGACTCTCACTCTGTTCGAGGACCGGGAAATGATCGAGGAGAGGCTTAA |
| | GACCTACGCCCATCTGTTCGACGATAAAGTGATGAAGCAACTTAAACGGAGAAGATA |
| | TACCGGATGGGGACGCCTTAGCCGCAAACTCATCAACGGAATCCGGGACAAACAGA |
| | GCGGAAAGACCATTCTTGATTTCCTTAAGAGCGACGGATTCGCTAATCGCAACTTCA |
| | TGCAACTTATCCATGATGATTCCCTGACCTTTAAGGAGGACATCCAGAAGGCCCAAG |
| | TGTCTGGACAAGGTGACTCACTGCACGAGCATATCGCAAATCTGGCTGGTTCACCCG |
| | CTATTAAGAAGGGTATTCTCCAGACCGTGAAAGTCGTGGACGAGCTGGTCAAGGTGA |
| | TGGGTCGCCATAAACCAGAGAACATTGTCATCGAGATGGCCAGGGAAAACCAGACT |
| | ACCCAGAAGGGACAGAAGAACAGCAGGGAGCGGATGAAAAGAATTGAGGAAGGGA |
| | TTAAGGAGCTCGGGTCACAGATCCTTAAAGAGCACCCGGTGGAAAACACCCAGCTTC |
| | AGAATGAGAAGCTCTATCTGTACTACCTTCAAAATGGACGCGATATGTATGTGGACC |
| | AAGAGCTTGATATCAACAGGCTCTCAGACTACGACGTGGACCACATCGTCCCTCAGA |
| | GCTTCCTCAAAGACGACTCAATTGACAATAAGGTGCTGACTCGCTCAGACAAGAACC |
| | GGGGAAAGTCAGATAACGTGCCCTCAGAGGAAGTCGTGAAAAAGATGAAGAACTAT |
| | TGGCGCCAGCTTCTGAACGCAAAGCTAATCACTCAGCGGAAGTTCGACAATCTCACT |
| | AAGGCTGAGAGGGGCGGACTGAGCGAACTGGACAAAGCAGGATTCATTAAACGGCA |
| | ACTTGTGGAGACTCGGCAGATTACTAAACATGTCGCCCAAATCCTTGACTCACGCAT |
| | GAATACCAAGTACGACGAAAACGACAAACTTATCCGCGAGGTGAAGGTGATTACCC |
| | TGAAGTCCAAGCTGGTCAGCGATTTCAGAAAGGACTTTCAATTCTACAAAGTGCGGG |
| | AGATCAATAACTATCATCATGCTCATGACGCATATCTGAATGCCGTGGTGGGAACCG |
| | CCCTAATCAAGAAGTACCCAAAGCTGGAAAGCGAGTTCGTGTACGGAGACTACAAG |
| | GTCTACGACGTGCGCAAGATGATTGCCAAATCTGAGCAGGAGATCGGAAAGGCCAC |
| | CGCAAAGTACTTCTTCTACAGCAACATCATGAATTTCTTCAAGACCGAAATCACCCTT |
| | GCAAACGGTGAGATCCGGAAGAGGCCGCTCATCGAGACTAATGGGGAGACTGGCGA |
| | AATCGTGTGGGACAAGGGCAGAGATTTCGCTACCGTGCGCAAAGTGCTTTCTATGCC |
| | TCAAGTGAACATCGTGAAGAAAACCGAGGTGCAAACCGGAGGCTTTTCTAAGGAAT |
| | CAATCCTCCCCAAGCGCAACTCCGACAAGCTCATTGCAAGGAAGAAGGATTGGGACC |
| | CTAAGAAGTACGGCGGATTCGATTCACCAACTGTGGCTTATTCTGTCCTGGTCGTGGC |
| | TAAGGTGGAAAAAGGAAAGTCTAAGAAGCTCAAGAGCGTGAAGGAACTGCTGGGTA |
| | TCACCATTATGGAGCGCAGCTCCTTCGAGAAGAACCCAATTGACTTTCTCGAAGCCA |
| | AAGGTTACAAGGAAGTCAAGAAGGACCTTATCATCAAGCTCCCAAAGTATAGCCTGT |
| | TCGAACTGGAGAATGGGCGGAAGCGGATGCTCGCCTCCGCTGGCGAACTTCAGAAG |
| | GGTAATGAGCTGGCTCTCCCCTCCAAGTACGTGAATTTCCTCTACCTTGCAAGCCATT |
| | ACGAGAAGCTGAAGGGGAGCCCCGAGGACAACGAGCAAAAGCAACTGTTTGTGGAG |
| | CAGCATAAGCATTATCTGGACGAGATCATTGAGCAGATTTCCGAGTTTTCTAAACGC |
| | GTCATTCTCGCTGATGCCAACCTCGATAAAGTCCTTAGCGCATACAATAAGCACAGA |
| | GACAAACCAATTCGGGAGCAGGCTGAGAATATCATCCACCTGTTCACCCTCACCAAT |
| | CTTGGTGCCCCTGCCGCATTCAAGTACTTCGACACCACCATCGACCGGAAACGCTAT |
| | ACCTCCACCAAAGAAGTGCTGGACGCCACCCTCATCCACCAGAGCATCACCGGACTT |
| | TACGAAACTCGGATTGACCTCTCACAGCTCGGAGGGGATGAGGGAGCTTAGTTCGCG |
| | GCCGC |
| 56 | TTCTTTGAAGTATCAGGAGG |
| 57 | ATGATTATTGCAATTCCAAC |
| 58 | TAGCTATTTTTAGTGGTATGGC |
| 59 | ACCATGTAAATATTGTGAACC |
| 60 | GCATGCGGACAATCTTTGAAAAGATAATGTATGATTATGCTTTCACTCATATTTATAC |
| | AGAAACTTGATGTTTTCTTTCGAGTATATACAAGGTGATTACATGTACGTTTGAAGTA |
| | CAACTCTAGATTTTGTAGTGCCCTCTTGGGCTAGCGGTAAAGGTGCGCATTTTTTCAC |
| | ACCCTACAATGTTCTGTTCAAAAGATTTTGGTCAAACGCTGTAGAAGTGAAAGTTGG |
| | TGCGCATGTTTCGGCGTTCGAAACTTCTCCGCAGTGAAAGATAAATGATCATGATTAT |
| | TGCAATTCCAACGTTTTAGAGCTAGAAATAGCAAGTTAAAATAAGGCTAGTCCGTTA |
| | TCAACTTGAAAAAGTGGCACCGAGTCGGTGGCGCAAGTGGTTTAGTGGTAAAATCCA |
| | ACGTTGCCATCGTTGGGCCCCCGGTTCGATTCCGGGCTTGCGCATTCTTTGAAGTATC |
| | AGGAGGGTTTTAGAGCTAGAAATAGCAAGTTAAAATAAGGCTAGTCCGTTATCAACT |
| | TGAAAAAGTGGCACCGAGTCGGTGTTTTTTTGTTTTTTATGTCTTCGAGTCATGTAAT |
| | TAGTTATAAGCATGTGAGCTAAGACACTGTAATTGCCAATCTAAACGATACCACGGC |
| | CGCTCTAGAGAAATGGGGAGCGATTTGCAGGCATTTGCTCGGTGCAGTATAGCGACC |
| | AGCATTCACATACGATTGACGCATGATATTACTTTCTGCGCACTTAACTTCGCATCTG |
| | GGCAGATGATGTCGAGGCGAAAAAAAATATAAATCACGCTAACATTTGATTAAAAT |
| | AGAACAACTACAATATAAAAAAACTATACAAATGACAAGTTCTTGAAAACAAGAAT |
| | CTTTTTATTGTCAGTCTCGAGGCGGCCGC |
| 61 | GCATGCGGACAATCTTTGAAAAGATAATGTATGATTATGCTTTCACTCATATTTATAC |
| | AGAAACTTGATGTTTTCTTTCGAGTATATACAAGGTGATTACATGTACGTTTGAAGTA |
| | CAACTCTAGATTTTGTAGTGCCCTCTTGGGCTAGCGGTAAAGGTGCGCATTTTTTCAC |
| | ACCCTACAATGTTCTGTTCAAAAGATTTTGGTCAAACGCTGTAGAAGTGAAAGTTGG |
| | TGCGCATGTTTCGGCGTTCGAAACTTCTCCGCAGTGAAAGATAAATGATCTAGCTATT |
| | TTTAGTGGTATGGCGTTTTAGAGCTAGAAATAGCAAGTTAAAATAAGGCTAGTCCGT |
| | TATCAACTTGAAAAAGTGGCACCGAGTCGGTGGCGCAAGTGGTTTAGTGGTAAAATC |
| | CAACGTTGCCATCGTTGGGCCCCCGGTTCGATTCCGGGCTTGCGCAACCATGTAAAT |
| | ATTGTGAACCGTTTTAGAGCTAGAAATAGCAAGTTAAAATAAGGCTAGTCCGTTATC |
| | AACTTGAAAAAGTGGCACCGAGTCGGTGTTTTTTTGTTTTTTATGTCTTCGAGTCATG |
| | TAATTAGTTATAAGCATGTGAGCTAAGACACTGTAATTGCCAATCTAAACGATACCA |
| | CGGCCGCTCTAGAGAAATGGGGAGCGATTTGCAGGCATTTGCTCGGTGCAGTATAGC |
| | GACCAGCATTCACATACGATTGACGCATGATATTACTTTCTGCGCACTTAACTTCGCA |
| | TCTGGGCAGATGATGTCGAGGCGAAAAAAAATATAAATCACGCTAACATTTGATTAA |
| | AATAGAACAACTACAATATAAAAAAACTATACAAATGACAAGTTCTTGAAAACAAG |
| | AATCTTTTTATTGTCAGTCTCGAGGCGGCCGC |
| 62 | GCATGCGGACAATCTTTGAAAAGATAATGTATGATTATGCTTTCACTCATATTTATAC |
| | AGAAACTTGATGTTTTCTTTCGAGTATATACAAGGTGATTACATGTACGTTTGAAGTA |
| | CAACTCTAGATTTTGTAGTGCCCTCTTGGGCTAGCGGTAAAGGTGCGCATTTTTTCAC |
| | ACCCTACAATGTTCTGTTCAAAAGATTTTGGTCAAACGCTGTAGAAGTGAAAGTTGG |
| | TGCGCATGTTTCGGCGTTCGAAACTTCTCCGCAGTGAAAGATAAATGATCGCTATTTT |
| | TAGTGGTATGGCGTTTTAGAGCTAGAAATAGCAAGTTAAAATAAGGCTAGTCCGTTA |
| | TCAACTTGAAAAAGTGGCACCGAGTCGGTGGCGCAAGTGGTTTAGTGGTAAAATCCA |
| | ACGTTGCCATCGTTGGGCCCCCGGTTCGATTCCGGGCTTGCGCATGAGACC |
| 63 | GGTCTCACGCACCATGTAAATATTGTGAACCGTTTTAGAGCTAGAAATAGCAAGTTA |
| | AAATAAGGCTAGTCCGTTATCAACTTGAAAAAGTGGCACCGAGTCGGTGTTTTTTTGT |
| | TTTTTATGTCTTCGAGTCATGTAATTAGTTATAAGCATGTGAGCTAAGACACTGTAAT |
| | TGCCAATCTAAACGATACCACGGCCGCTCTAGAGAAATGGGGAGCGATTTGCAGGCA |
| | TTTGCTCGGTGCAGTATAGCGACCAGCATTCACATACGATTGACGCATGATATTACTT |
| | TCTGCGCACTTAACTTCGCATCTGGGCAGATGATGTCGAGGCGAAAAAAAATATAAA |
| | TCACGCTAACATTTGATTAAAATAGAACAACTACAATATAAAAAAACTATACAAATG |
| | ACAAGTTCTTGAAAACAAGAATCTTTTTATTGTCAGTCTCGAGGCGGCCGC |
| 64 | GCTATTTTTAGTGGTATGGC |
| 65 | CCATGTAAATATTGTGAACC |
| 66 | GCATGCGGACAATCTTTGAAAAGATAATGTATGATTATGCTTTCACTCATATTTATAC |
| | AGAAACTTGATGTTTTCTTTCGAGTATATACAAGGTGATTACATGTACGTTTGAAGTA |
| | CAACTCTAGATTTTGTAGTGCCCTCTTGGGCTAGCGGTAAAGGTGCGCATTTTTTCAC |
| | ACCCTACAATGTTCTGTTCAAAAGATTTTGGTCAAACGCTGTAGAAGTGAAAGTTGG |
| | TGCGCATGTTTCGGCGTTCGAAACTTCTCCGCAGTGAAAGATAAATGATCGCTATTTT |
| | TAGTGGTATGGCGTTTTAGAGCTAGAAATAGCAAGTTAAAATAAGGCTAGTCCGTTA |
| | TCAACTTGAAAAAGTGGCACCGAGTCGGTGGCGCAAGTGGTTTAGTGGTAAAATCCA |
| | ACGTTGCCATCGTTGGGCCCCCGGTTCGATTCCGGGCTTGCGCACCATGTAAATATTG |
| | TGAACCGTTTTAGAGCTAGAAATAGCAAGTTAAAATAAGGCTAGTCCGTTATCAACT |
| | TGAAAAAGTGGCACCGAGTCGGTGTTTTTTTGTTTTTTATGTCTTCGAGTCATGTAAT |
| | TAGTTATAAGCATGTGAGCTAAGACACTGTAATTGCCAATCTAAACGATACCACGGC |
| | CGCTCTAGAGAAATGGGGAGCGATTTGCAGGCATTTGCTCGGTGCAGTATAGCGACC |
| | AGCATTCACATACGATTGACGCATGATATTACTTTCTGCGCACTTAACTTCGCATCTG |
| | GGCAGATGATGTCGAGGCGAAAAAAAATATAAATCACGCTAACATTTGATTAAAAT |
| | AGAACAACTACAATATAAAAAAACTATACAAATGACAAGTTCTTGAAAACAAGAAT |
| | CTTTTTATTGTCAGTCTCGAGGCGGCCGC |
| 67 | AAGCTTTCCATGGAATGGTACAAAGATGATTATATTCAACAAATGCAAAAGATATTG |
| | CAGTATTATATTTTATGTTAGCTATTTTTAGTGGTATGTTATCAGGTTCTGCTGGTTCA |
| | GCAGCTGGTATGACACATTTAGAAAGAAGTAGACAAATGTCAAAAGGTGAAGAATT |
| | ATTCACTGGAGTAGTACCTATCTTAGTAGAATTAGATGGTGATGTAAATGGTCATAA |
| | ATTCTCAGTATCAGGTGAAGGTGAAGGTGATGCTACATATGGTAAATTAACATTAAA |
| | ATTCATCTGTACAACAGGTAAATTACCTGTACCTTGACCTACATTAGTAACAACATTC |
| | GGATATGGAGTACAATGTTTCGCAAGATATCCTGATCATATGAAACAACATGATTTC |
| | TTCAAATCAGCAATGCCTGAAGGTTACGTACAAGAAAGAACAATCTTCTTCAAAGAT |
| | GATGGTAATTATAAAACAAGAGCTGAAGTAAAATTCGAAGGTGATACATTAGTAAAT |
| | AGAATCGAGTTAAAAGGTATCGATTTCAAAGAAGATGGTAATATCTTAGGTCATAAA |
| | TTAGAATATAATTATAATTCACATAATGTATATATCATGGCTGATAAACAAAAAAAT |
| | GGTATCAAAGTAAATTTCAAAATCAGACATAATATCGAAGACGGTTCAGTACAATTA |
| | GCAGATCATTATCAACAAAATACACCTATCGGTGATGGTCCTGTATTATTACCTGATA |
| | ATCATTACTTAAGTACACAATCAGCTTTATCAAAAGATCCTAATGAAAAAAGAGATC |
| | ATATGGTATTATTAGAATTTGTAACAGCTGCTGGTATCACACATGGTATGGATGAATT |
| | ATATAAATAAACATGGTAATTCACAATTATTTAATGGTGCGCCTCTCAGTGCGTATAT |
| | TTCGTTGATGCGTCTAGCATTAGTATTATGAATCATCAATAGATACTTAAAAACAGTC |
| | CATGTTCTGCAG |
| 68 | AGAATCAGGTGCTGGTACAGGGTGA |
| 69 | CTATTCAGGCACATTCAGGACC |
| 70 | AGAGGTATACCAACACAAGATTC |
| 71 | AGATAGATAATCAATTCAACCATCTGT |
| 72 | ATTAGTTCGGTTTAGTTGGTATTTTGTAATGAGTAAAAAGT |
| 73 | CCTACACTAATCATAGGTGTTTTATGACATGCTA |
| 74 | AGATTATGAAAGAGAGTATTAATATCA |
| 75 | TAAAGTTAGCCCCTACTGAGTTA |
| 76 | CAGGTGAAGGTGAAGGTGATGC |
| 77 | GATCTGCTAATTGTACTGAACCG |
| 78 | CAGCAATGCCTGAAGGTTACGTAC |
| 79 | ACTAATGTAGGTCAAGGTACAGG |
| 80 | AAGCTTCCATCGAATGGTACAAAGATGATTATATTCAACAAATGCAAAAGATATTGC |
| | AGTATTATATTTTATGTTAGCTATTTTTAGTGGTATGTTATCAGGTTCTGCTGGTTCAG |
| | CAGCTGGTTTCAAAAGATATTTATCATCAACATCATCAAGAAGATTCACATCAATCTT |
| | AGAAGAAAAAGCATTTCAAGTAACAACATATTCAAGACCTGAAGATTTATGTATCAC |
| | AAGAGGTAAAAATGCTAAATTATATGATGATGTAAATGGTAAAGAATATATCGATTT |
| | CACAGCTGGTATTGCTGTAACAGCTTTAGGTCATGCTAATCCTAAAGTAGCTGAAAT |
| | CTTACATCATCAAGCTAATAAATTAGTACATTCATCAAATTTATATTTCACAAAAGAA |
| | TGTTTAGATTTATCAGAAAAAATCGTAGAAAAAACAAAACAATTCGGTGGTCAACAT |
| | GATGCTTCAAGAGTATTCTTATGTAATTCAGGTACCGAAGCTAATGAAGCTGCATTA |
| | AAATTCGCTAAAAAACATGGTATCATGAAAAATCCTAGTAAACAAGGTATTGTAGCT |
| | TTCGAAAATTCATTTCATGGTAGAACAATGGGTGCTTTATCAGTAACATGAAATTCA |
| | AAATATAGAACACCTTTCGGTGATTTAGTACCTCATGTATCATTCTTAAATTTAAATG |
| | ATGAAATGACAAAATTACAATCATATATCGAAACAAAAAAAGATGAAATCGCTGGT |
| | TTAATCGTAGAACCTATTCAAGGTGAAGGTGGTGTATTTCCTGTAGAAGTAGAAAAA |
| | TTAACTGGTTTAAAAAAAATCTGTCAAGATAATGATGTAATCGTAATTCATGATGAA |
| | ATTCAATGTGGTTTAGGTAGATCAGGTAAATTATGAGCTCATGCTTATTTACCTTCAG |
| | AAGCTCATCCTGATATTTTCACATCAGCTAAAGCATTAGGTAATGGTTTCCCTATTGC |
| | TGCTACAATCGTAAATGAAAAAGTAAATAATGCTTTAAGAGTAGGTGATCATGGTAC |
| | AACATATGGTGGTAATCCTTTAGCTTGTTCAGTATCAAATTATGTATTAGATACAATT |
| | GCTGATGAAGCATTCTTAAAACAAGTATCAAAAAAATCAGATATCTTACAAAAAAGA |
| | TTAAGAGAAATCCAAGCTAAATATCCTAATCAAATCAAAACAATCAGAGGTAAAGG |
| | TTTAATGTTAGGTGCTGAATTTGTAGAACCTCCAACAGAAGTAATCAAAAAAGCTAG |
| | AGAATTAGGTTTATTAATCATCACAGCTGGTAAATCAACAGTAAGATTCGTACCTGC |
| | TTTAACAATCGAAGATGAATTAATCGAAGAAGGTATGGATGCTTTCGAAAAAGCTAT |
| | CGAAGCTGTATATGCTTAAACATGGTAATTCACAATTATTTAATGGTGCGCCTCTCAG |
| | TGCGTATATTTCGTTGATGCGTCTAGCATTAGTATTATGAATCATCAATAGATACTTA |
| | AAAACAGTCCATGG |
| 81 | FKRYLSSTSSRRFTSILEEKAFQVTTYSRPEDLCITRGKNAKLYDDVNGKEYIDFTAGIAV |
| | TALGHANPKVAEILHHQANKLVHSSNLYFTKECLDLSEKIVEKTKQFGGQHDASRVFLC |
| | NSGTEANEAALKFAKKHGIMKNPSKQGIVAFENSFHGRTMGALSVTWNSKYRTPFGDL |
| | VPHVSFLNLNDEMTKLQSYIETKKDEIAGLIVEPIQGEGGVFPVEVEKLTGLKKICQDNDV |
| | IVIHDEIQCGLGRSGKLWAHAYLPSEAHPDIFTSAKALGNGFPIAATIVNEKVNNALRVG |
| | DHGTTYGGNPLACSVSNYVLDTIADEAFLKQVSKKSDILQKRLREIQAKYPNQIKTIRGK |
| | GLMLGAEFVEPPTEVIKKARELGLLIITAGKSTVRFVPALTIEDELIEEGMDAFEKAIEAVY |
| | A |
| 82 | GCGGCCGCTATATATTATGTATTATTATATAAATATATATATATATTATATTATAAGT |
| | AATAATAAGTATTATATTATATATAGCTTTTATAGCTTAGTGGTAAAGCGATAAATTG |
| | AAGATTTATTTACATGTAGTTCGATTCTCATTAAGGGCAATAGCTATTTTTAGTGGTA |
| | TGGCGTTTTAGAGCTAGAAATAGCAAGTTAAAATAAGGCTAGTCCGTTATCAACTTG |
| | AAAAAGTGGCACCGAGTCGGTGCTAGGAGATTAGCTTAATTGGTATAGCATTCGTTT |
| | TACACACGAAAGATTATAGGTTCGAACCCTATATTTCCTAAATCCATGTAAATATTGT |
| | GAACCGTTTTAGAGCTAGAAATAGCAAGTTAAAATAAGGCTAGTCCGTTATCAACTT |
| | GAAAAAGTGGCACCGAGTCGGTGCTGAGCTTGTATAGTTTAATTGGTTAAAACATTT |
| | GTCTCATAAATAAATAATGTAAGGTTCAATTCCTTCTACAAGTATTATTAATAATTAA |
| | CAATAATTAATATATTATAATTTATATATATATATTTTATATTATTATAATAATATTCT |
| | TACAAATATAATTATTATATATTATTCCTTCAAAACTCCTAACGGCACCATGG |
| 83 | TCTAGATCAAATATATAAGTAATAGGGGGAGGGGGTGGGTGATAATAACCAGAATA |
| | TTAAATAAATACAGAGCACACATTTGTTAATATTTAATAATATAATCAATAAATATAT |
| | TATAATAATATAATATAATTAATAATAGATATAAAGTATAAACAATATAATAAATTA |
| | TATAAAATAAATATAAATTAAAAATAATAACCAAATAATTAATATAATAAATGATAA |
| | ACAAGAAGATATCCGGGTCCCAATAATAATTATTATTGAAAATAATAATTGGGACCC |
| | CCACAATGCGGCCGC |
| 84 | GAAAAGTGCCACCTGGGTCCTTTTCATCACG |
| 85 | GACGAAAGGGCCTCGTGATACGCCTAT |
| 86 | CACATTCAGGACCTAGTGTAGATTTAGCAATTTTTGCATTACATTTAACATCAATTTC |
| | ATCATTATTAGGTGCTATTAATTTCATTGTAACAACATTAAATATGAGAACAAATGGT |
| | ATGACAATGCATAAATTACCATTATTTGTATGATCAATTTTCATTACAGCGTTCTTAT |
| | TATTATTATCATTACCTGTATTATCTGCTGGTATTACAATGTTATTATTAGATAGAAA |
| | CTTCAATACTTCATTTTTCGGAGTTTCTGGTGGAGGTGGTGGAATGACACATTTAGAA |
| | AGAAGTAGACAAATGTCAAAAGGTGAAGAATTATTCACTGGAGTAGTACCTATCTTA |
| | GTAGAATTAGATGGTGATGTAAATGGTCATAAATTCTCAGTATCAGGTAAAGGTGAA |
| | GGTGATGCTACATATGGTAAATTAACATTAAAATTCATCTGTACAACAGGTAAATTA |
| | CCTGTACCTT |
| 87 | GAGGTTACGTACAAGAAAGAACAATCTTCTTCAAAGATGATGGTAATTATAAAACAA |
| | GAGCTGAAGTAAAATTCGAAGGTGATACATTAGTAAATAGAATCGAGTTAAAAGGT |
| | ATCGATTTCAAAGAAGATGGTAATATCTTAGGTCATAAATTAGAATATAATTATAAT |
| | TCACATAATGTATATATCATGGCTGATAAACAAAAAAATGGTATCAAAGTAAATTTC |
| | AAAATCAGACATAATATCGAAGACGGTTCAGTACAATTAGCAGATCATTATCAACAA |
| | AATACACCTATCGGTGATGGTCCTGTATTATTACCTGATAATCATTACTTAAGTACAC |
| | AATCAGCTTTATCAAAAGATCCTAATGAAAAAAGAGATCATATGGTATTATTAGAAT |
| | TTGTAACAGCTGCTGGTATCACACATGGTATGGATGAATTATATAAATAACAACAGG |
| | AATTAAAATTTTCTCATGATTAATAAATCCCTTTAGCAAGGATAAAAATAAAAATAA |
| | AAATAAAAAGTTGATCAGAAATTATCAAAAAATAAATAATAATAATATAATAAAAA |
| | CATATTTAAATAATAATAATATAATTATAATAAATATATATAAAGGTAATTTATATGA |
| | TATTTATCCAAGATCAAATAGAAATTATATTCAACCAAATAATATTAATAAAGAATT |
| | AGTAGTATATGGTTATAATTTAGAATCTT |

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A and FIG. 1B show results of PCR reactions to detect junctions of integrated donor DNA at sites recognized by gRNAs 1 and 2. FIG. 1A and FIG. 1B: lanes 1 & 2 show two independent positive samples (pHS97×HS100); lanes 3 & 4 show two independent negative control samples (pNY93×HS100); and lane 5 shows a PCR control without any cells. The flanking lanes show a 1 kb Plus DNA Ladder. FIG. 1A: shows a right junction amplified by primers F1-11 and F-13. FIG. 1B: shows a left junction amplified by primers 1-12 and C-15.

FIG. 2A and FIG. 2B show results of PCR reactions to detect junctions of integrated donor DNA at sites recognized by gRNAs 1 and 2. FIG. 2A and FIG. 2B: lane 1 is a negative control sample (pYES2×HS100); lanes 2 & 3 are two independent positive samples (pHS97×HS100 and pDM97×HS100); and lane 4 is a PCR control without any cells. The flanking lanes show a 1 kb Plus DNA Ladder (New England BioLabs, Inc.). FIG. 2A: shows a right junction amplified by primers F1-11 and F-13. FIG. 2B: shows a left junction amplified by primers I-12 and C-15.

### DETAILED DESCRIPTION

The present disclosure now will be described more fully hereinafter but should not be construed as limited to the embodiments set forth herein.

The meaning of abbreviations can be as follows: “sec” can mean second(s), “min” can mean minute(s), “h” can mean hour(s), “d” can mean day(s), “μL” can mean microliter(s), “ml” can mean milliliter(s), “L” can mean liter(s), “μM” can mean micromolar, “mM” can mean millimolar, “M” can mean molar, “mmol” can mean millimole(s), “μmole” can mean micromole(s), “g” can mean gram(s), “μg” can mean microgram(s), “ng” can mean nanogram(s), “U” can mean unit(s), “nt” can mean nucleotide(s); “bp” can mean base pair(s), “kb” can mean kilobase(s) and “kbp” can mean kilobase pair(s).

“Transgenic” can refer to any cell, cell line, callus, tissue, organism part or whole organism (e.g., plant), the genome of which has been altered by the presence of a heterologous nucleic acid, such as a recombinant DNA construct. Transgenic events can include those created by sexual crosses or asexual propagation. In some embodiments, the term “transgenic” may not encompass the alteration of the genome (e.g., chromosomal or extra-chromosomal) by breeding methods or by naturally occurring events such as random cross-fertilization, non-recombinant viral infection, non-recombinant bacterial transformation, non-recombinant transposition, or spontaneous mutation. In some embodiments, the term “transgenic” may encompass the alteration of the genome (e.g., chromosomal or extra-chromosomal) by breeding methods or by naturally occurring events such as random cross-fertilization, non-recombinant viral infection, non-recombinant bacterial transformation, non-recombinant transposition, or spontaneous mutation.

“Genome”, for example, of a cell or whole organism can encompass chromosomal DNA found within the nucleus (nuclear DNA), and organellar DNA (e.g., mitochondrial DNA, plastid DNA) found within subcellular components of the cell. Methods and compositions of the disclosure can be used for editing of the nuclear genome, organellar genome (e.g., mitochondria, chloroplasts), or both.

The terms “full complement” and “full-length complement” can be used interchangeably herein, and can refer to a complement of a given nucleotide sequence. In some aspects, the complement and the nucleotide sequence comprise of the same number of nucleotides. In some aspects, the complement and the nucleotide sequence can comprise 100% complementary. The complement and the nucleotide sequence can differ in the number of nucleotides. Complementarity (e.g., between the complement and the nucleotide sequence) can be at least about 10%, at least about 20%, at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. Complementarity (e.g., between the complement and the nucleotide sequence) can be at most about 10%, at most about 20%, at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%.

“Polynucleotide”, “nucleic acid”, “nucleic acid sequence”, “nucleotide sequence”, or “nucleic acid fragment”, which can be used interchangeably, can refer to a polymer of a nucleic acid (e.g., RNA, DNA, or both, and analogs thereof) that can be single-stranded or double-stranded, optionally containing synthetic, non-natural or altered nucleotide bases. Nucleotides (e.g., in their 5′-monophosphate form) can be referred to by their single letter designation as follows (for RNA or DNA, respectively): “A” for adenylate or deoxyadenylate, “C” for cytidylate or deoxycytidylate, “G” for guanylate or deoxyguanylate, “U” for uridylate, “T” for deoxythymidylate, “R” for purine-based nucleotides (A or G), “Y” for pyrimidine-based nucleotides (C or T), “K” for G or T, “H” for A or C or T, “I” for inosine, and “N” for any nucleotide.

“Polypeptide”, “peptide”, “amino acid sequence” and “protein”, which can be used interchangeably herein, can refer to a polymer of amino acid residues. The terms can apply to amino acid polymers in which one or more amino acid residue can be, for example, an artificial chemical analogue of a corresponding naturally occurring amino acid and/or to naturally occurring amino acid polymers. The terms “polypeptide”, “peptide”, “amino acid sequence”, and “protein” can be inclusive of modifications including, but not limited to, glycosylation, lipid attachment, sulfation, gamma-carboxylation of glutamic acid residues, hydroxylation and ADP-ribosylation.

A “functional fragment” of a polynucleotide or polypeptide can refer to any subset of contiguous nucleotides or contiguous amino acids, respectively, in which the original (e.g., wild type) activity (or substantially similar activity) of the polynucleotide or polypeptide can be retained. The terms “functional fragment”, “functional subfragment”, “fragment that is functionally equivalent”, “subfragment that is functionally equivalent”, “functionally equivalent fragment” and “functionally equivalent subfragment” can be used interchangeably herein.

The terms “functional variant”, “variant that is functionally equivalent” and “functionally equivalent variant” can be used interchangeably herein. In the context of a polynucleotide or a polypeptide, these terms can refer to a variant of the nucleic acid sequence or the amino acid sequence, respectively, in which the original activity (or substantially similar activity) of the polynucleotide or polypeptide can be retained. Fragments and variants can be obtained via methods such as site-directed mutagenesis and synthetic construction.

The activity of the functional fragment or function variant can be, for example, about: 100%, 95%, 90%, 85%, 80%, 75%, 70%, 65%, 60%, 55%, 50%, 40%, 30%, 20%, 10%, or less than 10% of that of the original (e.g., wild type) activity.

“RNA transcript” can refer to the product resulting from RNA polymerase-catalyzed transcription of a DNA sequence. When the RNA transcript is a perfect complimentary copy of the DNA sequence, it can be referred to as the primary transcript. An RNA transcript can be referred to as the mature RNA, for example, when it is an RNA sequence derived from post-transcriptional processing of the primary transcript.

“Messenger RNA” or “mRNA” can refer to the RNA that is without introns and that can be translated into protein by the cell.

“Sense” RNA can refer to the RNA transcript that includes the mRNA. Sense RNA can be translated into protein within a cell or in vitro.

“Antisense RNA” can refer to an RNA transcript that can be complementary to all or part of a target RNA (e.g., a primary transcript or mRNA). Antisense RNA can be used to block expression of a target gene. The complementarity of an antisense RNA may be with any part of the specific gene transcript, i.e., at the 5′ non-coding sequence, 3′ non-coding sequence, introns, or the coding sequence. “Functional RNA” can refer to antisense RNA, ribozyme RNA, or other RNA that may not be translated but yet can have an effect on cellular processes. The terms “complement” and “reverse complement” can be used interchangeably herein, for example, with respect to mRNA transcripts and may be used to define the antisense RNA of the message.

“cDNA” can refer to a DNA that can be complementary to and synthesized from a mRNA template using the enzyme reverse transcriptase. The cDNA can be single-stranded or converted into the double-stranded form using the Klenow fragment of DNA polymerase I.

“Coding region” can refer to the portion of a messenger RNA (or the corresponding portion of another nucleic acid molecule such as a DNA molecule) which can encode a protein or polypeptide. “Non-coding region” can refer to a portion of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” can be used interchangeably herein. The terms “non-coding region” and “non-coding sequence” can be used interchangeably herein.

“Coding sequence” can be abbreviated “CDS”. “Open reading frame” can be abbreviated “ORF”.

An “Expressed Sequence Tag” (“EST”) can be a DNA sequence derived from a cDNA library. An EST can be a sequence which has been transcribed. An EST can be obtained by a single sequencing pass of a cDNA insert. The sequence of an entire cDNA insert can be termed the “Full-Insert Sequence” (“FIS”). A “Contig” sequence can be a sequence assembled from two or more sequences that can be selected from, but not limited to, the group consisting of an EST, an FIS, a PCR sequence, or any combination thereof. A sequence encoding an entire or functional protein can be termed a “Complete Gene Sequence” (“CGS”). A CGS can be derived from an FIS or a contig.

“Gene” can refer to a nucleic acid fragment that can express a functional molecule such as, but not limited to, a specific protein, including: introns, exons, regulatory sequences preceding (5′ non-coding sequences) and following (3′ non-coding sequences) the coding sequence. “Native gene” can refer to a gene as found in nature, for example, with its own regulatory sequences.

A “mutated gene” can be a gene that has been altered relative to the corresponding naturally occurring gene; e.g., through human intervention. Such a “mutated gene” can have a sequence that differs from the sequence of the corresponding non-mutated gene by at least one nucleotide addition, deletion, or substitution. In certain embodiments of the disclosure, the mutated gene can comprise an alteration that results from a polynucleotide guided polypeptide system as disclosed herein. A mutated organism can be an organism comprising a mutated gene; e.g., a mutated plant with an organellar genome comprising a mutated gene. The terms “mutated gene” and “mutant gene” can be used interchangeably herein.

A “silent mutation” can refer to a mutated sequence that has the same functionality as the wild-type sequence; e.g., replacement of a codon in a protein-coding region with a synonymous codon that can encode the same amino acid.

As used herein, a “targeted mutation” can be a DNA modification made at or near a specific target site in the genome. The targeted mutation may be as small as a single nucleotide change in a native gene. The targeted mutation may involve a larger DNA modification such as the insertion of one or more heterologous DNAs; e.g., a heterologous regulatory element, a heterologous protein-coding sequence, or an expression cassette coding for a heterologous protein or functional RNA. The targeted mutation may also involve a change in the sequence of a target site.

The term “SDN” can refer to “site-directed nuclease”. The following are non-limiting examples of SDN-induced mutations: (1) induction of site-specific random mutations; (2) the induction of mutations in a predefined sequence of a particular gene; and (3) the replacement or the insertion of an entire gene. These SDN-induced mutations can be referred to as SDN-1, SDN-2 and SDN-3, respectively.

A “codon-modified gene” or “codon-preferred gene” or “codon-optimized gene” can be a gene having its frequency of codon usage designed to mimic the frequency of preferred codon usage of the host cell in the compartment of interest, e.g., the nucleus, the mitochondria or the chloroplast.

“Mature” protein can refer to a post-translationally processed polypeptide; for example, one from which any pre- or pro-peptides present in the primary translation product have been removed.

“Precursor” protein can refer to the primary product of translation of an mRNA; for example, with pre- and pro-peptides still present. Pre- and pro-peptides may, for example, comprise intracellular localization signals.

“Isolated” can refer to materials, such as nucleic acid molecules, proteins, and cells that may be substantially free or otherwise removed from components that normally accompany or interact with the materials in a naturally occurring environment. Isolated polynucleotides may be purified from a host cell in which they naturally occur. Nucleic acid purification methods can be used to obtain isolated polynucleotides. Isolated polynucleotides can include, for example, recombinant polynucleotides and chemically synthesized polynucleotides.

“Heterologous”, for example, with respect to sequence, can mean a sequence that originates from a foreign species, or, if from the same species, is substantially modified from its native form in composition and/or genomic locus by deliberate human intervention. The terms “heterologous nucleotide sequence”, “heterologous sequence”, “heterologous nucleic acid fragment”, and “heterologous nucleic acid sequence” can be used interchangeably herein.

“Recombinant” can refer to an artificial combination of two or more otherwise separated segments of sequence, e.g., by chemical synthesis or by the manipulation of isolated segments of nucleic acids by genetic engineering techniques. “Recombinant” can also include reference to a cell or vector, for example, that has been modified by the introduction of a heterologous nucleic acid or a cell derived from a cell so modified.

“Recombinant DNA construct” can refer to a combination of nucleic acid fragments that may not normally be found together in nature. A recombinant DNA construct may comprise, for example, regulatory sequences and coding sequences that are derived from different sources, or regulatory sequences and coding sequences derived from the same source. The sequences in a recombinant DNA construct can be arranged in a manner different than that normally found in nature. The terms “recombinant DNA construct”, “recombinant DNA molecule”, “recombinant construct”, “DNA construct” and “construct” can be used interchangeably herein.

“Expression” can refer to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Expression cassette” can refer to a construct containing, for example, a polynucleotide, a regulatory element(s), and a polynucleotide that allow for expression of the polynucleotide in a host. The terms “expression cassette” and “expression construct” can be used interchangeably herein.

The terms “entry clone” and “entry vector” can be used interchangeably herein. A vector is a polynucleotide (e.g., DNA or RNA) used as a vehicle to artificially carry genetic material into a cell, where it can be replicated and/or expressed. Such a polynucleotide can be in the form of a plasmid, YAC, cosmid, phagemid, BAC, virus, or linear DNA (e.g., linear PCR product), for example, or any other type of construct useful for transferring a polynucleotide sequence into another cell. A vector (or portion thereof) can exist transiently (i.e., not integrated into the genome) or stably (i.e., integrated into the genome) in the target cell.

The terms “regulatory domain”, “regulatory element” and “regulatory sequence” can be used interchangeably herein. Regulatory sequences refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to: promoters, translation leader sequences, 5′ untranslated sequences (5′-UTR), 3′ untranslated sequences (3′-UTR), introns, polyadenylation target sequences, RNA processing sites (e.g., RNA editing sites), effector binding sites, and stem-loop structures. A regulatory element may act in “cis” or “trans”, and generally it acts in “cis”; i.e., it activates expression of genes located on the same nucleic acid molecule (e.g., a chromosome or a plasmid DNA) where the regulatory element is located. The nucleic acid molecule regulated by a regulatory element does not necessarily have to encode a functional peptide or polypeptide; e.g., the regulatory element can modulate the expression of a short interfering RNA or an anti-sense RNA.

“Promoter” can refer to a nucleic acid fragment that can control transcription of another nucleic acid fragment. A promoter can include a core promoter (also known as minimal promoter) sequence. A core promoter can be a minimal sequence for direct transcription initiation. A core promoter can optionally include enhancers or other regulatory elements. Promoters may be derived in their entirety from a native gene, or be composed of different elements derived from different promoters found in nature, or even comprise synthetic DNA segments. Different promoters may direct the expression of a gene in different tissues or cell types, or at different stages of development, or in response to different environmental conditions.

“Promoter functional in a plant” can be a promoter that can control transcription in plant cells. The promoter can be from any suitable origin, which can include plant cells and non-plant cells.

“Tissue-specific promoter” and “tissue-preferred promoter” can be used interchangeably, and can refer to a promoter that can be expressed predominantly in one tissue, one organ or one cell type. A tissue-specific promoter may not be necessarily exclusive in one tissue, one organ or one cell type. Root-preferred promoters include, for example, the following: soybean root-specific glutamine synthase gene; cytosolic glutamine synthase (GS); root-specific control element in the GRP 1.8 gene of French bean; root-specific promoter of A. tumefaciens mannopine synthase (MAS); root-specific promoters isolated from Parasponia andersonii and Trema tomentosa; A. rhizogenes rolC and rolD root-inducing genes; Agrobacterium wound-induced TR1′ and TR2′ genes; VfENOD-GRP3 gene promoter; and rolB promoter. Seed-preferred promoters include both seed-specific promoters active during seed development, as well as seed-germinating promoters active during seed germination. Seed-preferred promoters include, but are not limited to, the following: Cim1 (cytokinin-induced message); cZ19B1 (maize 19 kDa zein); milps (myo-inositol-1-phosphate synthase); END1; and END2. For dicots, seed-preferred promoters include, but are not limited to, the following: bean β-phaseolin; napin; β-conglycinin; soybean lectin; cruciferin; and the like. For monocots, seed-preferred promoters include, but are not limited to, the following: maize 15 kDa zein; 22 kDa zein; 27 kDa gamma zein; waxy; shrunken 1; shrunken 2; globulin 1; oleosin; nud; and Zea mays-Rootmet2 promoter. Leaf-preferred promoters include, but are not limited to, the following: plant rbcS promoters, such as the soybean rbcS promoter and the maize rbcS promoter; Zea mays PEPC1 promoter.

“Developmentally regulated promoter” can refer to a promoter whose activity can be determined by developmental events.

“Inducible promoter” can refer to a promoter that selectively expresses an operably linked DNA sequence in response to the presence of an endogenous or exogenous stimulus, for example by chemical compounds (e.g., chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners. Pathogen-inducible promoters induced following infection by a pathogen include, but are not limited to those regulating expression of PR proteins, SAR proteins, beta-1,3-glucanase, chitinase, etc. Stress-inducible promoters include plant RAB17 promoters, such as the maize RAB17 promoter. Chemical-inducible promoters include, but are not limited to, the following: the maize ln2-2 promoter, activated by benzene sulfonamide herbicide safeners; the maize GST promoter, activated by hydrophobic electrophilic compounds used as pre-emergent herbicides; and the tobacco PR-1a promoter, activated by salicylic acid. Other chemical-regulated promoters include steroid-responsive promoters, for example, the glucocorticoid-inducible promoter, and tetracycline-inducible and tetracycline-repressible promoters.

“Constitutive promoter” can refer to promoters active in all or most tissues or cell types of an organism at all or most developing stages. As with other promoters classified as “constitutive” (e.g. ubiquitin), some variation in absolute levels of expression can exist among different tissues or stages. The term “constitutive promoter” or “tissue-independent promoter” can be used interchangeably herein. Constitutive promoters include the following: the core promoter of the Rsyn7 promoter; the core CaMV 35S promoter; plant actin promoter, such as a rice actin promoter and a maize actin promoter; plant ubiquitin promoter, such as a maize ubiquitin promoter and a soybean ubiquitin promoter; pEMU; MAS promoter; ALS promoter; plant GOS2 promoter, such as a maize GOS2 promoter; soybean GM-EF1 A2 promoter; plant U6 polymerase III promoter, such as a maize U6 polymerase III promoter and a soybean U6 polymerase III promoter (GM-U6-9.1 and GM-U6-13.1).

An enhancer element can be any nucleic acid molecule that increases transcription of a nucleic acid molecule when functionally linked to a promoter regardless of its relative position. An enhancer may be an innate element of the promoter or a heterologous element inserted to enhance the level or tissue-specificity of a promoter.

A repressor (also sometimes called herein silencer) can be defined as any nucleic acid molecule which inhibits the transcription when functionally linked to a promoter regardless of relative position.

“Translation leader sequence” can refer to a polynucleotide sequence located between the promoter sequence of a gene and the coding sequence. The translation leader sequence can be present in the fully processed mRNA upstream of the translation start sequence. The translation leader sequence may affect processing of the primary transcript to mRNA, mRNA stability or translation efficiency.

“Transcription terminator”, “termination sequence”, or “terminator” can refer to DNA sequences that, when operably linked to the 3′ end of a polynucleotide sequence that is to be expressed, can terminate transcription from the polynucleotide sequence. Transcription termination can refer to the process by which RNA synthesis by RNA polymerase can be stopped and both the RNA and the enzyme are released from the DNA template.

The term “operably linked” refers to the association of two (or more) molecules in which the function of one is influenced by the association with the other(s). The association of the molecules may be in a covalent or non-covalent manner, or both. In a non-limiting example, two nucleic acid sequences may be present on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). In another non-limiting example, a nucleic acid may be associated with a protein, so that the function of one is regulated by the other. For example, a guide RNA may be associated with a polynucleotide-guided polypeptide (e.g., Cas9), such that the complex is capable of cleaving a DNA target site.

“Phenotype” can refer to the detectable characteristics of a cell or organism.

The term “introduced” can mean providing a polynucleic acid (e.g., expression construct) or protein into a cell. Introduced can include reference to the incorporation of a nucleic acid into a eukaryotic or prokaryotic cell, for example, where the nucleic acid may be incorporated into the genome of the cell. Introduced can include reference to the transient provision of a nucleic acid or protein to the cell. Introduced can include reference to stable or transient transformation methods. Introduced can include sexually crossing. Introduced, for example, in the context of inserting a nucleic acid fragment (e.g., a recombinant DNA construct) into a cell, can include “transfection” or “transformation” or “transduction”. Introduced can include reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” can be any cell into which a nucleic acid fragment (e.g., a recombinant DNA construct) has been introduced.

“Transformation” as used herein can refer to stable transformation. Transformation can refer to transient transformation.

“Stable transformation” can refer to the introduction of a nucleic acid fragment into a genome of a host organism resulting in genetically stable inheritance. Once stably transformed, the nucleic acid fragment can be stably integrated in the genome of the host organism and any subsequent generation.

“Transient transformation” can refer to the introduction of a nucleic acid fragment into the nucleus, or DNA-containing organelle, of a host organism resulting in gene expression without genetically stable inheritance.

Host organisms containing the transformed nucleic acid fragments can be referred to as “transgenic” organisms.

“Transformation cassette” can refer to a construct having elements that facilitates transformation of a particular host cell. The terms “transformation cassette” and “transformation construct” can be used interchangeably herein.

“Allele” can be one of several alternative forms of a gene occupying a given locus on a chromosome. When the alleles present at a given locus on a pair of homologous chromosomes in a diploid plant are the same that plant can be homozygous at that locus. If the alleles present at a given locus on a pair of homologous chromosomes in a diploid plant differ, that plant can be heterozygous at that locus. If a transgene is present on one of a pair of homologous chromosomes in a diploid plant that plant can be hemizygous at that locus.

“Organelle-specific and “organelle-preferred” can be used interchangeably, and when used to describe a regulatory element (e.g., an organelle-specific promoter), refer to a regulatory element that is functional within a given cell (e.g., a plant cell) predominantly but not necessarily exclusively in an organelle.

An organelle-specific regulatory domain may be derived from an organellar polynucleotide of interest. An organelle-specific regulatory domain may comprise all or part of the nucleic acid sequence of an organellar polynucleotide of interest. The organelle-specific regulatory domain may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the organellar polynucleotide of interest.

“Mitochondrial-specific and “mitochondrial-preferred” can be used interchangeably, and when used to describe a regulatory element (e.g., a mitochondrial-specific promoter), refer to a regulatory element that is functional within a given cell (e.g., a plant cell) predominantly but not necessarily exclusively in mitochondria.

“Plastid-specific and “plastid-preferred” can be used interchangeably, and when used to describe a regulatory element (e.g., a plastid-specific promoter), refer to a regulatory element that is functional within a given cell (e.g., a plant cell) predominantly but not necessarily exclusively in plastids.

“Chloroplast-specific and “chloroplast-preferred” can be used interchangeably, and when used to describe a regulatory element (e.g., a chloroplast-specific promoter), refer to a regulatory element that is functional within a given cell (e.g., a plant cell) predominantly but not necessarily exclusively in chloroplasts.

The term “mitochondrial genome” and “genome of a mitochondrion” can be used interchangeably and refer to the nucleic acid sequences present within endogenous mitochondrial genetic elements. The mitochondrial genome may be edited by the addition of a sequence (e.g., a heterologous sequence) into an endogenous mitochondrial genetic element. An autonomously replicating heterologous episomal element (e.g., a plasmid DNA) introduced into a mitochondrion is considered to be an independent genetic element and is not considered to be part of the mitochondrial genome.

The terms “plastid genome”, “chloroplast genome”, “genome of a plastid” and “genome of a chloroplast” can be used interchangeably and refer to the nucleic acid sequences present within endogenous plastid genetic elements. The plastid genome may be edited by the addition of a sequence (e.g., a heterologous sequence) into an endogenous plastid genetic element. An autonomously replicating heterologous episomal element (e.g., a plasmid DNA) introduced into a plastid is considered to be an independent genetic element and is not considered to be part of the plastid genome.

A “chloroplast transit peptide” can be an amino acid sequence that can direct a protein to the chloroplast or other plastid types present in the cell. The chloroplast transit peptide can be translated in conjunction with the protein in the cell in which the protein can be made. The terms “chloroplast transit peptide”, “plastid transit peptide”, “chloroplast targeting peptide” and “plastid targeting peptide” can be used interchangeably herein. “Chloroplast transit sequence” can refer to a nucleotide sequence that can encode a chloroplast transit peptide.

A “signal peptide” can be an amino acid sequence that can direct a protein to the secretory system. The signal peptide can be translated in conjunction with a protein. For example, if the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present may be removed and a nuclear localization signal can be included.

A “mitochondrial signal peptide” can be an amino acid sequence which can direct a precursor protein into the mitochondria. The terms “mitochondrial signal peptide”, “mitochondrial transit peptide” and “mitochondrial targeting peptide” can be used interchangeably herein.

An “organelle targeting polynucleotide” can be a nucleotide sequence which can direct import of the polynucleotide into an organelle. The terms “organelle targeting polynucleotide”, “organelle targeting nucleic acid” and “organelle targeting nucleic acid sequence” can be used interchangeably herein. An organelle targeting polynucleotide may be directed to, for example, the plastid (“plastid targeting polynucleotide”) or the mitochondria (“mitochondria targeting polynucleotide”). The polynucleotide may be RNA (“organelle targeting RNA”), DNA (“organelle targeting DNA) or a combination of RNA and DNA. An organelle targeting RNA directed to the plastid can be termed a “plastid targeting RNA”. The terms “plastid targeting RNA”, “chloroplast targeting RNA” and “transit RNA” are used interchangeably herein. An organelle targeting RNA directed to the mitochondria can be termed a “mitochondria targeting RNA”.

RNA can be introduced into organelles by fusion with organelle-targeting nucleotide sequences.

RNAs can be imported into mitochondria. One such mitochondrial targeting RNA can be the yeast tRNA^{Lys}. The yeast tRNA^{Lys} and its variants can be imported into human mitochondria. Additional RNAs that can be imported into mitochondria can be 5S rRNA, and the RNA components of RNase P and MRP RNA. These RNAs can function as vectors for delivering heterologous RNA sequences into mitochondria (U.S. Pat. No. 8,883,755 B2). PNPASE protein can augment RNase P, 5S rRNA, and MRP RNA import into yeast mitochondria (U.S. Pat. No. 9,238,041 B2). RNA can be imported into mitochondria of human cells when the RNA contains: (1) a mitochondria localization sequence (such as MRPS12 3′-UTR) to localize the RNA to be in the proximity of a mitochondrion, and (2) an RNA import sequence (such as a RP import sequence from the RNA component of RNase P) to cause it to be internalized by mitochondria (U.S. Pat. No. 9,238,041 B2). RNA can be imported into the mitochondria of plant cells when the RNA contains a tRNA-like structure from the genome of Turnip yellow mosaic virus (TYMV; U.S. Pat. No. 9,441,243 B2).

RNAs can be imported into plastids. Plastid targeting RNAs that can mediate import of attached heterologous RNA can include vd-5′UTR (e.g., viroid-derived ncRNA sequence acting as 5′UTR; Gomez and Pallas 2010 PLOS One 5:e12269) and eIF4E1 mRNA (US Patent Publication No. US 20090178161 A1).

Nucleic acids can be introduced into organelles by association with proteins containing organelle-targeting peptides. Nucleic acids within an Adenoassociated virus (AAV) virion can be directed to the mitochondria of human cells by addition of a mitochondrial targeting signal to the capsid protein (U.S. Pat. No. 8,278,428 B2). Nucleic acids can also be introduced into plant mitochondria and plastids by association with peptide-based DNA carriers (Yoshizumi et al. 2018 Biomacromolecules 19: 1582-1591). The peptide-based DNA carrier contains two functional units: a polycationic DNA-binding domain (KH) and an organellar targeting peptide (e.g., chloroplast transit peptide or mitochondria targeting peptide). RNA containing sequences that bind to a specific protein (e.g., LtrB intron) can be imported into plant mitochondria by association with an RNA-binding protein (e.g., LtrA protein) in which the RNA-binding protein has been modified to include a mitochondrial targeting peptide (U.S. Pat. No. 9,663,792 B2).

Methods are presented herein in which nucleic acids are introduced into organelles (e.g., mitochondria, plastids) directly and without prior association of the nucleic acids with organellar targeting peptides or organellar targeting polynucleotides.

As used herein, “fusion” can refer to a protein and/or nucleic acid comprising one or more non-native sequences (e.g., moieties). Any of the molecules described herein (e.g., nucleic acids, proteins, polypeptides, polynucleic acid, Cas protein, guide polynucleotide) can be engineered as fusions. A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the site-directed polypeptide. A fusion can provide a non-native sequence (e.g., affinity tag) that can be used to track or purify. A fusion can be a small molecule such as biotin or a dye such as alexa fluor dyes, Cyanine3 dye, and Cyanine5 dye.

A fusion can refer to any protein with a functional effect. For example, a fusion protein can comprise deaminase activity, cytidine deaminase activity (US Patent Publication No. US20150166980, herein incorporated by reference), adenine deaminase activity (US Patent Publication No. US20180073012, herein incorporated by reference), uracil glycosylase inhibitor activity (US Patent Publication No. US20170121693, herein incorporated by reference), methyltransferase activity, demethylase activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, remodeling activity, protease activity, oxidoreductase activity, transferase activity, hydrolase activity, lyase activity, isomerase activity, synthase activity, synthetase activity, or demyristoylation activity. An effector protein can modify a genomic locus. A fusion protein can be a fusion in a Cas protein. The Cas protein may be a modified form that has nickase activity or that has no substantial nucleic acid-cleaving activity. A fusion protein can be a non-native sequence in a Cas protein.

As used herein, a “nucleic acid” can refer to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g. altered backgone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or flurescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine.

### RNA Editing

The RNA editing process is an essential post-transcriptional event in plant mitochondrial gene expression (Choury et al. 2004 Nucleic Acids Res 32: 6397-6406). RNA editing usually occurs by specific C-to-U nucleotide conversions. RNA editing has functional consequences since the nucleotide conversion usually alters the coding property of the mRNA, resulting in a change in amino acid or the creation of a start or stop codon in the mRNA. In higher plants, RNA editing in mitochondria and chloroplasts share some common features. In both organelles, RNA editing occurs by deamination of a C residue. Also, the sequences surrounding the editing site have no common characteristics. However, differences are apparent in RNA editing between the organelles; a chloroplast sequence containing a promiscuous editing site is not edited in mitochondria and a mitochondrial sequence containing an editing site is not edited in chloroplasts. Consequently, the RNA editing process is specific for each organelle.

The cis elements involved in the recognition of two RNA editing sites in the cox2 transcript from wheat mitochondria have been examined (Choury et al. 2004 Nucl Acids Res 32: 6397-6406). A minimal region of 23 nucleotides is involved in the recognition of the C residues at positions 77 and 259 (i.e., the C77 and C259 RNA editing sites). Additionally, these 23 nucleotide regions allow for mitochondrial RNA editing when placed elsewhere in the transcript. As such, these 23 nucleotide regions can be useful for targeted RNA editing of heterologous transcripts in plant mitochondria. 5′ and 3′ UTRs

Translation of an mRNA in an organelle can be impacted by the sequence of the 5′-UTR and the 3′-UTR. In chloroplasts, the 5′ UTR has been shown to influence translation and/or stability of the mRNAs for the psbA, petA and atpH genes (Zoschke and Bock 2018 Plant Cell 30: 745-770). Additionally, base-pairing between the psaC coding region and the 5′-UTR of ndhD represses translation of the ndhD coding region when both coding regions are present on a dicistronic transcript (Hirose and Sugiura 1997 EMBO J 16: 6804-6811). Although chloroplast regulatory elements have been shown to function in chloroplasts of different species, use of an endogenous promoter, 5′-UTR and 3′-UTR has been shown to result in much higher gene expression of certain heterologous coding regions (Ruhlman et al. 2010 Plant Physiol 152: 2088-2104). In yeast mitochondria the 5′-UTR of the COX/mRNA has been shown to bind two specific translational activators, Pet309 and Mss51 (Fontanesi 2013 IUBMB Life 65: 397-408), and binding of Atp22 to the 5′-UTR of the ATP 6 mRNA is required for its translation (Zeng et al. 2007 Genetics 175: 55-63).

### Suppression of Gene Expression

“Silencing,” as used herein with respect to the target gene, can refer to the suppression of levels of mRNA or protein/enzyme expressed by the target gene, and/or the level of the enzyme activity or protein functionality. The terms “suppression”, “suppressing” and “silencing”, which can be used interchangeably herein, can include lowering, reducing, declining, decreasing, inhibiting, eliminating or preventing. “Silencing” or “gene silencing” can occur by any suitable mechanism. Non-limiting examples of silencing can include approaches that use any of the following: antisense, co-suppression, viral-suppression, hairpin suppression, stem-loop suppression, ribozymes, double-stranded RNA (dsRNA), RNAi (RNA interference) and small RNAs such as siRNA (short interfering RNA) and miRNA (microRNA), or any combination thereof

The sequence used for silencing of a gene of interest may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand, or both) of the gene of interest. A suppression DNA construct may comprise 20, 30, 40, 50, 60, 70, 80, 90, 100, 200, 300, 400, 500, 600, 700, 800, 900 or 1000 contiguous nucleotides of the sense strand (or antisense strand, or both) of the gene of interest, and combinations thereof.

### Sequence Identity, Similarity and Variation

Sequence alignments and percent identity or similarity calculations may be determined using a variety of comparison methods designed to detect homologous sequences including, but not limited to, the MEGALIGN™ program of the LASERGENE™ bioinformatics computing suite (DNASTAR™ Inc., Madison, Wis.). In some embodiments, where sequence analysis software is used for analysis, the results of the analysis can be based on the “default values” of the program referenced. As used herein “default values” can mean any set of values or parameters that originally load with the software when first initialized.

The “Clustal V method of alignment” can correspond to the alignment method labeled Clustal V and, for example, found in the MEGALIGN™ program of the LASERGENE™ bioinformatics computing suite (DNASTAR™ Inc., Madison, Wis.). For multiple alignments, the default values can correspond to GAP PENALTY=10 and GAP LENGTH PENALTY=10. Default parameters for pairwise alignments and calculation of percent identity of protein sequences using the Clustal method can be, for example, KTUPLE=1, GAP PENALTY=3, WINDOW=S and DIAGONALS SAVED=5. For nucleic acids these parameters can be for example KTUPLE=2, GAP PENALTY=5, WINDOW=4 and DIAGONALS SAVED=4. After alignment of the sequences using the Clustal V program, “percent identity” and “divergence” values can be obtained by viewing the “sequence distances” table in the same program.

The “Clustal W method of alignment” can correspond to the alignment method labeled Clustal W and, for example, found in the MEGALIGN™ v6.1 program of the LASERGENE™ bioinformatics computing suite (DNASTAR™ Inc., Madison, Wis.). Default parameters for multiple alignments can correspond to for example: GAP PENALTY=10, GAP LENGTH PENALTY=0.2, Delay Divergence Sequences=30%, DNA Transition Weight=0.5, Protein Weight Matrix=Gonnet Series, DNA Weight Matrix=IUB. After alignment of the sequences using the Clustal W program, “percent identity” values can be obtained by viewing the “sequence distances” table in the same program.

Sequence identity/similarity values can also be obtained using GAP Version 10 (GCG, Accelrys, San Diego, Calif.) using for example the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix. GAP can use an algorithm to find an alignment of two complete sequences that can maximize the number of matches and minimizes the number of gaps. GAP can consider all possible alignments and gap positions. GAP can create the alignment with the largest number of matched bases and the fewest gaps, using, for example, a gap creation penalty and a gap extension penalty in units of matched bases.

“BLAST” can be a searching algorithm provided by the National Center for Biotechnology Information (NCBI) that can be used to find regions of similarity between biological sequences. The program can compare nucleotide or protein sequences to sequence databases. The program can calculate the statistical significance of matches to identify sequences having sufficient similarity to a query sequence such that the similarity may not be predicted to have occurred randomly. BLAST can report the identified sequences and their local alignment to the query sequence.

The term “conserved domain” or “motif” can mean a set of amino acids conserved at specific positions along an aligned sequence of evolutionarily related proteins.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These can refer to polypeptide or nucleic acid fragments wherein changes in one or more amino acids or nucleotide bases may not affect the function of the molecule, such as the ability to mediate gene expression or to produce a certain phenotype.

Substantially similar nucleic acid sequences encompassed may be defined by their ability to hybridize (for example, under moderately stringent conditions, e.g., 0.5×SSC, 0.1% SDS, 60° C.) with the sequences exemplified herein, or to any portion of the nucleotide sequences disclosed herein. Post-hybridization washes can determine stringency conditions.

The term “selectively hybridizes” can include reference to hybridization, for example under stringent hybridization conditions.

In some embodiments, stringent conditions can be those in which the salt concentration is less than about 1.5 M Na ion, for example, about 0.01 to 1.0 M Na ion concentration (or other salt(s)) at pH 7.0 to 8.3, and, for example, at least about 30° C. for short probes (e.g., 10 to 50 nucleotides) and, for example, at least about 60° C. for long probes (e.g., greater than 50 nucleotides). Stringent conditions may also be achieved with the addition of destabilizing agents such as formamide. Exemplary low stringency conditions can include hybridization with a buffer solution of, for example, 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions can include hybridization in 40 to 45% formamide, 1 M NaCl, 1% SDS at 37° C., and a wash in 0.5× to 1×SSC at 55 to 60° C. Exemplary high stringency conditions can include hybridization in, for example, 50% formamide, 1 M NaCl, 1% SDS at 37° C., and a wash in 0.1×SSC at 60 to 65° C.

“Sequence identity” or “identity” in the context of nucleic acid or polypeptide sequences can refer to the nucleic acid bases or amino acid residues in two sequences that are the same when aligned for maximum correspondence over a specified comparison window.

The term “percentage of sequence identity” can refer to the value determined by comparing two optimally aligned sequences over a comparison window. The portion of the polynucleotide or polypeptide sequence in the comparison window may comprise additions or deletions (i.e., gaps) as compared to the reference sequence (which may or may not comprise additions or deletions) for optimal alignment of the two sequences. The percentage can be calculated by, for example, determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Percent sequence identities can include, but are not limited to, 50%, 55%, 60%, 65%, 70%, 75%, 80%, 85%, 90% or 95%, or any percentage from 50% to 100%. Sequence identity can include any integer percentage from 50% to 100%. These identities can be determined using any of the programs described herein.

Sequence identity can be useful in identifying polypeptides from other species or modified naturally or synthetically wherein such polypeptides have the same or similar function or activity. Percent identities can include, but are not limited to, about at least 50%, 55%, 60%, 65%, 70%, 75%, 80%, 85%, 90% or 95%. Sequence identity (e.g, amino acid sequence identity) can include an integer percentage from 50% to 100%. Sequence (e.g., amino acid) identity can include, for example, about at least: 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

### Definitions, Traits and Processes Relevant to Plants

“Plant” can include reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, stems, shoots, flowers, fruit, gametophytes, sporophytes, pollen, and microspores.

“Propagule” can include products of meiosis and/or mitosis able to propagate a new plant. Propagule can include seeds, spores and parts of a plant that can serve as a means of vegetative reproduction, such as corms, tubers, offsets, or runners. Propagule can include grafts where one portion of a plant can be grafted to another portion of a different plant (even one of a different species) to create a living organism. Propagule can include plants and seeds produced by cloning or by bringing together meiotic products, or allowing meiotic products to come together to form an embryo or fertilized egg (naturally or with human intervention).

“Progeny” can comprise any subsequent generation of a plant.

The terms “monocot” and “monocotyledonous plant” can be used interchangeably herein. A monocot can include the Gramineae.

The terms “dicot” and “dicotyledonous plant” can be used interchangeably herein. A dicot can include, for example, the following families: Brassicaceae, Leguminosae, and Solanaceae.

“Transgenic plant” can include reference to a plant which comprises within its genome a heterologous polynucleotide. For example, the heterologous polynucleotide may be stably integrated within the genome (e.g., nuclear, plastid, mitochondrial) such that the polynucleotide can be passed on to successive generations. The heterologous polynucleotide may be integrated into the genome alone or as part of a recombinant DNA construct.

“Transgenic plant” can include reference to plants which can comprise more than one heterologous polynucleotide within their genome. Each heterologous polynucleotide may confer a different trait to the transgenic plant.

Multiple traits can be introduced into crop plants, and can be referred to as a gene stacking approach. Gene stacking can be used, for example, for development of genetically improved germplasm. In this approach, multiple genes conferring different characteristics of interest can be introduced into a plant. Gene stacking can be accomplished by many means including but not limited to co-transformation, retransformation, and crossing lines with different transgenes. As used herein, the term “stacked” can include having multiple traits present in the same plant (e.g., both traits are incorporated into the nuclear genome, one trait is incorporated into the nuclear genome and one trait is incorporated into the genome of an organelle, or both traits are incorporated into the genome of an organelle).

The term “crossed” or “cross” or “crossing” in the context of the disclosure can mean the fusion of gametes (e.g., via pollination) to produce progeny (e.g., cells, seeds, or plants). The term can encompass both sexual crosses (e.g., the pollination of one plant by another) and selfing (e.g., self-pollination; when the pollen and ovule are from the same plant or genetically identical plants).

The term “maternal inheritance” can refer to the transmission of traits that can be solely dependent on properties of the genome of the female gamete.

The term “paternal inheritance” can refer to the transmission of traits that are solely dependent on properties of the genome of the male gamete.

The term “introgression” can refer to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene or a selected allele of a marker or QTL.

“A plant-optimized nucleotide sequence” can be a nucleotide sequence that has been optimized for increased expression in plants, particularly for increased expression in plants or in one or more plants of interest. For example, a plant-optimized nucleotide sequence can be synthesized by modifying a nucleotide sequence encoding a protein such as, for example, a double-strand-break-inducing agent (e.g., an endonuclease) as disclosed herein, using one or more plant-preferred codons for improved expression. A host-preferred codon usage can be utilized for codon optimization.

Plant-preferred genes can be synthesized. Additional sequence modifications can enhance gene expression in a plant host. These can include, for example, elimination of: one or more sequences encoding spurious polyadenylation signals, one or more exon-intron splice site signals, one or more transposon-like repeats, and sequences that may be deleterious to gene expression. The G-C content of the sequence may be adjusted, for example, to levels average for a given plant host, as calculated by reference to genes expressed in the host plant cell. When possible, the sequence can be modified to avoid one or more predicted hairpin secondary mRNA structures. Thus, “a plant-optimized nucleotide sequence” of the present disclosure can comprise one or more of such sequence modifications.

A “trait” can refer to, for example, a physiological, morphological, biochemical, or physical characteristic of a plant or particular plant material or cell. In some instances, this characteristic can be visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” can be a measurable parameter including but not limited to, abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

Particular phenotypes may include, but are not limited to kernel number, kernel area, grain weight, and predicted weight of the grain on the ear (based on the calibration of kernel area to grain weight).

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS).

“Increased stress tolerance” of a plant can be measured relative to a reference or control plant, and can be a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Stress tolerance activity” of a polypeptide can indicate that over-expression of the polypeptide in a transgenic plant can confer increased stress tolerance to the transgenic plant relative to a reference or control plant.

Increased biomass can be measured, for example, as an increase in plant height, plant total leaf area, plant fresh weight, plant dry weight or plant seed yield, as compared with control plants.

The ability to increase the biomass or size of a plant can have several important commercial applications. Crop species may be generated that can produce larger cultivars, generating higher yield in, for example, plants in which the vegetative portion of the plant can be useful as food, biofuel or both.

Increased leaf size can be produced by the methods and composition of the disclosure. Increasing leaf biomass can be used to increase production of plant-derived pharmaceutical or industrial products. An increase in total plant photosynthesis can be achieved by, for example, increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of a tissue, such as root tissue, may be useful to improve a plant's ability to grow under harsh environmental conditions, including drought or nutrient deprivation. Larger roots may better reach water or nutrients or take up water or nutrients.

The ability to provide larger varieties can be highly desirable, for example, for some ornamental plants. For many plants, including fruit-bearing trees, trees that are used for lumber production, or trees and shrubs that serve as view or wind screens, increased stature can provide improved benefits in the forms of greater yield or improved screening.

### Herbicide Resistance in Plants

An “herbicide resistance protein” or a protein resulting from expression of an “herbicide resistance-encoding nucleic acid molecule” can include proteins that can confer upon a cell the ability to tolerate a higher concentration of an herbicide, for example, compared with cells that do not express the protein. An herbicide resistance protein or a protein resulting from expression of a herbicide resistance-encoding nucleic acid molecule can include proteins that can confer upon a cell the ability to tolerate a concentration of a herbicide for a longer period of time than cells that do not express the protein. Herbicide resistance traits may be introduced into plants by, for example, genes coding for resistance to herbicides. Genes coding for resistance to herbicides include, for example, genes that act to inhibit the action of acetolactate synthase (ALS), such as the sulfonylurea-type herbicides, genes that act to inhibit the action of glutamine synthase, such as phosphinothricin or basta (e.g., the bar gene), glyphosate (e.g., the EPSP synthase gene), HPPD inhibitors (e.g, the HPPD gene).

Herbicide resistance proteins can include the following: a 4-hydroxphenylpyruvate dioxygenase (HPPD), a sulfonylurea-tolerant acetolactate synthase (ALS), an imidazolinone-tolerant acetolactate synthase (ALS), an ALS inhibitor-tolerant polypeptide, a glyphosate-tolerant 5-enolpyruvylshikimate-3-phosphate synthase (EPSPS), a glyphosate-tolerant glyphosate oxidoreductase (GOX), a glyphosate N-acetyltransferase (GAT), a phosphinothricin acetyl transferase (PAT), a protoporphyrinogen oxidase (PROTOX), a dicamba monooxygenase, an auxin enzyme or receptor, AAD1, AAD12, a P450 polypeptide (e.g., NSF1) and an acetyl coenzyme A carboxylase (ACCase). Non-limiting examples of genes useful for conferring herbicide resistance in plants can include genes that encode the above proteins.

### Pest Resistance in Plants by Gene Silencing

A “plant pest” can mean any living stage of an entity that can directly or indirectly injure, cause damage to, or cause disease in any plant or plant product. A plant pest can include a protozoan, a nonhuman animal, a parasitic plant, a bacterium, a fungus, a virus, a viroid, an infectious agent, a pathogen, or any article similar to or allied thereof.

Plant pest invertebrates can include, but are not limited to, pest nematodes, pest mollusks (slugs and snails), and pest insects. Plant pathogens can include fungi and nematodes.

The plant pathogen can be a eukaryotic plant pathogen. This includes for example, a fungal pathogen, such as a phytopathogenic fungus.

Non-limiting examples of fungal plant pathogens include, e.g., the fungi that cause powdery mildew, rust, leaf spot and blight, damping-off, root rot, crown rot, cotton boll rot, stem canker, twig canker, vascular wilt, smut, or mold, including, but not limited to, Fusarium spp., Phakospora spp., Rhizoctonia spp., Aspergillus spp., Gibberella spp., Pyricularia spp., Alternaria spp., and Phytophthora spp. Specific examples of fungal plant pathogens include Phakospora pachirhizi (Asian soy rust), Puccinia sorghi (corn common rust), Puccinia polysora (corn Southern rust), Fusarium oxysporum and other Fusarium spp., Alternaria spp., Penicillium spp., Pythium aphanidermatum and other Pythium spp., Rhizoctonia solani, Exserohilum turcicum (Northern corn leaf blight), Bipolaris maydis (Southern corn leaf blight), Ustilago maydis (corn smut), Fusarium graminearum (Gibberella zeae), Fusarium verticilliodes {Gibberella moniliformis), F. proliferatum (G. fujikuroi var. intermedia), F. sub glutinous (G. subglutinans), Diplodia maydis, Sporisorium holci-sorghi, Colletotrichum graminicola, Setosphaeria turcica, Aureobasidium zeae, Phytophthora infestans, Phytophthora sojae, Sclerotinia sclerotiorum, and fungal species.

Non-limiting examples of invertebrate pests can include cyst nematodes Heterodera spp. such as soybean cyst nematode Heterodera glycines, root knot nematodes Meloidogyne spp., lance nematodes Hoplolaimus spp., stunt nematodes Tylenchorhynchus spp., spiral nematodes Helicotylenchus spp., lesion nematodes Pratylenchus spp., ring nematodes Criconema spp., foliar nematodes Aphelenchus spp. or Aphelenchoides spp., corn rootworms, Lygus spp., aphids and similar sap-sucking insects such as Phylloxera (Daktulosphaira vitifoliae), corn borers, cutworms, armyworms, leafhoppers, Japanese beetles, grasshoppers, and other pest coleopterans, dipterans, and lepidopterans. Additional examples of invertebrate pests can include pests that can infest the root systems of crop plants, e.g., northern corn rootworm (Diabrotica barberi), southern corn rootworm (Diabrotica undecimpunctata), Western corn rootworm (Diabrotica virgifera), corn root aphid (Anuraphis maidiradicis), black cutworm (Agrotis ipsilon), glassy cutworm (Crymodes devastator), dingy cutworm (Feltia ducens), claybacked cutworm (Agrotis gladiaria), wireworm (Melanotus spp., Aeolus mellillus), wheat wireworm (Aeolus mancus), sand wireworm (Horistonotus uhlerii), maize billbug (Sphenophorus maidis), timothy billbug (Sphenophorus zeae), bluegrass billbug (Sphenophorus parvulus), southern corn billbug (Sphenophorus callosus), white grubs (Phyllophaga spp.), seedcorn maggot (Delia platura), grape colaspis (Colaspis brunnea), seedcorn beetle (Stenolophus lecontei), and slender seedcorn beetle (Clivinia impressifrons), and parasitic nematodes.

The target gene (e.g., for gene silencing) may be an essential gene of the plant pest or plant pathogen. Essential genes can include genes that may be required for development of the pest or pathogen to a fertile reproductive adult.

Target genes (e.g., from pests) can include invertebrate genes for major sperm protein, alpha tubulin, beta tubulin, vacuolar ATPase, glyceraldehyde-3-phosphate dehydrogenase, PvNA polymerase ττ, chitin synthase, cytochromes, miRNAs, miRNA precursor molecules and miRNA promoters. Target genes (e.g., from pathogens) can include genes for miRNAs, miRNA precursor molecules, fungal tubulin, fungal vacuolar ATPase, fungal chitin synthase, fungal MAP kinases, fungal Pad Tyr/Thr phosphatase, enzymes involved in nutrient transport (e.g., amino acid transporters or sugar transporters), enzymes involved in fungal cell wall biosynthesis, cutinases, melanin biosynthetic enzymes, polygalacturonases, pectinases, pectin lyases, cellulases, proteases, genes that interact with plant avirulence genes, and genes involved in invasion and replication of the pathogen in the infected plant.

Plants may be transformed (e.g., in the nucleus, an organelle, or both) with an expression cassette encoding, for example, a dsRNA, a siRNA or a miRNA. The dsRNA, siRNA, or miRNA can suppress (e.g., expression of) at least one (e.g., at least 1, at least 2, at least 3, at least 4, at least 5, at least 6, at least 7, at least 8, at least 9, or at least 10) target gene present in a plant pest. The dsRNA, siRNA, or miRNA can suppress, for example, 1, 2, 3, 4, 5, 6, 7, 8, 9, 10, 15, 20 or more target genes of a plant pest. Suppression of a target gene present in the plant pest can provide complete or nearly complete protection from the plant pest. “Complete protection” can mean that no (e.g., substantial) damage can be caused to the plant by the plant pest.

The dsRNA, the siRNA or the miRNA may be designed for suppression of a gene selected from the group consisting of: proteasome A-type subunit peptide (Pas-4), ACT, SHR, EPIC2B and PnPMAI.

### Resistance to Plant Pests

Resistance to pests in plants can be achieved by, for example, transgenic control. In-plant transgenic control of, for example, insect pests, can be achieved through, for example, plant expression of crystal (Cry) delta endotoxin genes and/or Vegetative Insecticidal Proteins (VIP) such as from Bacillus thuringiensis. Non-limiting examples of Cry toxins include, for example, the 60 main groups of “Cry” toxins (e.g., Cry1-Cry59) and VIP toxins. Cry toxins can include subgroups of Cry toxins, for example, Cry 1a.

An expression cassette for use in transformation (e.g, into an organelle) may be constructed using, for example, a Cry sequence. The Cry sequence can include, for example, the wild-type (e.g, native) nucleic acid sequence encoding at least one protein selected from the group consisting of: Cry1Ac, Cyt1Aa, Cry1Ab, Cry2Aa, Cry1I, Cry1C, Cry1D, Cry1E, Cry1Be, Cry1Fa and Vip3A. The Cry sequence can include, for example, a modified (e.g, truncated or fusion) nucleic acid sequence encoding at least one protein selected from the group consisting of: Cry1Ac, Cyt1Aa, Cry1Ab, Cry2Aa, Cry1I, Cry1C, Cry1D, Cry1E, Cry1Be, Cry1Fa and Vip3A. A modified such as a truncated nucleic acid sequence can encode a modified such as a truncated protein fragment that can retain insecticidal activity. The nucleic acid sequence encoding the full-length, or modified (e.g., truncated) protein may be codon-optimized for the organelle of interest. The Cry protein can be a Cyt1Aa protein (e.g., from Bacillus thuringiensis serovar israelensis; Gene ID: 5759908).

Accessory proteins, for example, for a Cry protein, can be introduced into a cell (e.g., into an organelle). An accessory protein can, for example, increase expression, stability, and/or function of, for example, a Cry protein. Non-limiting examples of accessory proteins include 20 kDa accessory proteins (e.g., from Bacillus thuringiensis serovar israelensis) and 19 kDa accessory proteins (e.g., from Bacillus thuringiensis serovar israelensis).

Polynucleotides that encode proteins useful in conferring insect resistance to a plant may be included in an expression cassette as a polycistronic unit or may be expressed from separate expression cassettes.

### Genome Modification

The disclosure provides compositions and methods that can be used for, for example, genome modification of a target sequence in the genome (e.g., a plastid or a mitochondrial genome) of an organism or cell (e.g., a plant or plant cell), for selecting the modified organism or cell, for gene editing, and for inserting a donor polynucleotide into the genome of an organism or cell. The methods can employ a polynucleotide guided polypeptide system; e.g., a guide polynucleotide/Cas protein system. The Cas protein can be guided by the guide polynucleotide to recognize a target polynucleic acid. The Cas protein can introduce a single strand or double strand break at a specific target site into the genome of a cell. The guide polynucleotide/Cas polypeptide system can provide for an effective system for modifying target sites within the genome of a plant, plant cell or seed.

A variety of methods can be employed to further modify a target site to introduce a donor polynucleotide of interest. The nucleotide sequence to be edited (e.g., the nucleotide sequence of interest) can be located within or outside a target site that is recognized by a polynucleotide guided polypeptide.

Further provided are methods and compositions employing a polynucleotide guided polypeptide system for modification of multiple target sites within the genome of an organelle. Modification of multiple target sites within the genome of an organelle can facilitate the creation of homoplastic transformation events.

### Polynucleotide Guided Polypeptide Systems

A polynucleotide-guided polypeptide can be a polypeptide that can bind to a target nucleic acid. A polynucleotide-guided polypeptide can be a nuclease. A polynucleotide-guided polypeptide can be an endonuclease (e.g., a Cas protein, a MAD protein, an Argonaut protein). A polynucleotide guided polypeptide can form a complex with a guide polynucleotide. A polynucleotide guided polypeptide can be directed to a target nucleic acid by a guide polynucleotide. A polynucleotide guided polypeptide can complex with a guide polynucleotide to recognize a target nucleic acid. A polynucleotide guided polypeptide can introduce a single strand or double strand break at a specific target site (e.g., the genome of a cell).

a. CRISPR Loci

CRISPR loci (Clustered Regularly Interspaced Short Palindromic Repeats) (also known as SPIDRs-SPacer Interspersed Direct Repeats) can constitute a family of DNA loci. CRISPR loci can consist of short and highly conserved DNA repeats (e.g., 24 to 40 bp, repeated from 1 to 140 times—also referred to as CRISPR-repeats). CRISPR DNA repeats can be partially palindromic. The repeated sequences (e.g., usually specific to a species) can be interspaced by variable sequences of constant length (e.g., 20 to 58 by depending on the CRISPR locus.

b. Cas Protein

A Cas protein can be a protein of a CRISPR/Cas system. A Cas protein can be a Class 1 or a Class 2 Cas protein. A Cas protein can be a Type I, Type II, Type III, Type IV, Type V, or Type VI Cas protein.

“Cas gene” can refer to a gene that encodes a Cas protein. The terms Cas protein and Cas polypeptide can be used interchangeably herein. Cas gene can be coupled, associated or close to or in the vicinity of flanking CRISPR loci. The terms “Cas gene”, “CRISPR-associated (Cas) gene” can be used interchangeably herein.

A Cas protein can bind to a target nucleic acid. A Cas protein can be a Cas nuclease. A Cas protein can be a Cas endonuclease. A Cas protein can complex with a guide polynucleotide. A Cas protein can be directed to a target nucleic acid by a guide polynucleotide. A Cas protein can complex with a guide polynucleotide to recognize a target nucleic acid. A Cas protein can introduce a single strand or double strand break at a target nucleic acid sequence (e.g., DNA or RNA). A Cas protein can be enabled by the guide polynucleotide to recognize and introduce a single strand or double strand break at a specific target site into the genome of a cell.

A polynucleotide guided polypeptide (e.g., a Cas protein) can comprise one or more domains. Non-limiting examples of domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. A guide nucleic acid recognition and/or binding domain can interact with a guide nucleic acid. A nuclease domain can comprise catalytic activity for nucleic acid cleavage. A nuclease domain can lack catalytic activity to prevent nucleic acid cleavage. A polynucleotide guided polypeptide can be a chimeric protein that is fused to other proteins or polypeptides. A polynucleotide guided polypeptide can be a chimera of various Cas proteins, for example, comprising domains from different Cas proteins (e.g., homologues).

Non-limiting examples of Cas proteins include c2c1, C2c2, c2c3, Cas1, Cas1B, Cast, Cas3, Cas4, Cas5, Cas5e (CasD), Cas6, Cas6e, Cas6f, Cas7, Cas8a, Cas8a1, Cas8a2, Cas8b, Cas8c, Cas9 (Csn1 or Csx12), Cas10, Cas10d, Cas10, Cas10d, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cul966, and homologs or modified versions thereof.

A Cas protein may be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism can be Streptococcus pyogenes (S. pyogenes).

A polynucleotide guided polypeptide (e.g., a Cas protein) as used herein can be a wild-type or a modified form of a Cas protein. A Cas protein can be an active variant, inactive variant, or fragment of a wild type or modified Cas protein. A Cas protein can comprise an amino acid change such as a deletion, insertion, substitution, variant, mutation, fusion, chimera, or any combination thereof relative to a wild-type version of the Cas protein. A Cas protein can be a polypeptide with at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type exemplary Cas protein (e.g., Cas9 from S. pyogenes). A Cas protein can be a polypeptide with at most about 5%, 10%, 20%, 30%, 40%, 50%, 60%, 70%, 80%, 90%, 100% sequence identity and/or sequence similarity to a wild type exemplary Cas protein. Variants or fragments can comprise at least about 5%, 10%, 20%, 30%, 40%, 50%, 60%, 70%, 80%, 90%, 91%, 92%, 93%, 94%, 95%, 96%, 97%, 98%, 99%, or 100% sequence identity or sequence similarity to a wild type or modified Cas protein or a portion thereof. Variants or fragments can be targeted to a nucleic acid locus in complex with a guide nucleic acid while lacking nucleic acid cleavage activity.

A polynucleotide guided polypeptide (e.g., a Cas protein) can comprise one or more nuclease domains, such as DNase domains. For example, a Cas9 protein can comprise a RuvC-like nuclease domain and/or an HNH-like nuclease domain. The RuvC and HNH domains can each cut a different strand of double-stranded DNA to make a double-stranded break in the DNA. A Cas protein can comprise only one nuclease domain (e.g., Cpf1 comprises RuvC domain but lacks HNH domain)

A polynucleotide guided polypeptide (e.g., a Cas protein) can be modified to optimize activity e.g., cleavage, regulation of gene expression. A polynucleotide guided polypeptide can be modified to increase or decrease nucleic acid binding affinity, nucleic acid binding specificity, and/or enzymatic activity. Polynucleotide guided polypeptides can also be modified to change any other activity or property of the protein, such as stability. For example, one or more nuclease domains of a Cas protein can be modified, deleted, or inactivated, or a Cas protein can be truncated to remove domains that are not essential for the function of the protein or to optimize (e.g., enhance or reduce) the activity of the Cas protein.

A polynucleotide guided polypeptide (e.g., a Cas protein) can be a fusion protein. For example, a Cas protein can be fused to a cleavage domain, an epigenetic modification domain, a transcriptional activation domain, or a transcriptional repressor domain. A Cas protein can also be fused to a heterologous polypeptide providing increased or decreased stability. The fused domain or heterologous polypeptide can be located at the N-terminus, the C-terminus, or internally within the Cas protein.

A polynucleotide guided polypeptide (e.g., a Cas protein) can comprise a heterologous polypeptide for ease of tracking or purification, such as a fluorescent protein, a purification tag, or an epitope tag.

A polynucleotide guided polypeptide (e.g., a Cas protein) can be provided in any form. For example, a Cas protein can be provided in the form of a protein, such as a Cas protein alone or complexed with a guide nucleic acid. A Cas protein can be provided in the form of a nucleic acid encoding the Cas protein, such as an RNA (e.g., messenger RNA (mRNA)) or DNA.

The nucleic acid encoding the polynucleotide guided polypeptide (e.g., a Cas protein) can be codon optimized for efficient translation into protein in a particular cell, organelle, or organism.

Nucleic acids encoding a polynucleotide guided polypeptide (e.g., a Cas protein) can be stably integrated in the genome of an organelle or a cell. Nucleic acids encoding a polynucleotide guided polypeptide can be operably linked to a promoter active in the cell, e.g., in an expression construct. Expression constructs can include any nucleic acid constructs that can direct expression of a gene or other nucleic acid sequence of interest (e.g., a Cas gene). Expression constructs can include any nucleic acid constructs that can transfer such a nucleic acid sequence of interest to a target cell (e.g., into an organelle).

Cas9 can refer to a polypeptide with at least about 5%, 10%, 20%, 30%, 40%, 50%, 60%, 70%, 80%, 90%, 100% sequence identity and/or sequence similarity to a wild type exemplary Cas9 polypeptide (e.g., Cas9 from S. pyogenes). Cas9 can refer to a polypeptide with at most about 5%, 10%, 20%, 30%, 40%, 50%, 60%, 70%, 80%, 90%, 100% sequence identity and/or sequence similarity to a wild type exemplary Cas9 polypeptide (e.g., from S. pyogenes). Cas9 can refer to the wildtype or a modified form of the Cas9 protein that can comprise an amino acid change such as a deletion, insertion, substitution, variant, mutation, fusion, chimera, or any combination thereof.

In one embodiment, the polynucleotide guided polypeptide gene can be a Cas9 protein (SEQ ID NO:1), such as but not limited to, Cas9 sequences listed in SEQ ID NOs: 462, 474, 489, 494, 499, 505, and 518 of WO2007/025097 and incorporated herein by reference. Mutagenesis of Streptococcus pyogenes Cas9 catalytic domains can produce “nicking” enzymes (Cas9n) that can induce single-strand nicks rather than double-strand breaks.

In another embodiment, the polynucleotide guided polypeptide coding sequence can be modified to use codons preferred by the target organism, e.g., a plant, maize or soybean codon-optimized sequence encoding a Cas (e.g., Cas9) protein. In another embodiment, the sequence that encodes a polynucleotide guided polypeptide can be operably linked to one or more sequences encoding nuclear localization signals; e.g., to a SV40 nuclear targeting signal upstream of the Cas protein coding region and a bipartite VirD2 nuclear localization signal downstream of the Cas protein coding region.

In another embodiment, the polynucleotide guided polypeptide may be a MAD polypeptide, e.g., a MAD2 (SEQ ID NO:2) or a MAD7 polypeptide (SEQ ID NO:3), with amino acid sequence corresponding to SEQ ID NO:2 and SEQ ID NO:7 of U.S. Pat. No. 9,982,279, respectively (herein incorporated by reference). MAD7 is a Class 2 Type V-A CRISPR-Cas system isolated from Eubacterium rectale and re-engineered by INSCRIPTA™ (Boulder, Colo.). Analogous to Cas9, it is an RNA-guided nuclease with a diverse protein structure, mechanism of action, and has demonstrated gene editing activity in E. coli and yeast cells. Similar to Acidaminococcus sp. Cas12a, MAD7 does not require a tracrRNA and prefers T-rich PAMs (TTTV and CTTV).

In another embodiment, the polynucleotide guided polypeptide may be an Argonaute protein such as Natronobacterium gregoryi Argonaute (“NgAgo”). The Argonaute protein can be a DNA-guided endonuclease. Argonaute proteins can bind a guide DNA such as a 5′-phosphorylated single-stranded guide DNA (gDNA) of for example, 24 nucleotides. Argonaute proteins can create site-specific target nucleic acid (e.g., DNA) breaks (e.g., double-stranded breaks) when loaded with the gDNA. The Argonaute protein—gDNA system may not require a protospacer-adjacent motif (PAM) for recognition of a target nucleic acid.

In some aspects, the polynucleotide guided polypeptide can be a dead Cas protein, i.e., a protein that lacks nucleic acid cleavage activity, such as dCas or dCas9.

Enzymatically inactive can refer to a polypeptide that can bind to a nucleic acid sequence in a polynucleotide in a sequence-specific manner but may not cleave a target polynucleotide. An enzymatically inactive site-directed polypeptide can comprise an enzymatically inactive domain (e.g. nuclease domain). Enzymatically inactive can refer to no activity. Enzymatically inactive can refer to substantially no activity. Enzymatically inactive can refer to essentially no activity. Enzymatically inactive can refer to an activity less than 1%, less than 2%, less than 3%, less than 4%, less than 5%, less than 6%, less than 7%, less than 8%, less than 9%, or less than 10% activity compared to a wild-type exemplary activity (e.g., nucleic acid cleaving activity, wild-type Cas9 activity).

In another embodiment, the polynucleotide guided polypeptide can be a polypeptide moiety (e.g., a chimeric polypeptide) that can form a programmable nucleoprotein molecular complex with a specificity conferring nucleic acid (SCNA). The programmable nucleoprotein molecular complex can assemble in-vivo, in a target cell, or in an organelle. The programmable nucleoprotein molecular complex can interact with a predetermined target nucleic acid sequence. The programmable nucleoprotein molecular complex may comprise a polynucleotide molecule encoding a chimeric polypeptide. The chimeric polypeptide can comprise a functional domain that can modify a target nucleic acid site. The functional domain can be devoid of a specific nucleic acid binding site. The chimeric polypeptide can comprise a linking domain that can interact with a SCNA. The linking domain can be devoid of a specific target nucleic acid binding site. A SCNA can comprise a nucleotide sequence complementary to a region of a target nucleic acid flanking the target site. A SCNA can comprise a recognition region that can specifically attach to the linking domain of a chimeric polypeptide. Assembly of the chimeric polypeptide and the SCNA within the target cell can form a functional nucleoprotein complex. The nucleoprotein complex can specifically modify a target nucleic acid at the target site.

In another embodiment, the polynucleotide guided endonuclease gene can be a full-length polynucleotide guided endonuclease (e.g., Cas endonuclease, Cas9 endonuclease), or any functional fragment or functional variant thereof.

The terms “functional fragment”, “fragment that is functionally equivalent” and “functionally equivalent fragment” can be used interchangeably herein. In the context of a sequence encoding a polynucleotide guided polypeptide, these terms can refer to a portion or subsequence of the polynucleotide guided polypeptide sequence. The portion or subsequence of the polynucleotide guided polypeptide sequence can comprise the ability to create a single-strand or double-strand break.

The terms “functional variant”, “variant that is functionally equivalent” and “functionally equivalent variant” can be used interchangeably herein. In the context of a polynucleotide guided polypeptide, these terms can refer to a variant of the polynucleotide guided polypeptide. The variant can comprise the ability to create a single-strand or double-strand break. Fragments and variants can be obtained via methods such as site-directed mutagenesis and synthetic construction.

In one embodiment, the polynucleotide guided polypeptide coding sequence can be a plant codon-optimized Streptococcus pyogenes Cas9 coding sequence. The codon optimized Cas9 sequence can recognize any genomic sequence, for example, of the form N(12-30)NGG.

In one embodiment, the polynucleotide guided polypeptide can be introduced directly into a cell by any suitable method, for example, but not limited to transient introduction methods, transfection and/or topical application.

Compositions and methods of the disclosure can use endonucleases. Endonucleases can include meganucleases, also known as homing endonucleases (HEases). Meganucleases can bind and cut at a specific recognition site, which can be about 18 bp or more.

Compositions and methods of the disclosure can use a Transcription activator-like effector nucleases (TALEN; TAL effector nuclease). A TALEN can be used to cleave (e.g., double-strand breaks) at specific target sequences (e.g., in the genome of a plant or other organism).

Compositions and methods of the disclosure can use zinc finger nucleases (ZFNs). Each zinc finger can recognize, for example, three consecutive base pairs in the target DNA. For example, a 3-finger domain can recognize a sequence of 9 contiguous nucleotides, with a dimerization requirement of the nuclease, two sets of zinc finger triplets can be used to bind an 18 nucleotide recognition sequence.

c. Guide Polynucleic Acid

Bacteria and archaea can have evolved adaptive immune defenses termed clustered regularly interspaced short palindromic repeats (CRISPR)/CRISPR-associated (Cas) systems that can use short RNA to direct degradation of foreign nucleic acids. The type II CRISPR/Cas system from bacteria can employ a crRNA and tracrRNA to guide the Cas polypeptide to a nucleic acid target. The crRNA (CRISPR RNA) can contain the region complementary to one strand of the double strand DNA target. The crRNA can base pair with the tracrRNA (trans-activating CRISPR RNA) to form an RNA duplex that can direct the Cas polypeptide to recognize and optionally cleave the DNA target.

As used herein, the term “guide polynucleotide”, can refer to a polynucleotide sequence that can form a complex with a polynucleotide guided polypeptide (e.g., a Cas protein). The guide polynucleotide can direct the polynucleotide guided polypeptide to recognize and optionally cleave (or nick) a DNA target site. The terms “guide polynucleotide” and “guide polynucleic acid” can be used interchangeably herein. The guide polynucleotide can be comprised of a single molecule (unimolecular) or two molecules (bimolecular). The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids can also be referred to as a “guide RNA” (gRNA). In some embodiments, the guide polynucleic acid can be a guide RNA.

As used herein, the term “single guide RNA” (sgRNA) can refer to a synthetic fusion of two RNA molecules, for example, a crRNA (CRISPR RNA) comprising a variable targeting domain, and a tracrRNA. In one embodiment, the guide RNA can comprise a variable targeting domain of 12 to 30 nucleotide sequences and an RNA fragment that can interact with a Cas protein.

As used herein, “crRNA” can refer to a nucleic acid with at least about 5%, 10%, 20%, 30%, 40%, 50%, 60%, 70%, 80%, 90%, 100% sequence identity and/or sequence similarity to a wild type exemplary crRNA (e.g., a crRNA from S. pyogenes). crRNA can refer to a nucleic acid with at most about 5%, 10%, 20%, 30%, 40%, 50%, 60%, 70%, 80%, 90%, 100% sequence identity and/or sequence similarity to a wild type exemplary crRNA (e.g., a crRNA from S. pyogenes). crRNA can refer to a modified form of a crRNA that can comprise a nucleotide change such as a deletion, insertion, or substitution, variant, mutation, or chimera. A crRNA can be a nucleic acid having at least about 60% identical to a wild type exemplary crRNA (e.g., a crRNA from S. pyogenes) sequence over a stretch of at least 6 contiguous nucleotides. For example, a crRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical, to a wild type exemplary crRNA sequence (e.g., a crRNA from S. pyogenes) over a stretch of at least 6 contiguous nucleotides

As used herein, “tracrRNA” can refer to a nucleic acid with at least about 5%, 10%, 20%, 30%, 40%, 50%, 60%, 70%, 80%, 90%, 100% sequence identity and/or sequence similarity to a wild type exemplary tracrRNA sequence (e.g., a tracrRNA from S. pyogenes). tracrRNA can refer to a nucleic acid with at most about 5%, 10%, 20%, 30%, 40%, 50%, 60%, 70%, 80%, 90%, 100% sequence identity and/or sequence similarity to a wild type exemplary tracrRNA sequence (e.g., a tracrRNA from S. pyogenes). tracrRNA can refer to a modified form of a tracrRNA that can comprise a nucleotide change such as a deletion, insertion, or substitution, variant, mutation, or chimera. A tracrRNA can refer to a nucleic acid that can be at least about 60% identical to a wild type exemplary tracrRNA (e.g., a tracrRNA from S. pyogenes) sequence over a stretch of at least 6 contiguous nucleotides. For example, a tracrRNA sequence can be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical, to a wild type exemplary tracrRNA (e.g., a tracrRNA from S. pyogenes) sequence over a stretch of at least 6 contiguous nucleotides.

A guide polynucleotide can be bimolecular (i.e., two molecules; also referred to as “double molecule”, “dual” or “duplex” guide polynucleotide) comprising, for example, a first nucleotide sequence domain (referred to as Variable Targeting domain or VT domain) that is complementary to a nucleotide sequence in a target polynucleic acid (e.g., target DNA) and a second nucleotide sequence domain (referred to as Cas endonuclease recognition domain or CER domain) that interacts with a Cas polypeptide. The VT domain can refer to the spacer region of a guide polynucleic acid. The VT domain can comprise a spacer region of a guide polynucleic acid. The spacer region can interact with a protospacer region of a target nucleic acid in a sequence-specific manner via hybridization (e.g., base pairing). The CER domain of the bimolecular guide polynucleotide can comprise two separate molecules that can be hybridized along a region of complementarity to form, for example, a duplex or a partial duplex. The two separate molecules can be RNA, DNA, and/or RNA-DNA-combination sequences. In some embodiments, the first molecule of the duplex guide polynucleotide comprising a VT domain linked to a CER domain can be referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the crRNA naturally occurring in bacteria and archaea. In one embodiment, the size of the fragment of the crRNA naturally occurring in bacteria and archaea that can be present in a crNucleotide disclosed herein can range from, but is not limited to, 2, 3, 4, 5, 6, 7, 8, 9, 10, 11, 12, 13, 14, 15, 16, 17, 18, 19, 20 or more nucleotides. In some embodiments, the second molecule of the duplex guide polynucleotide comprising a CER domain can be referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In one embodiment, the RNA that guides the RNA/Cas9 polypeptide complex, can be a duplexed RNA comprising a duplex crRNA-tracrRNA.

Complementarity between a guide polynucleic acid (e.g., the VT domain, spacer region) and a target polynucleic acid (e.g., protospacer) can be perfect, substantial, or sufficient. Perfect complementarity between two nucleic acids can mean that the two nucleic acids can form a duplex in which every base in the duplex can be bonded to a complementary base by Watson-Crick pairing. Substantial or sufficient complementary can mean that a sequence in one strand may not be completely and/or perfectly complementary to a sequence in an opposing strand, but that sufficient bonding occurs between bases on the two strands to form a stable hybrid complex in a set of hybridization conditions (e.g., salt concentration and temperature).

A guide polynucleotide can also be a single molecule (i.e., unimolecular), comprising a first nucleotide sequence domain (referred to as Variable Targeting domain or VT domain) that can be complementary to a nucleotide sequence in a target polynucleic acid (e.g., target DNA) and a second nucleotide domain (referred to as Cas endonuclease recognition domain or CER domain) that interacts with a Cas polypeptide. For a single molecule guide polynucleotide, the CER domain can be formed from a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. In some embodiments, the single guide polynucleotide comprises crNucleotide (comprising a VT domain linked to a CER domain) linked to a tracrNucleotide (comprising a CER domain), wherein the linkage can be a nucleotide sequence comprising a RNA sequence, a DNA sequence, or a RNA-DNA combination sequence. The single guide polynucleotide being comprised of sequences from the crNucleotide and tracrNucleotide may be referred to as “single guide RNA” (sgRNA; when composed of a contiguous stretch of RNA nucleotides) or “single guide DNA” (sgDNA; when composed of a contiguous stretch of DNA nucleotides) or “single guide RNA-DNA” (sgDNA-RNA; when composed of a combination of DNA and RNA nucleotides). In one embodiment of the disclosure, the single guide RNA (sgRNA) comprises crRNA or crRNA fragment and a tracrRNA or tracrRNA fragment of the type II CRISPR/Cas system that can form a complex with a type II Cas polypeptide, wherein said guide RNA/Cas polypeptide complex can direct the Cas polypeptide to a plant genomic target site, enabling the Cas polypeptide to introduce a double strand break into the genomic target site.

The term “variable targeting domain” or “VT domain” can be used interchangeably herein and can refer to a nucleotide sequence that can be present in the guide polynucleotide. VT domain can be complementary to one strand of a double stranded DNA target site. The percent complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable target domain can comprise at least 17 nucleotides that are complementary to at least 17 nucleotides of a target polynucleic acid. In some embodiments, the variable targeting domain can comprise a contiguous stretch of nucleotides that are complementary to the target polynucleic acid. In some embodiments, the nucleotides of the guide polynucleic acid that are complementary to the target polynucleic acid can be non-contiguous. In some embodiments, the variable targeting domain can comprise a contiguous stretch of 12 to 30 nucleotides. The variable targeting domain can be composed of a DNA sequence, an RNA sequence, a modified DNA sequence, a modified RNA sequence, or any combination thereof.

A target polynucleotide can be identified by identifying a protospacer adjacent motif (PAM) within a region of interest and selecting a region of a desired size upstream or downstream of the PAM as the protospacer. A corresponding spacer sequence can be designed by determining the complementary sequence of the protospacer region.

The term “Cas endonuclease recognition domain” or “CER domain” of a guide polynucleotide can be used interchangeably herein and can refer to a nucleotide sequence (such as a second nucleotide sequence domain of a guide polynucleotide), that interacts with a Cas polypeptide. The CER domain can be composed of a DNA sequence, an RNA sequence, a modified DNA sequence, a modified RNA sequence (see for example modifications described herein), or any combination thereof.

The nucleotide sequence linking the crNucleotide and the tracrNucleotide of a single guide polynucleotide can comprise an RNA sequence, a DNA sequence, or an RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetranucleotide loop sequence, such as, but not limiting to a GAAA tetranucleotide loop sequence. Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide polynucleotide to a subcellular location, a modification or sequence that provides for tracking, a modification or sequence that provides a binding site for proteins, a Locked Nucleic Acid (LNA), a 5-methyl-2′-deoxycytodine (5mdC), a 2,6-Diaminopurine nucleotide, a 2′-Fluoroadenosine nucleotide, a 2′-Fluorouridine nucleotide; a 2′-O-Methyl RNA nucleotide, a phosphorothioate (PS) bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature can be a member selected from the group consisting of: modified or regulated stability, subcellular targeting, tracking, a fluorescent label, a binding site for a protein or protein complex, modified binding affinity to complementary target sequence, modified resistance to cellular degradation, and increased cellular permeability.

In one embodiment, the guide RNA and Cas polypeptide can form a complex that can enable the Cas polypeptide to introduce a single strand or double strand break at a DNA target site.

In one embodiment, the variable target domain can be 12, 13, 14, 15, 16, 17, 18, 19, 20, 21, 22, 23, 24, 25, 26, 27, 28, 29 or 30 nucleotides in length.

In one embodiment, the guide RNA can comprise a crRNA (or crRNA fragment) and a tracrRNA (or tracrRNA fragment) of the type II CRISPR/Cas system that can form a complex with a type II Cas polypeptide. The guide RNA/Cas polypeptide complex can direct the Cas polypeptide to a target nucleic acid site (e.g., DNA target). The Cas polypeptide can introduce a double strand break into the DNA target site.

In one embodiment the guide polynucleic acid can be introduced into a cell directly using any suitable method such as, but not limited to, particle bombardment or topical applications.

In another embodiment the guide polynucleic acid can be introduced indirectly by introducing a recombinant DNA molecule comprising a polynucleotide encoding the guide polynucleic acid operably linked to a nuclear or organellar promoter that can transcribe the polynucleotide in said nucleus or organelle, respectively.

In some embodiments, the guide polynucleic acid can be introduced into a plant cell via particle bombardment or Agrobacterium transformation of a recombinant DNA construct comprising a polynucleotide encoding the guide polynucleic acid operably linked to a promoter functional in a plant; e.g., a plant U6 polymerase III promoter, a CaMV 35S polymerase II promoter, a promoter functional in a plant organelle.

In one embodiment, the guide polynucleic acid can be a duplexed RNA comprising a duplex crRNA-tracrRNA. A single guide polynucleic acid (e.g., single guide RNA) can require one expression cassette to express the single guide RNA. A duplexed crRNA-tracrRNA can require one or more expression cassette needs to express the duplexed crRNA-tracrRNA.

A plurality of polynucleic acids can be multiplexed to target multiple target nucleic acids. For example, 2, 3, 4, 5, 6, 7, 9, 10, or more than 10 target nucleic acids can be targeted simultaneously or iteratively. Multiplexing can be used, as non-limiting examples, to generate large genomic deletions, modify multiple different sequences at once, and/or in conjunction with dual-nickases to target a gene. In some examples, more than one CRISPR/Cas system can be delivered to target two or more nucleic acid sequence targets. Homologous Cas proteins can be used for multiplexing applications.

Target Sites for Genome Modification

The terms “target site”, “target sequence”, “target polynucleotide”, “target polynucleic acid”, “target locus”, “genomic target site”, “genomic target sequence”, and “genomic target locus” can be used interchangeably herein. Target polynucleic acid can refer to a polynucleotide sequence in the genome (e.g., plastid or mitochondrial genome) of, for example, a plant cell. Target polynucleic acid can refer to the site (e.g., in a genome) recognized by a guide polynucleic acid. Target polynucleic acid can refer to the site (e.g., in a genome) at which a single-strand or double-strand break can be induced (e.g., by a Cas polypeptide). The target site can be an endogenous site in the genome. The target site can be heterologous to the organism and thereby not be naturally occurring in the genome. Target site can be found in a heterologous genomic location compared to where it occurs in nature. As used herein, terms “endogenous target sequence” and “native target sequence” can be used interchangeably herein and can refer to a target sequence that can be endogenous or native to the genome of the organism. Endogenous target sequence can occur at the endogenous or native position of that target sequence in the genome of the organism.

A target polynucleic acid can be DNA, RNA, or both. In some embodiments, the target polynucleic acid can be DNA (e.g., target DNA). In some embodiments, the target polynucleic acid can be genomic DNA. In some embodiments, the target polynucleic acid can be nuclear genomic DNA. In some embodiments, the target polynucleic acid can be organelle genomic DNA. In some embodiments, the target polynucleic acid can be nuclear genomic DNA and organelle genomic DNA.

The terms “artificial target site” and “artificial target sequence” can be used interchangeably herein and can refer to a target sequence that has been introduced into the genome of a plant. Such an artificial target sequence can be identical in sequence to an endogenous or native target sequence in the genome of an organism but may be located in a different position (i.e., a non-endogenous or non-native position) in the genome of the organism.

An “altered target site”, “altered target sequence”, “modified target site”, “modified target sequence” can be used interchangeably herein and can refer to a target sequence as disclosed herein that can comprise at least one alteration when compared to the non-altered target sequence. Such “alterations” can include, for example: (i) replacement of at least one nucleotide, (ii) a deletion of at least one nucleotide, (iii) an insertion of at least one nucleotide, or (iv) any combination of (i)-(iii).

The length of the target site can vary and includes, for example, target sites that are at least 12, 13, 14, 15, 16, 17, 18, 19, 20, 21, 22, 23, 24, 25, 26, 27, 28, 29, 30 or more nucleotides in length. The target site can be palindromic, that is, the sequence on one strand reads the same in the opposite direction on the complementary strand. The nick/cleavage site can be within the target sequence. The nick/cleavage site can be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs.

The target nucleic acid sequence can be 5′ or 3′ of the PAM. The target nucleic acid sequence can be, for example, 16, 17, 18, 19, 20, 21, 22, or 23 bases immediately 5′ of the first nucleotide of the PAM. The target nucleic acid sequence can be, for example, 16, 17, 18, 19, 20, 21, 22, or 23 bases immediately 3′ of the last nucleotide of the PAM. The target nucleic acid sequence can be 20 bases immediately 5′ of the first nucleotide of the PAM. The target nucleic acid sequence can be 20 bases immediately 3′ of the last nucleotide of the PAM.

Site-specific cleavage of a target nucleic acid by a polynucleotide guided polypeptide (e.g., Cas protein) can occur at locations determined by base-pairing complementarity between the guide nucleic acid and the target nucleic acid. Site-specific cleavage of a target nucleic acid by a polynucleotide guided polypeptide (e.g., Cas protein) can occur at locations determined by the protospacer adjacent motif (PAM). For example, the cleavage site of Cas (e.g., Cas9) can be about 1 to about 25, or about 2 to about 5, or about 19 to about 23 base pairs (e.g., 3 base pairs) upstream or downstream of the PAM sequence. In some embodiments, the cleavage site of Cas (e.g., Cas9) can be 3 base pairs upstream of the PAM sequence. In some embodiments, the cleavage site of Cas (e.g., Cpf1) can be 19 bases on the (+) strand and 23 base on the (−) strand, producing a 5′ overhang 5 nt in length. In some cases, the cleavage can produce blunt ends. In some cases, the cleavage can produce staggered or sticky ends with 5′ overhangs. In some cases, the cleavage can produce staggered or sticky ends with 3′ overhangs.

Different organisms can comprise different PAM sequences. Different Cas proteins can recognize different PAM sequences. For example, in S. pyogenes, the PAM can be a sequence in the target nucleic acid that comprises the sequence 5′-NRR-3′, where R can be either A or G, where N can be any nucleotide and N can be immediately 3′ of the target nucleic acid sequence targeted by the spacer sequence. The PAM sequence of S. pyogenes Cas9 (SpyCas9) can be 5′-NGG-3′, where N can be any DNA nucleotide and can be immediately 3′ of the CRISPR recognition sequence of the non-complementary strand of the target DNA. The PAM of Cpf1 can be 5′-TTN-3′, where N can be any DNA nucleotide and can be immediately 5′ of the CRISPR recognition sequence.

The consensus PAM sequence for various MAD polypeptides has been determined (U.S. Pat. No. 9,982,279). The consensus PAM for MAD1-MADS, and MAD10-MAD12 was determined to be TTTN. The consensus PAM for MADS was determined to be NNG. The consensus PAM for MAD13-MAD15 was determined to be TTN. The consensus PAM for MAD16-MAD18 was determined to be TA. The consensus PAM for MAD19-MAD20 was determined to be TTCN.

Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site. The active variants can retain biological activity. The active variants can be recognized by a polynucleotide guided polypeptide (e.g., Cas protein). The active variants can be cleaved by a polynucleotide guided polypeptide (e.g., Cas protein). Assays can be used to measure the double-strand break of a target site by an endonuclease. Assays can measure the overall activity and/or specificity of an endonuclease on DNA substrates containing recognition sites (e.g., target sites, active variants).

### Methods for Integrating a Donor Polynucleotide

The disclosure provides methods to obtain an organelle comprising a donor polynucleotide. Such methods can employ homologous recombination to provide integration of the polynucleotide at the target site. A polynucleotide of interest can be provided to the organelle in a donor DNA molecule.

A donor polynucleotide can be a nucleic acid sequence (e.g., DNA, RNA, or both) that can be integrated into a target nucleic acid, for example, the genome of an organelle. The donor polynucleotide can be inserted into a genome e.g., at a cleavage site of a polynucleotide guided polypeptide. The donor polynucleotide can be inserted into a genome by homologous recombination. In some embodiments, the donor polynucleotide can comprise DNA and can be referred to as donor DNA.

A donor polynucleotide of any suitable size can be integrated into a genome. In some embodiments, the donor polynucleotide integrated into a genome can be less than 1, about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 or more than 500 kilobases (kb) in length. In some embodiments, the donor polynucleotide integrated into a genome can be at least about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 or more than 500 (kb) in length. In some embodiments, the donor polynucleotide integrated into a genome can be up to about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 or more than 500 (kb) in length.

A donor polynucleotide can comprise a polynucleotide of interest, a polynucleotide modification template, a heterologous expression cassette, or any combination. A donor polynucleotide (e.g. donor DNA) can be flanked by a first and a second region of homology. The polynucleotide modification template can be, for example, a single nucleotide change to create a different allele in the organelle genome. The first and second regions of homology of the donor polynucleotide (e.g. donor DNA) can share homology to a first and a second genomic region, respectively, present in or flanking the target site (e.g., of the organellar genome).

“Homology” can mean DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% homology or identity. For example, a “region of homology to a genomic region” can be a region of DNA that has a similar sequence to a given “genomic region” in the organellar genome. A region of homology can be of any length that can be sufficient to promote homologous recombination at the cleaved target site. For example, the region of homology can comprise at least 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo homologous recombination with the corresponding genomic region. “Sufficient homology” can indicate that two polynucleotide sequences can have sufficient structural similarity to act as substrates for a homologous recombination reaction.

The donor polynucleotide (e.g., donor DNA) may comprise an expression cassette (e.g., encoding a heterologous polynucleotide of interest). The donor polynucleotide may comprise multiple expression cassettes. The expression cassette may be a polycistronic expression cassette; e.g., where multiple protein-coding regions, functional RNAs, or a combination of both, are expressed under control of a single promoter.

A “donor RNA” can be a corresponding RNA molecule that comprises, for example, the same nucleic acid sequence as a donor DNA; i.e., with uridylate (“U”) in place of deoxythymidylate (“T”). A “donor polynucleotide” may be either a donor DNA or a donor RNA, or a combination of DNA and RNA. The donor polynucleotide may be either single-stranded or double-stranded.

An alternative method for modification of an organellar genome can be the replacement of part or all of the organelle DNA with a “replacement DNA”. Endogenous organellar DNA can be reduced or eliminated by use of site-specific endonucleases such as polynucleotide guided polypeptides (e.g., Cas polypeptide, Cas9 polypeptide, MAD polypeptide, MAD7 polypeptide). At the same time or subsequently, a replacement DNA may be introduced. The term “replacement DNA” can refer to fragments of organellar DNA or complete organellar DNA that can convey a new genotype and corresponding trait(s) when transformed into the organelle. The terms “replacement DNA” and “replacement organellar DNA” can be used interchangeably herein. In the case of organellar DNA fragments, they can be integrated into the remaining endogenous organellar DNA by homologous recombination. In the case of complete organellar DNA replacement, the replacement DNA can be isolated from cultivars, lines, sub species and other species which possess DNA compositions distinct from the endogenous organellar DNA of recipient cells. The replacement DNA can also be partially and/or completely synthesized in vitro. A replacement DNA can comprise both native and non-native sequences. When replacement DNA is created in vitro, it can be a linear DNA with the repeat sequence at the ends. The repeat sequences can be direct repeats or inverted repeats. The ends can facilitate homologous recombination in vitro or in vivo to create circular DNA for replication of organellar DNA in cells. The DNA created in vitro can also include exogenous DNA elements such as ones to allow selected amplification in bacterial cells. In some embodiments, the replacement DNA can comprise a DNA element functioning as a DNA replication origin in the recipient organelles. In some embodiments, the replacement DNA can comprise multiple DNA fragments that are capable of recombination within the organelle to result in the complete replacement DNA.

A sequence functional as an origin of replication can be included with the compositions (e.g., polynucleotides, constructs, cassettes) of the disclosure. Such sequences can include origin of replication for an organelle. The origin of replication sequence can be a plastid origin of replication (e.g., plastid rRNA intergenic region) sequence. The origin of replication sequence can be a mitochondrial origin of replication sequence.

As used herein, a “genomic region” can refer to a segment of a chromosome in the genome of, for example, an organelle. Genomic region can be present on either side of the target site. Genomic region can comprise a portion of the target site. The genomic region can comprise at least 5, 10, 15, 20, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases. The genomic region can comprise sufficient homology to undergo homologous recombination with the corresponding region of homology.

Donor polynucleotides, polynucleotides of interest and/or traits can be stacked together in a complex trait locus. The guide polynucleotide/polypeptide system can be used to generate double strand breaks and for stacking traits in a complex trait locus.

Two or more polynucleotides encoding RNA and/or proteins can be included in a cassette as a polycistronic unit. Polynucleotides encoding RNA can be expressed from separate cassettes.

In one embodiment, the guide polynucleotide/polypeptide system can be used for introducing one or more donor polynucleotides or one or more traits of interest into one or more target sites by providing one or more guide polynucleotides, one or more polynucleotide guided polypeptides (e.g., Cas polypeptides), and optionally one or more donor polynucleotides (e.g. donor DNA) to a plant cell. An organism can be produced from the cell that comprises an alteration at said one or more target sites of the organellar DNA, wherein the alteration can be a member selected from the group consisting of (i) replacement of at least one nucleotide, (ii) a deletion of at least one nucleotide, (iii) an insertion of at least one nucleotide, and (iv) any combination of (i)-(iii).

The structural similarity between a given genomic region and the corresponding region of homology flanking the donor polynucleotide (e.g. donor DNA) can be any degree of sequence identity that allows for homologous recombination to occur. For example, the amount of homology or sequence identity shared by the “region of homology” flanking the donor polynucleotide (e.g. donor DNA) and the “genomic region” of the plant genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology flanking the donor polynucleotide (e.g. donor DNA) can have homology to any sequence flanking the target site. While in some embodiments, the regions of homology share significant sequence homology to the genomic sequence immediately flanking the target site, the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” can refer to the exchange of DNA fragments between two DNA molecules at the sites of homology. The frequency of homologous recombination can be influenced by a number of factors. The length of the region of homology can affect the frequency of homologous recombination events, for example, the longer the region of homology, the greater the frequency. The length of the homology region needed to observe homologous recombination may vary among species.

Intermolecular recombination can occur in plastids, for example, transplastomic plants can arise through site-specific integration of foreign sequences by homologous recombination with the flanking sequence on the transformation vector.

Intramolecular recombination between repeated sequences can generate, for example, inversions when repeats are palindromic or deletions when direct.

To achieve efficient foreign sequence integration by homologous recombination endogenous plastome sequences can be used to target insertions. A positive correlation can be present between the rate of recombination and the length and/or degree of sequence homology.

The minimum flanking sequence length for homologous recombination with the organellar genome can be influenced by the introduction of single-stranded or double-stranded breaks (or both) in the organellar genome, e.g., by polynucleotide guided polypeptide(s).

In some embodiments, the efficiency of the disclosed methods for genome engineering or modification can be at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or 100%.

In one embodiment provided herein, the method can comprise contacting an organelle of a plant cell with the donor polynucleotide (e.g. donor DNA), the guide polynucleic acid and the polynucleotide guided polypeptide. At least one single-strand or double-strand break can be introduced in the target site by the polynucleotide guided polypeptide, the first and second regions of homology flanking the donor polynucleotide (e.g. donor DNA) can undergo homologous recombination with their corresponding genomic regions of homology resulting in exchange of DNA between the donor and the genome. As such, the provided methods can result in the integration of the donor polynucleotide (e.g. donor DNA) into the single-strand or double-strand break(s) in the target site in the organellar genome, thereby altering the original target site and producing an altered genomic target site.

The donor polynucleotide (e.g. donor DNA) may be introduced by any suitable means. For example, a plant having a target site can be provided. The donor polynucleotide (e.g. donor DNA) may be provided by any suitable transformation method including, for example, Agrobacterium-mediated transformation or biolistic particle bombardment. The donor polynucleotide (e.g. donor DNA) may be present transiently in the cell or it could be introduced via a viral replicon. In the presence of the guide polynucleotide (e.g., guide RNA), the polynucleotide guided polypeptide (e.g., Cas polypeptide, MAD polypeptide) and the target site, the donor polynucleotide (e.g. donor DNA) can be inserted into the organellar genome.

### Methods for Modulating Gene Expression

In some aspects, the present disclosure provides a method of selectively modulating transcription of a target nucleic acid (e.g., a gene) in a host cell. The method can involve introducing into the host cell an enzymatically inactive polynucleotide guided polypeptide (e.g., dead Cas) and a guide polynucleic acid. The guide nucleic acid and the dead Cas protein can form a complex in the host cell. The complex can selectively modulate transcription of a target polynucleic acid (e.g., target DNA) in the host cell or organelle.

In some aspects, the present disclosure provides for selective transcription modulation (e.g., reduction or increase) of a target nucleic acid in a host cell. Selective modulation of transcription of a target nucleic acid can reduce or increase transcription of the target nucleic acid, but may not substantially modulate transcription of a non-target nucleic acid or off-target nucleic acid, e.g., transcription of a non-target nucleic acid may be modulated by less than 1%, less than 5%, less than 10%, less than 20%, less than 30%, less than 40%, or less than 50% compared to the level of transcription of the non-target nucleic acid in the absence of the guide nucleic acid/enzymatically inactive or enzymatically reduced Cas protein complex. For example, selective modulation (e.g., reduction or increase) of transcription of a target nucleic acid can reduce or increase transcription of the target nucleic acid by at least about 10%, at least about 20%, at least about 30%, at least about 40%, at least about 50%, at least about 60%, at least about 70%, at least about 80%, at least about 90%, or greater than 90%, compared to the level of transcription of the target nucleic acid in the absence of a guide nucleic acid/enzymatically inactive or enzymatically reduced Cas protein complex.

In some aspects, the disclosure provides methods for increasing transcription of a target nucleic acid. The transcription of a target nucleic acid can increase by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 3.5 fold, at least about 4 fold, at least about 4.5 fold, at least about 5 fold, at least about 6 fold, at least about 7 fold, at least about 8 fold, at least about 9 fold, at least about 10 fold, at least about 12 fold, at least about 15 fold, at least about 20-fold, at least about 50-fold, at least about 70-fold, or at least about 100-fold compared to the level of transcription of the target polynucleic acid (e.g., target DNA) in the absence of a guide nucleic acid/enzymatically inactive or enzymatically reduced Cas protein complex. Selective increase of transcription of a target nucleic acid increases transcription of the target nucleic acid, but may not substantially increase transcription of a non-target polynucleic acid, e.g., transcription of a non-target nucleic acid can be increased, if at all, by less than about 5-fold, less than about 4-fold, less than about 3-fold, less than about 2-fold, less than about 1.8-fold, less than about 1.6-fold, less than about 1.4-fold, less than about 1.2-fold, or less than about 1.1-fold compared to the level of transcription of the non-targeted DNA in the absence of the guide nucleic acid/enzymatically inactive or enzymatically reduced Cas protein complex.

In some aspects, the disclosure provides methods for decreasing transcription of a target nucleic acid. The transcription of a target nucleic acid can decrease by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 3.5 fold, at least about 4 fold, at least about 4.5 fold, at least about 5 fold, at least about 6 fold, at least about 7 fold, at least about 8 fold, at least about 9 fold, at least about 10 fold, at least about 12 fold, at least about 15 fold, at least about 20-fold, at least about 50-fold, at least about 70-fold, or at least about 100-fold compared to the level of transcription of the target polynucleic acid (e.g., target DNA) in the absence of a guide nucleic acid/enzymatically inactive or enzymatically reduced Cas protein complex. Selective decrease of transcription of a target nucleic acid decreases transcription of the target nucleic acid, but may not substantially decrease transcription of a non-target DNA, e.g., transcription of a non-target nucleic acid can be decreased, if at all, by less than about 5-fold, less than about 4-fold, less than about 3-fold, less than about 2-fold, less than about 1.8-fold, less than about 1.6-fold, less than about 1.4-fold, less than about 1.2-fold, or less than about 1.1-fold compared to the level of transcription of the non-targeted DNA in the absence of the guide nucleic acid/enzymatically inactive or enzymatically reduced Cas protein complex.

Transcription modulation can be achieved by fusing the enzymatically inactive Cas protein to a heterologous sequence. The heterologous sequence can be a suitable fusion partner, e.g., a polypeptide that provides an activity that indirectly increases, decreases, or otherwise modulates transcription by acting directly on the target nucleic acid or on a polypeptide (e.g., a histone or other DNA-binding protein) associated with the target nucleic acid. Non-limiting examples of suitable fusion partners include a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity.

A suitable fusion partner can include a polypeptide that directly provides for increased transcription of the target nucleic acid. For example, a transcription activator or a fragment thereof, a protein or fragment thereof that recruits a transcription activator, or a small molecule/drug-responsive transcription regulator. A suitable fusion partner can include a polypeptide that directly provides for decreased transcription of the target nucleic acid. For example, a transcription repressor or a fragment thereof, a protein or fragment thereof that recruits a transcription repressor, or a small molecule/drug-responsive transcription regulator.

The heterologous sequence or fusion partner can be fused to the C-terminus, N-terminus, or an internal portion (i.e., a portion other than the N- or C-terminus) of the dead Cas protein.

### Methods for Delivery

Any suitable delivery method can be used for introducing the compositions and molecules of the disclosure into a host cell or organelle. The compositions (e.g., Cas protein, polynucleotide-guided polypeptide, guide polynucleic acid, donor polynucleotide) and/or nucleic acids encoding the compositions can be delivered simultaneously or temporally separated. The choice of method of genetic modification can be dependent on the type of cell being transformed and/or the circumstances under which the transformation is taking place (e.g., in vitro, ex vivo, in vivo, in planta).

Non-limiting examples of delivery methods or transformation include, for example, viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery and vacuum infiltration.

In some aspects, the present disclosure provides methods comprising delivering one or more polynucleotides, or one or more vectors as described herein, or one or more transcripts thereof, and/or one or proteins transcribed therefrom, to a host cell or organelle. In some aspects, the disclosure further provides cells produced by such methods, and organisms (such as animals, plants, or fungi) and organelles comprising or produced from such cells. In some embodiments, a polynucleotide guided polypeptide in combination with, and optionally complexed with, a guide sequence can be delivered to a cell or organelle.

Viral and non-viral based gene transfer methods can be used to introduce nucleic acids. Such methods can be used to administer nucleic acids encoding compositions of the disclosure to cells in culture, or in a host organism. Non-viral vector delivery systems can include DNA plasmids, RNA (e.g. a transcript of a vector described herein), naked nucleic acid, and nucleic acid complexed with a delivery vehicle, such as a liposome. Viral vector delivery systems can include DNA and RNA viruses, which can have either episomal or integrated genomes after delivery to the cell. Viral based systems can include retroviral, lentivirus, adenoviral, adeno-associated and herpes simplex virus vectors for gene transfer.

An adenoviral-based system can be used. Adenoviral-based systems can lead to transient expression of the transgene. Adenoviral based vectors can have high transduction efficiency in cells and may not require cell division. High titer and levels of expression can be obtained with adenoviral based vectors. Adeno-associated virus (“AAV”) vectors can be used to transduce cells with target nucleic acids, e.g., in the in vitro production of nucleic acids and peptides, and for in vivo and ex vivo gene therapy procedures.

In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a CRISPR complex, can be used to establish a new cell line comprising cells containing the modification but lacking any other exogenous sequence.

In some embodiments, compositions of the disclosure can be provided as RNA. In such cases, the compositions of the disclosure can be produced by direct chemical synthesis or may be transcribed in vitro from a DNA. The compositions of the disclosure can be synthesized in vitro using an RNA polymerase enzyme (e.g., T7 polymerase, T3 polymerase, SP6 polymerase, etc.). Once synthesized, the RNA can directly contact a target polynucleic acid (e.g., target DNA) or can be introduced into a cell using any suitable technique for introducing nucleic acids into cells (e.g., microinjection, electroporation, transfection, etc.).

Nucleotides encoding a guide nucleic acid (introduced either as DNA or RNA) and/or a polynucleotide guided polypeptide (introduced as DNA or RNA) can be provided to the cells using a suitable transfection technique. Nucleic acids encoding the compositions of the disclosure may be provided on vectors or cassettes (e.g., DNA vectors). Many vectors, e.g. plasmids, cosmids, minicircles, phage, viruses, etc., useful for transferring nucleic acids into target cells are available. The vectors comprising the nucleic acid(s) can be maintained episomally, e.g. as plasmids, minicircle DNAs, viruses such cytomegalovirus, adenovirus, etc., or they may be integrated into the target cell genome, through homologous recombination or random integration, e.g. retrovirus-derived vectors such as MMLV, HIV-1, and ALV.

The compositions of the disclosure may be fused to a polypeptide permeant domain to promote uptake by the cell. A number of polypeptide permeant domains can be used in the non-integrating polypeptides of the present disclosure, including peptides, peptidomimetics, and non-peptide carriers. The terms “permeant peptide”, “cell penetrating peptide”, “CPP”, “protein transduction domain” and “PTD” are used interchangeably herein. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 4). In some embodiments, a CPP can comprise the amino acid sequence of any one of SEQ ID NO: 4, 36, 37, 38, 39, 40, 41, 42, 43, 44, 45, 46, 47, 48, 49, 50, 51, 52 or any combination thereof. In some embodiments, a CPP can comprise at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO: 4, 36, 37, 38, 39, 40, 41, 42, 43, 44, 45, 46, 47, 48, 49, 50, 51, or 52. As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine (SEQ ID NO: 38), and octa-arginine (SEQ ID NO: 88). The nona-arginine (R9) sequence (SEQ ID NO: 38) can be used. Other cell penetrating peptides include the following: Pep-1, MPG, gamma-ZEIN, Transporant, MAP, Pept 1, Pept 2, IVV-14, Ig(v), Amphiphilic model peptide, pVEC, HRSV, Bp100 TAT2 or any combination thereof. The compositions of the disclosure may be fused to a combination of polypeptide permeant domains. The site at which the fusion can be made may be selected in order to optimize the biological activity, secretion or binding characteristics of the polypeptide.

The compositions of the disclosure may be fused to a polypeptide permeant domain to promote uptake by the cell. A number of polypeptide permeant domains can be used in the non-integrating polypeptides of the present disclosure, including peptides, peptidomimetics, and non-peptide carriers. The terms “permeant peptide”, “cell penetrating peptide”, “CPP”, “protein transduction domain” and “PTD” are used interchangeably herein. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 4). In some embodiments, a CPP can comprise the amino acid sequence of any one of SEQ ID NO: 4, 36, 37, 38, 39, 40, 41, 42, 43, 44, 45, 46, 47, 48, 49, 50, 51, 52 or any combination thereof. In some embodiments, a CPP can comprise at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to any one of SEQ ID NO: 4, 36, 37, 38, 39, 40, 41, 42, 43, 44, 45, 46, 47, 48, 49, 50, 51, or 52. As another example, the permeant peptide can comprise the HIV-1 tat basic region amino acid sequence, which may include, for example, amino acids 49-57 of naturally-occurring tat protein. Other permeant domains can include poly-arginine motifs, for example, the region of amino acids 34-56 of HIV-1 rev protein, nona-arginine, and octa-arginine. The nona-arginine (R9) sequence can be used. Other cell penetrating peptides include the following: Pep-1, MPG, gamma-ZEIN, Transporant, MAP, Pept 1, Pept 2, IVV-14, Ig(v), Amphiphilic model peptide, pVEC, HRSV, Bp100 TAT₂ or any combination thereof. The compositions of the disclosure may be fused to a combination of polypeptide permeant domains. The site at which the fusion can be made may be selected in order to optimize the biological activity, secretion or binding characteristics of the polypeptide.

The compositions of the disclosure may be associated with a peptide-based DNA carrier that comprises an organellar targeting signal. For organelle-specific delivery, a peptide-based DNA carrier can comprise two functional units: a polycationic DNA-binding domain (e.g., KH) and an organelle-targeting peptide (e.g., a chloroplast transit peptide, a mitochondrial targeting peptide).

The compositions of the disclosure may be prepared by in vitro synthesis. Various commercial synthetic apparatuses can be used. By using synthesizers, naturally occurring amino acids can be substituted with unnatural amino acids. The particular sequence and the manner of preparation can be determined by convenience, economics, and purity required.

In cases in which two or more different targeting complexes are provided to the cell (e.g., two different guide nucleic acids that are complementary to different sequences within the same or different target DNA), the complexes may be provided simultaneously (e.g., as two polypeptides and/or nucleic acids). Alternatively, they may be provided consecutively, e.g. the targeting complex being provided first, followed by the second targeting complex, or vice versa.

In cases in which targeting complex (es) and donor DNA are provided to the cell, the targeting complex (es) and donor DNA may be provided simultaneously. Alternatively, they may be provided consecutively, e.g., the targeting complex (es) being provided first, followed by the donor DNA, or vice versa.

### Genome Editing Using a Polynucleotide Guided Polypeptide System

As described herein, the polynucleotide guided polypeptide system can be used in combination with a co-delivered polynucleotide modification template to allow for editing of an organellar nucleotide sequence of interest. Also, as described herein, for each embodiment that uses an RNA guided polypeptide system, a similar polynucleotide guided polypeptide system can be deployed where the guide polynucleotide may not solely comprise ribonucleic acids but wherein the guide polynucleotide comprises a combination of RNA-DNA molecules or solely comprises DNA molecules.

Genome modification methods can rely on the homologous recombination system. Homologous recombination (HR) can provide molecular means for finding genomic DNA sequences of interest and modifying them according to the experimental specifications. Homologous recombination can be enhanced by introducing double-strand breaks (DSBs) at selected endonuclease target sites. Described herein is the use of a polynucleotide guided polypeptide system which can provide flexible genome cleavage specificity and can result in a high frequency of double-strand breaks at an organellar DNA target site. This specific cleavage can enable efficient gene editing of a nucleotide sequence of interest. The nucleotide sequence of interest to be edited can be located within or outside the target site recognized and/or cleaved by a polynucleotide guided polypeptide (e.g., a Cas polypeptide, a MAD polypeptide).

The term “polynucleotide modification template” can refer to a polynucleotide that can comprise at least one nucleotide modification when compared to the nucleotide sequence to be edited. A nucleotide modification can be at least one nucleotide substitution, addition or deletion. Examples of minor genome modifications created by use of a polynucleotide modification template include creation of a mutant allele (e.g., antibiotic resistant rRNA gene) and removal of a target site for a polynucleotide guided polypeptide. Optionally, the polynucleotide modification template can be flanked by homologous nucleotide sequences, wherein the flanking homologous nucleotide sequences can provide sufficient homology to the desired nucleotide sequence to be edited. The polynucleotide modification template can be a donor polynucleotide.

In one embodiment, the disclosure provides a method for editing a nucleotide sequence in the organellar genome of a cell. The method can comprise providing at least one guide polynucleotide (e.g., guide RNA), a polynucleotide modification template, and at least one polynucleotide guided polypeptide (e.g., Cas polypeptide, MAD polypeptide) to an organelle. The guide polynucleotide and the polynucleotide guided polypeptide can form a complex that can enable the polynucleotide guided polypeptide to introduce at least one single-strand or double-strand break at a target sequence in the organellar genome of the cell. The polynucleotide modification template can include at least one nucleotide modification of said nucleotide sequence. Cells include, but are not limited to, human, animal, bacterial, fungal, insect, and plant cells as well as organisms and tissues, e.g., plants and seeds, produced by the methods described herein. Cell can be an isolated and purified human cell. The nucleotide to be edited can be located within or outside a target site recognized and cleaved by a polynucleotide guided polypeptide. In one embodiment, the at least one nucleotide modification may not be a modification at a target site recognized and cleaved by a polynucleotide guided polypeptide. In another embodiment, there can be at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the organellar DNA target site.

In another embodiment, the at least one polynucleotide guided polypeptide (e.g., Cas9 polypeptide, MAD7 polypeptide) may be synthesized from a nucleic acid that is codon-optimized for expression in the nucleus or the organelle (e.g., the mitochondria or the plastid).

The nucleotide sequence to be edited can be a sequence that can be endogenous, artificial, pre-existing, or transgenic to the cell that is being edited. For example, the nucleotide sequence in the organellar genome of a cell can be a transgene that is stably incorporated into the organellar genome of a cell. Editing of such transgene may result in a further desired phenotype or genotype. The nucleotide sequence in the genome of a cell can also be a mutated or pre-existing sequence that was either endogenous or artificial from origin such as an endogenous gene or a mutated gene of interest.

In one embodiment, the region of interest can be flanked by two independent guide polynucleotide/polypeptide target sequences. Cutting can be done concurrently. The deletion event can be the repair of the two chromosomal ends without the region of interest. Alternative results can include inversions of the region of interest, mutations at the cut sites and duplication of the region of interest.

### Methods for Identifying at Least One Plant Cell Comprising in its Organellar Genome a Polynucleotide of Interest Integrated at the Target Site.

Further provided are methods for identifying at least one plant cell comprising in its organellar genome a polynucleotide of interest integrated at the target site. A donor polynucleotide can comprise a polynucleotide of interest. A variety of methods can be used for identifying those plant cells with an insertion into the genome at or near to the target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

The method can also comprise recovering a plant from the plant cell comprising a polynucleotide of interest integrated into its organellar genome. The plant may be sterile or fertile.

Polynucleotides/polypeptides of interest include, but are not limited to, herbicide-tolerance coding sequences, insecticidal coding sequences, nematicidal coding sequences, antimicrobial coding sequences, antifungal coding sequences, antiviral coding sequences, abiotic and biotic stress tolerance coding sequences, or sequences modifying plant traits such as yield, grain quality, nutrient content, starch quality and quantity, nitrogen fixation and/or utilization, and oil content and/or composition. polynucleotides of interest can include, but are not limited to, genes that improve crop yield, polypeptides that improve desirability of crops, genes encoding proteins conferring resistance to abiotic stress, such as drought, nitrogen, temperature, salinity, toxic metals or trace elements, or those conferring resistance to toxins such as pesticides and herbicides, or to biotic stress, such as attacks by fungi, viruses, bacteria, insects, and nematodes, and development of diseases associated with these organisms. Genes of interest can include, for example, those genes involved in information, such as zinc fingers, those involved in communication, such as kinases, and those involved in housekeeping, such as heat shock proteins. Polynucleotides of interest can include genes encoding important traits for agronomics, insect resistance, disease resistance, herbicide resistance, fertility or sterility, grain characteristics, and commercial products. Genes of interest can include, generally, those involved in oil, starch, carbohydrate, or nutrient metabolism as well as those affecting photosynthesis, photorespiration and ATP metabolism.

Commercial traits can also be obtained by expression of proteins encoded on a polynucleotide. A commercial use of transformed plants can be the production of polymers and bioplastics. Polynucleotides of interest can include genes encoding proteins such as (3-ketothiolase, PHBase (polyhydroxybutyrate synthase), and acetoacetyl-CoA reductase which can facilitate expression of polyhydroxyalkanoates (PHAs). Another commercial use can be expression of a gene or genes that can increase starch for ethanol production.

Polynucleotides/polypeptides that can influence amino acid biosynthesis include, for example, anthranilate synthase (AS; EC 4.1.3.27) which can catalyze the first reaction branching from the aromatic amino acid pathway to the biosynthesis of tryptophan in plants, fungi, and bacteria. In plants, the chemical processes for the biosynthesis of tryptophan can be compartmentalized in the chloroplast. Additional donor sequences of interest can include Chorismate Pyruvate Lyase (CPL) which can refer to a gene encoding an enzyme can which catalyze the conversion of chorismate to pyruvate and pHBA. Once example of CPL gene is from E. coli and bears the GenBank accession number M96268.

Polynucleotide sequences of interest may encode proteins involved in providing disease or pest resistance. By “disease resistance” or “pest resistance” can be intended that the plants can avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin; avirulence (avr) and disease resistance (R) genes; and the like. Insect resistance genes may encode resistance to pests that have great yield drag such as rootworm, cutworm, European Corn Borer, and the like. Such genes include, for example, Bacillus thuringiensis toxic protein genes; and the like.

The donor polynucleotide may also encode an RNA or double-stranded RNA that can be complementary to a target gene from a plant pest or plant pathogen. A method of alleviating pest infestation of plants can comprise, for example, a) identifying a DNA sequence from said pest which can be critical either for its survival, growth, proliferation or reproduction, b) cloning said sequence or a fragment thereof in a suitable vector relative to one or more promoters that can transcribe said sequence to RNA or dsRNA upon binding of an appropriate transcription factor to said promoters, and/or c) introducing said vector into the plant. The plant pest can be a nematode. Another method for alleviating pest infestation can include, for example, providing: a) DNA sequences which when transcribed yield a double-stranded RNA molecule that can reduce the expression of an essential gene of a plant sap-sucking insect; b) methods of using such DNA sequences and plants or plant cells transformed with such DNA sequences; and c) the use of cationic oligopeptides that facilitate the entry of dsRNA or siRNA molecules in insect cells, such as plant sap-sucking insect cells.

The donor polynucleotide may comprise and/or lead to expression of antisense sequences complementary to at least a portion of the messenger RNA (mRNA) for a targeted gene sequence of interest; e.g., a target gene from a plant pest or plant pathogen. Antisense nucleotides can be constructed to hybridize with the corresponding mRNA. Antisense nucleotides can be targeted to bind a splicing site on a pre-mRNA and modify the exon content of an mRNA, thereby modulating (e.g., disrupting) expression of a target gene.

Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

An “herbicide resistance protein” or a protein resulting from expression of an “herbicide resistance-encoding nucleic acid molecule” can include proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS), for example, the sulfonylurea-type herbicides, genes coding for resistance to herbicides that can act to inhibit the action of glutamine synthase, such as phosphinothricin or basta (e.g., the bar gene), glyphosate (e.g., the EPSP synthase gene and the GAT gene), HPPD inhibitors (e.g, the HPPD gene) or other such genes. The bar gene can encode resistance to the herbicide basta, the aadA can encode resistance to spectinomycin and streptomycin, the nptII gene can encode resistance to the antibiotics kanamycin and geneticin, and certain ALS-gene mutants can encode resistance to the herbicide chlorsulfuron.

In some crop species the use of hybrid plants has been shown to dramatically increase crop yield. Hybrid crop systems require male sterile lines that serve as the female parent to produce hybrid seed through fertilization with pollen donor plants. One method to convey male sterility without manual or mechanical intervention can be the use of cytoplasmic male sterility (CMS) genes. CMS is a maternally inherited trait conferred by the mitochondrial genome that results in a failure to produce functional pollen and/or male reproductive organs except in the presence of restorer-of-fertility (RF) genes (Chen et al. 2017 Critical Rev Plant Sci 36: 55-69). Chimeric mitochondrial ORFs can be found to lead to male sterility, producing unisex-female plants. The creation of these chimeric CMS genes may be a consequence of the highly recombinogenic, repetitive nature of plant mitochondrial genomes. The methods described herein could be used to introduce custom-designed, CMS ORFs into mitochondria of various monocot (e.g., wheat, maize, rice, barley, sorghum and rye) and dicot (e.g., soybean) species.

The donor polynucleotide can also be a phenotypic marker. A phenotypic marker can be screenable or a selectable marker that includes visual markers and selectable markers whether it is a positive or negative selectable marker. Any phenotypic marker can be used. Specifically, a selectable or screenable marker can comprise a DNA segment that can allow one to identify or select for or against a molecule or a cell that contains it, e.g., under particular conditions. These markers can encode an activity, such as, but not limited to, production of RNA, peptide, or protein, or can provide a binding site for RNA, peptides, proteins, inorganic and organic compounds or compositions and the like.

Examples of selectable and screenable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that can confer resistance to herbicidal compounds, such as glyphosate, sulfonylureas, glufosinate ammonium, bromoxynil, imidazolinones, and 2,4-dichlorophenoxyacetate (2,4-D).

The transgenes, recombinant DNA molecules, DNA sequences of interest, and donor polynucleotides can comprise one or more DNA sequences for gene silencing of a target gene; e.g., a target gene in a plant pest or plant pathogen. Methods for gene silencing involving the expression of DNA sequences in plant can include, but are not limited to, cosuppression, antisense suppression, double-stranded RNA (dsRNA) interference, hairpin RNA (hpRNA) interference, intron-containing hairpin RNA (ihpRNA) interference, transcriptional gene silencing, and microRNA (miRNA) interference.

In certain embodiments, a fertile plant can be a plant that can produce viable male and female gametes and can be self-fertile. Such a self-fertile plant can produce a progeny plant without the contribution from any other plant of a gamete and the genetic material contained therein. Other embodiments may involve the use of a plant that may not be self-fertile, for example, because the plant may not produce male gametes, or female gametes, or both, that are viable or otherwise capable of fertilization. As used herein, a “male-sterile plant” can be a plant that does not produce male gametes that are viable or otherwise capable of fertilization. As used herein, a “female-sterile plant” can be a plant that does not produce female gametes that are viable or otherwise capable of fertilization. Male-sterile and female-sterile plants can be female-fertile and male-fertile, respectively. A male-fertile (but female-sterile) plant can produce viable progeny when crossed with a female-fertile plant and that a female-fertile (but male-sterile) plant can produce viable progeny when crossed with a male-fertile plant.

### Methods Utilizing a Two Component RNA Guide and Polynucleotide Guided Polypeptide System

The polynucleotide guided polypeptide system described herein can be especially useful for genome engineering in circumstances where endonuclease off-target cutting can be toxic to the targeted cells. In one embodiment of the polynucleotide guided polypeptide system described herein, the constant component, a polynucleotide encoding an organelle targeted polynucleotide guided polypeptide, can be stably integrated into the nuclear genome of the cell. The polynucleotide can encode a modified polynucleotide guided polypeptide comprising an enzymatically active polynucleotide guided polypeptide (e.g., Cas polypeptide) fused to an organellar transport sequence (e.g., a mitochondrial targeting peptide or a chloroplast targeting peptide). Expression of the polynucleotide encoding the modified polynucleotide guided polypeptide can be under control of a promoter. The promoter can be a constitutive promoter, a tissue-specific promoter or an inducible promoter, e.g. a temperature-inducible, stress-inducible, developmental stage inducible, or chemically inducible promoter. In the absence of the variable component (e.g., the guide RNA or crRNA), the polynucleotide guided polypeptide may not cut the target nucleic acid. In the absence of the variable component (e.g., the guide RNA or crRNA) the presence of the polynucleotide guided polypeptide in the plant cell may have little or no consequence. A polynucleotide guided polypeptide system can be used to create and/or maintain a cell line or transgenic organism capable of efficient expression of the polynucleotide guided polypeptide. Expression of the polynucleotide guided polypeptide in the cell line or transgenic organism may have little or no consequence to cell viability.

In order to induce cutting at desired genomic sites to achieve targeted genetic modifications, guide polynucleotides (e.g., guide RNAs or crRNAs) can be introduced by a variety of methods into cells containing the stably-integrated and expressed expression cassette for the polynucleotide guided polypeptide. For example, guide polynucleotide (e.g., guide RNAs or crRNAs) can be chemically or enzymatically synthesized, and introduced into the polynucleotide guided polypeptide expressing cells via direct delivery methods such a particle bombardment or electroporation. A guide polynucleic acid may be fused to an RNA molecule that allows for transport into an organelle. Alternatively, a guide polynucleic acid may be fused to an RNA molecule that allows for binding to a protein that facilitates transport into the organelle. Alternatively, a guide polynucleic acid may be transported into the organelle by association with the modified polynucleotide guided polypeptide comprising an enzymatically active polynucleotide guided polypeptide fused to an organellar transport sequence.

Alternatively, genes that can efficiently express guide polynucleotides (e.g., guide RNAs or crRNAs) in the target cells can be synthesized chemically, enzymatically or in a biological system. These genes can be introduced into the polynucleotide guided polypeptide expressing cells, for example, via direct delivery methods such a particle bombardment, electroporation, vacuum infiltration or biological delivery methods such as Agrobacterium-mediated DNA delivery.

One embodiment of the disclosure can be a method for selecting a plant comprising an altered organellar genome. A suitable method can be used to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

Sufficient homology or sequence identity can indicate that two polynucleotide sequences have sufficient structural similarity to act as substrates for a homologous recombination reaction. The structural similarity can include overall length of each polynucleotide fragment, and the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of homology or sequence identity shared by a target and a donor polynucleotide can vary. For example, the length of sequence homology may be at least one of the following: 20 bp, 50 bp, 100 bp, 150 bp, 250 bp, 300 bp, 400 bp, 500 bp, 600 bp, 700 bp, 800 bp, 900 bp, 1000 bp, 1250 bp, 1500 bp, 1750 bp, 2000 bp, 2.5 kb, 3 kb, 4 kb, 5 kb, 6 kb, 7 kb, 8 kb, 9 kb or 10 kb. The amount of homology can also be described by percent sequence identity over the full aligned length of the two polynucleotides which includes percent sequence identity of at least any of the following: 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology can include any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions.

Protocols for introducing polynucleotides and polypeptides into plants may vary depending on the type of plant or plant cell targeted for transformation, such as monocot or dicot. Suitable methods of introducing polynucleotides and polypeptides into plant cells and subsequent insertion into the plant genome include microinjection, meristem transformation, electroporation, Agrobacterium-mediated transformation, direct gene transfer, vacuum infiltration and ballistic particle acceleration.

Alternatively, polynucleotides may be introduced into plants by contacting plants with a virus or viral nucleic acids. Such methods can involve incorporating a polynucleotide within a viral DNA or RNA molecule. In some examples a polypeptide of interest may be initially synthesized as part of a viral polyprotein, which can be later processed by proteolysis in vivo or in vitro to produce the desired recombinant protein. Methods for introducing polynucleotides into plants and expressing a protein encoded therein, can involve viral DNA or RNA molecules.

Transient transformation methods include, but are not limited to, the introduction of polypeptides, such as a double-strand break inducing agent, directly into the organism, the introduction of polynucleotides such as DNA and/or RNA polynucleotides, and the introduction of the RNA transcript, such as an mRNA encoding a double-strand break inducing agent, into the organism. Such methods include, for example, microinjection or particle bombardment.

DNA transformation of organellar genomes can be performed in, for example, plastids and mitochondria. Selectable marker genes can include, for example, photosynthesis (atpB, tscA, psaA/B, petB, petA, ycf3, rpoA, rbcL), antibiotic resistance (rrnS, rrnL, aadA, nptII, aphA-6), herbicide resistance (psbA, bar, AHAS (ALS), EPSPS, HPPD, sul) and metabolism (BADH, codA, ARG8, ASA2) genes. The sul gene from bacteria has herbicidal sulfonamide-insensitive dihydropteroate synthase activity and can be used as a selectable marker when the protein product is targeted to plant mitochondria (U.S. Pat. No. 6,121,513).

In some embodiments, the sequence encoding the marker may be incorporated into the genome of the organelle. In some embodiments, the incorporated sequence encoding the marker may by subsequently removed from the transformed organellar genome. Removal of a sequence encoding a marker may be facilitated by the presence of direct repeats before and after the region encoding the marker. Removal of the sequence encoding the marker can occur via the endogenous homologous recombination system of the organelle or by use of a site-specific recombinase system such as cre-lox or FLP/FRT (U.S. Pat. No. 6,849,778).

Caspase Activatable-GFP (CA-GFP) is a modified version of GFP in which fluorescence is completely quenched by appendage of a hydrophobic quenching peptide that tetramerizes GFP and prevents maturation of the chromophore (Nicholls et al. 2011 J Biol Chem 286: 24977-24986). The sequence of CA-GFP protein corresponds to GFP with a fusion of DEVDFQGPCNDSSDPLVVAASIIGILHLILWILDRL (SEQ ID NO: 5) at the carboxy terminus. A caspase recognition sequence comprising the amino acids DEVD (SEQ ID NO: 6) is present in CA-GFP between the fluorescence and the quenching domains. GFP fluorescence can be fully restored in vivo by catalytic removal of the quenching peptide by cleavage with caspase. In one embodiment, the nucleic acid sequence encoding CA-GFP can be modified by replacement of the caspase recognition sequence with a mitochondrial RNA editing sequence. The RNA editing sequence is selected such that a C-to-U conversion results in creation of a stop codon in the mRNA. Consequently, expression of the nucleic acid sequence encoding the modified CA-GFP would result in quenching in the cytoplasm or in plastids but would produce fluorescence in mitochondria, thus providing a screenable marker. Candidate RNA editing sequences for this purpose are present in the wheat mitochondrial cox2 gene at positions 449, 587 and 620 of the gene, where the A residue of the initiation codon is the first base. These sequences are presented as SEQ ID NO: 7, SEQ ID NO: 8 and SEQ ID NO: 9, respectively.

DNA transformation of, for example, the yeast nuclear genome can be facilitated by the development of shuttle vectors that can replicate in E. coli and yeast as autonomous plasmids. Vector systems can include low-copy-number plasmids and integrative DNA through homologous recombination.

Methods of the invention can provide transformation efficiency into an organelle (e.g., mitochondria, plastids) of, for example, at least about: 1%, 2%, 5%, 10%, 20%, 30%, 40%, 50%, 60%, 70%, 80%, 90%, or 100% transformation efficiency.

In one embodiment, an expression construct of the current disclosure may comprise a promoter operably linked to a nucleotide sequence encoding a polynucleotide guided polypeptide gene and a promoter operably linked to a guide RNA. The promoter can drive expression of an operably linked nucleotide sequence in a cell. The promoter can be a constitutive promoter, a tissue-specific promoter or an inducible promoter, e.g. a temperature-inducible, stress-inducible, developmental stage inducible, or chemically inducible promoter.

The cells having the introduced sequence may be grown or regenerated into plants. These plants may then be grown, and either pollinated with the same transformed strain or with a different transformed or untransformed strain, and the resulting progeny having the desired characteristic and/or comprising the introduced polynucleotide or polypeptide identified. Two or more generations may be grown to ensure that the polynucleotide can be stably maintained and inherited, and seeds harvested.

Any plant can be used, including monocot and dicot plants. Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), maize, wheat (Triticum aestivum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses. Examples of dicot plants that can be used include, but are not limited to, soybean (Glycine max), canola (Brassica napus and B. campestris), alfalfa (Medicago sativa), tobacco (Nicotiana tabacum), Arabidopsis (Arabidopsis thaliana), sunflower (Helianthus annuus), cotton (Gossypium arboreum), and peanut (Arachis hypogaea), tomato (Solanum lycopersicum), potato (Solanum tuberosum) etc.

The transgenes, recombinant DNA molecules, DNA sequences of interest, and donor polynucleotides can comprise one or more genes of interest. Such genes of interest can encode, for example, a protein that can provide an agronomic advantage to the plant.

Also, as described herein, for each example or embodiment that cites a guide RNA, a similar guide polynucleotide can be designed wherein the guide polynucleotide does not solely comprise ribonucleic acids but wherein the guide polynucleotide comprises a combination of RNA-DNA molecules or solely comprises DNA molecules.

After creating a designed change in organellar DNA, the next step can be to maintain the edited organellar DNA in the pool of unmodified organellar DNA and to shift the balance among organellar DNA to favor the maintenance of genome edited organellar DNA. This can be achieved by reducing the amplification of unmodified organellar DNA. In one approach, guide polynucleic acids can be designed for multiple target sites in the unmodified organelle genome. The donor polynucleotide (e.g. donor DNA) can be designed such that these target sites have been altered to no longer be recognized by the relevant polynucleotide guided polypeptide system(s). Expression of the polynucleotide guided polypeptides can result in the introduction of single-strand or double-strand breaks into the unmodified organellar DNA and can thereby increase the proportion of modified genomes. In one variation, cells may be pretreated with relevant polynucleotide guided polypeptide systems to introduce cleavages in organellar DNA. The pretreatment can reduce the number of organelle DNA molecules available for homologous recombination.

Embodiments can involve a single guide RNA (sgRNA), i.e., where the variable targeting domain can be fused to a polynucleotide that contains a tracrRNA sequence. Alternatively, embodiments may involve a duplex guide RNA, i.e., where the variable targeting domain and the tracrRNA sequence are present on separate RNA molecules. The terms “duplex guide RNA” and “dual guide RNA” are used interchangeably herein.

In some cases, protein and/or RNA expression levels can be higher when transformed into an organelle (e.g., plastid, mitochondria) compared with that in nucleus. For example, protein expression level can be at least about: 1%, 5%, 10%, 20%, 30%, 40%, 50%, 60%, 70%, 80%, or 90% higher with organelle transformation when compared with nuclear transformation. The expression stability of a transcript can be higher with organelle transformation compared with nuclear transformation.

### SPECIFIC EMBODIMENTS

Embodiment 1. A method for altering an organellar genome, the method comprising:
a. introducing into a nucleus of a cell:
   i. a first polynucleotide encoding at least in part a modified polynucleotide guided polypeptide, wherein the modified polynucleotide guided polypeptide comprises a polynucleotide guided polypeptide operably linked to an organellar targeting peptide, wherein the polynucleotide guided polypeptide when associated with a guide RNA, cleaves at least one target sequence present in the organellar genome; and
   ii. a second polynucleotide comprising at least in part at least one guide RNA, wherein the at least one guide RNA directs the polynucleotide guided polypeptide to cleave the at least one target sequence present in the organellar genome;
b. introducing into an organelle of the cell, a third polynucleotide comprising at least in part at least one homologous organellar DNA sequence, wherein the at least one homologous organellar DNA is capable of homologous recombination, wherein integration of the at least one homologous organellar DNA sequence into the organellar genome results in a recombined organellar genome lacking the at least one target sequence;
c. growing a cell comprising the nucleus of (a) and the organelle of (b) under conditions in which the first polynucleotide and the second polynucleotide are expressed; and
d. selecting a cell comprising an altered organellar genome.

Embodiment 2. A method for altering an organellar genome, the method comprising:
a. introducing into a nucleus of a cell:
   i. a first polynucleotide encoding at least in part a modified polynucleotide guided polypeptide, wherein the modified polynucleotide guided polypeptide comprises a polynucleotide guided polypeptide operably linked to an organellar targeting peptide, wherein the polynucleotide guided polypeptide when associated with a guide RNA, cleaves at least one target sequence present in the organellar genome;
b. introducing into an organelle of the cell:
   1. a second polynucleotide comprising at least in part at least one guide RNA, wherein the at least one guide RNA directs the polynucleotide guided polypeptide to cleave the at least one target sequence present in the organellar genome;
c. growing a cell comprising the nucleus of (a) and the organelle of (b) under conditions in which the first polynucleotide and the second polynucleotide are expressed; and
d. selecting a cell comprising an altered organellar genome.

Embodiment 3. The method of embodiment 2, wherein (b) further comprises introducing into the organelle of the cell:
ii. a third polynucleotide comprising at least in part at least one homologous organellar DNA sequence, wherein the at least one homologous organellar DNA is capable of homologous recombination, wherein integration of the at least one homologous organellar DNA sequence into the organellar genome results in a recombined organellar genome lacking the at least one target sequence.

Embodiment 4. The method of any one of embodiments 1-3, wherein the polynucleotide guided polypeptide comprises at least one member selected from the group consisting of: a Cas9 protein, a MAD2 protein, a MAD7 protein, a CRISPR nuclease, a nuclease domain of a Cas protein, a Cpf1 protein, an Argonaute, modified versions thereof, and any combination thereof.

Embodiment 5. The method of any one of embodiments 1-4, wherein the at least one guide RNA is processed from a polycistronic RNA after transcription by use of at least one member selected from the group consisting of: an RNA cleavage site, a ribozyme cleavage site, a polynucleotide guided polypeptide cleavage site, a presence of a tRNA sequence, and any combination thereof.

Embodiment 6. The method of embodiment 5, comprising the presence of the tRNA sequence, wherein the at least one guide RNA is processed from a polycistronic RNA by having a first tRNA sequence 5′ to the at least one guide RNA and a second tRNA sequence 3′ to the at least one guide RNA.

Embodiment 7. A method for altering an organellar genome, the method comprising:
a. introducing into a nucleus of a cell: a first polynucleotide encoding a modified site-directed nuclease, wherein the modified site-directed nuclease comprises a site-directed nuclease operably linked to an organellar targeting peptide, wherein the site-directed nuclease cleaves at least one target sequence present in the organellar genome; and
b. introducing into an organelle of the cell, a third polynucleotide comprising at least one homologous organellar DNA sequence, wherein the at least one homologous organellar DNA is capable of homologous recombination, wherein integration of the at least one homologous organellar DNA sequence into the organellar genome results in a recombined organellar genome lacking the at least one target sequence;
c. growing a cell comprising the nucleus of (a) and the organelle of (b) under conditions in which the first polynucleotide is expressed; and
d. selecting a cell comprising an altered organellar genome.

Embodiment 8. The method of embodiment 7, wherein the site-directed nuclease comprises at least one member selected from the group consisting of: a TALEN, a Zinc-Finger Nuclease, a Meganuclease, a restriction enzyme, and any combination thereof.

Embodiment 9. The method of any one of embodiments 1-8, wherein (a) and (b) occur in separate cells.

Embodiment 10. The method of embodiment 9, wherein the nucleus of (a) and the organelle of (b) are brought together into a cell by sexual crossing, cell fusion, microinjection, or any combination thereof.

Embodiment 11. The method of any one of embodiments 1-10, wherein the method further comprises: (e) selecting a cell that is homoplasmic for the altered organellar genome.

Embodiment 12. The method of any one of embodiments 1-11, wherein the third polynucleotide encoding the at least one homologous organellar DNA sequence is operably linked to an origin of replication that is functional in the organelle.

Embodiment 13. The method of any one of embodiments 1-12, wherein the third polynucleotide encoding the at least one homologous organellar DNA sequence comprises a fourth polynucleotide encoding at least one selectable marker or at least one screenable marker, or both.

Embodiment 14. The method of embodiment 13, wherein the fourth polynucleotide, after integration into the organellar genome, is operably linked to a promoter that is functional in the organelle.

Embodiment 15. The method of any one of embodiments 1-14, wherein the third polynucleotide encoding the at least one homologous organellar DNA sequence comprises a fifth polynucleotide and a sixth polynucleotide, wherein the fifth polynucleotide and the sixth polynucleotide each comprise a region of homology in the organellar genome.

Embodiment 16. The method of embodiment 15, wherein the region of homology in the fifth polynucleotide and the region of homology in the sixth polynucleotide correspond to two adjacent regions of homology in the organellar genome.

Embodiment 17. The method of embodiment 15 or 16, wherein the fifth polynucleotide and the sixth polynucleotide are separated by a seventh polynucleotide.

Embodiment 18. The method of embodiment 17, wherein the seventh polynucleotide comprises a sequence that is heterologous to the organellar genome.

Embodiment 19. The method of embodiment 17 or 18, wherein the seventh polynucleotide encodes an RNA that is heterologous to the organelle.

Embodiment 20. The method of embodiment 17, 18 or 19, wherein the seventh polynucleotide encodes: a cytoplasmic male sterility factor, a dsRNA, a siRNA, a miRNA, or any combination thereof.

Embodiment 21. The method of embodiment 20, wherein the dsRNA, the siRNA or the miRNA suppresses at least one target gene necessary for male fertility in a plant.

Embodiment 22. The method of embodiment 17 or 18, wherein the seventh polynucleotide encodes: a herbicide tolerance protein, a pesticidal protein, an accessory protein that binds to a pesticidal protein, a dsRNA, a siRNA, a miRNA or any combination thereof.

Embodiment 23. The method of embodiment 22, wherein the seventh polynucleotide encodes a herbicide tolerance protein.

Embodiment 24. The method of embodiment 23, wherein the herbicide tolerance protein comprises a 4-hydroxphenylpyruvate dioxygenase (HPPD), a sulfonylurea-tolerant acetolactate synthase (ALS), an imidazolinone-tolerant acetolactate synthase (ALS), a glyphosate-tolerant 5-enolpyruvylshikimate-3-phosphate synthase (EPSPS), a glyphosate-tolerant glyphosate oxidoreductase (GOX), a glyphosate N-acetyltransferase (GAT), a phosphinothricin acetyl transferase (PAT), a protoporphyrinogen oxidase (PROTOX), an auxin enzyme or receptor, a P450 polypeptide, an acetyl coenzyme A carboxylase (ACCase), or any combination thereof.

Embodiment 25. The method of embodiment 22, wherein the seventh polynucleotide encodes the pesticidal protein.

Embodiment 26. The method of embodiment 25, wherein the pesticidal protein comprises Cry1Ac, Cyt1Aa, Cry1Ab, Cry2Aa, Cry1I, Cry1C, Cry1D, Cry1E, Cry1Be, Cry1Fa, Vip3A, or any combination thereof.

Embodiment 27. The method of embodiment 22, wherein the seventh polynucleotide encodes the accessory protein.

Embodiment 28. The method of embodiment 27, wherein the accessory protein binds to the pesticidal protein.

Embodiment 29. The method of embodiment 28, wherein the pesticidal protein comprises a 20 kDa accessory protein, a 19 kDa accessory protein or any combination thereof.

Embodiment 30. The method of embodiment 22, wherein the dsRNA, the siRNA or the miRNA suppress at least one target gene present in a plant pest.

Embodiment 31. The method of embodiment 17 or 18, wherein the seventh polynucleotide encodes: β-ketothiolase, polyhydroxybutyrate synthase, acetoacetyl-CoA reductase, anthranilate synthase, chorismate pyruvate lyase, large subunit of a RUBISCO, or any combination thereof.

Embodiment 32. The method of any one of embodiments 17-31, wherein the seventh polynucleotide is operably linked to at least one regulatory element that is active in an organelle.

Embodiment 33. The method of embodiment 32, wherein the regulatory element comprises a maize clpP promoter combined with a maize clpP 5′-UTR, a maize clpP promoter combined with a 5′-UTR from gene 10 of bacteriophage T7, a tomato psbA promoter combined with a 5′-UTR from gene 10 of bacteriophage T7, a tomato rrn16 promoter combined with a modified accD 5′-UTR, and any combination thereof.

Embodiment 34. The method of any one of embodiments 13-33, wherein the fourth polynucleotide comprises a first sequence encoding a positive selectable marker.

Embodiment 35. The method of any one of embodiments 13-34, wherein the fourth polynucleotide comprises a second sequence encoding a negative selectable marker.

Embodiment 36. The method of embodiment 35, wherein the first sequence and the second sequence are each operably linked to a promoter that is functional in the organelle.

Embodiment 37. The method of any one of embodiments 1-36, wherein the third polynucleotide further comprises an eighth polynucleotide and a ninth polynucleotide, wherein the eight polynucleotide and the ninth polynucleotide have 100 percent sequence identity to each other or to an endogenous sequence in the organellar genome, and have sufficient length for homologous recombination.

Embodiment 38. The method of embodiment 37, wherein a length for the homologous recombination is at least 10, at least 15, at least 20, at least 25, at least 30, at least 35, at least 40, at least 50, at least 60, at least 70, at least 80, at least 90, at least 100, at least 150, at least 200, or at least 250 nucleotides.

Embodiment 39. The method of embodiment 37 or 38, wherein a length for the homologous recombination can be at most 10, at most 15, at most 20, at most 25, at most 30, at most 35, at most 40, at most 50, at most 60, at most 70, at most 80, at most 90, at most 100, at most 150, at most 200, or at most 250 nucleotides.

Embodiment 40. The method of embodiment 37-39, wherein the eighth polynucleotide and the ninth polynucleotide are arranged as direct repeats in a recombinant DNA construct.

Embodiment 41. The method of embodiment 40, wherein the direct repeats comprise a site-specific recombinase site.

Embodiment 42. The method of embodiment 41, wherein the site-specific recombinase comprises loxP, attP, attB or a combination thereof.

Embodiment 43. The method of any one of embodiments 1-42, wherein the growing the cell is under conditions wherein a site-specific recombinase is expressed in the organelle.

Embodiment 44. The method of embodiment 43, wherein the site-specific recombinase comprises Cre, phiC31, Bxb1 or a combination thereof.

Embodiment 45. The method of any one of embodiments 1-44, wherein the third polynucleotide is linear.

Embodiment 46. The method of any one of embodiments 1-44, wherein the third polynucleotide is circular.

Embodiment 47. The method of any one of embodiments 1-46, wherein the third polynucleotide comprises RNA, DNA or a combination thereof.

Embodiment 48. The method of any one of embodiments 1-47, wherein the third polynucleotide is single stranded.

Embodiment 49. The method of any one of embodiments 1-47, wherein the third polynucleotide is double stranded.

Embodiment 50. The method of any one of embodiments 1-49, wherein the third polynucleotide comprises a length of at least 100, 150, 200, 250, 300, 400, 500, 100, 1500 or 2000 nucleotides.

Embodiment 51. The method of any one of embodiments 37-50, wherein the eighth polynucleotide and the ninth polynucleotide are present at the 5′ and 3′ ends of the third polynucleotide.

Embodiment 52. The method of any one of embodiments 1-51, wherein the cell is selected from the group consisting of: a yeast cell, an algal cell, a plant cell, an insect cell, a non-human animal cell, an isolated and purified human cell, a mammalian tissue culture cell, and any combination thereof.

Embodiment 53. The method of any one of embodiments 1-52, wherein the alteration of the organellar genome comprises an insertion of an expression cassette.

Embodiment 54. The method of embodiment 53, wherein the expression cassette is a polycistronic expression cassette.

Embodiment 55. The method of embodiment 54, wherein the polycistronic expression cassette encodes a selectable marker or a screenable marker, or both.

Embodiment 56. The method of any one of embodiments 1-55, wherein the organelle is a mitochondrion.

Embodiment 57. The method of any one of embodiments 1-55, wherein the organelle is a plastid.

Embodiment 58. The method of any one of embodiments 1-55, wherein the organelle is a chloroplast.

Embodiment 59. The method of any one of embodiments 1-58, wherein the second polynucleotide comprises at least 17 nucleotides.

Embodiment 60. The method of any one of embodiments 1-59, wherein the at least one guide RNA comprises at least one single guide RNA or at least one duplex guide RNA.

Embodiment 61. The method of any one of embodiments 1-60, wherein the at least one guide RNA comprises one guide RNA or a plurality of guide RNAs.

Embodiment 62. The method of embodiment 61, wherein the plurality of guide RNAs are encoded on separate transcription units or on a polycistronic transcription unit.

Embodiment 63. The method of embodiment 62, wherein the plurality of guide RNAs are encoded on a polycistronic transcription unit.

Embodiment 64. The method of any one of embodiments 1-63, wherein the at least one guide RNA is processed from a polycistronic RNA after transcription by use of an RNA cleavage site, a ribozyme cleavage site, a polynucleotide guided polypeptide cleavage site, a presence of a tRNA sequence, or any combination thereof.

Embodiment 65. The method of any one of embodiments 1-64, wherein the at least one guide RNA is processed from a polycistronic RNA by having a first tRNA sequence 5′ to the guide RNA and a second tRNA sequence 3′ to the guide RNA.

Embodiment 66. The method of any one of embodiments 1-65, wherein the polynucleotide guided polypeptide is codon-optimized for a human, a yeast, an alga, or a plant species.

Embodiment 67. The method of any one of embodiments 1-66, wherein at least one member selected from the group consisting of: the first polynucleotide, the second polynucleotide, the third polynucleotide, and any combination thereof is introduced into the cell via at least one method selected from the group consisting of: microinjection, meristem transformation, electroporation, Agrobacterium-mediated transformation, viral based gene transfer, transfection, vacuum infiltration, biolistic particle bombardment, and any combination thereof.

Embodiment 68. The method of any one of embodiments 1-67, wherein at least one member selected from the group consisting of: the first polynucleotide, the second polynucleotide, the third polynucleotide, and any combination thereof is introduced into the cell as a peptide-polynucleotide complex.

Embodiment 69. The method of embodiment 68, wherein at least one peptide of the peptide-polynucleotide complex comprises at least one member selected from the group consisting of: a cell penetrating peptide (CPP), an organellar targeting peptide, a histidine rich peptide, a lysine-rich peptide, and any combination thereof.

Embodiment 70. The method of embodiment 69, wherein the at least one peptide of the peptide-polynucleotide complex comprises a CPP.

Embodiment 71. The method of embodiment 70, wherein the CPP comprises penetratin, TAT, R9, Pep-1, MPG, gamma-ZEIN, Transporant, MAP, Pept 1, Pept 2, IVV-14, Ig(v), Amphiphilic model peptide, pVEC, HRSV, Bp100, TAT₂ or any combination thereof.

Embodiment 72. The method of embodiment 70, wherein the CPP comprises at least 80%, 90%, 95%, 100% sequence identity to SEQ ID NO: 4, 36, 37, 38, 39, 40, 41, 42, 43, 44, 45, 46, 47, 48, 49, 50, 51, 52 or any combination thereof.

Embodiment 73. The method of any one of embodiments 1-72, further comprising introducing into the organelle a polynucleotide encoding a marker.

Embodiment 74. The method of embodiment 73, wherein the marker is a positive selectable marker, a negative selectable marker, a screenable marker, or any combination thereof.

Embodiment 75. The method of embodiment 74, wherein the marker is a positive selectable marker.

Embodiment 76. The method of embodiment 75, wherein the positive selectable marker comprises an herbicide tolerance protein.

Embodiment 77. A method of growing a cell produced by the method of any one of embodiments 1-76.

Embodiment 78. The method of embodiment 77, further comprising growing the cell in a presence of a positive selection agent and selecting a cell that is homoplasmic for the altered organellar genome.

Embodiment 79. The method of embodiment 77, further comprising growing the cell in an absence of a positive selection agent, followed by selecting a cell that lacks a non-integrated recombinant DNA construct.

Embodiment 80. The method of embodiment 77, further comprising growing the cell in an absence of a positive selection agent, followed by growing the cell in a presence of a negative selection agent, followed by selecting a cell that lacks a non-integrated recombinant DNA construct.

Embodiment 81. A cell produced by the method of any one of embodiments 1-80, wherein the cell is a yeast cell, an algal cell, a plant cell, an insect cell, a non-human animal cell, an isolated and purified human cell, a mammalian tissue culture cell, and any combination thereof.

Embodiment 82. A plant, seed, root, stem, leaf, flower, or fruit produced from the cell of embodiment 80, wherein the plant, seed, root, stem, leaf, flower, or fruit comprises the altered organellar genome.

Embodiment 83. A method for altering an organellar genome, the method comprising:
a. introducing into a nucleus of a cell:
   i. a first polynucleotide encoding a modified polynucleotide guided polypeptide, wherein the modified polynucleotide guided polypeptide comprises a polynucleotide guided polypeptide operably linked to an organellar targeting peptide, wherein the polynucleotide guided polypeptide when associated with a guide RNA, cleaves at least one target sequence present in an organellar genome; and
   ii. a second polynucleotide encoding at least one guide RNA, wherein the at least one guide RNA directs the polynucleotide guided polypeptide to cleave the at least one target sequence present in the organellar genome;
b. introducing into an organelle of the cell a replacement DNA;
c. growing a cell comprising the nucleus of (a) and the organelle of (b) under conditions in which the first polynucleotide and the second polynucleotide are expressed; and
d. selecting a cell comprising an altered organellar genome.

Embodiment 84. A method for altering an organellar genome, the method comprising:
a. introducing into a nucleus of a cell a first polynucleotide encoding a modified polynucleotide guided polypeptide, wherein the modified polynucleotide guided polypeptide comprises a polynucleotide guided polypeptide operably linked to an organellar targeting peptide, wherein the polynucleotide guided polypeptide when associated with a guide RNA, cleaves at least one target sequence present in an organellar genome;
b. introducing into an organelle of the cell:
   i. a second polynucleotide encoding at least one guide RNA, wherein the at least one guide RNA directs the polynucleotide guided polypeptide to cleave the at least one target sequence present in the organellar genome; and
   ii. a replacement DNA;
c. growing a cell comprising the nucleus of (a) and the organelle of (b) under conditions in which the first polynucleotide and the second polynucleotide are each expressed; and
d. selecting a cell comprising an altered organellar genome

Embodiment 85. The method of embodiment 83 or 84, wherein the replacement DNA comprises fragments of organellar DNA or a complete organellar DNA from a cultivar, line, sub-species or other species.

Embodiment 86. The method of any one of embodiments 83-85, wherein the replacement DNA is distinct from the organellar genome of the cell.

Embodiment 87. The method of any one of embodiments 83-86, wherein the at least one target sequence is not present in the replacement DNA.

Embodiment 88. The method of any one of embodiments 83-87, wherein after (a) and prior to (b), a cell is selected in which a native organellar genome has been eliminated.

Embodiment 89. The method of any one of embodiments 83-88, wherein the organelle is a mitochondrion.

Embodiment 90. The method of any one of embodiments 83-88, wherein the organelle is a plastid.

Embodiment 91. The method of any one of embodiments 83-88, wherein the organelle is a chloroplast.

Embodiment 92. A cell produced by the method of any one of embodiments 83-91, wherein the cell is a yeast cell, an algal cell, a plant cell, an insect cell, a non-human animal cell, an isolated and purified human cell, or a mammalian tissue culture cell.

Embodiment 93. A plant, seed, root, stem, leaf, flower, or fruit produced from the cell of embodiment 92, wherein the plant, seed, root, stem, leaf, flower, or fruit comprises an altered organellar genome.

In another embodiment, in any of the methods and compositions of matter described herein involving a guide polynucleic acid, the guide polynucleic acid may comprise the following: i) at least about 17 nucleotides that are complementary to at least about 17 nucleotides of a target polynucleic acid, wherein the target polynucleic acid can be located in the genome of an organelle; and ii) a region that can contact a polynucleotide-guided polypeptide. In some embodiments, the guide polynucleic acid may comprise one or more RNA bases. In some embodiments, the guide polynucleic acid may be a single guide RNA (unimolecular) or a duplex guide RNA (bimolecular). In any embodiment involving multiple guide RNAs, the multiple guide RNAs may be single guide RNAs, duplex guide RNAs, or both.

In another embodiment, in any of the methods and compositions of matter described herein involving multiple guide RNAs, the multiple guide RNAs (and/or other heterologous RNAs) may be encoded on separate transcription units or may be encoded on a polycistronic transcription unit. In some embodiments, a guide RNA may be processed from a polycistronic RNA after transcription; e.g., by use of an RNA cleavage site (e.g., Csy4; C2c2), a ribozyme cleavage site, a polynucleotide guided polypeptide cleavage site or the presence of a tRNA sequence. In some embodiments, a guide RNA may be processed from a polycistronic RNA by having a first tRNA sequence 5′ to the guide RNA and a second tRNA sequence 3′ to the guide RNA. In some embodiments, multiple guide RNAs may be arrayed with multiple tRNA sequences (at each guide RNA 5′ and 3′ end) for processing from a polycistronic RNA.

In another embodiment, in any of the methods and compositions of matter described herein that involve a polynucleotide guided polypeptide, the sequence encoding a polynucleotide guided polypeptide may be codon-optimized for a human, a yeast, an alga, or a plant species.

In another embodiment, a method for altering the genome of an organelle disclosed herein may comprise using both a site-directed nuclease (e.g., TALENS, Zinc-Finger Nuclease or Meganuclease) and a polynucleotide guided polypeptide. In some embodiments, an initial cleavage of the organelle genome may be done by a site-directed nuclease (e.g., TALENS, Zinc-Finger Nuclease, Meganuclease) to facilitate homologous recombination with a donor polynucleotide. In some embodiments, the donor polynucleotide may contain modified target sites that are not recognized by a polynucleotide guided polypeptide. In some embodiments, a homoplasmic state may be facilitated by cleavage of the unmodified organelle genomes at the target sites by treatment with a polynucleotide guided polypeptide. In some embodiments, the site-directed nuclease may be a modified site-directed nuclease. In some embodiments, a modified site-directed nuclease can comprise a site-directed nuclease operably linked to an organellar targeting peptide. In some embodiments, an organelle can be a mitochondrion. In some embodiments, an organelle can be a plastid. In some embodiments, an organelle can be a chloroplast.

In another embodiment, in any of the above methods disclosed herein, polynucleotides may be introduced into a cell by use of at least one method selected from the group consisting of: microinjection, meristem transformation, electroporation, Agrobacterium-mediated transformation, viral based gene transfer, transfection (e.g., liposome-mediated transfection, calcium phosphate transfection, polyethyleneimine (PEI)-mediated transfection, DEAE-dextran mediated transfection), vacuum infiltration, biolistic particle bombardment and any combination thereof.

In another embodiment, in any of the above methods disclosed herein, a polynucleotide may be introduced into the cell as a peptide-polynucleotide complex. In some embodiments, a peptide of the peptide-polynucleotide complex may comprise at least one peptide selected from the group consisting of: a cell penetrating peptide (CPP), an organellar targeting peptide (e.g., a mitochondrial targeting peptide, a chloroplast transit peptide), a histidine and lysine-rich peptide (e.g., 18 amino acids of alternating H and K residues) and any combination thereof. A CPP may be at least one member selected from the group consisting of: penetratin, TAT, R9, Pep-1, MPG, gamma-ZEIN, Transporant, MAP, Pept 1, Pept 2, IVV-14, Ig(v), Amphiphilic model peptide, pVEC, HRSV, Bp100, TAT₂ and any combination thereof.

In another embodiment, any of the above methods disclosed herein may further comprise introducing into an organelle a polynucleotide encoding at least one marker selected from the group consisting of: a positive selectable marker, a negative selectable marker, a screenable marker, and any combination thereof. In some embodiments, a positive selectable marker may be an herbicide tolerance protein. In some embodiments, a method may further involve growing the cell in the presence of a positive selection agent and selecting a cell that is homoplasmic for the altered genome of the organelle. In some embodiments, a method may further involve growing the cell in the absence of the positive selection agent, followed by selecting a cell that lacks a non-integrated recombinant DNA construct. In some embodiments, a method may further involve growing the cell in the absence of the positive selection agent, followed by growing the cell in the presence of a negative selection agent, followed by selecting a cell that lacks a non-integrated recombinant DNA construct. In some embodiments, the cell may be a plant cell, the organelle may be a plastid, and the method may further involve regenerating a plant from the plant cell comprising an altered organelle genome. In some embodiments, a plant cell may be monocot cell, e.g., a maize cell, a wheat cell. In some embodiments, a plant cell may be a dicot cell, e.g., a soybean cell.

In another embodiment, in any of the methods described herein that involve a guide polynucleic acid and a polynucleotide guided polypeptide, the method may comprise an increase in transformation efficiency of at least 10%, 20%, 30%, 40%, 50%, 60%, 70%, 80%, 90%, 100%, 200%, 300%, 400%, or 500%, as compared to the corresponding method lacking the guide polynucleic acid, the polynucleotide guided polypeptide, or lacking both.

In another embodiment, in any of the methods described herein that involve a guide polynucleic acid and a polynucleotide guided polypeptide, the method may comprise a decrease in the amount of time required to achieve a homoplasmic state, wherein the decrease is at least 10%, 20%, 30%, 40%, 50%, 60%, 70%, 80% or 90%, as compared to the amount of time required for the corresponding method lacking the guide polynucleic acid, the polynucleotide guided polypeptide, or lacking both.

### EXAMPLES

The present disclosure is further defined in the following Examples, in which parts and percentages are by weight and degrees are Celsius, unless otherwise stated. It should be understood that these Examples, while indicating embodiments, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Such modifications are also intended to fall within the scope of the appended claims.

### Example 1

In this example, the genes encoding a site-specific endonuclease (such as Cas9) and guide RNA(s) are transformed into the nucleus and the plasmid for organelle transformation only encodes donor DNA that can be integrated into the site(s) on organelle DNA that are targeted by the corresponding site-specific endonuclease such as Cas9 and guide RNA.

The experiment is performed as follows. The Cas9 gene for a site-specific endonuclease is fused with a mitochondrial targeting peptide sequence at the amino terminal end of Cas9 ORF and cloned into a galactose-inducible vector with the URA3 selectable gene (e.g., pSF-GAL1-URA3, SIGMA-ALDRICH® Company). The organelle targeting peptides of the ATPase beta subunit and the 70KD protein are used for the modification, creating mCAS9-A (encoded by SEQ ID NO: 10) and mCAS9-B (encoded by SEQ ID NO: 11), respectively.

The reference sequence used is a complete mitochondrial genome sequence available at the Saccharomyces Genome Database (SGD) web site (https://www.yeastgenome.org/). The targeted gene was the COX1 gene (also called oxi3 gene). Mutants of this gene previously have been shown to have a respiration-defective phenotype, as described at the yeast genome web site (https://www.yeastgenome.org/locus/S000007260). The following four guide RNA target sites in the COX1 gene are used (when the targeting sequence is on the reverse complement of the genic sequence, the term “reverse” is indicated):

| (SEQ ID NO: 12) | |
|---|---|
| 1) | TTCTTTGAAGTATCAGGAGGTGG; |
| (SEQ ID NO: 13) | |
| 2) | ATGATTATTGCAATTCCAACAGG; |
| (SEQ ID NO: 14) | |
| 3) | GCTATTTTTAGTGGTATGGCAGG; |
| and | |
| (SEQ ID NO: 15) | |
| 4) | ACCATGTAAATATTGTGAACCAGG (reverse). |

The last three nucleotides in each sequence correspond to the PAM sequence. The first target site resided in exon 5, the second in exon 4, the third one in exon 1 and the fourth one at the junction of 3′ end of exon 1 of the mitochondrial COX1 gene. A guide RNA expression cassette encoding guide RNA(s) directed to either one or two of the four COX1 target sites is cloned into the shuttle vector carrying the Cas9 gene. The variable targeting domain of each guide RNA did not contain the 3-nucleotide PAM sequence listed above. Guide RNAs targeting the COX1 sequences described above are created by fusion of each targeting sequence with the tracrRNA sequence (SEQ ID NO: 16). Each guide RNA expression cassette encoded either one or two guide RNAs, which are directed to the corresponding one or two of the four COX1 target sites. The expression cassette consists of the SNR52 promoter and the SUP4 termination element (SEQ ID NO: 17 and 18, respectively). The entire expression cassette is cloned into the vector that carries Cas9 gene as described above. The expression vector with Cas9 and guide RNA genes is transformed into the yeast strain NB80 [rho⁺, mating type a; lys2, arg8::HisG, ura3, leu2, his3] by using a conventional transformation method available at the SIGMA-ALDRICH® Company website (https://www.sigmaaldrich.com/technical-documents/protocols/biology/yeast-transformation-protocols.html).

In yeast, DNA in a circular form with bacterial vector sequence (pBR322) can be transformed into mitochondria by utilizing a biolistic method (WO 2019/040645 A1). In this example, donor DNA carrying the GFP gene is synthesized as described in WO 2019/040645 A1 and cloned into pBR322 with the COX3 fragment to allow screening of mitochondrial transformants. In addition, the expression cassette of the ARG8m gene is cloned into the vector to allow a positive selection of transformants carrying the plasmid in mitochondria. A positive selection system for yeast mitochondria has already been developed by the Fox lab in the past. They used a yeast line with a mutation in the nuclear ARG8 gene, whose protein product is imported into mitochondria and required for arginine biosynthesis (Steele et al. Proc Natl Acad Sci USA 93:5253-5257, 1996). They succeeded in complementing the loss-of-function mutation of the ARG8 gene by inserting the ARG8m gene, codon-optimized for expression in the mitochondria, into the mitochondrial cox3 gene by homologous recombination after biolistic transformation. The ARG8m expression cassette will contain the minimal cox2 promoter and terminator that we successfully used for stable inheritance of the Edit Plasmids (see WO 2019/040645 A1; herein incorporated by reference).

The corresponding constructs are transformed into DFS160 [rho⁰, mating type a; leu2, arg8::URA3, ura3, ade2, kar1-1] and screened for mitochondrial transformants by using the method as described in Bonnefoy and Fox (2001 Methods Enzymol 350:97-111).

The yeast cells (mating type a) transformed with the nuclear vector expressing Cas9 and guide RNA are crossed with the yeast cells (mating type a) carrying the donor DNA plasmid in mitochondria. Diploid cells are selected on the minimal medium with glucose as carbon source without any amino acid supplement. Isolated diploid cells are grown in the minimal medium with galactose as carbon source to induce Cas9 expression. Cells are sampled and analyzed for any event of donor DNA inserted into corresponding Cas9 target sites as described in WO 2019/040645 A1. For control, isolated diploid cells are grown in the minimal medium with glucose as sole carbon source. The efficacy of the method is shown by significant frequency of integration events observed in positive samples when compared with negative control samples.

### Example 2

As described in the example above, the expression cassette of the Cas9 gene with a mitochondrial targeting sequence is cloned in a yeast shuttle vector and transformed into NB80 strain. Separately, the guide RNA expression cassette is cloned into the plasmid that carries donor DNA with a COX3 fragment. Guide RNAs targeting the COX1 sequences described above were created by fusion of each targeting sequence with the tracrRNA sequence (SEQ ID NO: 16). Each guide RNA expression cassette encoded either one or two guide RNAs, which are directed to the corresponding one or two of the four COX1 target sites. The guide RNA expression cassette contained the following elements in 5′ to 3′ orientation: a minimal COX3 promoter (SEQ ID NO: 19); a tRNA gene, tF(GAA) (SEQ ID NO: 20); a single guide RNA directed to a COX1 site; a second tRNA gene, tW(UCA) (SEQ ID NO: 21); and a minimal COX3 terminator element (SEQ ID NO: 22). The constructs with two guide RNAs are created by combining guide RNAs directed to COX1 sites 1 and 2, as well as to sites 3 and 4. When two guide RNA encoding sequences are present, the second one is fused directly after the tW(UCA) sequence and is flanked by a third tRNA gene, tM(CAU) (SEQ ID NO: 23) at the 3′ end and before the COX3 terminator. The guide RNA expression cassettes with promoter and terminator elements are synthesized and cloned into the pBR322 backbone that carries the donor DNA.

The resulting plasmid is transformed into the mitochondria of DFS 160 strain as described above. The efficacy of this method is demonstrated as described in the example above.

### Example 3

This is an alternative method for modification of an organellar genome. In this approach, the first step is to reduce or eliminate the endogenous organellar DNA by using site-specific endonucleases such as Cas9 systems, as described in Examples 1 and 2. At the same time or subsequently, instead of a plasmid containing a donor DNA, a replacement DNA is introduced into the organelle. The replacement DNA can be fragments of organellar DNA or complete organellar DNA that convey a new genotype and corresponding trait(s) when transformed into the organelle. In the case of organellar DNA fragments, they can be integrated into the remaining native organellar DNA by homologous recombination. In the case of complete organellar DNA replacement, the replacement DNA can be isolated from cultivars, lines, sub species and other species which possess DNA compositions distinct from the endogenous organellar DNA of recipient cells. One requirement of the replacement DNA can be to contain a DNA element functioning as a DNA replication origin in the recipient organelles. The replacement DNA can also be synthesized partially and/or completely. When replacement DNA is created in vitro, it can be a linear DNA with the repeat sequence at the ends. The ends can facilitate homologous recombination in vitro or in vivo to create circular DNA for replication of organellar DNA in cells. The DNA created in vitro can also include exogenous DNA elements such as ones to allow selected amplification in bacterial cells.

To reduce or eliminate mitochondrial DNA, yeast cells are exposed to prolonged expression of guide RNA and Cas9 protein that are designed to be imported into mitochondria as described in Examples 1 or 2. The target sites are chosen to be unique to the endogenous mitochondrial DNA and not present in nuclear genome to reduce the chance of any damage occurring on nuclear genomes when taking the method described in Example 1. The target sites are also chosen to not be present in the replacement DNA.

Multiple cleavage sites enhance the rate of displacing endogenous organellar DNA. This can be attained by expressing multiple guide RNAs targeting different unique sequences in the endogenous mitochondrial DNA. After Cas9/guide RNA treatment, yeast cells that have lost mitochondrial DNA are identified by lack of respiration, inability to grow on media with glycerin as sole carbon source and the lack of mitochondrial DNA. The resulting rho0 condition can also be confirmed by absence of the mitochondrial DNA band in a CsCl gradient. Once mitochondrial DNA is deleted, cells are then transformed with replacement DNA created in vitro or in vivo; e.g., mitochondrial DNA derived from different lines or species with traits distinct from the recipient cells. In this example, mitochondrial DNA from antibiotic resistant lines (e.g. IL8-8C/R53) is isolated and transformed into recipient cells that lack the resistant trait. Mitochondrial DNA for use in transformation can also be created by PCR amplification of organellar DNA by use of a primer set whose 3′ ends are complementary with each other, sufficient for annealing in vivo. The resulted linear DNA molecules are transformed into mitochondria. Homologous recombination activity present in the organelle creates circular organellar DNA upon transformation. Alternatively, DNA for transformation can be created synthetically in a linear as well as a circular form.

### Example 4

To target Cas9 into mitochondria, e.g., of human tissue culture cells, Cas9 protein without nuclear localization signal element is fused with a mitochondrial targeting peptide. One such peptide is NDUFV2 MTS which has 32 amino acid residues, NH2-MFFSAALRARAAGLTAHWGRHVRNLHKTVMQN—COOH (SEQ ID NO: 24). In this case, the NDUFV2 signal sequence is fused with the amino terminus of CAS9 to give a modified CAS9 (SEQ ID NO: 25). Alternatively, another signal peptide such as the one from citrate synthase (NH2-MALLTAAARLLGTKNASCLVLAARH—COOH; SEQ ID NO: 26) that can function in human cells can be used to create a modified CAS9 (SEQ ID NO: 27). A polynucleotide encoding a modified CAS9 gene (with a mitochondrial target sequence) is operably linked to a promoter element such as CMV by utilizing the human transfection vector, pSF-CMV-Amp, purchased from SIGMA-ALDRICH® Company or is operably linked to a inducible promoter such as the TET-inducible promoter of pTRE2hyg vector, which can be purchased from TAKARA BIO USA, Inc. An alternative option is to follow the construction of the transfection vector as described in Bian et al. (ACS Synth. Biol. 2019 8:621-632).

In this experiment, the sequence encoding the guide RNA is targeted to the mitochondria. The guide RNA is designed to target the COX3 gene (SEQ ID NO: 28). In the guide RNA, the variable targeting domain is fused with the tracrRNA sequence. The gRNA expression cassette consists of the polynucleotide encoding the guide RNA operably linked to a promoter and terminator that are functional in mitochondria of human cells. For the purpose, we make plasmid constructs that are capable of replication in mitochondria. Plasmid DNA carrying donor DNA with any exogenous DNA such as GFP gene and guide RNA expression cassette(s) is introduced into mitochondria either in a circular form or in a linear form that has the ability to circularize in mitochondria. The plasmid DNA can be the derivatives of pBR322, which has been shown to replicate in yeast mitochondria. To increase the efficacy of DNA replication, the plasmid DNA can also contain the rep/ori sequence of mammalian and/or microbial mitochondrial DNA. It can also encode at least one selectable marker to allow for selection after transformation into mitochondria. Such a selectable marker can be the active 16S rRNA gene with CAP^{R} mutation. In this case, the entire expression cassette of 16S rRNA gene is cloned into the plasmid DNA. To express guide RNA gene cassette(s), each target sequence of the guide RNA fused with tracrRNA is cloned between two tRNA genes and the promoter for the H- or L-strand messages is used. Biolistic or other gene delivery methods are used to transform plasmid DNA into mitochondria of human cells. The cell lines transformed with mitochondrial plasmid DNA is further transformed with the transvection construct(s) as described above. The resulting cells are analyzed to confirm the efficient insertion of donor DNA into the target sites in the mitochondrial DNA.

### Example 5

To target Cas9 into mitochondria, e.g., of human tissue culture cells, Cas9 protein without nuclear localization signal element is fused with a mitochondrial targeting peptide as described in Example 4. In one case, the NDUFV2 signal sequence is fused with the amino terminus of CAS9 to give a modified CAS9 (SEQ ID NO: 25). Alternatively, another signal peptide such as the one from citrate synthase (NH2-MALLTAAARLLGTKNASCLVLAARH—COOH; SEQ ID NO: 26) that can function in human cells can be used to create a modified CAS9 (SEQ ID NO: 27). A polynucleotide encoding a modified CAS9 gene (with a mitochondrial target sequence) is operably linked to a promoter element such as CMV by utilizing the human transfection vector, pSF-CMV-Amp, purchased from SIGMA-ALDRICH® Company or is operably linked to a inducible promoter such as the TET-inducible promoter of pTRE2hyg vector, which can be purchased from TAKARA BIO USA, Inc. An alternative option is to follow the construction of the transfection vector as described in Bian et al., ACS Synth. Biol. 2019, 8, 621-632 without guide RNA.

In this experiment, the sequence encoding the guide RNA is targeted to the nucleus. The guide RNA is designed to target the COX3 gene (SEQ ID NO: 28). In the guide RNA, the variable targeting domain is fused with the tracrRNA sequence. The gRNA expression cassette consists of the polynucleotide encoding the guide RNA operably linked to a promoter and terminator that are functional in nuclei of human cells. In this example, we use the U6 promoter for constitutive expression. For the 5S rRNA fusion, we also use the promoter and terminator of the 5S rRNA gene (SEQ ID NO: 29). Guide RNA expression cassette is cloned into the plasmids carrying the Cas9 expression cassettes or cloned into distinct transfection vectors. Constructed plasmids are transfected into human cell lines such as HeLa and HEK293 as well as HeLa and HepG2 Tet-Off cells for Cas9 inducible expression from pTRE2hyg based constructs. Transfected cells undergo selection in the presence of hygromycin. Preparation of cell culture and transfection are performed for inducible expression.

The plasmid DNA carrying donor DNA with any exogenous DNA such as GFP gene is introduced into mitochondria either in a circular form or in a linear form that has the ability to circularize in mitochondria. The plasmid DNA contains sequence that allows for autonomous replication in mitochondria. It can also encode at least one selectable marker to allow for selection after transformation into mitochondria. Such a selectable marker can be the active 16S rRNA gene with CAP^{R} mutation. The rep/ori and other elements for gene expression in mitochondria present on the plasmid DNA may be derived from species different from the target species for mitochondrial DNA editing.

The cell lines transformed with mitochondrial plasmid DNA are further transformed with the transfection construct(s) as described above. The resulting cells are analyzed to confirm the efficient insertion of donor DNA into the target sites in the mitochondrial DNA.

### Example 6

Plant mitochondria can be transformed with plasmids encoding donor DNA and expression cassettes for producing guide RNA(s). In this Example, an expression cassette encoding a modified Cas9 can be transformed into the plant nucleus. The modified Cas9 can contain a mitochondrial targeting peptide at its amino-terminus.

Plasmids capable of replicating in bacteria (e.g., pBR322) have been shown to replicate in mitochondria, as well (Fox et al. 1988 PNAS 85:7288-7292). Consequently, we use pBR322 as a backbone in a design of a plasmid for mitochondrial transformation in plants. To enhance its capability for replication in mitochondria, we optionally can add a rep/ori sequence such as the one described for yeast (Turk et al. 2013 PLOS One 8:e78105). To enable the screening of transformed events, a sequence encoding a marker (e.g., GFP) is cloned in an expression cassette of the plasmid. The expression cassette consists of a promoter element (e.g., wheat cox2 promoter including 5′ UTR that is present in the 1 kb fragment upstream of the translation initiation codon) and terminator element (e.g., wheat cox2 terminator that is present in the 0.5 kb fragment downstream of the termination codon).

To ensure mitochondrial-specific expression of the screenable marker, we include one or more of following strategies: 1) Insertion of group I or II intron(s) that can only be spliced by enzymes (maturases) present in mitochondria; e.g., intron A of the nad7 gene in wheat (Brown et al. 2014 Frontiers in Plant Science 5:1-13); and 2) Utilization of RNA editing specific to mitochondria to ensure that the gene is expressed only in mitochondria.

RNA transcripts of most genes encoded in wheat mitochondria are edited at a small number of specific C residues. Those residues are converted to U through deamination. The site of RNA editing is determined by the surrounding sequence context. Often, 20-30 nucleotides of sequence at the editing site is sufficient to induce correct editing, even when placed into different locations of the mRNA (Choury et al. 2004 Nucleic Acids Res 32:6397-6406).

In one approach, mitochondrial RNA editing can create the translation start codon AUG by C-to-U editing of an ACG sequence. There are three such editing sites present in the wheat cox2 gene, at positions 169, 449 and 704, with editing efficiency of 80-100% (Choury et al. 2004 Nucleic Acids Res 32:6397-6406). We will test these sites by fusion with the GFP gene. In another approach, mitochondrial RNA editing can create a termination codon by C-to-U editing. There are four editing sites in the wheat cox2 gene for this purpose, at the following positions: 449 (CGA-to-UGA); 467 (UAG-to-UAA); 587 (CAG-to-UAG) and 620 (CAA-to-UAA) (Choury et al. 2004 Nucleic Acids Res 32:6397-6406). By using a 30 nucleotide element from each cox2 editing site in ORF, we can create a termination codon that is only recognized in mitochondria. For this scheme of expression regulation, the read-through protein needs to be nonfunctional or inactive. One way to design such a protein is by fusion with a repressor or quencher domain. One such fusion is a caspase activatable-GFP (Nicholls and Hardy, 2012 Protein Science 22:247-257). We can insert an RNA editing site to release GFP from a C-terminal quencher domain by introducing a translational stop codon between GFP and the quencher. This will allow production of active GFP protein without the quencher only in mitochondria. Plasmid constructs encoding an expression cassette for the screenable marker are transformed into the cells of mature wheat embryos using a microprojectile bombardment apparatus (BIO-RAD® Laboratories Inc.). Transformation is preformed essentially as described in Hamada et al. (2017 Scientific Reports 7:11443). Variants of transformation conditions, such as varying particle sizes, are tested to maximize the efficiency of mitochondrial transformation. Mitochondrial transformation is confirmed by the presence of GFP signals as analyzed by use of a dissection microscope capable of capturing GFP fluorescence.

Cas9 based genome editing can facilitate the integration of the sequence encoding the screenable marker into the mitochondrial genome and the subsequent selection of the integrated DNA over wild-type mitochondrial DNA (U.S. patent application Ser. No. 16/109,523). To allow such features, we include the guide RNA expression cassette(s) and donor DNA in the plasmids described above, to create “Edit Plasmids”, and expression cassette of Cas9-like gene in a separate construct for the nuclear expression. Each expression cassette includes promoter and terminator elements functional in corresponding sites in the plant cells. Promoters and terminator sequences for wheat mitochondrial transformation are identified by analysis of the Triticum aestivum cultivar Chinese Yumai complete mitochondrial genome (GenBank Accession: EU534409) and other sequence information available in the public database. For the expression of guide RNAs, each guide RNA (fused with a tracrRNA) is flanked by tRNA sequences to facilitate correct cleavage of each guide RNA molecule from a polycistronic transcript. To facilitate the integration of the screenable marker gene, the marker gene is flanked by mitochondrial DNA fragments that have sufficient length for homologous recombination and that encompass the corresponding Cas9/guide RNA target sites. In addition, the donor DNA (i.e., marker gene flanked by mitochondrial DNA fragments) also encodes sequence variants of the Cas9/guide RNA target sites to prevent further cleavage of replaced DNA. The two target sites are designed such that the expression of the corresponding endogenous genes is affected.

The expression cassette of modified Cas9 is made with promoter such as a maize ubiquitin promoter and terminator such as NOS terminator and cloned into a binary vector to allow plant transformation carrying a selectable marker (e.g., hpt: hygromycin resistance gene) for plants as described in Zhang et al. (Plant Biotechnology Journal 2019 DOI: 10.1111/pbi.13088). Alternatively, the expression cassette can be directly transformed into plant cells, using a biolistic bombardment method (Hamada et al. 2017 Scientific Reports 7:11443). The nuclear transformation is performed into the plant cells that have already been transformed with the plasmid DNA in mitochondria, and vice versa or simultaneously.

After transformation, the stability of the screenable marker gene gained by the Cas9 system is analyzed at different periods of growth of the transformed plants. The efficacy of the invented method is demonstrated by the comparison with the control where no Cas9-like gene is expressed in the nucleus.

### Example 7

In this example, the experiments are performed as described above with the alteration that the expression cassette of guide RNA(s) is transformed into the nucleus together with the expression cassette of a modified Cas9 gene. For the expression of guide RNA(s), a U6 promoter is used as described in in Zhang et al. (Plant Biotechnology Journal 2019, DOI: 10.1111/pbi.13088). The plasmid for mitochondrial transformation does not carry any sequences encoding guide RNA gene(s).

After sequential and/or simultaneous transformation of the nuclear as well as the mitochondrial constructs, the insertion events of the donor DNA at the Cas9 target sites in mitochondria are analyzed at different periods of growth of the transformed plants. The efficacy of the invented method is demonstrated by the comparison with the control where no Cas9-like gene is expressed in the nucleus.

### Example 8

In this example, the experiments are performed as Example 7 with the alteration that the genome editing is targeted to the plastid instead of the mitochondrion. The guide RNA(s) are designed to cleave sequences specific to the wheat plastid genome and the plasmid DNA carries donor DNA to insert into the corresponding cleavage sites targeted by the guide RNAs. For the Cas9-like protein to localize into plastids, a transit peptide sequence specific to plastids such as RC2 (Shen et al., Scientific Reports 7:46231, 2017, DOI: 10.1038/srep46231) is fused to the amino terminal of the Cas9-like protein sequence. A screenable maker gene on the plasmid DNA is expressed under a promoter specific to plastid expression. Those include but not limited to the promoter of the gene encoding the RUBISCO large subunit.

### Example 9

DNA is delivered to organelles of yeast and plant cells by way of uptake of peptide-DNA complexes. Uptake is performed according to methods presented in the following: Chuah J A et. al. 2015 Sci Rep 5:7751; Chuah J A et al. 2016 Biomacromolecules 17:3547-3557; and Lakshmanan M et al. 2013 Biomacromolecules 14:10-16.

The following peptides are used in this Example:

| (SEQ ID NO: 30) | |
|---|---|
| 1) | MLSLRQSIRFFKKHKHKHKHKHKHKHKHKH; |
| (SEQ ID NO: 31) | |
| 2) | KKLFKKILKLYLKHKHKHKHKHKHKHKHKH. |

Peptide 1 consists of the first 12 amino acids of yeast cytochrome C oxidase (MTS)(in bold) and 18 amino acids of alternating H and K residues.

Peptide 2 consists of first 12 amino acids of cell penetrating peptide (in bold) and 18 amino acids of alternating H and K residues.

Peptides 1 and 2 are synthesized by commercial vendors. Then they are combined singly and in combination with a reporter donor DNA marker in different ratios before delivery.

The following reporter donor DNAs are used in this Example:
1) COX2 gene fragment (SEQ ID NO: 32) that covers the cox2-62 mutant plus 150 bp flanking homology to complement cox2-62 mutation in strain NB41.
2) COX 3 gene fragment (SEQ ID NO: 33) that covers the cox3-10 mutant plus 150 bp flanking homology to complement cox3-10 mutation in strain MCC125.
3) DNA fragment containing cox2P:Arg8m:cox2T::Cox3 fragment in edit plasmids 2299 bp to be used with strain MCC125.
4) Edit plasmids pNY5 (6741 bp) will be used with strain MCC125 to demonstrate DNA uptake and with other edit plasmids pNY4, pNY43, pNY45.

Methods to deliver the peptide-DNA complexes into yeast and plant cells include (but are not limited to) the following:
a) Biolistic (gene gun) bombardment as known in the art;
b) Standard yeast transformation methods using chemically competent cells or by electroporation; and
c) Vacuum infiltration; e.g., use of the gene gun apparatus without bombardment.

### Example 10

In this example, a green alga, Chlamydomonas reinhardtii, is used to transform expression cassettes of a modified Cas9 (e.g., Cas9 fused to a plastid transit peptide) and guide RNAs into the nucleus using a known method such as the one described in Kindle 1990 PNAS 87:1228-1232. The actin and U6 promoters are used to express a modified Cas9 and guide RNAs in the nucleus, respectively, as described in Guzman-Zapata et al. 2019 Int J Mol Sci 20:1247 DOI: 10.3390/ijms20051247. When multiple guide RNAs, each fused with tracrRNA sequence, are expressed under the U6 promoter, each guide RNA unit is separated by a tRNA gene to allow proper processing and release of each unit from the polycistronic transcript. The donor DNA is cloned into a plasmid DNA as described in our previous patent publication (Example 21 of WO 2019/040645 A1).

### Example 11

In this example, the Cas9 protein and guide RNAs were expressed in the nucleus of yeast cells and a donor DNA in a form of a plasmid was introduced into mitochondria through biolistic transformation.

For this experiment, constructs for nuclear expression of a Cas9 protein and guide RNAs were made as follows. The Cas9 coding sequence, which was published in Laughery et al. 2015 (Yeast 32:711-20, DOI: 10.1002/yea.3098) was used and modified by deletion of a nuclear localization signal of the protein and addition of a mitochondrial transit sequence (MTS) at an N-terminus of the Cas9 protein. The MTS used corresponded to the first 25 N-terminal residues (MLSLRQSIRFFKPATRTLCSSRYLL; SEQ ID NO: 53) of a precursor of subunit IV of cytochrome c oxidase of yeast. An entire gene ORF was flanked by HindIII and NotI restriction sites at the 5′ and 3′ ends, respectively. An amino acid sequence of an MTS-Cas9 protein and a corresponding nucleotide sequence encoding the protein are presented as SEQ ID NO: 54 and SEQ ID NO: 55, respectively.

A designed DNA encoding an MTS-Cas9 protein was synthesized. A synthesized fragment was cloned between a HindIII and a NotI site of yeast expression vector pYES2, resulting in plasmid pNY93. An MTS-Cas9 fusion protein encoding pNY93 was expressed under control of a galactose-inducible GAL1 promoter and CYC1 terminator of yeast.

A guide RNA expression cassette was added to pNY93 as follows. Two guide RNAs were designed to be expressed under control of SNR52 promoter and terminated by a SUP4 terminator of a SupF tRNA gene of yeast. Each guide RNA was fused with a gRNA scaffold and flanked by tRNA genes to ensure correct processing and function. An entire expression cassette had a configuration, SphI site-SNR52 promoter-gRNA variable region A-gRNA scaffold-Gly tRNA-gRNA variable region B-gRNA scaffold-SUP4 terminator-TEF1 terminator-NotI site. A TEF1 terminator was included next to a NotI site so that transcription of a MTS-Cas9 gene could be terminated properly and did not extend into a guide RNA expression cassette.

Two versions of guide RNA expression cassettes were synthesized. One cassette encoded gRNAs 1 and 2 to target exon 4 and 5 of a mitochondrial COX1 gene, and the other cassette encoded gRNAs 3 and 4 to target exon 1 of the same gene. Sequences encoding variable regions of the gRNAs were as follows:

| (SEQ ID NO: 56) | |
|---|---|
| gRNA 1: | TTCTTTGAAGTATCAGGAGG |
| (SEQ ID NO: 57) | |
| gRNA 2: | ATGATTATTGCAATTCCAAC |
| (SEQ ID NO: 58) | |
| gRNA 3: | TAGCTATTTTTAGTGGTATGGC |
| (SEQ ID NO: 59) | |
| gRNA 4: | ACCATGTAAATATTGTGAACC |

An expression cassette for gRNAs 1 and 2 is presented as SEQ ID NO: 60. An expression cassette for gRNAs 3 and 4 is presented as SEQ ID NO: 61.

Each synthesized DNA contained NotI and SphI sites at the ends. These DNAs were digested NotI and SphI and cloned into pNY93 that had been digested with the same restriction enzymes. The resulting constructs were named pHS97 (encoding gRNAs 1 and 2) and pHS95 (encoding gRNAs 3 and 4), respectively.

Alternatively, an expression cassette for gRNAs 3 and 4 was synthesized as two DNA fragments. A first fragment (SphI-BsaI DNA; SEQ ID NO: 62) contained the following: SNR52 promoter-gRNA 3ny variable region-gRNA scaffold-Gly tRNA. A second fragment (BsaI-NotI DNA; SEQ ID NO: 63) contained the following: gRNA 4ny variable region-gRNA scaffold-SUP4 terminator of SupF tRNA gene-TEF terminator. SphI-BsaI and NotI-BsaI fragments were cloned into NotI-SphI digested pNY93 in a three-way ligation resulting in pNY95.

For pNY95, guide RNAs were designed with variable regions of 20 nt, which were shorter than ones carried in pHS95, as follows:

| (SEQ ID NO: 64) | |
|---|---|
| gRNA 3ny: | GCTATTTTTAGTGGTATGGC |
| (SEQ ID NO: 65) | |
| gRNA 4ny: | CCATGTAAATATTGTGAACC |

The sequence of the guide RNA expression cassette pf pNY95 is presented as SEQ ID NO: 66.

Furthermore, to investigate the effect of copy numbers of nuclear constructs in mitochondrial genome editing, low-copy-number constructs were made of pYES2, pNY93, pHS95 and pHS97 by replacing a 2 μm replication element with an ARS-CEN element of a plasmid pRS316. For this purpose, an ARS-CEN element was amplified with the following primers:

| (SEQ ID NO: 84) | |
|---|---|
| PRS316-FP: | GAAAAGTGCCACCTGGGTCCTTTTCATCACG |
| (SEQ ID NO: 85) | |
| PRS316-RP: | GACGAAAGGGCCTCGTGATACGCCTAT |

The amplified DNA was inserted into a linearized recipient construct without a 2 μm replication element, which were produced by a digestion with SwaI and NruI. The cloning was performed by using a DNA Assembly Master Mix. Resulting low-copy number constructs of pYES2, pNY93, pHS95 and pHS97 were named pDMYES2, pDM93, pDM95 and pDM97, respectively (Table 2). Table 2 lists various plasmids described herein. Also presented for each plasmid are corresponding sites of plasmid replication, a selectable marker, a plasmid copy number, and a presence or absence of various sequence elements. Where appropriate, a plasmid origin (ARS-CEN or ori5) and a mitochondrial selectable marker (ARG8m) are indicated.

TABLE 2
Table of plasmids.

| | | | | | | | Donor | |
|---|---|---|---|---|---|---|---|---|
| | | | | | | | DNA | Donor |
| | | | | | | | for | DNA for |
| Construct | Replication | Selection | Copy | | gRNA | gRNA | gRNA | gRNA |
| Name | Site | Marker | # | Cas9 | 1&2 | 3&4 | 1&2 | 3&4 |
| pYES2 | nucleus | URA3 | high | — | — | — | — | — |
| pNY93 | nucleus | URA3 | high | + | — | — | — | — |
| pNY95 | nucleus | URA3 | high | + | — | + | — | — |
| pHS95 | nucleus | URA3 | high | + | — | + | — | — |
| pHS97 | nucleus | URA3 | high | + | + | — | — | — |
| pDMYES2 | nucleus | URA3 | low | — | — | — | — | — |
| | | | (ARS- | | | | | |
| | | | CEN) | | | | | |
| pDM93 | nucleus | URA3 | low | + | — | — | — | — |
| | | | (ARS- | | | | | |
| | | | CEN) | | | | | |
| pDM95 | nucleus | URA3 | low | + | — | + | — | — |
| | | | (ARS- | | | | | |
| | | | CEN) | | | | | |
| pDM97 | nucleus | URA3 | low | + | + | — | — | — |
| | | | (ARS- | | | | | |
| | | | CEN) | | | | | |
| HS6 | mitochondria | COX3 | low | — | + | — | + | — |
| HS8 | mitochondria | COX3 | low | + | + | — | + | — |
| HS90 | mitochondria | COX3 | low | — | — | — | — | + |
| HS100 | mitochondria | COX3 | low | — | — | — | + | — |
| pNY45 | mitochondria | COX3, | low | + | — | + | — | + |
| | | ARG8m | (ori5) | | | | | |
| pNYAGc | mitochondria | COX3 | low | — | — | + | — | + |
| | | | | | | | | (ARG8m) |
| pNY71 | mitochondria | COX3 | low | + | — | + | — | + |
| | | | | | | | | (ARG8m) |
| pNY72 | mitochondria | COX3 | low | + | — | + | — | + |
| | | | (ori5) | | | | | (ARG8m) |
| pNY74 | mitochondria | COX3 | low | — | — | + | — | + |
| | | | (ori5) | | | | | (ARG8m) |
| pNY75 | mitochondria | COX3 | low | — | — | — | — | + |
| | | | (ori5) | | | | | (ARG8m) |

In some cases, constructs for nuclear expression of Cas9 and gRNAs were paired with plasmids containing donor DNA in mitochondria. In some cases, mitochondrial-targeted plasmids were designed to integrate donor DNA between two sites of a mitochondrial genome that were expected to be cleaved by gRNAs carried in nuclear-targeted constructs as described above. In some cases, mitochondrial plasmids were derived from an Edit Plasmid that contained Cas9 and gRNA expression cassettes and donor DNA, and previously had been shown to replicate in mitochondria and function to cleave mitochondrial DNA and integrate donor DNA. In some cases, a plasmid, HS100, that carried a donor DNA targeted to a cleavage site created by gRNAs 1 and 2 was created by deleting a gRNA expression cassette from an Edit Plasmid HS6. In some cases, for cleavage sites created by gRNAs 3 and 4, Edit Plasmid HS4 was constructed by introduction of a mCas9 expression cassette, a gRNA expression cassette and a corresponding donor DNA into a pHD6 backbone in a similar manner to how HS6 was constructed. By deletion of a mCas9 and gRNA expression cassettes from HS4, a plasmid HS90 was created to carry only a donor DNA targeted to cleavage sites created by gRNAs 3 and 4.

In some cases, a homologous region at both ends of a donor DNA for gRNAs 3 and 4 were short to suppress spontaneous homologous recombination. In some cases, a donor DNA was designed to carry modified sequences in cleavage sites to prevent recognition by gRNAs 3 and 4, so that altered organellar DNA would be resistant to further DNA cleavage by a Cas9 nuclease.

Donor DNA for gRNAs 3 and 4 (HindIII-PstI fragment) is presented as SEQ ID NO: 67.

In some cases, to assess a capability of mitochondrial genome editing, each nuclear expression construct was transformed into yeast strain CUY563 (MATα ade2 ade3 leu2 ura3 [rho+]) with the wild-type mitochondrial genome by using a transformation kit. In some cases, transformants of constructs that carried a URA3 gene were selected on Ura-dropout medium. In some cases, each mitochondrial-targeted construct was transformed into a yeast strain MCC109 (MATa ade2 ura3 kar1-1 [rho0]) that carried no mitochondrial DNA. In some cases, transformants were first selected for a nuclear-targeted plasmid (pYES2) that was co-transformed together with a mitochondrial-targeted construct. In some cases, mitochondrial transformants were identified among URA+ colonies by their capability of rescuing a mitochondrial deletion mutation in COX3. To analyze an effect of Cas9 and gRNA genes expressed in a nucleus on mitochondrial genome editing, a combination of nuclear and mitochondrial constructs was achieved by simple genetic crosses between CUY563 and MCC109 transformants. In some cases, to allow rapid induction of MTS-Cas9 gene, lines were grown in Ura-dropout medium containing raffinose or galactose as a carbon source. In some cases, crosses were performed on medium lacking both uracil and leucine and having galactose (with or without raffinose) as a carbon source. In some cases, since mitochondrial transformants in MCC109 background carried a nuclear kar1-1 mutation, mated cells did not undergo normal nuclear fusion to form diploid cells but separated from each other after cytoplasmic fusion, keeping a haploid nucleus intact. In some cases, cells of two strains would keep intermixing cytoplasms and mitochondria within. In some cases, in a presence of raffinose in a medium, cells carrying MCC109 nuclei could grow. In some cases, in a presence of galactose as a sole carbon source without leucine, neither parental line (CUY563 and MCC109) could grow (galactose metabolism requires respiration, i.e., wild-type mitochondrial genome). In some cases, under these conditions, growth would be limited to those cells that had intermixed cytoplasms.

In some cases, after each cross, cell samples were taken after time intervals and were subjected to PCR analysis to detect integration events where donor DNA present in a CUY563 background has recombined with mitochondrial DNA present in an MCC109 background.

In some cases, primers used for the PCR analysis were the following:

Primer I (recognizing the mitochondrial genome
region upstream of the site recognized by gRNA 1):
(SEQ ID NO: 68)
AGAATCAGGTGCTGGTACAGGGTGA
Primer C (recognizing the mitochondrial genome
region upstream of the site recognized by gRNA 1):
(SEQ ID NO: 69)
CTATTCAGGCACATTCAGGACC
Primer F (recognizing the mitochondrial genome
region downstream of the site recognized by
gRNA 2):
(SEQ ID NO: 70)
AGAGGTATACCAACACAAGATTC
Primer F1 (recognizing the mitochondrial genome
region downstream of the site recognized by
gRNA 2):
(SEQ ID NO: 71)
AGATAGATAATCAATTCAACCATCTGT
Primer 35 (recognizing the mitochondrial genome
region upstream of the site recognized by
gRNA 3):
(SEQ ID NO: 72)
ATTAGTTCGGTTTAGTTGGTATTTTGTAATGAGTAAAAAGT
Primer 36: (recognizing the mitochondrial genome
region downstream of the site recognized by
gRNA 4):
(SEQ ID NO: 73)
CCTACACTAATCATAGGTGTTTTATGACATGCTA
Primer 37 (recognizing the mitochondrial genome
region upstream of the site recognized by gRNA 3):
(SEQ ID NO: 74)
AGATTATGAAAGAGAGTATTAATATCA
Primer 38: (recognizing the mitochondrial
genome region downstream of the siterecognized
by gRNA 4):
(SEQ ID NO: 75)
TAAAGTTAGCCCCTACTGAGTTA
Primer 11 (recognizing the antisense strand of
the donor DNA):
(SEQ ID NO: 76)
CAGGTGAAGGTGAAGGTGATGC
Primer 12 (recognizing the sense strand of the
donor DNA):
(SEQ ID NO: 77)
GATCTGCTAATTGTACTGAACCG
Primer 13 (recognizing the antisense strand of
the donor DNA):
(SEQ ID NO: 78)
CAGCAATGCCTGAAGGTTACGTAC
Primer 15 (recognizing the sense strand of the
donor DNA):
(SEQ ID NO: 79)
ACTAATGTAGGTCAAGGTACAGG

The positions of primers are with respect to a direction of a GFP ORF encoded in a donor DNA, i.e., a direction of a COX1 gene when donor DNA is integrated into a mitochondrial genome.

For a detection of an integrated donor DNA at sites cleaved by gRNAs 1 and 2, we used primer pairs C-12 and F-11 under conditions described by Yoo et al. 2020. For sites cleaved by gRNAs 3 and 4, primer pairs 35-15 were designed for a 5′ junction of an integrated donor DNA and 36-13 for a 3′ junction of an integrated donor DNA. Further to detect an integrated donor DNA from a relatively small amount of cell samples, nested PCR reactions were applied where first reactions were performed with I-12 and F1-11 primer sets for gRNA 1 and 2 sites, respectively. We have designed primers 35-12 and 36-11 for gRNA 3 and 4 sites, respectively. Second PCR reactions were performed with C-15 and F-13 for gRNA 1 and 2 sites, respectively. Primer pairs 37-15 and 38-13 were designed for gRNA 3 and 4 sites, respectively.

In one experiment, two strains, pHS97 and HS100, were crossed together on a medium containing galactose as carbon source without uracil and leucine. In some cases, a strain pHS97 carried Cas9 and gRNA expression cassettes in the nucleus and an intact mitochondrial genome. In some cases, as a control, the strain pNY93, carrying no gRNA expression cassette, was crossed with HS100. In some cases, the strain HS100 carried a donor DNA in mitochondria that completely lacked a mitochondrial genome. After ten days of incubation at room temperature, cell samples were collected and subjected to a PCR reaction to detect junctions of integrated donor DNA at sites recognized by gRNA 1 and 2.

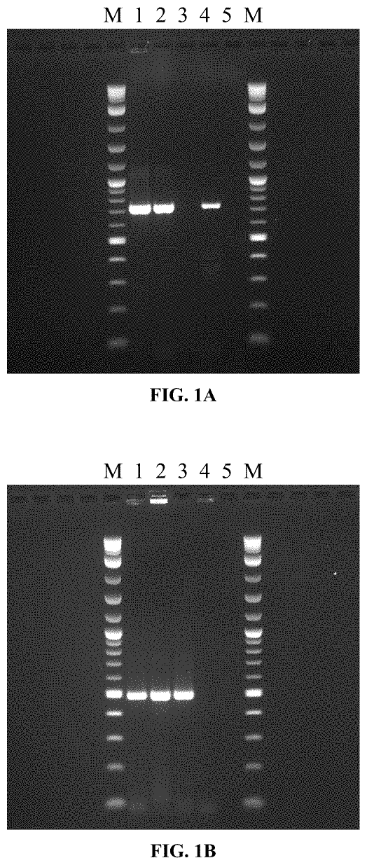

In some cases, two rounds of PCR were performed; a first round of 40 cycles with two primer sets, 1-12 and F1-11; and a second round with 20 cycles with a nested primer set, C-15 and F-13, respectively. In some cases, amplified DNA samples were separated on agarose gels (FIGS. 1A and 1B). In FIGS. 1A and 1B, lanes 1 & 2 are two independent positive samples (pHS97×HS100), lanes 3 & 4 are two independent control samples (pNY93×HS100), and lane 5 is a PCR control without any cells. Flanking lanes were loaded with 1 kb Plus DNA Ladder (New England BioLabs, Inc.). Samples for lanes 1 & 3 were derived from crosses in which strains carrying corresponding nuclear constructs (pHS97 and pNY93 as control) were grown in a Ura-dropout medium with galactose before crosses, so that Cas9 expression of pHS97 was active before a cross was made. Samples for lanes 2 & 4 were derived from crosses in which strains carrying a corresponding nuclear construct (pHS97 and pNY93 as control) were grown in a Ura-dropout medium with glycerol before crossing, so that Cas9 expression of pHS97 was induced immediately after a cross. A top panel (FIG. 1A) shows a right junction amplified by primers F1-11 and F-13 and a bottom panel (FIG. 1B) shows a left junction amplified by primers 1-12 and C-15. Each positive sample showed an amplification of both junctions with a size expected from an integration of a donor DNA (741 bp for a PCR fragment from a right junction, and 493 bp for a PCR fragment from a left junction), whereas each negative control produced only one end under amplification conditions. Amplified DNA fragments of positive samples derived from lane 1 PCR reactions were isolated from a gel using a QIAquick Gel Extraction Kit and were sequenced with corresponding PCR primers. In some cases, sequences were assembled and analyzed using computer software.

In some cases, a sequence of a right junction was presented as SEQ ID NO: 86:

cacattcaggaccTAGTGTAGATTTAGCAATTTTTGCATTACATTTAAC
ATCAATTTCATCATTATTAGGTGCTATTAATTTCATTGTAACAACATTA
AATATGAGAACAAATGGTATGACAATGCATAAATTACCATTATTTGTAT
GATCAATTTTCATTACAGCGTTCTTATTATTATTATCATTACCTGTATT
ATCTGCTGGTATTACAATGTTATTATTAGATAGAAACTTCAATACTTCA
TTTTTCGGAGTTTCTGGTGGAGGTGGTGGAATGACACATTTAGAAAGAA
GTAGACAAATGTCAAAAGGTGAAGAATTATTCACTGGAGTAGTACCTAT
CTTAGTAGAATTAGATGGTGATGTAAATGGTCATAAATTCTCAGTATCA
GGTAAAGGTGAAGGTGATGCTACATATGGTAAATTAACATTAAAATTCA
TCTGTACAACAGGTAAATTAcctgtacctt

In some cases, sequence of a left junction was presented as SEQ ID NO: 87:

gaggttacgtacAAGAAAGAACAATCTTCTTCAAAGATGATGGTAATTATAA

AACAAGAGCTGAAGTAAAATTCGAAGGTGATACATTAGTAAATAGAATC
GAGTTAAAAGGTATCGATTTCAAAGAAGATGGTAATATCTTAGGTCATA
AATTAGAATATAATTATAATTCACATAATGTATATATCATGGCTGATAA
ACAAAAAAATGGTATCAAAGTAAATTTCAAAATCAGACATAATATCGAA
GACGGTTCAGTACAATTAGCAGATCATTATCAACAAAATACACCTATCG
GTGATGGTCCTGTATTATTACCTGATAATCATTACTTAAGTACACAATC
AGCTTTATCAAAAGATCCTAATGAAAAAAGAGATCATATGGTATTATTA
GAATTTGTAACAGCTGCTGGTATCACACATGGTATGGATGAATTATATA
AATAACAACAGGAATTAAAATTTTCTCATGATTAATAAATCCCTTTAGC
AAGGATAAAAATAAAAATAAAAATAAAAAGTTGATCAGAAATTATCAAA
AAATAAATAATAATAATATAATAAAAACATATTTAAATAATAATAATAT
AATTATAATAAATATATATAAAGGTAATTTATATGATATTTATCCAAGA
TCAAATAGAAATTATATTCAACCAAATAATATTAATAAAGAATTAGTAG
TATATGGTTATAATTTAgaatctt

In some cases, sequences in lower case at ends show parts of primers used for sequencing. In some cases, sequences in bold font indicate a GFP ORF encoded in a donor DNA. In some cases, sequences in italic font were homologous regions that were adjacent to target sites of gRNA 1 and 2. In some cases, junction sequences between donor DNA and mitochondrial genomic sequences were identical with what was expected from an exact donor DNA integration at Cas9 cleavage sites, which was also consistent with results observed previously when Cas9 and gRNAs were encoded in an Edit Plasmid and expressed in a mitochondria.

In some cases, active Cas9 and gRNA expressed in a nucleus facilitated complete integration of donor DNA at both cleavage sites while an inactive Cas9 without gRNA preferentially induced an integration only at one end. In some cases, spontaneous homologous recombination events can occur frequently only at one end of a donor DNA without resulting in an integration of an entire donor DNA at both sites. In some embodiments, nuclear expression of Cas9 nuclease and gRNAs can help induce precise integration of donor DNA between two distinct sites cleaved by Cas9 nuclease.

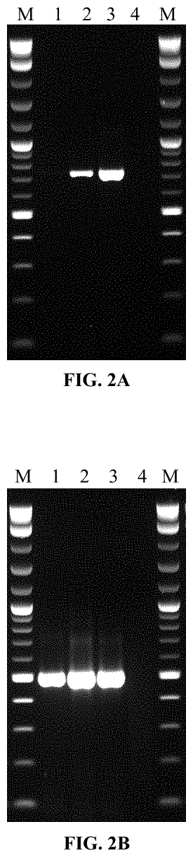

In some cases, an experiment was repeated with additional strains that were transformed with different constructs. In one experiment, a strain was used as a control that was transformed with pYES2 without any expression cassette. For a positive construct, pDM97 was used instead of pHS97. In some cases, cells were first grown in a Ura-dropout medium containing glucose as a carbon source. In some cases, cells were then inoculated in a Ura-dropout medium containing raffinose as a carbon source. In some cases, cells were grown in raffinose and primed for immediate induction of gene expression under a GAL1 promoter when their media were changed to ones with galactose in tan absence of glucose. In some cases, strains with nuclear constructs were crossed with a strain carrying HS100 in mitochondria on a Ura-Leu dropout medium with 2% galactose and 1% raffinose. Under these conditions, cells of nuclear transformants could not grow but cells of mitochondrial transformants could grow. After 24 hours of incubation at 30° C., cell samples were collected and subjected to PCR analysis as described above. In some cases, amplified DNA samples were separated on agarose gels, as shown in FIG. 2A and FIG. 2B. In FIG. 2A and FIG. 2B, lane 1 was a negative sample (pYES2×HS100), lanes 2 & 3 were two independent positive samples (pHS97×HS100 and pDM97×HS100), and lane 4 was a PCR control without any cells. Flanking lanes were loaded with 1 kb Plus DNA Ladder. FIG. 2A shows a right junction amplified by primers F1-11 and F-13 and FIG. 2B shows a left junction amplified by primers 1-12 and C-15. As with a first experiment described above, each positive sample showed an amplification of both junctions with a size expected from an integration of a donor DNA (741 bp for a PCR fragment from a right junction, and 493 bp for a PCR fragment from a left junction), whereas each negative control produced only one end under these amplification conditions. In some cases, when comparing two positive samples, cells crossed with pDM97 appeared to produce more junction DNA under these PCR conditions. In some cases, this can indicate that a low-copy number plasmid (pDM97) exhibited better Cas9 activity upon import into mitochondria than a high-copy number plasmid (pHS97).

In some cases, overexpression of Cas9 and gRNAs in a nucleus induced reduction of a mitochondrial DNA amount in target regions and also could induce replacement of mitochondrial DNA with short single-stranded DNA molecules. In some cases, results showed that a double-stranded donor DNA encoded in a plasmid capable of replication in mitochondria could serve as a vehicle for precise integration at designed target sites in a presence of Cas9 and gRNA expression in a nucleus.

### Example 12

In this example, an organellar genome editing strategy was designed to use integration of a mitochondrial donor DNA encoding an ARG8m gene to allow for positive selection of recombination events. In some cases, an ARG8m gene can be employed as a marker to select mitochondrial transformants in yeast. In some cases, donor DNA was modified such that an ARG8m gene lacked its native initiation codon and was expressed by translational fusion to an exon 1 of a COX1 gene upon its integration. In some cases, an integration was designed to occur between cleavage sites created by an activity of MTS-Cas9 and gRNAs 3 and 4 as described above. In some cases, ARG8m ORF was flanked at each end by short regions of homology with a genomic region of a mitochondrial COX1 gene.

In some cases, a donor DNA carrying an ARG8m ORF with homologous regions (HindIII-NcoI fragment) was synthesized and was presented as SEQ ID NO: 80.

In some cases, an amino acid sequence of an ARG8m protein encoded in SEQ ID NO: 80 was presented as SEQ ID NO: 81.

In some cases, donor DNA encoding ARG8m was cloned into an Edit Plasmid background (HS6) together with a gRNA expression cassette. In some cases, HS6 construct first was digested with HindIII and NotI to delete gene editing components and then ligated to a donor DNA (HindIII-NcoI fragment) and an expression cassette for gRNAs 3 and 4 (NcoI-NotI fragment), resulting in pNYAGc.

In some cases, an expression cassette for gRNAs 3 and 4 was synthesized and is presented as SEQ ID NO: 82.

In some cases, in a pNYAGc construct, the expression cassette for a Cas9m gene was added back by isolating a PstI-NotI fragment from Edit Plasmid HS8 and ligation of that fragment with pNYAGc that had been digested with PstI and NotI. A resulting construct was named pNY71.

In some cases, an ARG8m ORF was amplified by PCR with PstI and SpeI sites at two ends and cloned into HS4 to yield pNY4.

In some cases, an attempt to improve a retention possibility of mitochondrial constructs in yeast mitochondria, an ori5 element of yeast mitochondria was added to pNY71. In some cases, an ori5 element is one of an active yeast mitochondrial replication origin sequence. In some cases, an ori5 element was synthesized with XbaI and NotI sites at the ends and a nucleotide sequence is presented as SEQ ID NO: 83.

In some cases, an ori5 element was ligated with pNY4 that had been digested with restriction enzymes XbaI and NotI, to create pNY45. By exchanging SpeI-NotI fragments between pNY71 and pNY45, a construct pNY72 was made that carried all components of pNY71 in addition to ori5.

In some cases, a construct pNY74, lacking a Cas9m expression cassette, was made from pNY72 by deletion of a SpeI-XbaI fragment.

In some cases, a construct pNY75, lacking a gRNA expression cassette, was made from pNY74 by deletion of a NotI-NcoI fragment.

### Example 13

In some cases, to allow for positive selection of gene editing events that results in an expression of an ARG8m gene in mitochondria, a yeast strain NB80 (MATa lys2 leu2 ura3 his3AHinDIII arg8::hisG [rho+]) that has a defective ARG8 nuclear gene can be used. In some cases the following combinations of nuclear and mitochondrial constructs can be transformed into NB80 using a biolistic transformation method as described in Bonnefoy & Fox, 2000 Methods in Molecular Biology 372: 153-166; Yoo et al., 2020:
a pNY93 (nuclear MTS-Cas9 expression)-pNY74 (mitochondrial gRNA cassette and donor DNA).
a pNY95 (nuclear MTS-Cas9 & gRNA expression)-pNY75 (mitochondrial gRNA cassette and donor DNA).
a pYES2 (empty nuclear vector)-pNY75 (mitochondrial gRNA cassette and donor DNA)(control).

In some cases, NB80 cells were grown in galactose medium and can be subjected to transformation by a biolistic method on sorbitol medium with galactose as a carbon source, and lacking uracil and arginine. In some cases, a transformation procedure was used as described in Bonnefoy et al., 2007 Methods in Cell Biology 80:525-54, and Yoo et al. 2020. Peer J 8:e8362 DOI 10.7717/peerj.8362.

In some cases, as an example of experimental conditions for transformation, the following DNAs can be mixed on ice:
1. 120 μg/36 μl pNY74 plus 20 μg/7 μl pNY93.
2. 60 μg/30 μl pNY75 plus 20 μg/6 μl of pNY95.
3. 120 μg/36 μl of pNY74 plus 20 μg/5 μl of pYES2.

Then each tube can have the following added, in order:
400 μl of sterile tungsten particles in 50% glycerol,
400 μl of 2.5 M Cacl2, and
160 μl 0.1M spermidine.
11 μl of this cold mix can be added to the flying disc.

For each of mixes 1, 2 and 3, fifteen to twenty Ura+Arg dropout plates with galactose as carbon source and plated with NB80 cells can be bombarded.

In some cases, after about 20 hours, bombarded cells can be suspended in 2 ml saline solution, pelleted through centrifugation, and resuspended in a 200 μl saline solution making a final volume of about 300 μl. In some cases, about 200 μl of each resuspension can be plated on three plates of minimal medium with 2% galactose and 1% raffinose as a carbon source, lacking uracil and arginine and incubated at 30° C. After 6 days (or longer) plates can be checked for colonies. In some cases, successful gene editing in mitochondria can be assessed by a number of colonies that grow on a medium lacking arginine.

As an alternative to an NB80 strain, an experiment also can be performed by crossing strains together as described in Example 11 where nuclear constructs are transformed in NB80 background and mitochondrial constructs are transformed in DFS160p0 (MATα leu2 arg8ΔURA3 ura3 kar1-1 ade2 [rho0]).

1. A method for altering an organellar genome, the method comprising:
a. introducing into a nucleus of a cell:
i. a first polynucleotide encoding at least in part a modified polynucleotide guided polypeptide, wherein the modified polynucleotide guided polypeptide comprises a polynucleotide guided polypeptide operably linked to an organellar targeting peptide, wherein the polynucleotide guided polypeptide when associated with a guide RNA, cleaves at least one target sequence present in the organellar genome; and
ii. a second polynucleotide comprising at least in part at least one guide RNA, wherein the at least one guide RNA directs the polynucleotide guided polypeptide to cleave the at least one target sequence present in the organellar genome;
b. introducing into an organelle of the cell, wherein the organelle is a mitochondrion or a plastid, a third polynucleotide comprising at least in part at least one homologous organellar DNA sequence, wherein the at least one homologous organellar DNA is capable of homologous recombination, wherein integration of the at least one homologous organellar DNA sequence into the organellar genome results in a recombined organellar genome lacking the at least one target sequence;
c. growing a cell comprising the nucleus of (a) and the organelle of (b) under conditions in which the first polynucleotide and the second polynucleotide are expressed; and
d. selecting a cell comprising an altered organellar genome.

2. A method for altering an organellar genome, the method comprising:
a. introducing into a nucleus of a cell a first polynucleotide encoding at least in part a modified polynucleotide guided polypeptide, wherein the modified polynucleotide guided polypeptide comprises a polynucleotide guided polypeptide operably linked to an organellar targeting peptide, wherein the polynucleotide guided polypeptide when associated with a guide RNA, cleaves at least one target sequence present in the organellar genome;
b. introducing into an organelle of the cell, wherein the organelle is a mitochondrion or a plastid, a second polynucleotide comprising at least in part at least one guide RNA, wherein the at least one guide RNA directs the polynucleotide guided polypeptide to cleave the at least one target sequence present in the organellar genome;
c. growing a cell comprising the nucleus of (a) and the organelle of (b) under conditions in which the first polynucleotide and the second polynucleotide are expressed; and
d. selecting a cell comprising an altered organellar genome.

3. The method of claim 2, wherein (b) further comprises introducing into the organelle of the cell a third polynucleotide comprising at least in part at least one homologous organellar DNA sequence, wherein the at least one homologous organellar DNA is capable of homologous recombination, wherein integration of the at least one homologous organellar DNA sequence into the organellar genome results in a recombined organellar genome lacking the at least one target sequence.

4. The method of claim 3, wherein the polynucleotide guided polypeptide comprises at least one selected from the group consisting of: a Cas9 protein, a MAD2 protein, a MAD7 protein, a CRISPR nuclease, a nuclease domain of a Cas protein, a Cpf1 protein, an Argonaute, modified versions thereof, and any combination thereof.

5. The method of claim 3, wherein the at least one guide RNA is processed from a polycistronic RNA after transcription by use of at least one member selected from the group consisting of: an RNA cleavage site, a ribozyme cleavage site, a polynucleotide guided polypeptide cleavage site, a presence of a tRNA sequence, and any combination thereof.

6. The method of claim 5, wherein the at least one guide RNA is processed from a polycistronic RNA after transcription by use comprising the presence of the tRNA sequence, wherein the at least one guide RNA is processed from a polycistronic RNA by having a first tRNA sequence 5′ to the at least one guide RNA and a second tRNA sequence 3′ to the at least one guide RNA.

7. A method for altering an organellar genome, the method comprising:
a. introducing into a nucleus of a cell: a first polynucleotide encoding a modified site-directed nuclease, wherein the modified site-directed nuclease comprises a site-directed nuclease operably linked to an organellar targeting peptide, wherein the site-directed nuclease cleaves at least one target sequence present in the organellar genome; and
b. introducing into an organelle of the cell, wherein the organelle is a mitochondrion or a plastid, a third polynucleotide comprising at least one homologous organellar DNA sequence, wherein the at least one homologous organellar DNA is capable of homologous recombination, wherein integration of the at least one homologous organellar DNA sequence into the organellar genome results in a recombined organellar genome lacking the at least one target sequence;
c. growing a cell comprising the nucleus of (a) and the organelle of (b) under conditions in which the first polynucleotide is expressed; and
d. selecting a cell comprising an altered organellar genome.

8. The method of claim 7, wherein the site-directed nuclease comprises at least one member selected from the group consisting of: a TALEN, a Zinc-Finger Nuclease, a Meganuclease, a restriction enzyme, and any combination thereof.

9. The method of claim 3, wherein (a) and (b) occur in separate cells.

10. The method of claim 9, wherein the nucleus of (a) and the organelle of (b) are brought together into a cell by sexual crossing, cell fusion, microinjection, or any combination thereof.

11. The method of claim 3, wherein the method further comprises: (e) selecting a cell that is homoplasmic for the altered organellar genome.

12. The method of claim 3, wherein the third polynucleotide comprising the at least one homologous organellar DNA sequence is operably linked to an origin of replication that is functional in the organelle.

13. The method of claim 3, wherein the third polynucleotide comprising the at least one homologous organellar DNA sequence comprises a fourth polynucleotide encoding at least one selectable marker or at least one screenable marker, or both.

14. The method of claim 13, wherein the fourth polynucleotide, after integration into the organellar genome, is operably linked to a promoter that is functional in the organelle.

15. The method of claim 3, wherein the third polynucleotide comprising the at least one homologous organellar DNA sequence comprises a fifth polynucleotide and a sixth polynucleotide, wherein the fifth polynucleotide and the sixth polynucleotide each comprise a region of homology in the organellar genome, further wherein the region of homology in the fifth polynucleotide and the region of homology in the sixth polynucleotide correspond to two adjacent regions of homology in the organellar genome.

16. The method of claim 15, wherein the fifth polynucleotide and the sixth polynucleotide are separated by a seventh polynucleotide, wherein the seventh polynucleotide comprises a sequence that is heterologous to the organellar genome.

17. The method of claim 16, wherein the seventh polynucleotide encodes at least one selected from the group consisting of: a cytoplasmic male sterility factor, a dsRNA, a siRNA, a miRNA, and any combination thereof.

18. The method of claim 17, wherein the dsRNA, the siRNA or the miRNA suppresses at least one target gene necessary for male fertility in a plant.

19. The method of claim 13, wherein the fourth polynucleotide comprises a first sequence encoding a positive selectable marker.

20. The method of claim 19, wherein the fourth polynucleotide comprises a second sequence encoding a negative selectable marker.

21. The method of claim 20, wherein the first sequence and the second sequence are each operably linked to a promoter that is functional in the organelle.

22. The method of claim 3, wherein the third polynucleotide is single stranded.

23. The method of claim 3, wherein the third polynucleotide is double stranded.

24. The method of claim 3, wherein the third polynucleotide comprises a length of at least 100, 150, 200, 250, 300, 400, 500, 100, 1500 or 2000 nucleotides.

25. The method of claim 3, wherein the cell is selected from the group consisting of: a yeast cell, an algal cell, a plant cell, an insect cell, a non-human animal cell, an isolated and purified human cell, and a mammalian tissue culture cell.

26. (canceled)

27. (canceled)

28. (canceled)

29. The method of claim 3, wherein at least one member selected from the group consisting of: the first polynucleotide, the second polynucleotide, the third polynucleotide, and any combination thereof, is introduced into the cell via at least one method from the group consisting of: microinjection, meristem transformation, electroporation, Agrobacterium-mediated transformation, viral based gene transfer, transfection, vacuum infiltration, biolistic particle bombardment, and any combination thereof.

30. The method of claim 3, wherein at least one member selected from the group consisting of: the first polynucleotide, the second polynucleotide, the third polynucleotide, and any combination thereof, is introduced into the cell as a peptide-polynucleotide complex.

31. The method of claim 30, wherein at least one peptide of the peptide-polynucleotide complex comprises at least one member selected from the group consisting of: a cell penetrating peptide (CPP), an organellar targeting peptide, a histidine rich peptide, a lysine-rich peptide, and any combination thereof.

32. A method comprising growing a cell produced by the method of claim 13.

33. The method of claim 32, further comprising growing the cell in a presence of a positive selection agent and selecting a cell that is homoplasmic for the altered organellar genome.

34. The method of claim 33, further comprising growing the cell in an absence of a positive selection agent, followed by selecting a cell that lacks a non-integrated recombinant DNA construct.

35. The method of claim 33, further comprising growing the cell in an absence of a positive selection agent, followed by growing the cell in a presence of a negative selection agent, followed by selecting a cell that lacks a non-integrated recombinant DNA construct.

36. A composition comprising a cell produced by the method of claim 3, wherein the cell is selected from the group consisting of: a yeast cell, an algal cell, a plant cell, an insect cell, a non-human animal cell, an isolated and purified human cell, and a mammalian tissue culture cell.

37. A composition comprising a plant, a seed, a root, a stem, a leaf, a flower, a fruit, or any combination thereof produced from the cell of claim 36 wherein the cell is a plant cell, wherein the plant, the seed, the root, the stem, the leaf, the flower, the fruit, or the combination thereof comprises the altered organellar genome.

38. (canceled)

39. (canceled)

40. The method of claim 38, wherein the third polynucleotide comprises fragments of organellar DNA or a complete organellar DNA from a cultivar, line, sub-species or other species.

41.-48. (canceled)
